SEQUENCE LISTING (113 sequences)

SEQ ID NO: 1 - protein, 856 aa, Human Immunodefficiency Virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 184 aa, Homo sapiens
DAASDLKSRLDKVSSFGAGFTQKVTDVQEGQGALAVKRPNLFAWHMTQPDESILVSDGKTLWFYNPFVEQATATWLKDATGNTPFMLIARNQSSDWQQYNIKQNGDDFVLTPKASNGNLKQFTINVGRDGTIHQFSAVEQDDQRSSYQLKAQENLEVDKWAFLFGPPQGVTVDDQRKSGLVPRG

SEQ ID NO: 3 - protein, 81 aa, Homo sapiens
SKMLQHIDYRMRCIGGAGGIAIGTFKAFGAGMGLILCDCDAFAKIKPKNSKQAEREEKAVGELLELDKWALLSMTVEGPPP

SEQ ID NO: 4 - protein, 240 aa, Thermus aquaticus
SDPVRQYLHEIGEVLELDKWAELGAAAKVEEGMEAIKKLSEATGLDQELIREVVRAKILGTAAIQKIPGLKEKPDPKTVEEVDGKLKSLPKELKRYLHIAREGEAARQHLIEANLRLVVSIAKKYTGRGLSFLDLIQEGNQGLIRAVEKFEYKRGFAFSTYATWWIRQAINRAIADQARTIRIPVHMVETINKLSRTARQLQQELGREPSYEEIAEAMGPGWDAKRVEETLKIAQEPVSL

SEQ ID NO: 5 - protein, 265 aa, Rhizopus niveus
EVLEADKWAILGATKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPANLSIFTVGGPRVGNPTFAYYVESTGIPFARTVHKRDIVPHVPPQSFGFLHPGVESWIKSGTSNVQVCGSAIETKDCSNSIVPFTSILDHLSYFDINEGSCL

SEQ ID NO: 6 - protein, 262 aa, Escherichia coli
EILELDKWAILGMRVAGRLAAEVLEMIEPYVKPGVSTGELDRICNDYIVNEQHAVSACLGYHGYPKSVCISINEVVCHGIPDDAKLLKDGDIVNIDVTVIKAGAHGDTSKMFIVGKPTIMGERLCRITQESLYLALRMVKPGINLREIGAAIQKFVEAEGFSVVREYCGHGIGGGFHEEPQVLHYDSRETNVVLKPGMTFTIEPMVNAGKKEIRTMKDGWTVKTKDRSLSAQYEHTIVVTDNGCEILTLRKDDTIPAIISHD

SEQ ID NO: 7 - protein, 556 aa, Klebsiella Sp. LX3
EYPAWWKEAVFYQIYPRSFKDTNDDGIGDIRGIIEKLDYLKSLGIDAIWINPHYDSPNTDNGYDISNYRQIMKEYGTMEDFDSLVAEMKKRNMRLMIDVVINHTSDQHPWFIQSKSDKNNPYRDYYFWRDGKDNQPPNNYPSFFGGSAWQKDAKSGQYYLHYFARQQPDLNWDNPKVREDLYAMLRFWLDKGVSGMRFDTVATYSKGQEFLELDKWAQLAFAAGYTGGANIHRYIQEMNRKVLSRYDVATAGEIFGVPLAASSQFFDRRRHELNMAFMFDLIRLDRDAAERWRHKSWSLSQFRQIISKMDVTVGKYGWNTFFLDNHDNPRAVSHFGDDRPQWREASAKALATITLTQRATPFIYQGSELGMTNYPFRQLNEFDDIEVKGFWQDYVQSGKVTATEFLDNVRLTSRDNSRTPFQWNDTLNAGFTRGKPWFHINPNYVEINAEREETREDSVLNYYKKMIQLRHHIPALVYGAYQDLNPQDNTVYAYTRTLGNERYLVVVNFKEYPVRYTLPANDAIEEVVIDTQQQAAAPHSTSLSLSPWQAGVYKLR

SEQ ID NO: 8 - protein, 226 aa, Homo sapiens
APCQNHHCKHGKVCELDENNTPMCVCQDPTSCPAPIGEFEKVCSNDNKTFDSSCHFFATKCTLEGTKKGHKLHLDYIGPCKEILECDKWALLGFPLAMRDWLKNVLVTLYERDEDNNLLTEKQKLRVKKIHENEKRLEAGDHPEKNYNMYIFPVHWQFGQLDQHPIDGYLSHTELAPLRAPLIPGEGCTTAFFETCDLDNDKYIALDEWAGCFGIKQKDIDKDLVI

SEQ ID NO: 9 - protein, 14 aa, Human immunodeficiency virus 1
AQELLELDKWASLW

SEQ ID NO: 10 - protein, 14 aa, Human immunodeficiency virus
EAELLELDKWASLW

SEQ ID NO: 11 - protein, 14 aa, Human immunodeficiency virus 1
EQALLELDKWASLW

SEQ ID NO: 12 - protein, 14 aa, Human immunodeficiency virus 1
EQEALELDKWASLW

SEQ ID NO: 13 - protein, 14 aa, Human immunodeficiency virus 1
EQELAELDKWASLW

SEQ ID NO: 14 - protein, 14 aa, Human immunodeficiency virus 1
EQELLALDKWASLW

SEQ ID NO: 15 - protein, 14 aa, Human immunodeficiency virus 1
EQELLEADKWASLW

SEQ ID NO: 16 - protein, 14 aa, Human immunodeficiency virus 1
EQELLELAKWASLW

SEQ ID NO: 17 - protein, 14 aa, Human immunodeficiency virus 1
EQELLELDAWASLW

SEQ ID NO: 18 - protein, 14 aa, Human immunodeficiency virus 1
EQELLELDKAASLW

SEQ ID NO: 19 - protein, 14 aa, Human immunodeficiency virus 1
EQELLELDKWASLW

SEQ ID NO: 20 - protein, 14 aa, Human immunodeficiency virus 1
EQELLELDKWAALW

SEQ ID NO: 21 - protein, 14 aa, Human immunodeficiency virus 1
EQELLELDKWASAW

SEQ ID NO: 22 - protein, 14 aa, Human immunodeficiency virus 1
EQELLELDKWASLA

SEQ ID NO: 23 - protein, 14 aa, Human immunodeficiency virus 1
EQELLELDKWASLW

SEQ ID NO: 24 - protein, 8 aa, Human immunodeficiency virus 1
ELLELDKW

SEQ ID NO: 25 - protein, 8 aa, Human immunodeficiency virus 1
LLELDKWA

SEQ ID NO: 26 - protein, 7 aa, Human immunodeficiency virus 1
LLELDKW

SEQ ID NO: 27 - protein, 6 aa, Human immunodeficiency virus 1
LELDKW

SEQ ID NO: 28 - protein, 5 aa, Human immunodeficiency virus 1
ELDKW

SEQ ID NO: 29 - protein, 4 aa, Human immunodeficiency virus 1
LDKW

SEQ ID NO: 30 - protein, 3 aa, Human immunodeficiency virus 1
DKW

SEQ ID NO: 31 - protein, 4 aa, Human immunodeficiency virus 1
DKWA

SEQ ID NO: 32 - protein, 5 aa, Human immunodeficiency virus 1
DKWAS

SEQ ID NO: 33 - protein, 6 aa, Human immunodeficiency virus 1
DKWASL

SEQ ID NO: 34 - protein, 7 aa, Human immunodeficiency virus 1
ELDKWAS

SEQ ID NO: 35 - protein, 11 aa, Human immunodeficiency virus 1
ELLELDKWASL

SEQ ID NO: 36 - protein, 179 aa, Human immunodeficiency virus 1
SDPVRQYLHEIGEVLELDKWAELGAAAKVEEGMEAIKKLSEATGLDQELIREVVRAKILGTAAIQKIPGLKEKPDPKTVEEVDGKLKSLPKELKRYLHIAREGEAARQHLIEANLRLVVSIAKKYTGRGLSFLDLIQEGNQGLIRAVEKFEYKRGFAFSTYATWWIRQAINRAIADQAR

SEQ ID NO: 37 - protein, 123 aa, Human immunodeficiency virus 1
SKMLQHIDYRMRCIGGAGGIAIGTFKAFGAGMGLILCDCDAFAKIKPKNSKQAEREEKAVGELLELDKWALLSMTVEGPPPGGAKFVAAWTLKAAASGLVPRGSGSHHHHHHGGTETSQVAPA

SEQ ID NO: 38 - protein, 305 aa, Human immunodeficiency virus 1
MPMGSLQPLATLYLLGMLVASVLADPVRQYLHEIGEVLELDKWAELGAAAKVEEGMEAIKKLSEATGLDQELIREVVRAKILGTAAIQKIPGLKEKPDPKTVEEVDGKLKSLPKELKRYLHIAREGEAARQHLIEANLRLVVSIAKKYTGRGLSFLDLIQEGNQGLIRAVEKFEYKRGFAFSTYATWWIRQAINRAIADQARTIRIPVHMVETINKLSRTARQLQQELGREPSYEEIAEAMGPGWDAKRVEETLKIAQEPVSLGGAKFVAAWTLKAAASGLVPRGSGSHHHHHHGGTETSQVAPA

SEQ ID NO: 39 - protein, 13 aa, Homo sapiens
AKFVAAWTLKAAA

SEQ ID NO: 40 - DNA, 363 bp, Artificial Sequence
  b12 Scaffold
atgtatagcatgcagctggccagctgtgtgacactgacactggtgctgctggtgaattccggccctaggcatctgaatgtgctggccaaagccctgtacgatgctagcggaggagatcctgagatcctgagcttcagaaagggagacatcatgacagtgctggaacaggatacacagggactggatggagcttggctgtgtagcctgcatggaagacagggaatcgtgcctggaaacgatctgaagatcctggtgggaatgtacgacaagaagccttccggactggtgcctagaggaagcggaagccatcatcatcatcatcatggaggaacagaaacaagccaggtggctcctgcttgatag

SEQ ID NO: 41 - DNA, 786 bp, Artificial Sequence
  b12 Scaffold
atgtatagcatgcagctggccagctgtgtgacactgacactggtgctgctggtgaattccggccctagggccaacaagcagcagaacttcaacacaggaatcaaggacttcgacttctggctgagcgaagtggaagctctgctggctagcgaagattacggaaaagatctggctagcgtgaacaacctgctgaagaagcaccagctgctggaagctgatatcagcgctcatgaagatagactgaaggatctgaacagccaggctgatagcctgatgacaagcagcgctttcgatacaagccaggtgaaggataagagagagaccatcaacggaaggttccagagaatcaagagcatggctgctgctagaagagctaagctgaacgaaagccacagactgcatcagttcttcagagacatggatgatgaagaaagctggatcaaggagaagaagctgctggtgagcagcaatggaagcggaggagatcctgaaatcgtgcaggccctgagaaagcagcataagagactggaagctgaactggctgctcatgaacctgctatccagggagtgctggatacaggaaagaagctgagcgacgacaacacaatcggaaaggaagagatccagcagagactggctcagttcgtggaccattggaaggagctgaagcagctggccgccgccaggggccagaggctggagtccggactggtgcctagaggaagcggaagccatcatcatcatcatcatggaggaacagaaacaagccaggtggctcctgcttga

SEQ ID NO: 42 - DNA, 483 bp, Artificial Sequence
  b12 Scaffold
atgtatagcatgcagctggccagctgtgtgacactgacactggtgctgctggtgaattccggccctagggctaagctgaacgaaagccacagactgcatcagttcttcagagacatggatgatgaagaaagctggatcaaggagaagaagctgctggtgagcagcaatggaagcggaggagatcctgaaatcgtgcaggccctgagaaagcagcataagagactggaagctgaactggctgctcatgaacctgctatccagggagtgctggatacaggaaagaagctgagcgacgacaacacaatcggaaaggaagagatccagcagagactggctcagttcgtggaccattggaaggagctgaagcagctggccgccgccaggggccagaggctggagtccggactggtgcctagaggaagcggaagccatcatcatcatcatcatggaggaacagaaacaagccaggtggctcctgcttga

SEQ ID NO: 43 - protein, 118 aa, Artificial Sequence
  b12 Scaffold
MYSMQLASCVTLTLVLLVNSGPRHLNVLAKALYDASGGDPEILSFRKGDIMTVLEQDTQGLDGAWLCSLHGRQGIVPGNDLKIVGMYDKKPSGLVPRGSGSHHHHHHGGTETSQVAPA

SEQ ID NO: 44 - protein, 255 aa, Artificial Sequence
  b12 Scaffold
MYSMQLASCVTLTLVLLVNSGPRANKQQNFNTGIKDFDFWLSEVEALLASEDYGKDLASVNNKKHQEADISAHEDRLKDLNSQADSLMTSSAFDTSQVKDKRETINGRFQRIKSMAAARRAKLNESHRLHQFFRDMDDEESWIKEKKVSSNGSGGDPEIVQALRKQHKRLEAELAAHEPAIQGVIDTGKKLSDDNTIGKEEIQQRLAQFVDHWKELKQLAAARGQRLESGIVPRGSGSHHHHHHGGTETSQVAPA

SEQ ID NO: 45 - protein, 160 aa, Artificial Sequence
  b12 Scaffold
MYSMQLASCVTLTLVLLVNSGPRAKLNESHRLHQFFRDMDDEESWIKEKKLLVSSNGSGGDPEIVQALRKQHKRLEAELAAHEPAIQGVIDTGKKLSDDNTIGKEEIQQRLAQFVDHWKELKQLAAARGQRLESGIVPRGSGSHHHHHHGGTETSQVAPA

SEQ ID NO: 46 - DNA, 678 bp, Artificial Sequence
  b12 Scaffold
atgtatagcatgcagctggccagctgtgtgacactgacactggtgctgctggtgaattccggccctaggcgccccgtggtgagcacccagctgctgctgaacggcagcctggccaatgagacggtggtgatccgcagcgtgaacttcaccgacaacgccaagaccatcatcgtgcagctgaacaccagcgtggagatcaactgcacccgccccaacaacggcggcagcaacagcaccggcaacatgcgccaggcccactgcaacatcagccgcgccaagtggaacaacaccctgaagcagatcgccagcaagctgcgcgagcagttcggcaacaacaagaccatcatcttcaagcagagcagcggcggcgaccccgagatcgtgacccacagcttcaactgcggcggcgagttcttctactgcaacagcacccagctgttcaacagcacctggttcaacagcacctggagcaccgagggcagcaacaacaccgagggcagcgacaccatcaccctgccctgccgcatcaagggaggagccaacatcagcggccagatccgctgcagcagcaacatcaccggcctgctgctgacccgcgacggcggcaacagcaacaacgagagcgagatcttccgtccgggcggcggcgacatgaacgacacctggcgcagcgagtga

SEQ ID NO: 47 - protein, 225 aa, Artificial Sequence
  b12 Scaffold
MYSMQLASCVTLTLVLLVNSGPRRPVVSTQLLLNGSLANETVVIRSVNFTDNAKTIIVQLNTSVEINCTRPNNGGSNSTGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKGGANISGQIRCSSNITGLLLTRDGGNSNNESEIFRPGGGDMNDTWRSE

SEQ ID NO: 48 - DNA, 717 bp, Artificial Sequence
  b12 Scaffold
atgtatagcatgcagctggccagctgtgtgacactgacactggtgctgctggtgaattccggccctaggcgccccgtggtgagcacccagctgctgctgaacggcagcctggccaatgagacggtggtgatccgcagcgtgaacttcaccgacaacgccaagaccatcatcgtgcagctgaacaccagcgtggagatcaactgcacccgccccaacaacggcggcagcaacagcaccggcaacatgcgccaggcccactgcaacatcagccgcgccaagtggaacaacaccctgaagcagatcgccagcaagctgcgcgagcagttcggcaacaacaagaccatcatcttcaagcagagcagcggcggcgaccccgagatcgtgacccacagcttcaactgcggcggcgagttcttctactgcaacagcacccagctgttcaacagcacctggttcaacagcacctggagcaccgagggcagcaacaacaccgagggcagcgacaccatcaccctgccctgccgcatcaagcagatcatcaacatgtggcagaatgtgaccaagaacatgaccgccccccccatcagcggccagatccgctgcagcagcaacatcaccggcctgctgctgacccgcgacggcggcaacagcaacaacgagagcgagatcttccgtccgggcggcggcgacatgaacgacacctggcgcagcgagtga

SEQ ID NO: 49 - protein, 238 aa, Artificial Sequence
  b12 Scaffold
MYSMQLASCVTLTLVLLVNSGPRRPVVSTQLLLNGSLANETVVIRSVNFTDNAKTIIVQLNTSVEINCTRPNNGGSNSTGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQNVTKNMTAPPISGQIRCSSNITGLLLTRDGGNSNNESEIFRPGGGDMNDTWRSE

SEQ ID NO: 50 - DNA, 834 bp, Artificial Sequence
  b12 Scaffold
atgtatagcatgcagctggccagctgtgtgacactgacactggtgctgctggtgaattccgcccctaggagggccaacgtgagcacccagctgctgctgaacggcagcctggccaacgagaccgtgaacatcaccagcgtgaacttcaccgacaacgccaagaccatcatcgtgcagctgaacaccagcgtggagatcaactgcaccagggccaacaacggcaccagcaacagcaccggcaacatgacccaggcccactgcaacatcagcagggccaagtggaacaacaccctgaagcagatcgccagcaagctgagggagcagttcggcaacaacaagaccatcatcttcaagcagagcagcggcggcgaccccgagatcgtgacccacagcttcaactgcaacggcaccttcttctactgcaacagcacccagctgttcaacagcacctggttcaacagcacctggagcaccgagggcagcaacaacaccgagggcagcgacaccatcaccctgccctgcaggatcaagggcggcgccaacatcagcggcaacatcacctgcagcagcaacatcaccggcctgctgctgaccagggacggcggcaacagcaccaacgagagcgagatcttcaggcccggcggcggcgacatgaacgacacctggaggagcgagggatccggaggaggaagcggcagcctggtgcctcgaggcagccctggcagcggctacatccccgaggctccacgcgacggccaggcctacgtgcgcaaggacggcgagtgggtgctgctgagcaccttcctgggcggccaccaccaccaccaccactga

SEQ ID NO: 51 - protein, 277 aa, Artificial Sequence
  b12 Scaffold
MYSMQLASCVTLTLVLLVNSAPRRANVSTQLLLNGSLANETVNITSVNFTDNAKTIIVQLNTSVEINCTRANNGTSNSTGNMTQAHCNISRAKWNNTLKQIASKLREQFGNNKTIIFKQSSGGDPEIVTHSFNCNGTFFYCNSTQLFNSTWFNSTWSTEGSNNTEGSDTITLPCRIKGGANISGNITCSSNITGLLLTRDGGNSTNESEIFRPGGGDMNDTWRSEGSGGGSGSLVPRGSPGSGYIPEAPRDGQAYVRKDGEWVLLSTFLGGHHHHHH

SEQ ID NO: 52 - DNA, 834 bp, Artificial Sequence
  b12 Scaffold
atgtatagcatgcagctggccagctgtgtgacactgacactggtgctgctggtgaattccgcccctaggagggccaacgtgagcacccagctgctgctgaacggcagcctggccaacgagaccgtgaacatcaccagcgtgaacttcaccgacaacgccaagaccatcatcgtgcagctgaacaccagcgtggagatcaactgcaccagggccaacaacggcaccagcaacagcaccggcaacatgagacaggcccactgcaacatcagcagggccaagtggaacaacaccctgaagcagatcgccagcaagctgagggagcagttcggcaacaacaagaccatcatcttcaagcagagcagcggcggcgaccccgagatcgtgacccacagcttcaactgcggcggcgaattcttctactgcaacagcacccagctgttcaacagcacctggttcaacagcacctggagcaccgagggcagcaacaacaccgagggcagcgacaccatcaccctgccctgcaggatcaagggcggcgccaacatcagcggccaaatccgctgcagcagcaacatcaccggcctgctgctgaccagggacggcggcaacagcaacaacgagagcgagatcttcaggcccggcggcggcgacatgaacgacacctggaggagcgagggatccggaggaggaagcggcagcctggtgcctcgaggcagccctggcagcggctacatccccgaggctccacgcgacggccaggcctacgtgcgcaaggacggcgagtgggtgctgctgagcaccttcctgggcggccaccaccaccaccaccactga

SEQ ID NO: 53 - protein, 277 aa, Artificial Sequence
  b12 Scaffold
MYSMQLASCVTLTLVLLVNSAPRRANVSTQLLLNGSLANETVNITSVNFTDNAKTIIVQLNTSVEINCTRANNGTSNSTGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKGGANISGQIRCSSNITGLLLTRDGGNSNNESEIFRPGGGDMNDTWRSEGSGGGSGSLVPRGSPGSGYIPEAPRDGQAYVRKDGEWVLLSTFLGGHHHHHH

SEQ ID NO: 54 - protein, 276 aa, Artificial Sequence
  b12 Scaffold
TVFRQENVDDYYDTGEELGSGQFAVVKKAREKSTGLQYAAKFIKKRRTKSSRRGVSREDIEREVSILKEIQHPNVITLHEVYENKTDVILILELVAGGELFDFLAEKESLTEEEATEFLKQILNGVYYLHSLQIAHFDLSPTNIMLLDRNVPKPRIKIIDFGLAHKIDFGNEFKNIFGGPTFVAPEIVNYEPLGLEADMWSIGVITYILLSGASPFSGGDPQITLAAVSAVAYEFGDGYFSNTSALAKDFIRRLLVKDPKKRMTIQDSLQHPWIKP

SEQ ID NO: 55 - DNA, 838 bp, Artificial Sequence
  b12 Scaffold
taggaccgtgttccggcaggaaaacgtcgacgactactacgataccggcgaggaactgggcagcggccagtttgccgtggtgaagaaggccagagagaagtctaccggcctgcagtatgccgccaagttcatcaagaagcggcggaccaagtctagcagacggggcgtgagcagagaggatatcgagcgggaggtgtccatcctgaaagagatccagcaccccaacgtgatcacactgcacgaggtgtacgagaacaagaccgacgtgatcctgatcctggaactggtggctggcggcgagctgtttgatttcctggccgagaaagagagcctgacagaggaagaggccaccgagtttctgaagcagatcctgaacggcgtgtactatctgcacagcctgcagatcgcccactttgatctgagccccaccaacatcatgctgctggacaggaacgtgcccaagccccggatcaagatcatcgatttcggcctggcccacaagatcgacttcggcaacgagttcaagaacatcttcggcggacctacatttgtggcccccgagatcgtgaattacgagcccctgggactggaagctgacatgtggagcatcggcgtgatcacctacatcctgctgtctggcgcctctcccttcagcggcggagatcctcagatcaccctggccgccgtgagcgccgtggcctatgagtttggcgacggctacttcagcaatacaagcgccctggccaaggactttatcagacggctgctggtgaaggaccccaagaaacggatgaccatccaggatagcctgcagcacccttggatcaagcctggatcc

SEQ ID NO: 56 - protein, 127 aa, Artificial Sequence
  b12 Scaffold
MVRVGMRAAPRVSLEALKAALGGLKLSEAKVYLITDSGGDPDIVRAALLLHTGKKDLLVPDAFGPAFPGGEEALSELVGLLLAQGARRFYGAVVSPGEMTALLDLPPEELLKRVMAIANPGDPGSAL

SEQ ID NO: 57 - DNA, 390 bp, Artificial Sequence
  b12 Scaffold
cctagggtgagagtgggaatgagagctgctcctagagtgagcctggaagctctgaaagctgctctgggaggactgaagctgagcgaagccaaagtgtatctgattacagattccggaggagatcctgacatcgtgagagctgccctgctgctgcatacaggaaaaaaagatctgctggtgcctgatgcttttggacctgcttttcctggaggagaagaagctctgtccgaactggtgggactgctgctggctcagggagccagaagattttacggagctgtggtgagccctggagaaatgacagctctgctggatctgcctcctgaagaactgctgaagagagtgatggctatcgccaatcctggagatcctggaagcgctctgggatcc

SEQ ID NO: 58 - protein, 182 aa, Artificial Sequence
  b12 Scaffold
AQLDSIGFSIIKKAIHAVETRGINEQGLYSIVGVDSRVQKLLSILMDPETEISAEWEIKTITSALKTYLRMLPGPLMMYQFQRSFIKAASGGDPEIVTSEIHSLVHRLPEKNRQMLHLLMNHLAKVADNHKQNLMTGANLGVVFGPTLLRPTVAAIMDIKFQNAVIGVLIGNHEKIFNTVPE

SEQ ID NO: 59 - DNA, 555 bp, Artificial Sequence
  b12 Scaffold
agggcccagctggatagcatcggcttcagcatcatcaagaaggccatccacgccgtcgagaccagaggcatcaatgagcagggcctgtatagcatcgtgggcgtggatagcagagtgcagaagctgctgtccatcctgatggaccctgagacagagatctctgccgagtgggagatcaagaccatcaccagcgccctgaaaacctacctgagaatgctgcctggccccctgatgatgtaccagttccagcggagctttatcaaagccgccagcggcggagaccccgagatcgtgacaagcgagatccacagcctggtgcacagactgcccgagaagaacaggcagatgctgcacctgctgatgaatcacctggccaaggtggccgataaccacaagcagaacctgatgaccggcgctaacctgggcgtggtgtttggccccaccctgctgagacccaccgtggccgccatcatggacatcaagttccagaacgccgtgatcggagtgctgatcggcaaccacgagaagatcttcaacaccgtgcccgagggatcc

SEQ ID NO: 60 - protein, 129 aa, Artificial Sequence
  b12 Scaffold
HKCDITLQQIIKTLNSLTEQKTGCTELTVTDIFAASKNTTEKETFCRAATVLAQFYSHHEKDTACSGGDPQIVTAHAQLIRDLKALDANLWGLAGLNSCPVKEANQSTLENFLERLKTIMREKYSKCSS

SEQ ID NO: 61 - DNA, 396 bp, Artificial Sequence
  b12 Scaffold
aggcacaagtgcgacatcaccctgcagcagatcatcaagaccctgaacagcctgaccgagcagaaaaccggctgtaccgagctgaccgtgaccgatatctttgccgccagcaagaacaccaccgagaaagagaccttttgcagagccgccaccgtgctggcccagttttatagccaccacgagaaggatacagcctgtagcggcggagatcctcagattgtgacagcccacgcccagctgatcagagatctgaaggccctggacgctaacctgtggggcctggccggcctgaactcttgtcctgtgaaagaggccaaccagagcaccctggaaaactttctggaacggctgaaaaccatcatgcgggagaagtacagcaagtgcagcagcggatcc

SEQ ID NO: 62 - protein, 201 aa, Artificial Sequence
  b12 Scaffold
RILVAVLISGTGSNLQALIDSTREPNSSAQIDIVISGKAAVAGLDKAERAGIPTRVINAKSGGDPVIAASAIDLVLEEFSIDIVLLAGFMGILSGPFVQKWNGKMLNIHPSLLPSFKGSNAHEQALETGVTVTGATVHFVAEDVDAGQIILQEAVPVKRGDTVATLSERVKLAEHKIFPAALQLVASGTVQLGENGKITWV

SEQ ID NO: 63 - DNA, 612 bp, Artificial Sequence
  b12 Scaffold
aggagaatcctggtcgccgtcctgattagcggcaccgggtccaatctgcaggctctgatcgactctacaagggagcccaattcaagtgcacaaattgatatcgtgattagcggaaaggccgctgtcgcaggactggataaagccgaaagagctggcatccctactagagtgattaacgcaaagtccgggggtgacccagtgatcgctgcatctgctattgacctggtcctggaagaattttcaatcgatattgtgctgctggccggttttatgggcatcctgagtgggcccttcgtccagaaatggaatggaaagatgctgaatattcaccctagcctgctgccatcctttaaaggatctaatgcccatgagcaggccctggaaaccggtgtgacagtgactggggcaaccgtccacttcgtggccgaagatgtcgatgctggacaaattatcctgcaagaggcagtgcccgtgaagagaggtgatacagtcgccactctgtcagaacgggtgaaactggctgaacataagatctttcctgccgcactgcagctggtcgctagtggaacagtgcaactgggcgaaaacggaaaaatcacctgggtgggatcc

SEQ ID NO: 64 - protein, 211 aa, Artificial Sequence
  b12 Scaffold
QERANYEKLQQKFQMLMSKHQAHVRPQFESLEKINKDIVGWIKLSGTSLNYPVLQGKTNHDYLNLDFEREHRRKGSIFMDFRNELKNLNANTILYGHHVGDNTMFDVLEDYLKQSFYEKHKIIEFDNKYGKYQLQVFSAYKTTTKDSYISTSGGDPQIYQGFLDETKRKSVINSDVNVTVTDTIMTLSTTEDAYSETTKRIVVVAKIIKVS

SEQ ID NO: 65 - DNA, 642 bp, Artificial Sequence
  b12 Scaffold
aggcaggagagagccaactacgaaaagctgcaacagaaattccagatgctgatgagcaagcaccaagctcatgtgaggccccagtttgagtccctggagaaaatcaataaggacattgtcggctggatcaaactgtctgggacctcactgaactatcctgtgctgcaaggtaagacaaatcacgattacctgaacctggacttcgagcgggaacatcgccgaaaaggtagtatctttatggatttcagaaatgagctgaagaacctgaatgcaaacactatcctgtatggtcaccatgtcggggacaataccatgtttgatgtgctggaagattacctgaaacaaagcttctatgagaagcacaaaattatcgagttcgacaacaagtacggaaagtaccagctgcaagtgttttccgcctataagaccacaaccaaagacagctatatctccaccagcggtggtgatccacaaatctaccaggggtttctggatgaaactaaaaggaagtccgtcattaacagcgacgtgaatgtcacagtgactgataccatcatgaccctgtctacaactgaggatgcttattccgagacaactaagcggattgtggtcgtggcaaaaatcattaaagtcagcggatcc

SEQ ID NO: 66 - protein, 193 aa, Artificial Sequence
  b12 Scaffold
YEPKSVKEIFIEMKDTVELMVDLAYASLLFGDKEIAEEVLELEERIDLLNYQLMMHSVLSGGDPKIVTQVITILQIANAIEDISNAAGDLAKMVLEGVELHPVIKETILEGEEIIGKIQVYPESVIVGKTLGELDLATNTGVWIIAVRRGKRWIFGPNENFKIRAGDVLIGRGTRTSIDHLKEIARGAIRVIG

SEQ ID NO: 67 - DNA, 579 bp, Artificial Sequence
  b12 Scaffold
tacgagcccaagagcgtgaaggagatcttcatcgagatgaaggacaccgtggagctgatggtggacctggcctacgccagcctgctgttcggcgacaaggagatcgccgaggaggtgctggagctggaggagaggatcgacctgctgaactaccagctgatgatgcacagcgtgctgagcggcggcgaccccaagatcgtgacccaggtgatcaccatcctgcagatcgccaacgccatcgaggacatcagcaacgccgccggcgacctggccaagatggtgctggagggcgtggagctgcaccccgtgatcaaggagaccatcctggagggcgaggagatcatcggcaagatccaggtgtaccccgagagcgtgatcgtgggcaagaccctgggcgagctggacctggccaccaacaccggcgtgtggatcatcgccgtgaggaggggcaagaggtggatcttcggccccaacgagaacttcaagatcagggccggcgacgtgctgatcggcaggggcaccaggaccagcatcgaccacctgaaggagatcgccaggggcgccatcagggtgatcggc

SEQ ID NO: 68 - protein, 211 aa, Artificial Sequence
  b12 Scaffold
EQAFLQDLDDFQAWLSITQKAVASEDMPESLPEAEQLLQQHAGIKDEIDGHQDSYQRVKESGEKVIQGQTDPEYLLLGQRLEGLDTGWDALGRMWESRSHTLAQALGFQEFQKDAKQAEAILSNFEYTLASLGSGGDPEIVTAGARKFEDFLGSMENNRDKVLSPVDSGNKLVAEGNLYSDKIKEKVQLIEDRHRKNNEKAQEASVLLRDN

SEQ ID NO: 69 - DNA, 633 bp, Artificial Sequence
  b12 Scaffold
gagcaggccttcctgcaggacctggacgacttccaggcctggctgagcatcacccagaaggccgtggccagcgaggacatgcccgagagcctgcccgaggccgagcagctgctgcagcagcacgccggcatcaaggacgagatcgacggccaccaggacagctaccagagggtgaaggagagcggcgagaaggtgatccagggccagaccgaccccgagtacctgctgctgggccagaggctggagggcctggacaccggctgggacgccctgggcaggatgtgggagagcaggagccacaccctggcccaggccctgggcttccaggagttccagaaggacgccaagcaggccgaggccatcctgagcaacttcgagtacaccctggccagcctgggcagcggcggcgaccccgagatcgtgaccgccggcgccaggaagttcgaggacttcctgggcagcatggagaacaacagggacaaggtgctgagccccgtggacagcggcaacaagctggtggccgagggcaacctgtacagcgacaagatcaaggagaaggtgcagctgatcgaggacaggcacaggaagaacaacgagaaggcccaggaggccagcgtgctgctgagggacaac

SEQ ID NO: 70 - protein, 211 aa, Artificial Sequence; identical to SEQ ID NO: 68
  b12 Scaffold

SEQ ID NO: 71 - DNA, 633 bp, Artificial Sequence; identical to SEQ ID NO: 69
  b12 Scaffold

SEQ ID NO: 72 - protein, 214 aa, Artificial Sequence
  b12 Scaffold
IFMDYYENRKVMAEAQNIYEKSPMEEQSQDGEVRKQFKALQQINQEIVGWITMDDTQINYPIVQAKDNDYYLFRNYKGEDMRAGSIFMDYRNDVKSGNANTILYGHRMKDGSMFGSLKKMLDEEFFMSHRKLYYDTLFEGYDLEVFSVYTTTTDFYYISTSGGDPTIYTSFLEKIQEKSLYKTDTTVTAGDAIVTLSTADAGRLVVHAKLVKRQ

SEQ ID NO: 73 - DNA, 642 bp, Artificial Sequence
  b12 Scaffold
atcttcatggactactacgagaacaggaaggtgatggccgaggcccagaacatctacgagaagagccccatggaggagcagagccaggacggcgaggtgaggaagcagttcaaggccctgcagcagatcaaccaggagatcgtgggctggatcaccatggacgacacccagatcaactaccccatcgtgcaggccaaggacaacgactactacctgttcaggaactacaagggcgaggacatgagggccggcagcatcttcatggactacaggaacgacgtgaagagcggcaacgccaacaccatcctgtacggccacaggatgaaggacggcagcatgttcggcagcctgaagaagatgctggacgaggagttcttcatgagccacaggaagctgtactacgacaccctgttcgagggctacgacctggaggtgttcagcgtgtacaccaccaccaccgacttctactacatcagcaccagcggcggcgaccccaccatctacaccagcttcctggagaagatccaggagaagagcctgtacaagaccgacaccaccgtgaccgccggcgacgccatcgtgaccctgagcaccgccgacgccggcaggctggtggtgcacgccaagctggtgaagaggcag

SEQ ID NO: 74 - protein, 57 aa, Artificial Sequence
  b12 Scaffold
VDYIVEYDGSGGDPDILTIRVGEIIRNVKKLQSEGSLEGELNGRRGGFGDGSVKEIK

SEQ ID NO: 75 - DNA, 186 bp, Artificial Sequence
  b12 Scaffold
agggtggactacatcgtggagtacgacggcagcggcggcgaccccgacatcctgaccatcagggtgggcgagatcatcaggaacgtgaagaagctgcagagcgagggcagcctggagggcgagctgaacggcaggaggggcggcttcggcgacggcagcgtgaaggagatcaagggatccaagctt

SEQ ID NO: 76 - protein, 122 aa, Artificial Sequence
  b12 Scaffold
SDGSGKAKEDLKGYVLEGTLTAEKTTLVVKEGGVTLSKNISKSGAVSVELNDSGGDPAIVKVAAWNSGTSTLTITVNSKKTKDLVFTSSNTITVQQYDSNGTSLEGSAVEITKLDEIKNALK

SEQ ID NO: 77 - DNA, 366 bp, Artificial Sequence
  b12 Scaffold
agcgacggcagcggcaaggccaaggaggacctgaagggctacgtgctggagggcaccctgaccgccgagaagaccaccctggtggtgaaggagggcggcgtgaccctgagcaagaacatcagcaagagcggcgccgtgagcgtggagctgaacgacagcggcggcgaccccgccatcgtgaaggtggccgcctggaacagcggcaccagcaccctgaccatcaccgtgaacagcaagaagaccaaggacctggtgttcaccagcagcaacaccatcaccgtgcagcagtacgacagcaacggcaccagcctggagggcagcgccgtggagatcaccaagctggacgagatcaagaacgccctgaag

SEQ ID NO: 78 - protein, 321 aa, Human immunodeficiency virus 1
GARSEVVLVNVTENFNMWKNDMVEQMHEDIISLWDQSLKPCVKLTPLCVGAGSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTGAGHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIE

SEQ ID NO: 79 - protein, 300 aa, Human immunodeficiency virus 1
EIVLENVTESFNMWKNDMVDQMHQDVISLWDQSLKPCVKLTPLCVNCNTSAITQACPKVTLDPIPIHYCAPAGYAILKCNNKTFNGTGPCNNVSTVQCTHGIKPVISTQLLLNGSIAEEEIIIRSENLTNNAKIIIVQLNKSVEINCAYCNISRNEWNITLQWVREKLKRHFPNKTINFTQPSGGDLEITTHSFNCRGEFFYCNTSSLFNSSDNNNSTIITLPCRIKQIINMWQGVGRAMYAPPIKGKITCRSNITGLLLTRDGGETSETNSTETFRPGGGDMRDNWRSELYKYKVVEVK

SEQ ID NO: 80 - protein, 313 aa, Human immunodeficiency virus 1
EYPLHNVTDDFNIWKNYMVEQMQEDIISLWDQSLKPCVQMTFLCVNCNSTTITQACPKVSFEPIPIHYCAPAGYAIFKCNSTEFNGTGTCRNITVVTCTHGIRPTVSTQLILNGTLSKGKIRMMAKDILEGGKNIIVTLNSTLNMTCEYCKYNATDWGKILKQTAERYLELVNNTGSINMTFNHSSGGDLEVTHLHFNCHGEFFYCNTAKMFNYTFSCNGTTCSVSNVSQGNNGTLPCKLRQVVRSWIRGQSGLYAPPIKGNLTCMSNITGMILQMDNTWNSSNNNVTFRPIGGDMKDIWRTELFNYKVVRVK

SEQ ID NO: 81 - protein, 299 aa, Human immunodeficiency virus 2
EITLNVTEAFDAWNNTVTEQAIEDVWHLFETSIKPCVKLTPLCVHCNTSVITESCDKHYWDAIRFRYCAPPGYALLRCNDTNYSGFAPNCSKVVASTCTRMMETQTSTWFGFNGTRAENRTYIYWHGRDNRTIISLNKYYNLSLHCKWCWFKGKWKDAMQEVKETLAKHPRYRGTNDTRNISFAAPGKGSDPEVAYMWTNCRGEFLYCNMTWFLNWIENKTHRNYAPCHIKQIINTWHKVGRNVYLPPREGELSCNSTVTSIIANIDWQNNNQTNITFSAEVAELYRLELGDYKLVEIT

SEQ ID NO: 82 - protein, 320 aa, Simian immunodeficiency virus
EVPLNITEAFEAWDNPLVKQAESNIHLLFEQTMRPCVKLSPICIHCNDSVIKEACDKTYWDTLRVRYCAPAGYALLKCNDKDYRGFAPKCKNVSVVHCTRLINTTITTGIGLNGSRSENRTEIWQKGGNDNDTVIIKLNKFYNLTVRCRWCHFQGDWKGAWKEVREEVKKVKNLTEVSIENIHLRRIWGDPESANFWFNCQGEFFYCKMDWFINYLNNRTEDAEGTNRTCDKGKPGPGPCVQRTYVACHIRQVVNDWYTVSKKVYAPPREGHLECNSSVTALYVAIDYNNKSGPINVTLSPQVRSIWAYELGDYKLVEIT

SEQ ID NO: 83 - protein, 759 aa, Artificial sequence
  SIV-HIV MPER homolog scaffold
MPMGSLQPLATLYLLGMLVASVLAYCVQYVTVFYGVPAWKNATIPLFCATRNRDTWGTTQCLPDNDDYSELAVNITEAFDAWNNTVTEQAIEDVWNLFETSIKPCVKLTPLCIAMRCNKTETDRWGLTGRAETTTTAKSTTSTTTTTVTPKVINEGDSCIKNNSCAGLEQEPMIGCKFNMTGLKRDKKIEYNETWYSRDLICEQPANGSESKCYMQHCNTSVIQESCDKHYWDAIRFRYCAPPGYALLRCNDSNYSGFAPKCSKVVVSSCTRMMETQTSTWFGFNGTRAENRTYIYWHGNSNRTIISLNKYYNLTMKCRRPGNKTVLPVTIMSGLVFHSQPINERPKQAWCRFGGNWSEAIQEVKETLVKHPRYTGTNDTRKINLTAPAGGDPEVTFMWTNCRGEFLYCKMNWFLNWVEDRDQNSNRWKQQKKPEQQKRNYVPCHIRQNITTWHKVGKNVYLPPREGDLTCNSTVTSLIAEIDWINNNETNITMSAEVAELYRLELGDYKLVEITPIGLAPTDVRRYTTTGASSNKSGVFVLGFLGFLATAGSAMGAASLTLSAQSRTLLAGIVQQQQQLLDVVKRQHELLRLTVWGTKNLQTRVTAIEKYLKDQAQLNSWGCAFRQVCHTTVPWPNDSLVPNWDNMTWQEWEGKVDFLEANITQLLEEAQIQQEKNMQELLELDKWASLWNWFNITNWLWYIKYGVLIVLGVVGLRIVIYVVQMLARLRQGSGLVPRGSGSHHHHHHGGTETSQVAPA

SEQ ID NO: 84 - protein, 755 aa, Artificial sequence
  SIV-HIV MPER homolog scaffold
MPMGSLQPLATLYLLGMLVASVLAVTQYITVFYGIPVWKNSSVQAFCMTPNTNLWATTNCIPDDHDYTEVQLNVSEKFEAWKDRNPLVAQAESNIHLLFESTLKPCVKLTPMCIKMNCTKLTSTAPTSSTPTSSSTTDPCPNTDESSCNATLVTNSMDYENSSICSFAMAGYRRDVKKKYNSTWYDQELVCEKENNTTGTRGCYMIHCNDSVIKEACEKTYWDTLRLRYCAPAGFAILKCKDTNYTGFGVCRNVSVVSCTGLMNTTVSSAFGINGSQAENRTEIWQKHGVSNNSVIIKLNKHYKLKIVCRRPGNKTVLPVTIMAGLVFHSQQYNTKLRQAWCHFQGDWKGAWREVRKTIVELPKEKYRGTNNTRQIWLSRQWGDPEAANIWLNCQGEFFYCTPDWFVNWLNNESNSGRNVDVEGNNCTTGKDKRCYKRTYVPCHIRSNVTDWYTLSKKTYAPPREGHLECTSTVTSMMVSLDYNSKERTNVTLTANLENIWAYELGRYKLIEIEPIGFAPTEIRRYVGPTSEKSVPFVLGFLGFLGAAGAAMGATATALTVQSQQLLAGILQQQKNLLAAVEQQQQMLKLTIWGVKNLNARVTALEKYLEDQTRLNLWGCAFKQVCHTTVPWTFNNTPDWDNMTWQEWESQITALEGNISTTLVKAYEQEQKNMQELLELDKWASLWNWFNITNWLWYIKWGFYIVIGLILFRMAWLIWGCIARVRQGSGLVPRGSGSHHHHHHGGTETSQVAPA

SEQ ID NO: 85 - protein, 776 aa, Artificial sequence
  SIV-HIV MPER homolog scaffold
MPMGSLQPLATLYLLGMLVASVLAVKQYVTVFYGVPNWDDNVSVPLICASANTSLWVTTSCLPDLQSYAEVPIYNISENFTIPVKDNQVIQQAWSAMNAMVDSIMKPCVKINPYCVRMQCGEVTKTPTTTPKTTTQMPCFINEQVTVKNPGNETRLEEDLNCTRGLNETTERNAECQYNVTGLCRDCRTEIKQSFRYDDVTCSGERENRTCYMTHCNDSIITQDCNKGVMQNAYFRLCAPAGYMLLRCNEQLNFSKKCENITATPCTGYMLSSVSSFFGFNGTNHTRDELIPLTPNKMEDLNGAKFVYKVAGKWGLIIRCIRKGNRSEVSTISSTGYLFYYGLEHGSRLRLAQCKFEGQWGRMFNNLGKMLKELNAEAMNYTEGTGTCDSKKTTCGRKLKGLPIANMTRHGADLATEMLMHTCGEEMFFCNVTRIFQEWNNKNSDKWYPWANCHIKSNITDWATIGKKIYLPPTSGFNNRIRCTHRVTEMFFEMEKWEPHEDLGGNLSIKFLPPSWETNQFVAEGSKYKLIKLNPIGFAPTDEHRYAPRGSQTSAAPLALGALGLLSAAGTAMGLVSTILTVQAQAVLQGILQQQKQLLVLVEKQQELLRLTIWGVKNLQARLTALEEYVKHQALLASWGCQWKQVCHTNVEWTYNITPNWTKDTWREWESKVAIYDKNITSLLQEAYTTELENQQELLELDKWASLWNWFNITNWLWYIKYAVLIILVIIGLRVLSFIIQNVVKMCRGSGLVPRGSGSHHHHHHGGTETSQVAPA

SEQ ID NO: 86 - protein, 745 aa, Artificial sequence
  SIV-HIV MPER homolog scaffold
MPMGSLQPLATLYLLGMLVASVLAVKQKKQQYLTVYYGVPVWVDAKVDLFCTANSSESGWAVTACLPHALVREEVPMPNVTQNFNAFDNPIEEQLWQDMTSLYKQSFKPCVKLTPYCVSMQCIKTSTNPTPTNTSTTTTTIATTTKTTTDWSGENITMTQYWNCSFNVSGPYRDKKEKSSAVWLEDDIQWADNKDGSGNRTGYMKHCNDSVITQSCETSRFKPFKIRYCAPAGYGLLRCDDKNFNGTGLCNNVTAVACTNLIHTMASTWVQFNGSDEERAEELHIIRKEVKGEVQNGSITIRVPAKYNLTLTCVRPGNKTYRAIHMATGLSFYTTFIQRLRIKRAHCRLNGSWANATKEMRQKILEIFGKANRTNNLTIHYPKGDREVQSVWFQCHGEFFYCNISKALDLLLLQNNTRNSTWSDKWLMPCRINQNVTTWYTVGQHIYLPPKEGELKCSSHISAFVFDVDHYNGSITLTPSADIRAVWRADLFKYKIIEVKPIGFAPSAVRRYEGPESVSHKSAAGIAFGLVAFLSTAGAAMGAASTALTVQSRSLLSGIVQQQQELLKAVEAHGQLLTLTAWGVRNLNTRLTAIEKYLKDQAKLNEWGCAFKQICHTTVPWNNSLEDPDWDNMTWQEWEMKVANYTDEWEGALQRAQEQQERNVQELLELDKWASLWNWFNITNWLWYIKLVVYIIAALILLRIAMFGVNIGSKLCRGSGLVPRGSGSHHHHHHGGTETSQVAPA

SEQ ID NO: 87 - protein, 745 aa, Artificial sequence
  SIV-HIV MPER homolog scaffold
MPMGSLQPLATLYLLGMLVASVLAVNKWVTVYQGVPAWEEADVNDQQFFCFSSSPEIQQVLGCLPPPPGKPVEQNMPNVTEAFDLFKNSFSGEVWIITQTTLEQRLRPCAKLTAYCAPMICTKVNRTENGTSTVAPTTTNNSASDWDESNWKEYPWYNCRMNSTAFLLKDRKELELGFSVEDLTVLGNKNDSNSIRATMKDCANYTVTQVCDMTIVDPVRTGFCAAPGYMLLRCDDKKWDGTGACNNVTAVSCTHEFNITVMSHVLVNASKELSDWAKDREGVWKNDSGTIEYYWFPKDIALGCIRRGNSSHRNLNTANGAKFYYELIPYSKGIYGRCQFVPMTGQNKKNKTQFAIEIKKNLTAWLERISRKNITITPRNGNRTSDPEATFTFVICHRLFFYCNASSLWKHDSPVMNCTIRKNVTSWVTHARILYGPPPGGHLQCNWEKQPVIAFMGTIEGDNDGNGCAYPAAPNFKHALSTLELGRYKLVKMRTTTYVPTDIKRSVNVNWHHGRQKRGIFAFSILALLSGAGAAMGSASVALTIQAQSLNGRASASSNRMLLKLVETQSALLQLTVWGVKNLQVRVATIEGYLEEQAKLASIGCANMQICRTIVPWNKTWGEEDPWQNMTWKQWHERVRNYTDIIEADLVEAYDLQEENEQELLELDKWASLWNWFNITNWLWYIKYVLYAAYVVGGLIGLRIIMVVIACIRGAFRGSGLVPRGSGSHHHHHHGGTETSQVAPA

SEQ ID NO: 88 - protein, 317 aa, Artificial sequence
  SIV cloaked 8b
QLAVLNVTQSFDWCNHTMVQEAIDNVCQLFETTIKPCVKLSPLCVGAGHCNTSTVQESCDTHYWDAVRIRYCAPPGYAIMRCNNKTYNGTGMCSNVSVSSCTRGMETQTSSQLGLNGSEARTYIYWRSCNFTDNAKTIIVNLNTSVTINCTGAGWCNISGTQWNNTIREIAQTLVKHPGNNKTIDFKQSSGGDPEIVTHWFNCGGEFFYCNSTWLFNSTWFNSTWTSDGSNNTKGQKRIYLPCRIRQNVTTWCRVGKMVYLPPREGDLTCNSNITGLLLTRDGGNSNNESITFRPGGGDMRDNYRLDLGDYQLIEVT

SEQ ID NO: 89 - protein, 303 aa, Artificial sequence
  SIV cloaked wt
QLAVLNVTQSFDAFNHTVVQEMIDNIWQLFETTLKPCGAGACDTHYWDAVRFRYCAPPGYAIMRCNNKTYNGTGMCSNVSVSSCTRGIEPQTSTQLGFNGSEARTYVYWRSVNFTDNAKTIIVNLNTSVTINCTRPGNGGSGSGDRPKQAWCNISGAQWNNTIREIAQTLVKHPGNNKTIDFKQSSGGDPEIVTHWTNCGGEFFYCNSTWLFNSTWFNSTWTSDGSNNTKGQKRIYLPCRIRQGGYLPPREGDLTCNSNITGLLLTRDGGNSNNESITFRPGGGDMRDNYRLDLGDYQLIEVT

SEQ ID NO: 90 - protein, 374 aa, Artificial sequence
  SIV cloaked nc
SDTLYITVFYGIPAYRNSSIPLFCTSKARDYNKEAVHIWASTQCLPDNGDYQQLAVLNVTQSFDAFNDTMVQEMIDNIWQLFETSLKPCGAGACDTHYWDAVRIRYCAPPGYALLRCNNKTYNGTGMCSNVSVSSCTRGIEPQTSTQLGFNGSEARTYVYWRSVNFTDNAKTIIVNLNTSVTINCTRPGNGGSGSGDRPKQAWCNISGAQWNNTLREIAQTLVKHPGNNKTIDFKQSSGGDPEIVTHWTNCGGEFFYCNSTWLFNSTWFNSTWTSDGSNNTKGQKRIYLPCRIRQGGYLPPREGDLTCNSNITGLLLTRDGGNSNNESITFRPGGGDMRDNWRLDLGDYQLIEVTPIGLAPSDVRRYTTGGTSR

SEQ ID NO: 91 - protein, 317 aa, Artificial sequence
  HIV-2 cloaked 8B
EIALVNVTEAFDWCNNTMVEQAVDDVCNLFETSIKPCVKLSPLCVGAGHCNTSIIKESCDKHYWDALRFRYCAPPGYAIMRCDNKTYNGTGPCSNVSVSSCTRGMETQTSSQLGLNGSRAENRTYMRSCNFTDNAKTIIVNLNTSVTINCTGAGWCNISRGEWNNTMQEIAQTLITNDGRNKTITFEPSSGGDPEIVTHWFNCGGEFFYCNSTWLFNSTWFNSTWVENRTNNTEGTQHIYLPCRIRQNVTTWCKVGKMMYLPPREGELTCNSNITGLLLTRDGGNSNNESNTFRPGGGDMRDNYRLELGDYKLIEVT

SEQ ID NO: 92 - protein, 303 aa, Artificial sequence
  HIV-2 cloaked wt
EIALVNVTEAFDAWNNTVVEQMVDDVWNLFETSLKPCGAGSCDKHYWDALRFRYCTPPGYAIMRCDNKTYNGTGPCSNVSVSSCTRGIEPQTSTQLGFNGSRAENRVYMRSHNFTDNAKTIIVNLNTSVTINCTRPGNGGSGSGKRPRQAWCNISRGEWNNTLQEIAQTLITNDGRNKTITFKPSSGGDPEIVTHWTNCGGEFFYCNSTWLFNSTWFNSTWVENRTNNTEGTQHIYLPCRIRQGGYLPPREGELTCNSNITGLLLTRDGGNSNNESNTFRPGGGDMRDNYRLELGDYKLVEVT

SEQ ID NO: 93 - protein, 374 aa, Artificial sequence
  HIV-2 cloaked nc
TEQLYVTVFYGIPAWRNASIPLICASKARAYESEVHNTWATIQCLPDNDDYEEIALVNVTEAFDAWNNTMVEQMVDDVWNLFETSIKPCGAGSCDKHYWDALRIRYCAPPGYAILRCDNKTYNGTGPCSNVSVSSCTRGIEPQTSTQLGFNGSRAENRVYMRSHNFTDNAKTIIVNLNTSVTINCTRPGNGGSGSGKRPRQAWCNISRGEWNNTLQEIAQTLITNDGRNKTITFKPSSGGDPEIVTHWTNCGGEFFYCNSTWLFNSTWFNSTWVENRTNNTEGTQHIYLPCRIRQGGYLPPREGELTCNSNITGLLLTRDGGNSNNESNTFRPGGGDMRDNWRLELGDYKLVEVTPIGFAPTSERRYSSQTPGS

SEQ ID NO: 94 - protein, 317 aa, Artificial sequence
  SIV cloaked silent glycan
QLVLVNVTQSFDWCNHTMVQEAIDNVCQLFETTIKPCVKLSPLCVGAGHCNTSTVQESCDTHYWDAVRIRYCAPPGYAIMKCNNKTFNGTGPCTNVSVSSCTRGMRPQTSSQLLLNGSEAREEVVIRSCNFTDNAKTIIVQLNTSVEINCTGAGWCNISRAQWNNTLKQIAQKLRKEFGNNKTIIFKQSSGGDPEIVTHWFNCGGEFFYCNSTQLFNSTWFNSTWSSDGSNNTKGSDTITLPCRIRQNVTTWCRVGKMVYLPPREGDLTCSSNITGLLLTRDGGNSNNESITFRPGGGDMRDNYRLDLGDYQLIEVT

SEQ ID NO: 95 - protein, 215 aa, Artificial sequence
  SIV cloaked od
EPQTCTQLGFNGSEARTYVYWRSVNFTDNAKTIIVNLNTSVTINCTRPGNGGSGSGDRPKQAWCNISGAQWNNTIREIAQTLVKHPGNNKTIDFKQSSGGDPEIVTHWCNCGGEFFYCNSTWLFNSTWFNSTWTSDGSNNTKGQKRIYLPCRIRQLVTTWHRVGKNVYLPPREGDLTCNSNITGLLLTRDGGNSNNESITFRPGGGDMRDNHRNQ

SEQ ID NO: 96 - protein, 317 aa, Artificial sequence
  SIVmac239 cloaked core
QLAVLNVTQSFDAYNHTMVQEAIDNVWQLWETTIKPCVKLSPICIGAGHCNTSVVQESCDTHYWDAVRIRYCAPPGYAIMRCNNKTFNGTMPCSNVSVSSCTRMIEPVVSTQLLLNGSEARTYVYWRSVNFTENATIIIVNLNTSVTIKCRGAGWCNISGAQWNNTLKEIAQTLVKHPRNNKTIIFKQSSGGDPEIVTHSFNCGGEFFYCNSTWLFNSTWFNSTYTSDGTNNTKEQHRIYLPCRIRQIVTTWHRVGKNVYLPPREGDLTCNSNITGLLLTRDGGDGNNESITFRPGGGDMRDNWRSDLYDYQLVEIT

SEQ ID NO: 97 - protein, 317 aa, Artificial sequence
  HXB2 core 8b
EVVLVNVTENFNWCKNDMVEQMHEDICSLWDQSLKPCVKLTPLCVGAGSCNTSVITQACPKVSFEPIPIHYCAPAGFAILKCNNKTFNGTGPCTNVSTVQCTHGIRPVVSSQLLLNGSLAEEEVVIRSCNFTDNAKTIIVQLNTSVEINCTGAGHCNISRAKWNNTLKQIASKLREQFGNNKTIIFKQSSGGDPEIVTHWFNCGGEFFYCNSTQLFNSTWFNSTWSTEGSNNTEGSDTITLPCRIKQIINMWCKVGKMMYAPPISGQIRCSSNITGLLLTRDGGNSNNESEIFRPGGGDMRDNWRSELYKYKVVKIE

SEQ ID NO: 98 - protein, 689 aa, Human immunodeficiency virus 1
  VARIANT (126)..(126): Xaa = any amino acid
MPMGSLQPLATLYLLGMLVASVLAVTVYYGVPVWRDTETVLFCASDAKAHSTEAHNIWATQACVPTDPNPQEVPLINVTEHFDMWKNNMAEQMQEDIISLWEQSLKPCVKLTPLCVTMHCNNSNGXNRTVDNGELDIGYKQMKNCSFNVTTERKDKKKLAYSLFYAEDVVQLNESDSTNQTYRLISCKTTSVTQACPKTTFEPIPIHYCAPPGFAIMKCNEGNFSGKGECKNVSTVQCTHGIKPTISTQLILNGSLDTDDIVIRNDGDNMLVQWNETVSINCTRPGNNTGGQVQIGPAMTFYNIEKIIGDIRQAHCNVSEEWKSMWDRTKEKIKGLLGNNTTFKSRVNIGGDPEVRHFMFNCGGEFFLCNTSRLFDENGTVNGTIILPCRIKQIVNLWTRVGKGIYAPPIRGNITCNSSITGLILEVSGNSTVYPSGGNMVNLWRQELYKYKVVSIEPIGVAPGKAKRRTVNSEKSAAFGLGALFLGFLGAAGSTMGAASITLTVQARNLLSGIVQQQNNLLRAIEAQQQLLQLSIWGIKQLQAKVLAIERYLRDQQILSLWGCSGKTICYTTVPWNETWSNHTSYDSIWGNLTWQQWDEKVRNYSGVIFDLIEQAQEQQNTNEKSLLALDKWASLWNWFDITKWLWYIKIAIMVVAGIIGIRIISVIITIIARVRQGGGTETSQEAPA

SEQ ID NO: 99 - protein, 730 aa, Human immunodeficiency virus 1
  VARIANT (464)..(466): Xaa = any amino acid
MPMGSLQPLATLYLLGMLVASVLALNSEQLYATVYSGVPVWEDASPTLFCASDVNLTSTEQHNIWASQACVPTDPSPNEYDLKNVTDYFNIWKNYMVDQMHEDIISLWDQSLKPCVQMTFLCVQMNCTDVKDNTTNTTTAKTTNPEEETNPVKKCDFNVTTVVKDKQEKKQALFYVSDLLKIGNGTNIYTLINCNSSTIKQACPKVTFEPIPIHYCAPAGYAIFKCNETGFNGTGPCKNISVVTCTHGIKPTVSTHLIFNGTISKGKIRIMAKNISSNSDNILVTLNSTINMTCMRPGNNSVQEMRIGPMAWYSMGLGNGYTNRSRIAFCTYNATEWKETLQGIAERYLELVNYTEKINITFKNSTDGDIEVTHLHFNCHGEFFYCNTNQMFNYTFECNNSNCRIQNDNNTYENSTRTIYCRLRQVVRSWMRGGSGLYAPPIKGSLTCSSNITGLILTRDALIXRXSSNPNITFRPTGGDMKDIWRTQLYNYKVVRVKSFSVAPTKISRPVIGTNHQSEKSAVGLGMLFLGVLSAAGSTMGAAGITLSVRTHSLIRGIVQQQDNLLRAIQAQQHLLRLSVWGIRQLRARLQALETLMQNQQLLNLWGCKGKSICYTSVKWNETWGGNLSIWDSLTWQQWDQQVSNVSSFIYDKIQEAQEQQEENERALLALDKWASLWNWFDITKWLWYIKIAIIIVGALIGVRIVMIVLNLVKNIRQGGGTETSQEAPA

SEQ ID NO: 100 - protein, 718 aa, Human immunodeficiency virus 2
MPMGSLQPLATLYLLGMLVASVLASQYVTVFYGIPAWKNASIPLFCATKNRDTWGTIQCLPDNDDYQEIILNVTEAFDAWNNTVTEQAVEDVWHLFETSIKPCVKLTPLCVAMNCSRVQGNTTTPNPRTSSSTTSRPPTSAASIINETSNCIENNTCAGLGYEEMMQCEFNMKGLEQDKKRRYKDTWYLEDVVCDNTTAGTCYMRHCNTSIIKESCDKHYWDAMRFRYCAPPGFALLRCNDTNYSGFEPKCTKVVAASCTRMMETQTSTWFGFNGTRAENRTYIYWHGRDNRTIISLNKYYNLTMRCKRPGNKTVLPITLMSGLVFHSQPINTRPRQAWCRFGGRWREAMQEVKQTLVQHPRYKGINDTGKINFTKPGAGSDPEVAFMWTNCRGEFLYCNMTWFLNWVEDKNQTRRNYCHIKQIINTWHKVGKNVYLPPREGELACESTVTSIIANIDIDKNRTHTNITFSAEVAELYRLELGDYKLIEITPIGFAPTDQRRYSSTPVSNKSGVFVLGFLGFLATAGSAMGARSLTLSAQSRTLLAGIVQQQQQLLDVVKRQQEMLRLTVWGTKNLQARVTAIEKYLKHQAQLNSWGCAFRQVCHTTVPWVNDSLSPDWKNMTWQEWEKQVRYLEANISQSLEEAQIQQEKNMYELLALDKWASLWNWFDITKWLWYIKYGVHIVVGIIALRIAIYVVQLLSRFRKGGGTETSQEAPA

SEQ ID NO: 101 - protein, 733 aa, Simian immunodeficiency virus
MPMGSLQPLATLYLLGMLVASVLATLYVTVFYGVPAWRNATIPLFCATKNRDTWGTTQCLPDNGDYSEVALNVTESFDAWNNTVTEQAIEDVWQLFETSIKPCVKLSPLCITMRCNKSETDRWGLTKSITTTASTTSTTASAKVDMVNETSSCIAQDNCTGLEQEQMISCKFNMTGLKRDKKKEYNETWYSADLVCEQGNNTGNESRCYMNHCNTSVIQESCDKHYWDAIRFRYCAPPGYALLRCNDTNYSGFMPKCSKVVVSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRNTANQKPKEQHKRNYVPCHIRQIINTWHKVGKNVYLPPREGDLTCNSTVTSLIANIDWIDGNQTNITMSAEVAELYRLELGDYKLVEITPIGLAPTDVKRYTTGGTSSNKSGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMLALDKWASLWNWFDITKWLWYIKYIQYGVYIVVGVILLRIVIYIVQMLAKLRQGGGTETSQEAPA

SEQ ID NO: 102 - protein, 778 aa, Bovine immunodeficiency virus
MDQDLDGAERGERGGGSEELLQEEINEGRLTAREALQTWINNGEIHPWVLAGMLSMGVGMLLGVYCQLPDTLIWILMFQLCLYWGLGETSRELDKDSWQWVRSVFIIAILGTLTMAGTALADDDQSTLIPNITKIPTKDTEPGCTYPWILILLILAFILGILGIILVLRRSNSEDILAARDTIDWWLSANQEIPPKFAFPIILISSPLAGIIGYYVMERHLEIFKKGCQICGSLSSMWGMLLEEIGRWLARREWNVSRVMVILLISFSWGMYVNRVNASGSHVAMVTSPPGYRIVNDTSQAPWYCFSSAPIPTCSSSQWGDKYFEEKINETLVKQVYEQAAKHSRATWIEPDLLEEAVYELALLSANDSRQVVVENGTDVCSSQNSSTNKGHPMTLLKLRGQVSETWIGNSSLQFCVQWPYVLVGLNNSDSNISFNSGDWIATNCMHPITLNKSAQDLGKNFPRLTFLDGQLSQLKNTLCGHNTNCLKFGNKSFSTNSLILCQDNPIGNDTFYSLSHSFSKQASARWILVKVPSYGFVVVNDTDTPPSLRISKPSAVGLAIFLLVLAIMAITSSLVAATTLVNQHTTAKVVERVVQNVSYIAQTQDQFTHLFRNINNRLNVLHHRVSYLEYVEEIRQKQVFFGCKPHGRYCHFDFGPEEVGWNNSWNSKTWNDLQDEYDKIEEKILKIRVDWLNSSLSDTQDTFGLALDKWASLWNWFDITKWLWYIKIIIVIIVLWLLIKILLGMLRSCAKVSQNYQHLPAEEEDGGGTETSQEAPA

SEQ ID NO: 103 - protein, 835 aa, Feline immunodeficiency virus
MAEGFCQNRQWIGPEEAEELLDFDIATQVSEEGPLNPGINPFRQPGLTDGEKEEYCKILQPRLQALREEYKEGSLNSECAGKYRRVRYLRYSDLQVFSILYLFTGYIVYFLRRGGLGKQRQDIDIESKGTGEKFSKNEKGQTVNIRNCKILTIAICSLYIFLFIGIGIYAGQGKAQVIWRLPPLVVPVEDSEIIFWDCWAPEEPACQDFLGAMMHLKASTNISIQEGPTLGKWAKEIWATLFKKATRQCRRGKVWRKWNETITGPKGCANNTCYNVTVIIPDYQCYLDRVDTWLQGKVNISLCLTGGKMLYNKETKQLSYCTDPLQIPLINYTFGPNQTCMWNTSLIKNPDIPKCGWWNQAAYYNSCRWEKADVQFQCQRTQSQPGTWLRKISSWKQKNRWEWRPDFESERVKISLQCNSTKNLTFAMRSSSDYSDVVGAWIEFGCHRNKSRTHAAARFRIRCKWNVGSNTSLIDTCGKDQNVTGANPVDCTMTAKTLYNCSLQEGFTMKIEDLIMHFNMTKAVEMYEIAGNWSCKSDLPTDWGYMKCNCTSRNETDKMKCPAKDGILRNWYNPVAGLRQALDKYQVVKQPDYIVVPEEVLNYQSRQKRAAIHIMLALATVLSIAGAGTGATAIGMVTQYHQVLATHQEALDKITEALKINNLRLVTLEHQVLVIGLKVEAIEKFLYTAFAMQELGCNQNQFFCKIPGELWMRYNLTLNQTIWNHGNVTLQDWYRQTKQLQQKFYEIIMDIEQNNVQGTRLALDKWASLWNWFDITKWLWYIKGLLGGVLGIGLGILLLILCLPTLLDCMRNCINKVMGKILLGGGTETSQEAPA

SEQ ID NO: 104 - protein, 692 aa, Human immunodeficiency virus 1
MPMGSLQPLATLYLLGMLVASVLASAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTDVNATNNTTNNEEIKNCSFNITTEIRDKKKKVYALFYKLDVVPIDDNNSYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISRTKWNKTLQQVAKKLREHFNKTIIFNPSSGGDLEITTHSFNCGGEFFYCNTSELFNSTWNGTNNTITLPCRIKQIINMWQGVGQAMYAPPIEGKIRCTSNITGLLLTRDGGNNNTETFRPGGGDMRDNWRSELYKYKVVKIEPLGVAPTKAKRRVVESEKSAVGIGAVFLGFLGAAGSTMGAASITLTVQARQLLSGIVQQQSNLLRAIEAQQHLLQLTVWGIKQLQARVLAVERYLKDQQLLGIWGCSGKLICTTNVPWNSSWSNKSQDEIWDNMTWMEWDKEINNYTDIIYSLIEESQNQQEKNEQELLALDKWASLWNWFDITKWLWYIKIFIMIVGGLIGLRIVFAVLSIVNRVRQGGGTETSQEAPA

SEQ ID NO: 105 - protein, 25 aa, Human immunodeficiency virus 1
  VARIANT (7)..(7): Xaa = Dap (diaminopropionic acid)
EQELLEXDKWDSLWGGTETSQVAPA

SEQ ID NO: 106 - protein, 12 aa, Rhizopus niveus
EVLEADKWAILG

SEQ ID NO: 107 - protein, 12 aa, E. coli
EILELDKWAILG

SEQ ID NO: 108 - protein, 12 aa, Thermus aquaticus
EVLELDKWAELG

SEQ ID NO: 109 - protein, 13 aa, E. coli
QENLEVDKWAFLF

SEQ ID NO: 110 - protein, 13 aa, Klebsiella sp. LX3
QEFLELDKWAQLA

SEQ ID NO: 111 - protein, 12 aa, Homo sapiens
EILECDKWALLG

SEQ ID NO: 112 - protein, 12 aa, Homo sapiens
ELLELDKWALLS

SEQ ID NO: 113 - protein, 13 aa, Human immunodeficiency virus
QELLELDKWASLW

(19) United States
(12) Patent Application Publication
(10) Pub. No.: US 2010/0068217 A1
(43) Pub. Date: Mar. 18, 2010

(54) EPITOPE-TRANSPLANT SCAFFOLDS AND THEIR USE

(76) Inventors: Peter D. Kwong, Washington, DC (US); Gilad Ofek, Washington, DC (US); Javier Guenaga, Washington, DC (US); Richard Wyatt, Rockville, MD (US); Zhi-yong Yang, Potomac, MD (US); Tongqing Zhou, Boyds, MD (US); Gary J Nabel, Washington, DC (US); Min Tang, N. Potomac, MD (US); William Schief, Seattle, WA (US); David Baker, Seattle, WA (US)

Correspondence Address: KLARQUIST SPARKMAN, LLP, 121 S.W. SALMON STREET, SUITE #1600, PORTLAND, OR, 97204-2988, US

(21) Appl. No.: 12/438,913

(22) PCT Filed: Aug. 24, 2007
(86) PCT No.: PCT/US07/76824

§ 371 Date: Nov. 20, 2009

Related U.S. Application Data
(60) Provisional application No. 60/840,119, filed on Aug. 25, 2006.

(51) Int. Cl. A61K 39/00; G06G 7/58; C07K 14/00; C07H 21/04; G01N 33/53; A61P 31/18

(52) U.S. Cl. 424/185.1; 703/11; 530/350; 536/23.1; 435/7.1

(57) ABSTRACT

Computational protocols for the design of epitope-protein scaffolds which elicit selected neutralizing antibodies are disclosed, and related compositions and uses.

### RELATED APPLICATIONS

This application claims the benefit of U.S. Provisional Application No. 60/840,119 filed Aug. 25, 2006, which is hereby incorporated by reference in its entirety.

### BACKGROUND OF THE INVENTION

1. Field of the Invention

Embodiments of the present disclosure relate to immunization and, in particular, to algorithms for facilitating the transplant of selected epitopes recognized by selected antibodies into an appropriate scaffold, while preserving structure and antigenicity and the resultant epitope-scaffold systems.

2. Description of the Related Art

The immune system is a collection of mechanisms within an organism which protects the organism from infections by acting to identify and neutralize disease causing biological agents. Antibodies are a part of the immune system response found within the blood which performs the identification and neutralizing functions. While the structure of various antibodies is similar, generally “Y” shaped, a tip region at the surface of the branched arms is highly variable. Each of these variants can bind to different targets, referred to as antigens, allowing the antibody to recognize an equally wide variety of antigens. The region upon the antigen that is recognized by the antibody is referred to as an epitope. The tip region of the antibody precisely fits with the epitope region of the antigen, allowing the antibody to target only its corresponding epitope, providing the antibody with high specificity.

Immunizations are designed to take advantage of the immune response of an organism. The organism is exposed to an agent that stimulates an immune response, an antigen, in order to fortify the organism's immune system against that agent. For example, after the human immune system is exposed to an antigen once, the system can quickly develop a response to subsequent infection. Thus, administration of an antigenic composition, a vaccine, can provide controlled exposure to an antigen, allowing the body to protect itself from the antigen later in life and providing a degree of immunity.

In the case of certain pathogens, for example HIV, successful immunization strategies have yet to be realized. In general, HIV is roughly spherical viral envelope, through which the HIV protein protrudes. The HIV protein comprises a cap made of glycoprotein 120 (gp120) and a stem made from glycoprotein 41 (gp41) which anchors the cap to the HIV protein to the viral envelope. This glycoprotein complex enables HIV to attach to and fuse with target cells to initiate the infectious cycle.

Some strategies for developing vaccines for HIV have focused on eliciting antibodies directed towards gp120 or gp41. For example, there are a variety of broadly neutralizing anti-HIV antibodies known to target gp41, including, but are not limited to, 2F5, 2G12, B12, 4E10, and Z13. Vaccines to date, while generating high antibody titers, fail to produce a response from one or more of the neutralizing antibodies.

From the foregoing, then, there exists a need for new immunization strategies for pathogens which have proven resistant to conventional approaches.

### SUMMARY OF THE INVENTION

In an embodiment, a method of computationally designing a biological structure which evokes a selected immune response is provided. The method comprises:

obtaining a geometry of at least one of a first biological structure which is recognized by an immune system, a second biological structure which allows interaction between other biological molecules, and a third biological structure which forms a portion of the immune system capable of recognizing the first biological structure;

selecting at least a portion of first biological structure;

identifying at least a portion of the second biological structure which is a geometric match to the first biological structure;

positioning the first biological structure within the portion of the second biological structure to create a combination of the first and second biological structures;

removing from consideration the second biological structures which demonstrate a steric repulsion greater than a selected amount between at least one of the third biological structures and the first biological structure after the first biological structure is positioned within the portion of the second biological structure; and

removing from consideration the second biological structures which exhibit a binding energy with the third biological structure which is less than a first selected threshold.

In an embodiment, the present disclosure provides a method of computationally designing an epitope-protein scaffold to elicit selected neutralizing antibodies. The method comprises

obtaining three-dimensional structures of at least one protein, at least one epitope sub-range, and at least one complex of the at least one epitope sub-range and the selected antibody;

superimposing the backbone of the epitope sub-range on at least a portion of the surface of the backbone of the protein scaffold and designating the scaffold as a candidate scaffold protein if a deviation between the backbone atoms of the epitope and scaffold is less than a first selected threshold;

constructing an antibody-scaffold complex, where the scaffold-antibody rigid-body orientations are set by the known structure of the antibody-epitope complex and the superposition; and

selecting amino acid residues for a first group of non-epitope scaffold positions which contact the antibody and a second group of non-epitope scaffold positions which contact the epitope but do not contact the antibody.

In another embodiment, the present disclosure provides a method of computationally designing an epitope-protein scaffold to elicit selected neutralizing antibodies. The method comprises:

obtaining three-dimensional structures of at least one protein, at least one epitope sub-range, and at least one complex of the at least one epitope sub-range and the selected antibody;

superimposing the backbone of the epitope sub-range on at least a portion of the surface of the backbone of the protein scaffold and designating the scaffold as a candidate scaffold protein if a deviation between the backbone atoms of the epitope and scaffold is less than a first selected threshold;

removing a first portion of the candidate scaffold protein and attaching a first end of the at least one sub-range of the selected epitope to the protein at an edge of the first removed portion;

adjusting the protein backbone so as to allow bonding between the backbone atoms of the second end of the epitope and the second edge of the removed portion of the protein so as to form a grafted protein-epitope scaffold;

mutating the scaffold protein so as to maintain the grafted epitope in its antibody-bound conformation.

In a further embodiment, the present disclosure provides a method of computationally designing an epitope-protein scaffold to elicit selected neutralizing antibodies. The method comprises:

obtaining three-dimensional structures of at least one selected protein, at least one selected epitope sub-range, and at least one complex of the at least one epitope sub-range and the selected antibody;

selecting a plurality of sub-ranges of the selected epitope;

identifying a structural match between the selected epitope sub-range and the protein;

positioning the epitope within the portion of the selected protein to generate an epitope-protein scaffold;

removing from consideration those scaffolds which demonstrate a steric repulsion greater than a selected amount between the protein scaffold and at least one of the selected antibodies and the epitope after positioning the epitope within the selected protein; and

removing from consideration those scaffolds which exhibit a binding energy with the antibodies which is less than a first selected threshold.

Other embodiments are directed to related compositions and uses.

### BRIEF DESCRIPTION OF THE DRAWINGS

FIG. 1. A flowchart outlining an embodiment of a computational protocol for use in designing epitope scaffolds using the superposition and grafting processes;

FIG. 2. A schematic illustration of an embodiment of a superposition process for the design of epitope scaffolds; (2A) a schematic illustration of the outer surface of an embodiment of a protein, illustrating the conformation of the surface; (2B-2C) a schematic embodiment of superposition of an epitope sub-range over various portions of the backbone of the outer surface of the protein of FIG. 2A in order to find positions of similar conformation between the epitope and protein surface;

FIG. 3. A flowchart outlining an embodiment of a method of performing the structural matching operation of FIG. 1 for the case of superposition;

FIG. 4. A flowchart outlining an embodiment of a method of performing the non-epitope position design operation of FIG. 3 for the case of superposition;

FIG. 5. A schematic illustration of an embodiment of a grafting process for the design of epitope scaffolds; (5A) a schematic illustration of the outer surface of an embodiment of a protein scaffold; a graft region of the protein scaffold having similar conformation to the epitope is identified; (5B) the graft region of the protein scaffold is removed and a first end of the epitope is grafted onto a first edge of the graft region, leaving a break at the second edge of the protein; the protein scaffold backbone is further remodeled about the region of at least one of the first and second edges of the protein in order to close the break; (5C) mutation of protein scaffold in order to stabilize the epitope conformation and substantially reduce scaffold-antibody contact;

FIG. 6. A flowchart outlining the structural matching and epitope positioning operations of FIG. 1 for the case of grafting;

FIG. 7. gp160 Antibody Epitope Map. Gp160 sequence (SEQ ID NO: 1).

FIG. 8. Structures and targets of neutralizing antibodies that block HIV entry into host cells.

FIG. 9. Modeled structures of the epitope-scaffolds.

FIG. 10. gp41 scaffold amino acid sequences. 1IWL (SEQ ID NO: 2); ID3B, (SEQ ID NO: 3); 1KU2 (SEQ ID NO: 4); 1LGY (SEQ ID NO: 5); 2MAT (SEQ ID NO: 6); IM53 (SEQ ID NO: 7); 1NUB (SEQ ID NO: 8).

FIG. 11. Flow chart describing the basic scheme of the methodology from the identification and design of the scaffolds to antigenicity and immunogenicity.

FIG. 12. Envelope glycoproteins gp41 and gp120 with the imprint of broadly neutralizing antibody binding sites, including the broadly neutralizing 2F5.

FIG. 13. Molecular model of the 2F5 antibody bound to the carbon alpha trace of the gp41 17mer peptide. On the right 2F5e_scaffold_{—}1 and superimposed is the gp41 2F5 epitope in the extended conformation described by the crystal structure.

FIG. 14. Expression constructs for expression in mammalian and bacterial cells.

FIG. 15. Binding of 2F5 antibody to scaffolds.

FIG. 16. Specific responses against heterologous scaffold.

FIG. 17. Specific responses against a 2F5 peptide.

FIG. 18. Description of Non-Neutralizing Sera.

FIG. 19. Rationale for binding studies.

FIG. 20. MPER alanine scan peptides. (SEQ ID Nos: 9-22)

FIG. 21. Reactivity of alanine scan peptides to 2F5. EQELLELDKWASLW (SEQ ID NO: 23).

FIG. 22. Reactivity of alanine scan peptides to non-neutralizing sera from rabbits A and B. EQELLELDKWASLW (SEQ ID NO: 23).

FIG. 23. Reactivity of 2F5 to scaffolds and the flexible MPER wild type and mutant K665E peptides.

FIG. 24. Reactivities of non-neutralizing sera with epitope graft compared to flexible MPER peptides.

FIG. 25. Contrasting diagrams of the genetic scaffold, genetic native, and synthetic peptide platforms.

FIG. 26. Recognition by non-neutralizing sera of IKU2 compared to cyclized MPER peptide.

FIG. 27. Ribbon diagrams of gp41e-1KU2 structure based on the Rosetta Model and the crystal structure.

FIG. 28. Comparison of gp41e-1KU2 epitope graft with gp41. Epitope grafts: ELLELDKW (SEQ ID NO: 24); LLELDKWA (SEQ ID NO: 25); LLELDKW (SEQ ID NO: 26); LELDKW (SEQ ID NO: 27); ELDKW (SEQ ID NO: 28); LDKW (SEQ ID NO: 29); DKW (SEQ ID NO: 30); DKWA (SEQ ID NO: 31); DKWAS (SEQ ID NO: 32); DKWASL (SEQ ID NO: 33); ELDKWAS (SEQ ID NO: 34); ELLELDKWASL (SEQ ID NO: 35)

FIG. 29. Comparison of the electrostatic characteristics of the 2F5 bound surface of the gp41 MPER with the electrostatic characteristics of the exposed surface of the 1KU2 epitope graft.

FIG. 30. Superposition of the crystal structures of gp41e-1KU2 in the free form and in complex with 2F5.

FIG. 31. 2F5 induces a fit in a subset of the gp41 epitope graft. Alignment of epitope grafts in Rosetta Model, gp41e-1KU2, and gp41e-1KU2-2F5 crystal structures against gp41. Epitope grafts shown are the same as in FIG. 28.

FIG. 32. Sequence of gp41e-1KU2 Short (SEQ ID NO: 36).

FIG. 33. Study of immunogenicity of 2F5 epitope scaffolds: Schedule of immunizations.

FIG. 34. Study of immunogenicity of 2F5 epitope scaffolds: Chart detailing the assays used to characterized the anti-scaffold serum responses of guinea pigs immunized with scaffolds and the information obtainable by such assays.

FIG. 35. ELISA: Binding to homologous scaffold. Immunogen is displayed on top of each graph. (Black) 2F5 ab (10 μg/ml). (Gray) Pre-immune sera pool. (White) Anti-scaffold sera mean value after 2 immunizations of all animals in each group.

FIG. 36. ELISA: Binding to homologous scaffold. Immunogen is displayed on top of each graph. (Black) 2F5 ab (10 mg/ml). (Gray) Pre-immune sera pool. (White) Anti-scaffold sera mean value after 2 immunizations of all animals in each group.

FIG. 37. ELISA: Binding to homologous scaffold. Immunogen is displayed on top of each graph. (Black) 2F5 ab (10 μg/ml). (Gray) Pre-immune sera pool. (White) Anti-scaffold sera mean value after 2 immunizations of all animals in each group.

FIG. 38. ELISA binding to heterologous scaffold 2F5e_{—}1ku2. Immunogen is displayed on top of each graph. (Black) 2F5 ab (10 μg/ml). (Gray) Pre-immune sera pool. (White) Anti-scaffold sera mean value after 3 immunizations of all animals in each group.

FIG. 39. ELISA binding to captured 2F5 peptide. Immunogen is displayed on top of each graph. (Black) 2F5 ab (10 μg/ml). (White) Pre-immune sera pool. (Gray) Anti-scaffold serum after 3 immunizations of each animal of a group.

FIG. 40. ELISA binding to captured 2F5 peptide. Immunogen is displayed on top of each graph. (Black) 2F5 ab (10 μg/ml). (White) Pre-immune sera pool. (Gray) Anti-scaffold serum after 3 immunizations of each animal of a group.

FIG. 41. ELISA binding to captured 2F5 peptide. Immunogen is displayed on top of each graph. (Black) 2F5 ab (10 μg/ml). (White) Pre-immune sera pool. (Gray) Anti-scaffold serum after 3 immunizations of each animal of a group. (Arrow) b12 ab (10 μg/ml).

FIG. 42. ELISA binding to captured 2F5 peptide. Immunogen is displayed on top of each graph. (Black) 2F5 ab (10 μg/ml). (White) Pre-immune sera pool. (Gray) Anti-scaffold serum after 3 immunizations of each animal of a group. (Arrow) b12 ab (10 μg/ml).

FIG. 43. FACS measuring binding of anti-scaffold serum to WT gp160 (gray) and mutant gp160 (black) expressed in cell surface of 293 T cells.

FIG. 44. ELISA binding curves generated after 4 sequential immunizations with 2F5e_{—}1ku2 scaffold.

FIG. 45. ELISA binding curves generated after boosting 4 times with second scaffold 2F5e_{—}1lgy.

FIG. 46. FACS binding analysis of anti-scaffold sera (highest 2F5 epitope titer) to MPR peptide expressed on surface of 293 cells.

FIG. 47. ELISA binding curves. One line designates a fixed amount of 2F5 antibody binding to heterologous scaffold 2F5e_{—}1d3b. The other line designates a fixed amount of 2F5 antibody mixed with increasing amounts of anti-scaffold sera competing for binding to heterologous scaffold.

FIG. 48. ELISA binding curves. One line designates a fixed amount anti-scaffold sera (1:5000 dilution) binding to 2F5 peptide on plate. The other line designates a fixed amount anti-scaffold sera mixed with increasing amounts of 2F5 antibody competing for binding to 2F5 peptide on the plate.

FIG. 49. Sequences of scaffolds containing the T_{H} cell epitope. 2F5e_{—}1d3bb_TH (SEQ ID NO: 37); 2F5e_{—}1ku2_TH (SEQ ID NO: 38); heterologous T cell helper epitope (SEQ ID NO: 39).

FIG. 50. DNA and amino acid sequences of the b12 scaffolds (part 1). DNA sequences for: Scaffold1 (b12), (SEQ ID NO: 40); Scaffold2 (b12), (SEQ ID NO: 41); Scaffold2′ (b12), (SEQ ID NO: 42). Amino acid sequences for: Scaffold1 (b12), (SEQ ID NO: 43); Scaffold2 (b12), (SEQ ID NO: 44); Scaffold2′ (b12), (SEQ ID NO: 45).

FIG. 51. Nucleotide and amino acid sequences of the b12 scaffolds (part 2). OD252/482 delet-beta20-21 (V3/9C)-G4: nucleotide (SEQ ID NO: 46), amino acid (SEQ ID NO: 47); OD252/482 delet-beta20-21 V3/9C)-G5: nucleotide (SEQ ID NO: 48), amino acid (SEQ ID NO: 49); OD252/482 delet-beta20-21 V3/9C)-G8F-His: nucleotide (SEQ ID NO: 50), amino acid (SEQ ID NO: 51); OD252/482 delet-beta20-21 V3/9C)-G11F-His: nucleotide (SEQ ID NO: 52), amino acid (SEQ ID NO: 53); b12-Sca11: amino acid (SEQ ID NO: 54), nucleotide (SEQ ID NO: 55); b12-Sca12: amino acid (SEQ ID NO: 56), nucleotide (SEQ ID NO: 57); b12-Sca13: amino acid (SEQ ID NO: 58), nucleotide (SEQ ID NO: 59); b12-Sca14: amino acid (SEQ ID NO: 60), nucleotide (SEQ ID NO: 61); b12-Sca15: amino acid (SEQ ID NO: 62), nucleotide (SEQ ID NO: 63); b12-Sca16: amino acid (SEQ ID NO: 64), nucleotide (SEQ ID NO: 65); b12-Sca17: amino acid (SEQ ID NO: 66), nucleotide (SEQ ID NO: 67); b12-Sca18: amino acid (SEQ ID NO: 68), nucleotide (SEQ ID NO: 69); b12-Sca19: amino acid (SEQ ID NO: 70), nucleotide (SEQ ID NO: 71); b12-Sca20: amino acid (SEQ ID NO: 72), nucleotide (SEQ ID NO: 73); b12-Sca21: amino acid (SEQ ID NO: 74), nucleotide (SEQ ID NO: 75); b12-Sca22: amino acid (SEQ ID NO: 76), nucleotide (SEQ ID NO: 77).

FIG. 52. Specificity of interaction of immune serum with Sca2.

FIG. 53. Expression of IgG1 b12 scaffolds in 293 cells.

FIG. 54. Evaluation of antiserum against b12 Sca2.

FIG. 55. Diagram of epitope-transplants into heterologous scaffolds.

FIG. 56. Diagram of epitope-transplants into homologous scaffolds.

FIG. 57. A flowchart outlining computational antigenic cloaking.

FIG. 58. Structure of core gp120. A) Ribbon diagram, B) Topology diagram, C) Stereo plot and D) Structure-based sequence alignment, core gp120: HIV-1 clade B (SEQ ID NO: 78); HIV-1 clade C (SEQ ID NO: 79); HIV-1 clade 0 (SEQ ID NO: 80); HIV-2 (SEQ ID NO: 81); SIV (SEQ ID NO: 82).

FIG. 59. SIV-HIV homolog scaffolds containing gp140 sequence with the membrane proximal portion altered. sme543 HIV MPER (SEQ ID NO: 83), SIVagm_tan1_HIV MPER (SEQ ID NO: 84), SIVlho7_HIV_MPER (SEQ ID NO: 85), SIVdeb_CM40_HIV MPER (SEQ ID NO: 86) and SIVCOLCGU1_HIV_MPER (SEQ ID NO: 87).

FIG. 60. Representative antigenic cloaking constructs. SIV_CLOAKED_{—}8b (SEQ ID NO: 88); SIV_CLOAKED_wt (SEQ ID NO: 89); SIV_CLOAKED_nc (SEQ ID NO: 90); HIV-2_CLOAKED 8B (SEQ ID NO: 91); HIV-2 CLOAKED wt (SEQ ID NO: 92); HIV-2_CLOAKED_NC (SEQ ID NO: 93); SIV_CLOAKED_{—}8B_SILENT-glycan (SEQ ID NO: 94); SIV_CLOAKED_od (SEQ ID NO: 95).

FIG. 61. Cloaking Hx-8b with SIVmac239. New_SIVmac239_cloaked_core (SEQ ID NO: 96); HXB2_core_{—}8b (SEQ ID NO: 97).

FIG. 62. Vaccine immunization strategy.

FIG. 63. Replacement of the gp41 membrane proximal regions (MPER) of related but genetically diverse primate lentiviruses with the HIV-1 MPER from the YU2 HIV-1 Group M, clade B strain. These envelope glycoproteins are cleavage-defective by modification of known or putative precursor cleavage sites (underlined), truncated their cytoplasmic tails and have an appended C9 tag sequence. Group N Env-04CM_{—}1015_{—}04_DQ017382 (SEQ ID NO: 98), Group O Env-AF383260 (SEQ ID NO: 99), HIV-2 Env HIV-2BEN MK6 (SEQ ID NO: 100), SIV mac 239 Env (SEQ ID NO: 101), BIV-Env—Accession # AAA42762 (SEQ ID NO: 102), FIV-Env CABCpady00C or clone FIV-C36 (SEQ ID NO: 103) and HIV-1 M group-Consensus (SEQ ID NO: 104).

FIG. 64. ELISA data showing 1KU2 affinity purification yield (2F5).

FIG. 65. ELISA data showing 1KU2 affinity purification yield (human patient sera).

FIG. 66. Neutralization curve obtained for serum from animal I-003.

FIG. 67. Neutralization curve obtained for serum from animal E-325.

FIG. 68. 2F5 binding to gp41 epitope.

FIG. 69. 2F5 binding to 1KU2.

FIG. 70. 2F5 binding to a cyclized peptide.

### DETAILED DESCRIPTION OF THE PREFERRED EMBODIMENT

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3rd ed., J. Wiley & Sons, Chichester, N.Y., 2001, and Fields Virology 5th ed., Knipe D. M. and Howley P. M. eds, Lippincott Williams & Wilkins, Philadelphia, 2007.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

### Computational Protocols for Design of Epitope-Protein Scaffolds

Embodiments of the present disclosure provide novel computational protocols for the design of epitope-protein scaffolds which elicit selected neutralizing antibodies. In general, the protocols utilize searchable databases containing the three dimensional structure of proteins, epitopes, and epitope-antibody complexes to identify proteins that are capable of structurally accommodating at least one selected epitope on their surface. Protein folding energetic predictions are further utilized to make energetic predications. The predicted energies may be used to optimize the structure of the epitope-scaffold and filter results on the basis of energy criteria in order to reduce the number of candidate proteins and identify energetically stable epitope-scaffolds.

In one embodiment, a method of designing “superposition” epitope-scaffolds is disclosed. Superposition epitope-scaffolds are based upon scaffold proteins having an exposed segment on their surface with a similar conformation as a selected target epitope. The backbone atoms in this superposition region can be structurally superimposed onto the target epitope with less than a selected level of deviation from their native configuration. Candidate scaffolds are identified by computationally searching through a library of three-dimensional structures. The candidate scaffolds are further designed by putting epitope residues in the superposition region of the scaffold protein and making additional mutations on the surrounding surface of the scaffold to prevent undesirable interactions between the scaffold and the epitope or the scaffold and the antibody.

Superposition is advantageous in that it is a conservative technique. Epitope-scaffolds designed by superposition require only a limited number of mutations on the surface of known, stable proteins. Thus, the designs can be produced rapidly and a high fraction of the first round designs are likely to fold properly.

In another embodiment, a method of designing “grafting” epitope-scaffolds is disclosed. Grafting epitope scaffolds utilize scaffold proteins that can accommodate replacement of an exposed segment with the crystallized conformation of the target epitope. For each suitable scaffold identified by computationally searching through a database of known three-dimensional structures, an exposed segment is replaced by the target epitope. The surrounding protein side chains are further mutated to accommodate and stabilize the inserted epitope. Mutations are further made on the surface of the scaffold to avoid undesirable interactions between the scaffold and epitope or scaffold and antibody.

Advantageously, grafting epitope-scaffolds should substantially mimic the epitope-antibody interaction, as the epitope is presented in substantially its native conformation. As such, grafting may be utilized to treat complex epitopes which are more difficult to incorporate using superposition techniques.

In certain embodiments, protein and design calculations are performed using the ROSETTA computer program. ROSETTA is a software application, developed at least in part at the University of Washington which provides protein structure predictions. ROSETTA utilizes physical models of the macromolecular interactions and algorithms for finding the lowest energy structure for an amino acid sequence in order to predict the structure of a protein. Furthermore ROSETTA may use these models and algorithms to find the lowest energy amino acid sequence for a protein or protein-protein complex for protein design. The ROSETTA energy function and several modules of the ROSETTA protein structure modeling and design platform are employed in the protein scaffold design discussed below.

Advantageously, the embodiments of the present disclosure also overcome limitations previously encountered with the design of protein scaffolds. In one example, scaffolds were designed manually, rather than using comprehensive, automated searching of protein databases for optimal scaffold candidates. In another example, most scaffolds have been designed in the absence of protein folding predictions. With folding predictions, however, large numbers of mutations may be introduced into the scaffold without destroying its folding integrity. These and other objects and advantages of the present disclosure are described in greater detail below.

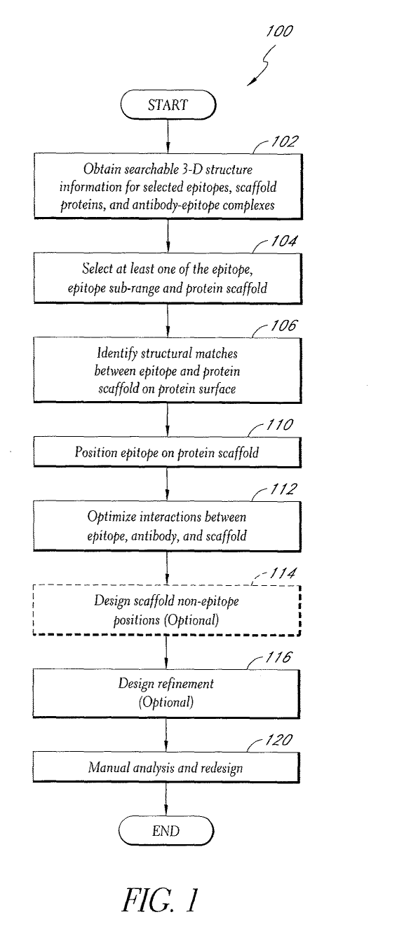

FIG. 1 illustrates one embodiment of a method 100 of epitope-scaffold design. It may be understood that the steps may be performed in any order and one or more of the steps may be repeated or omitted as necessary without departing from the spirit of the invention.

The method begins in Step 102, where three dimensional structures for at least one of selected epitopes, proteins, and the epitope in complex with a selected antibody are obtained. Proteins generally fold into unique three-dimensional structures. The shape that a protein naturally folds in is referred to as its native state. Proteins may further shift between several related structures, referred to as conformations, in the process of performing their biological function.

In one embodiment, the three-dimensional structure information may comprise crystal structures contained within a computer searchable database. In one example, such a database may comprise at least a portion of the Protein Data Bank (PDB) (www.rcsb.org). The PDB is a publicly available depository of information about the three-dimensional structures of large biological molecules, including but not limited to, proteins and nucleic acids. A variety of information associated with each structure is available through the PDB, including sequence details, atomic coordinates, crystallization conditions, 3-D structure neighbors computed using various methods, derived geometric data, structure factors, 3-D images and a variety of links to other resources. In certain embodiments, the entirety of the PDB may be employed. In alternative embodiments, a selected portion of the PDB may be employed. For example, only non-redundant portions of the PDB may be employed. In further alternative embodiments, three-dimensional structure information may be obtained from any other appropriate resource, such as private databases and research data.

In Step 104 of the method, at least one epitope, at least one epitope sub-range, and at least one protein scaffold are selected for design consideration. The epitope sub-range comprises at least a portion of the epitope. In certain embodiments, a plurality of possible sub-ranges of the epitope that might be immunogenically effective on the surface of the protein is identified for consideration. In one embodiment, the effective sub-ranges may be assessed by examining the important epitope-antibody contacts in the crystal structure and/or by consulting the literature for relevant data such as alanine-scanning or neutralization of pseudo-viruses. For example, for the 2F5 epitope, with atomic coordinates defined for 14 residues in complex with the antibody 2F5 in the pdb file named 1tji in the PDB, sub-ranges may comprise: for full length, residues 1-14; for 2-13-mers, residues 1-13 and 2-14; for three 12-mers, residues 1-12, 2-13, 3-14; for four 11-mers, residues 3-11, 4-12, 5-13. It may be understood that references herein to the epitope may refer to the full epitope or any selected sub-range of the epitope.

In Step 306, structural matches between the selected epitope-sub-range and the outer surface of the scaffold protein are identified. In general, the structure of the protein and epitope backbone of the epitope is superimposed upon the backbone of at least a portion of the protein which is present on the outer surface of the protein and a deviation between the backbone atoms of the protein and epitope is calculated. Matches are identified as those proteins which are measured to have a deviation less than a selected amount. Unless otherwise stated, “backbone atoms” and “backbone” refer to the amide nitrogen (N), the alpha carbon (CA), the carbonyl carbon (C), and the carbonyl oxygen (O) on a polypeptide chain.

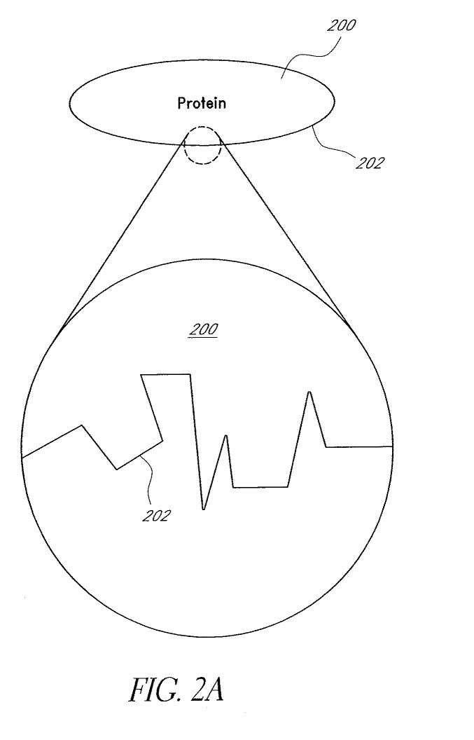

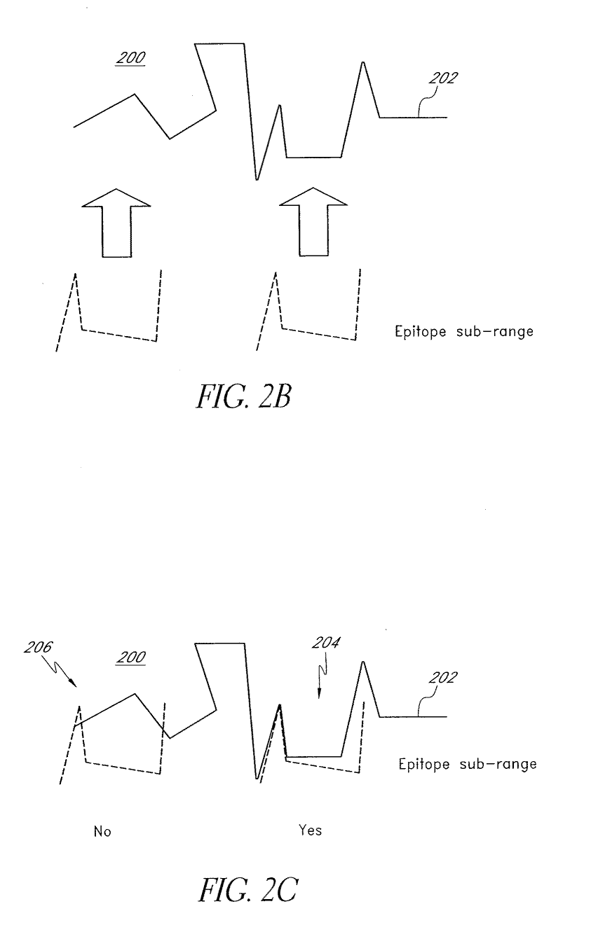

A schematic depiction of the outer surface 202 of the protein 200 is illustrated in FIG. 2A. While any location within the protein might be considered for matching with the epitope, in general, only the surface locations on the protein 200 may potentially interact with the antibody. Thus, generally, only sites on the outer surface 202 of the protein 200 are considered in the matching process.

Protein scaffold selection on the basis of the deviation reflects a physical selection criterion for the design of protein scaffolds. As illustrated in FIG. 2B, there are a wide variety of locations which an epitope might potentially be positioned on the surface of the protein. While some potential sites, such as site 204, possess a similar geometry to the epitope, other sites, such as site 206, possess a very different geometry to the epitope. The greater the deviation between the scaffold and the epitope, the greater the number of mutations of the protein which will ultimately be required. These mutations can potentially destabilize the epitope scaffold or reduce its immunogenicity. Therefore, greater similarity between the protein and the epitope, and thus a lower deviation, is preferred to reduce the mutations introduced into the protein.

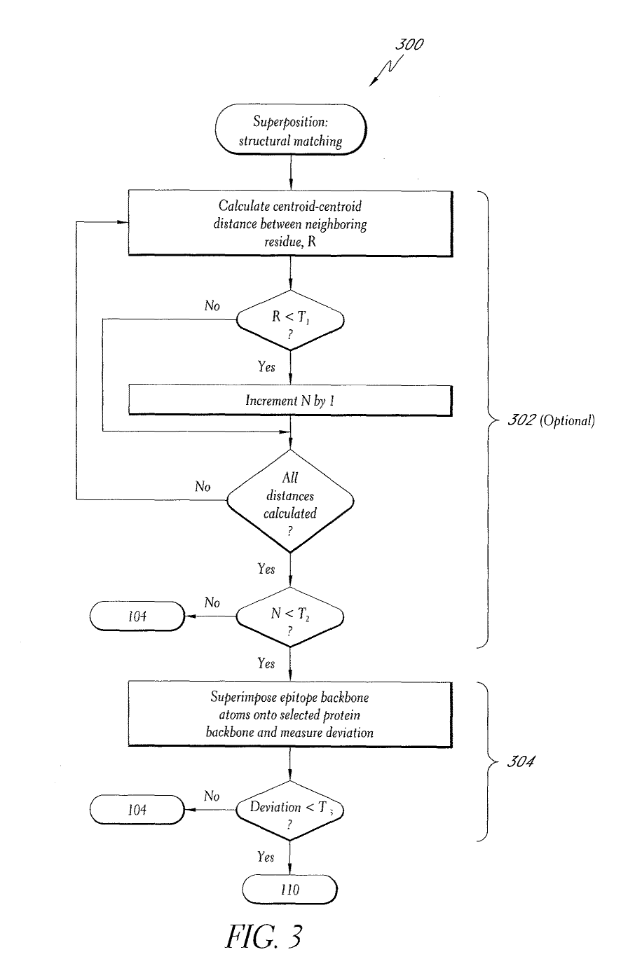

FIG. 3 illustrates one embodiment of a method 300 of structural matching of an epitope sub-range to a protein scaffold. The method optionally begins in Step 302, where it is determined whether or not a protein residue is on the surface of the protein. For each residue within the protein, the centroid to centroid distance between that residue and its nearest neighboring residues is calculated, Step 302. In this calculation, the centroid of the residue is considered to be the center of mass of the residue. However, alternative methods of designating the centroid may be employed, as necessary. For each neighboring residue having a centroid-centroid distance less than a first selected threshold distance, T₁, a counter N is incremented upwards. In one embodiment, the first selected threshold is about 20 nm. After all the centroid-centroid distances have been calculated for a given residue, N is compared to a second threshold value T₂, which is a user selected integer. Residues for which N is less than T₂ are designated as being on the surface of the protein and evaluation of the scaffold continues in Step 304. Residues for which N is less than T₂ are designated as not being on the surface of the protein and the method 300 returns to Step 104 for selection of a new residue for consideration.

In alternative embodiments, other approaches may be used to determine whether a region of the scaffold is present on the scaffold surface. For example, a solvent accessible surface area (SASA) approach may be employed.

In alternative embodiments, this surface detection step may be omitted altogether. For example in circumstances where the three dimensional structure or models of the antibody are known, “clash tests,” discussed in greater detail below, may also provide a test of whether a candidate position on the protein scaffold is on the surface of the scaffold.

In Step 304, the epitope backbone atoms are superimposed upon the surface backbone atoms identified in Step 302 (FIG. 2B) and the deviation between the two is calculated. In one embodiment, the deviation comprises a root mean square deviation (RMSD) calculation. Those scaffolds which are measured to have an RMSD greater than a selected third threshold deviation value, T₃, are removed from further consideration and the method 300 returns to Step 104 for selection of a new residue for consideration. Those protein scaffolds having an RMSD less than T₃ are further evaluated.

It may be understood that this superposition procedure of FIG. 3 is performed over at least a portion of the sequence of each candidate scaffold and as much as the entire sequence of the candidate scaffold using a sliding window. Superposition of the epitope is performed at each possible sliding window.

In step 110, for all protein matches determined in Step 106, the epitope is positioned on the protein scaffold backbone. Either one of the grafting or superposition methods described above may be employed. The following discussion addresses the superposition method, while later discussion will address the grafting method.

In the method of designing a superposition epitope-scaffold, protein scaffolds which have been identified as structural matches to the epitope are further examined to ensure that they do not substantially clash with the antibody. To perform this analysis, a model of the epitope-scaffold/antibody complex is generated. In one embodiment, the scaffold-antibody rigid body orientations, in one embodiment, are set by the crystal structure obtained in Step 102 and the superposition of Step 106. In another embodiment, the protein scaffold residues are mutated to glycine, alanine, or combinations thereof, while the antibody side-chains are kept in their native conformations.

In one embodiment, clash may refer to the steric repulsion across the epitope-scaffold/antibody interface. Using generally known methods, the repulsion can be measured and compared to a selected threshold repulsion, R. In one embodiment, R is about 1000 in arbitrary units, as measured by ROSETTA. If the repulsion is less than R, the scaffold is further evaluated. If the repulsion is greater than R, a new scaffold is chosen for evaluation in Step 104.

Advantageously, the glycine and/or alanine mutation simplifies the calculation of clash. In one aspect, glycine and alanine are relatively small. Thus, any calculations on the epitope-antibody complex are rendered easier. In another aspect, if an unacceptable level of clash is obtained for a protein having glycine and/or alanine residues, there is high likelihood that the protein structure with more complicated residues will also. Therefore, more complicated structures may be eliminated from consideration.

In Step 112, the epitope-antibody interaction is optimized. The optimization procedure comprises a first operation of computationally varying the conformation of at least one of the epitope-scaffold and antibody according to one of several methods. In a second operation, the total energy of the epitope-scaffold/antibody complex is measured in order to determine a local minimum in the total energy. The local minimum reflects a relatively stable state of the complex which may be further considered. The optimization is carried out multiple times for each sub-range of each epitope-scaffold having passed the matching and clashing filters.

In a first optimization protocol, referred to as “repacking,” the side chains of the epitope-scaffold and the antibody are allowed to vary between discrete rotamers. The rotamers are selected from a backbone dependent rotamers library. Non-limiting examples of such a library have been disclosed by R. L. Dunbrack and F. E. Cohen (Protein Sci 6, p. 1661-, August 1997) and B. Kuhlman and D. Baker (Proc Natl Acad Sci USA 97, 10383, Sep. 12, 2000). These libraries provide information on the possible side chains for amino acids, including the statistical preferences in bond angles for the proteins and how changes in one angle tend to affect other angles. Thus, calculations can be restricted to high probability, relatively stable rotamers, rather than those rotamers which are much less probable and less stable. As a result, the calculations are made easier, as there are less calculations to perform, and the results are likely to be more stable, since only relatively high probability rotamers are examined.

In a second optimization protocol, the side chains are first subject to a “minimizing” operation, followed by repacking. The minimizing operation is similar to the repacking operation, however, the chi angles are allowed to vary about their starting values until a local minimum in the total energy is found. Optionally, the minimization operation also comprises minimization of the rigid-body orientation of the epitope scaffold relative to a selected antibody. In further embodiments, this rigid-body minimization may be performed simultaneously with the minimization of chi angles.

In a third optimization protocol, the minimizing procedure is performed, followed by a “docking” procedure. The docking procedure comprises simultaneous optimization of the rigid-body and side chain conformation using Monte Carlo minimization (see J. J. Gray et al, J Mol Biol 331 281 (Aug. 1, 2003)).

The binding energy for each sub-range of the candidate scaffolds is also assessed in order to eliminate scaffolds exhibiting poor binding energy. The binding energy is calculated for the epitope-scaffold/antibody complex when in the complex is in a conformation providing a local minimum of total energy of the complex, as discussed above in reference to Step 112. The calculated binding energies are subsequently ranked and those scaffolds having a binding energy less than a selected threshold energy, E, are removed from consideration. Those scaffolds having an energy greater than about E are further evaluated in Step 314. Several rankings are considered for scaffolds optimized according to the first optimization protocol, including RMS, clash, and length of superposition.

For each candidate scaffold evaluated in the method 100 up to Step 114, the candidate proteins have possessed residues of glycine, alanine, or combinations thereof, except for those in the epitope region. Optionally, in Step 114, the glycine and/or alanine condition may be relaxed, allowing selection of non-glycine and/or alanine residues in the non-epitope scaffold residues.

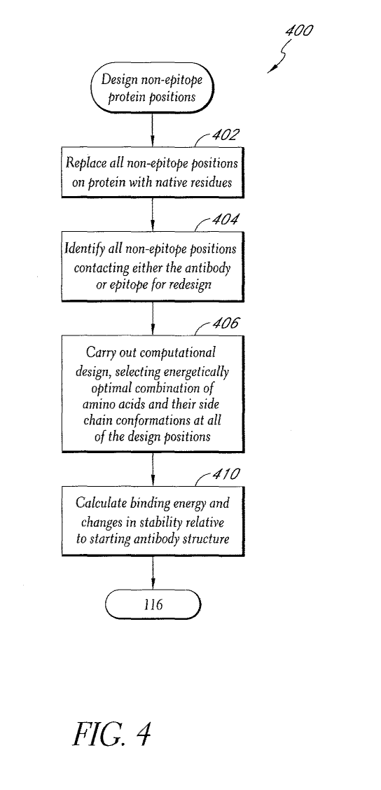

FIG. 4 illustrates one embodiment of a method 400 of selecting the non-epitope protein residues. In Step 402 of the design method 400, all non-epitope positions on the candidate-scaffold are set to their native residues. In Step 404, each of the non-epitope positions are examined and two types of positions are flagged for redesign. The first type of position is designated “inter” and comprises non-epitope positions in the scaffold which contact the antibody. The second type of position is designated “intra” and comprises non-epitope positions which contact the epitope but not the antibody.

Following the identification of the inter and intra positions, a computational design is carried out using ROSETTA_DESIGN, in Step 406, where energetically favorable optimal combinations of amino acids and their side chain conformations at substantially all of the identified design positions are identified. In one embodiment, this design is carried out using a Monte Carlo simulation, where different allowed amino acids are randomly put the scaffold in different conformation in each of the design positions. Changes are accepted when the energy decreases. The allowed amino acids may be selected on the basis of the type of position. For example, to avoid contacts between the antibody and non-epitope positions on the scaffold, inter positions may be selected to be small, polar amino acids. Examples include, but are not limited to Alanine (A), Glycine (G), Serine (S), and Threonine (T). Furthermore, intra positions may be allowed to be any amino acid, with the intention of stabilizing the energetically favorable conformation (local minima) of the epitope side chains determined in Step 312. Stabilizing in this context refers to substantially inhibiting the side chain from moving from the energetically conformation.

Multiple designs are computed for each candidate scaffold, for example, about 100, and each scaffold is ranked according to the binding energy of the epitope-scaffold to the antibody in Step 410. Changes in the internal scaffold stability relative to the native scaffold and changes in the internal antibody stability relative to the starting antibody structure may also be measured and ranked. The scaffolds optimized using the third optimization protocol may also be ranked by scaffold stability in the absence of antibody.

The epitope-scaffolds so designed may be subsequently examined in a post-design analysis, Step 116. The post-design may comprise at least gather of additional information regarding the candidate scaffolds and a manual analysis and redesign of the candidates. The rationale for the post-design analysis is that additional information can play an important role in selected which of the candidate scaffolds should be pursued in experimental testing. In one embodiment, one or more of the following types of information may be accumulated, as necessary: species origin, size, oligomerization state, number of disulfide bonds, average B-factor for backbone atoms over the entire scaffold and over the epitope region alone, hetero atoms present in the crystal structure of the native scaffold. The oligomerization state, in certain embodiments, may be obtained from one of the RCSB Biological Unit Database and the Protein Quaternary Server at the European Bioinformatics Institutes Information.

This information can be used to prioritize scaffolds for further consideration, as well as to target selected scaffolds for further processing. For example, if a scaffold is oligomeric (dimeric, trimeric, etc) then additional testing may be performed to determine if the oligomer will clash with the antibody. Alternatively additional mutations may be performed to render the scaffold monomeric. Furthermore, it may be important to know whether a particular scaffold requires a ligand to maintain the desired scaffold structure.

Additionally, epitope scaffolds may be trimmed to a minimal folding unit that presents the epitope. In one example, a scaffold may possess two globular domains linked only by a flexible peptide linker, in which the epitope onto one of the two domains by superposition, as described above, or by grafting, as discussed below. In certain embodiments, the full length scaffold may be pursued experimentally. In other embodiments, a trimmed version that includes substantially only the globular domain containing the epitope can also be pursued. In cases of trimming, additional design may be necessary on scaffold surfaces that become solvent-exposed as a result of trimming in order to maintain stability and solubility.

In the post-design analysis, manual examination and redesign may also be performed. In one aspect, manual examination allows prioritization of scaffolds based on the accumulated post-design information. In another aspect, manual examination allows visual inspection and validation of scaffold structural stability and epitope-antibody interaction. In a further aspect, manual examination may reveal that mutations back to wild type may be implemented. In another aspect, the literature on each prioritized scaffold may be examined for additional considerations.

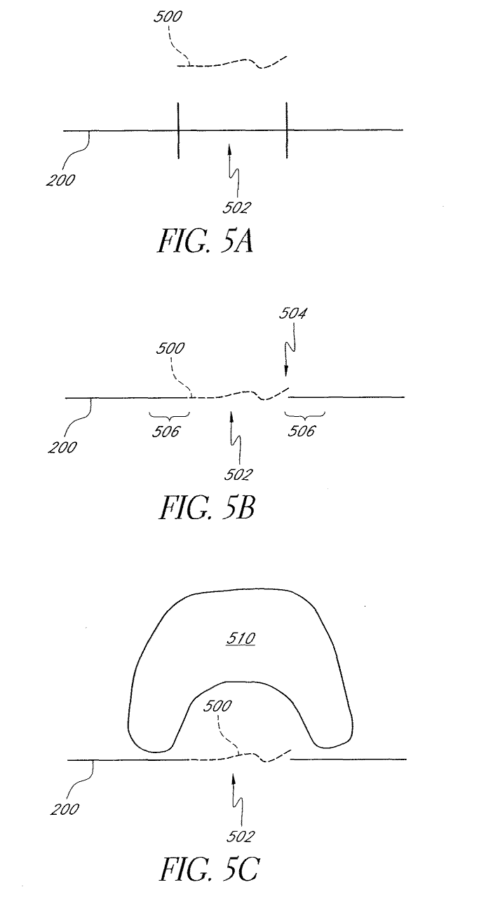

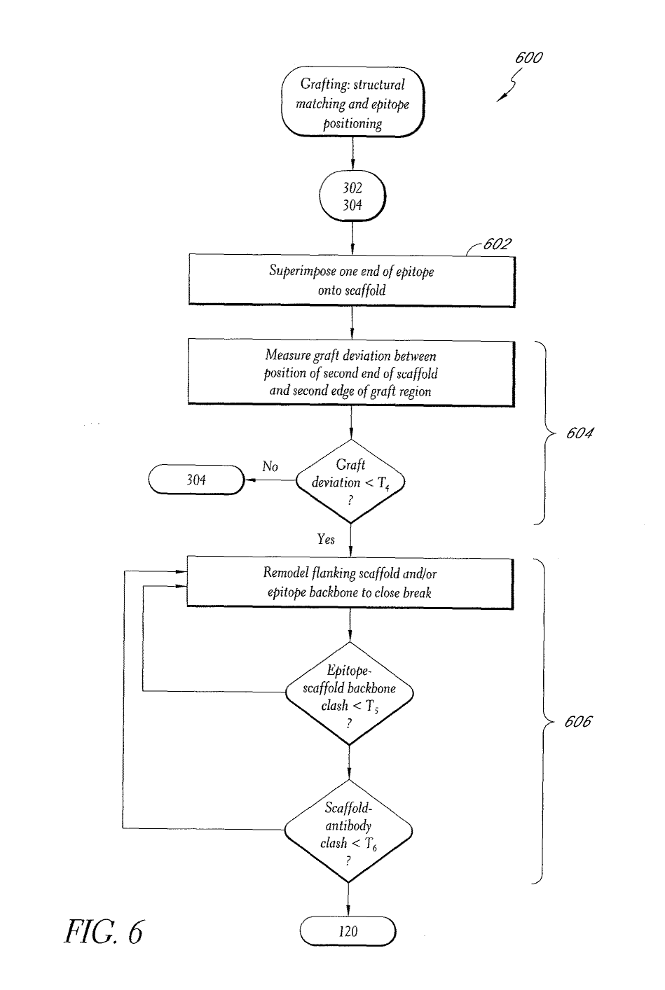

As discussed above, in alternative embodiments, the method 100 may also be employed in the design of epitope-scaffolds formed by grafting, where at least one epitope is grafted onto at least a portion of the scaffold protein. FIG. 5A illustrates one embodiment of an epitope 500 to be grafted onto the protein 200. The method 600 (FIG. 6) of identifying structural matches in the case of grafting both similar and different in some respects to method of structural matching 300 discussed above with respect to FIG. 3.

In one embodiment, the method 600 begins in a similar manner as the superposition matching method 300, determining the surface portions of the protein scaffold, as discussed in Step 302. In Step 602, a selected portion of the epitope backbone is superimposed upon the candidate scaffold for the deviation calculation and selected range of residues is deleted from the scaffold and the epitope is left in their place. In one embodiment, a portion of the epitope about one of the ends of the epitope 500 is selected. For example, as illustrated in FIG. 5B, a portion of the scaffold is removed, forming a graft region 502, and the left hand side of the epitope 500 is grafted to the left edge of the graft region 502. In alternative embodiments, the entire epitope backbone is superimposed on the protein backbone. In further alternative embodiments, just the two ends of the epitope backbone are superimposed on the protein backbone. The deviation between the superimposed portion of the epitope 500 and the protein scaffold 200 is calculated according to Step 304. Those scaffolds 200 having a deviation which is less than the third selected threshold, T₃ are retained for further consideration, while those having a deviation greater than T₃ are discarded.

The method 600 subsequently diverges from the method 300. As illustrated in FIG. 5B there is a break 504 between the right edge of the epitope 500 and the right edge of the graft region 502. A Step 604 is performed in order to measure a grafting deviation between the protein backbone 500 and the backbone of the portions of the epitope 500 which are not superimposed upon the scaffold 200 is calculated. For example, as shown in FIG. 5B, this calculation can determine how close the non-grafted right end of the epitope 500 is to the right edge of the graft region 502. In general, large grafting deviations are desirably avoided, as they indicate that extensive mutation of the scaffold will be required to bond the non-grafted right end of the epitope 500 is to the right edge of the graft region 502. In one embodiment, the grafting deviation calculation comprises measuring the root mean square of the position of a selected number of atoms at the right end of the epitope 500 and a corresponding number of atoms of the right edge of the graft region 502 and taking the difference between the two RMS positions. In an embodiment, the RMS positions are measured over about five atoms.

The grafting RMS deviations are subsequently compared to a fourth selected threshold distance, T₄. Those scaffolds 200 having a deviation which is less than T₄ are retained for further consideration, while those having a deviation greater than T₄ are discarded. In one embodiment, T₄ is less than about 6 Å. In another embodiment, T₄ is less than about 1.6 Å.

The candidate scaffolds 200 are then remodeled in order to close the break 504 in Step 606. Remodeling comprises adjusting at least one of the phi and psi angles, bond distance, bond length, bond angles, and dihedral angles of backbone atoms of the scaffold 200 within the regions 506 to either edge of the graft region. These calculations are performed in order to determine energetically favorable angles which allow closure of the break (FIG. 5C).

The epitope-scaffold/antibody complex so formed is subsequently modeled and evaluated for clash. Such clash may be evaluated between at least one of the scaffold 200 and epitope 500 and the scaffold 200 and the antibody 510. As discussed above, in one embodiment, clash may be evaluated on the basis of steric repulsion. For each of the clashes examined, a threshold is selected and compared to the calculated repulsion. For example, a fifth selected repulsion threshold, T₅, may be compared to the clash calculated for the epitope 500 and scaffold 200, while an sixth selected repulsion threshold, T₆, may be compared to the clash calculated for the scaffold and antibody. Those scaffolds 200 having a repulsion which is less than the appropriate thresholds are retained for further consideration, while those having a deviation greater than the appropriate threshold are discarded. The remaining candidate thresholds are retained for further optimization in Step 112.

The grafting optimization is performed in Step 112. In one embodiment, the optimization comprises mutation of the scaffold in order to stabilize the epitope-scaffold conformation. Because a portion of the scaffold backbone is removed in the grafting process, there is a probability that the side chains of the scaffold may fail to retain their conformation, which in turn may affect the conformation of the grafted epitope, causing it to deviate substantially from its antibody-bound configuration. As this deviation may result in reduced stability of the epitope-scaffold and/or as reduced immunogenicity, mutations to the scaffold may be introduced in order to maintain the epitope in its antibody bound configuration.

A refinement step 116 may also be performed with respect to the grafting approach after the design to further stabilize the epitope-scaffold conformation. The purpose of the refinements step is to examine the binding energy and stability of the epitope-scaffold/antibody complex. In one embodiment, the refinement may comprise a repacking operation as discussed above. In further embodiments, the epitope backbone may be moved with respect to the flanking scaffold regions

The binding energy and/or internal scaffold energy for each sub-range of the candidate scaffolds may also be assessed in order to eliminate scaffolds exhibiting poor binding energy. The calculated energies are subsequently ranked and those scaffolds having a binding energy less than a fifth selected threshold energy, B, and/or an internal scaffold energy less than a ninth selected threshold, I are removed from consideration.

Those scaffolds having a binding energy greater than B and/or an internal energy greater than I are further evaluated in Step 120.

Manual analysis and redesign may be performed, as necessary, in Step 120. In one aspect, this step may comprise any of the procedures discussed above with respect to superposition. In another aspect, the epitope-scaffold interaction with the antibody may be verified. In a further aspect, the manual analysis may examine the stability of the epitope-scaffold and the epitope-antibody interaction.

The grafting protocol may be further varied in a number of ways. In one alternative embodiment, the method 100 may be performed without knowledge of the structure of the antibody in complex with the epitope. In an approach, clash-checking and optimization with the antibody, as discussed above, are omitted from the method. In another approach, low resolution models for the antibody may be provided or constructed for use with clash checking, while omitting the optimization step. In each case, regardless, the scaffold positions are still designed around the epitope in order to support the ideal conformation of the epitope.

In additional embodiments, grafting may be performed using other approaches. In one approach, termed “S-matching,” superposition is not done at one end, rather over a selected range in the middle of the epitope. As a result, neither end of the epitope is initially closed. In one embodiment of S-matching, a selected sub-range of the epitope is superimposed on the scaffold. Subsequently, the corresponding residues on the scaffold are deleted, using the epitope background in their place. This leaves two chain breaks, one at each end of the epitope sub-range inserted into the scaffold.

In another approach, termed “end-matching,” the initial superposition is performed at the N-terminus and the C-terminus of the epitope.

In another embodiment, prior to measuring the deviation, the partially grafted epitope and scaffold may be relaxed and examined. For example, the relaxing may take the form of allowing the conformation selected portions of at least one of the non-fixed end of the epitope graft and the protein scaffold in regions 506 to be varied from their respective native conformations. An example of such a variation would be changes in the torsion angle along the respective backbones of the epitope and/or protein scaffold. Alternatively, other variations as known in the art may be performed. The selected regions of the epitope may be selected from those which are known to be non-critical to the immunogenicity of the epitope.

In additional embodiments, calculations may be performed in the absence of the antibody. When the antibody is present, it presents constraints on the conformation of the epitope-scaffold. As a result, design with the antibody present inherently carries the risk that the conformation of the epitopes so designed may not maintain their conformation in the absence of the antibody, which can potentially affect the immunogenicity of the epitope scaffold. Therefore, calculations which are performed absent the antibody may provide a more conservative approach to the design.

Embodiments of the above superposition and grafting methods may also be employed in combination in order to design complex epitope scaffolds having more than one epitope (more than one stretch of consecutive epitope residues) placed on the protein scaffold. For example, at least one epitope designed by superposition and at least one epitope designed by grafting may be generated on the same scaffold. Alternatively, at least two epitopes designed by superposition and/or at least two epitopes designed by grafting may be generated. In these cases of scaffolding a complex epitope, the rigid body orientation of the different components of the epitope relative to each other must be maintained.

In another embodiment, a sidechain or sidechains of an epitope can be grafted onto a scaffold without grafting the epitope backbone. This may be termed an “inverse rotamer graft”. This is useful in cases in which the antibody may contact only a sidechain or sidechains of an epitope. More generally it is useful in cases of complex epitopes in which in part of the epitope, the antibody may contact only a sidechain or sidechains of an epitope. In these cases it is necessary to present those particular epitope sidechain(s) in the antigen conformation, but it is not necessary to present the epitope backbone in the antigen conformation for those particular epitope residues. Grafting sidechains but not backbone atoms allows greater freedom in the graft matching and design process, since sidechains placed in the native antigen conformation (and held fixed relative to the rest of the epitope) can be connected to different backbone conformations via different rotamers. Connecting a grafted sidechain to a backbone is analogous to closing a backbone graft, but in the case of the sidechain, different ‘proxy’ sets of alpha carbon, amide nitrogen, and carbonyl carbon are built off the sidechain base in a physically realistic manner, and ‘closure’ requires that at least one of the proxy sets of alpha carbon, amide nitrogen, and carbonyl carbon can superimpose with an rms deviation of less than 0.5 Å onto corresponding atoms in at least one residue position on the scaffold backbone. Once a sidechain or sidechains are grafted this way, computational design of surrounding non-epitope positions can be utilized to ensure the grafted sidechains are maintained in the native antigen conformation. Note that in cases of complex epitopes, some parts of the epitope may require superposition or grafting to transfer backbone atoms to a scaffold, while other parts may only require grafting of sidechains.

### Compositions

The present invention provides various immunogenic compositions. As used herein an “immunogenic composition” refers to any composition that is capable of eliciting an immune response. The term “vaccine” refers to an immunogenic composition that reduces the risk of, or prevents, infection by an infectious agent (a “prophylactic vaccine”) or that ameliorates, to any extent, an existing infection (a “therapeutic vaccine”). If a vaccine protects an organism from subsequent challenge with the infectious agent, the vaccines is said to be “protective.”

### Chimeric Non-HIV Polypeptides and Polynucleotides Encoding Therefor

The present embodiment of the invention provides an immunogen comprising a chimeric non-HIV polypeptide that is not from HIV-1, HIV-2 or SIV that comprises at least one heterologous epitope that is recognized by an HIV-1 neutralizing antibody. Additional immunogens of the invention include polynucleotides comprising a nucleotide sequence encoding a chimeric non-HIV polypeptide or a variant thereof, wherein the non-HIV sequence is not from HIV-1, HIV-2 or SIV and wherein the nucleotide sequence further encodes a heterologous epitope recognized by an HIV-1 neutralizing antibody.

As used herein, “heterologous epitope” comprises a domain that is not present in the native polypeptide or encoded by the native polynucleotide encoding therefore. For example, a heterologous epitope for an HIV-1 neutralizing antibody comprises an epitope that is not present in the native non-HIV-1, non-HIV-2, nor non-SIV non-HIV polypeptide (or encoded by the polynucleotide encoding therefor). Polypeptides comprising such heterologous epitopes or polynucleotides encoding therefor are referred to herein as “chimeric polypeptides” or “chimeric polynucleotides”, respectively. Heterologous epitopes that can be employed in the chimeric polypeptides of the invention are discussed elsewhere herein, as are various methods to determine if such an epitope is present in the non-HIV polypeptide.

In specific embodiments, the chimeric polypeptides or polynucleotides encoding therefor of the invention are isolated or substantially purified polynucleotide or polypeptide compositions. An “isolated” or “purified” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the polypeptide of the invention or biologically active portion thereof is recombinantly produced, optimally culture medium represents less than about 30%, 20%, 10%, 5%, or 1% (by dry weight) of chemical precursors or non-protein-of-interest chemicals.

### Variant Polypeptides and Polynucleotides Encoding Therefor

As discussed throughout, the compositions disclosed herein can employ variant non-HIV polypeptides, polynucleotides encoding therefor, as well as variants of the heterologous epitopes recognized by the HIV-1 neutralizing antibodies. As used herein, “variants” is intended to mean substantially similar sequences. A “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. As used herein, a “native” polynucleotide or polypeptide comprises a naturally occurring nucleotide sequence or amino acid sequence, respectively. Variant proteins encompassed by the present invention are biologically active, that is they continue to possess the desired biological activity of the native protein activity as described herein for scaffold. Such variants may result from, for example, genetic polymorphism or from human manipulation. Biologically active variants of a native non-HIV polypeptide employed in the methods of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

A fragment of a biologically active portion of an non-HIV polypeptide of the invention will encode at least 15, 25, 30, 50, 100, 150, 200, 250, 300, 350, 400, 450, 500, 550, 600, 650, 700, 750 or 800 contiguous amino acids, or up to the total number of amino acids present in a full-length non-HIV polypeptide of the invention.

For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the non-HIV polypeptides of the invention. Naturally occurring allelic variants such as these can be identified with the use of well-known molecular biology techniques, such as, for example, with polymerase chain reaction (PCR) and hybridization techniques as outlined below. Variant polynucleotides also include synthetically derived polynucleotides, such as those generated, for example, by using site-directed mutagenesis but that still encode a non-HIV protein of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides has at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

A fragment of a non-HIV polynucleotide may encode a biologically active portion of a non-HIV polypeptide. A biologically active portion of a non-HIV polypeptide can be prepared by isolating a portion of one of the non-HIV polynucleotides of the invention, expressing the encoded portion of the non-HIV protein (e.g., by recombinant expression in vitro), and assessing the activity of the portion of the non-HIV polypeptide. Polynucleotides that are fragments of an non-HIV nucleotide sequence comprise at least 15, 30, 50, 100, 150, 200, 250, 300, 350, 400, 450, 500, 550, 600, 650, 700, 800, 900, 1,000, 1,100, 1,200, 1,300, 1,400 or more contiguous nucleotides, or up to the number of nucleotides present in a full-length non-HIV polynucleotide of the invention.

Variant non-HIV polypeptides of the invention, as well as polynucleotides encoding these variants, are known in the art and are discussed in further detail elsewhere herein. The polypeptide employed in the methods of the invention may be altered in various ways including amino acid substitutions, deletions, truncations, and insertions. Methods for such manipulations are generally known in the art. As discussed below, variant polypeptides or polynucleotides of the invention can comprise heterologous epitopes for HIV-1 binding antibodies. For example, amino acid sequence variants and fragments of the non-HIV polypeptide can be prepared by mutations in the DNA. Methods for mutagenesis and polynucleotide alterations are well known in the art. See, for example, Kunkel (1985) Proc. Natl. Acad. Sci. USA 82:488-492. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.). Conservative substitutions, such as exchanging one amino acid with another having similar properties, may be optimal.

Thus, the polypeptides and polynucleotides employed in the methods of the invention encompass naturally occurring sequences as well as variations and modified forms thereof. Such variants will continue to possess the desired activity for scaffold as discussed elsewhere herein. Obviously, the mutations that will be made in the DNA encoding the variant must not place the sequence out of reading frame and optimally will not create complementary regions that could produce disadvantageous secondary mRNA structure.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated for functional variants of the non-HIV polypeptides by the ability to behave as scaffolds.

Methods of alignment of sequences for comparison are well known in the art. Thus, the determination of percent sequence identity between any two sequences can be accomplished using a mathematical algorithm. As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48.443-453 to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

### Heterologous Epitopes

As used herein, an “HIV-1 binding antibody” comprises an antibody that specifically interacts with an epitope of HIV-1. An HIV-1 binding antibody that can neutralize a virus is referred to herein as an “HIV-1 neutralizing antibody.” In an alternative embodiment, any ligand that neutralizes the virus is contemplated. As discussed above, the chimeric non-HIV polypeptides, and polynucleotides encoding the same, are from a member of the protein database that is not HIV-1, HIV-2 or SIV and further comprises at least one heterologous epitope that is recognized by an HIV-1 neutralizing antibody. In the alternative embodiment, any neutralizing ligand is envisioned.

By “specifically interacts” is intended that the antibody that recognizes the epitope of an HIV envelope polypeptide forms a specific antibody-antigen complex with that epitope (either in an in vitro or in vivo setting) when the epitope is contained in a non-HIV polypeptide that is not from HIV-1, HIV-2 or SIV. Thus, the HIV-1 binding antibody binds preferentially to the non-HIV polypeptide comprising the heterologous HIV-1 epitope. By “binds preferentially” is meant that the antibody immunoreacts with (binds) substantially more of the non-HIV polypeptide comprising the HIV-1 epitope than the non-HIV polypeptide lacking the epitope, when both polypeptides are present in an immunoreaction admixture. Substantially more typically indicates at least greater than 10%, 20%, 30%, 40%, 50%, 60%, 70%, 80%, 90%, 95%, or greater of the immunoprecipitated material is the non-HIV polypeptide comprising the HIV-1 epitope.

The heterologous epitope can be native to the HIV-1 envelope polypeptide or alternatively, the epitope can be synthetically derived, so long as the epitope continues to be recognized by the HIV-1 neutralizing antibody. In addition, the heterologous epitope or the heterologous domain containing the epitope can be of any length including about 2 to about 7 amino acids, about 5 to about 10 amino acids, about 11 to about 20 amino acids, about 21 to about 30 amino acids, about 31 to about 40 amino acids, about 41 to about 50 amino acids, about 51 to about 60 amino acids, about 61 to about 70 amino acids, about 71 amino acids to about 80 amino acids, about 81 to about 90 amino acids, about 91 to about 100 amino acids, about 101 to about 110 amino acids, or longer.

The heterologous epitope can be placed anywhere in the non-HIV sequence, as long as the chimeric polypeptide retains the activity of the native scaffold polypeptide. In still further embodiments, the amino acid sequence of a non-HIV polypeptide is aligned with the amino acid sequence of an HIV-1 polypeptide. The chimeric polypeptide is then engineered to comprise the necessary amino acid substitutions, deletions and/or additions that result in the heterologous epitope from the HIV-1 polypeptide to be placed in the corresponding region of the non-HIV polypeptide. Determining such corresponding regions between two polypeptides or polynucleotides is routine in the art.

The nucleotide sequence encoding the heterologous epitope or the domain it is contained in can be of any length including about 15 to about 30 nucleotides, about 31 to about 60 nucleotides, about 61 to about 90 nucleotides, about 91 to about 120 nucleotides, about 121 to about 150 nucleotides, about 151 to about 180 nucleotides, about 181 to about 210 nucleotides, about 210 to about 240 nucleotides, about 241 to about 270, about 271 to about 300, about 301 to about 330 nucleotides, or longer. It is recognized that various methods can be employed to generate the chimeric polynucleotide having the heterologous epitope including nucleic acid substitutions, deletions, truncations, and insertions. Methods for such manipulations are generally known in the art.

Methods for determining if the heterologous epitope is recognized by an HIV-1 neutralizing antibody are disclosed in WO 2006/091455 and WO 2005/111621. In addition, the formation of an antibody-antigen complex can be assayed using a number of well-defined diagnostic assays including conventional immunoassay formats to detect and/or quantitate antigen-specific antibodies. Such assays include, for example, enzyme immunoassays, e.g., ELISA, cell-based assays, flow cytometry, radioimmunoassays, and immunohistochemical staining. Numerous competitive and non-competitive protein binding assays are known in the art and many are commercially available. Representative assays include, for example, various binding assays with chemokine receptors (CCR5 or CXCR4), gp41, characterized domains of these polypeptides, and competitive binding assays with characterized HIV-1 binding antibodies.

In addition, “neutralization” of the virus in the presence of an appropriate neutralizing antibody can be assayed. For example, a reduction in the establishment of HIV infection and/or reducing subsequent HIV disease progression in this sample when compared to a control sample that lacks the HIV-1 neutralizing antibody can also be monitored. A reduction in the establishment of HIV infection and/or a reduction in subsequent HIV disease progression encompass any statistically significant reduction in HIV activity in the sample. Methods to assay for the neutralization activity include, but are not limited to, a single-cycle infection assay as described in Martin et al. (2003) Nature Biotechnology 21:71-76. In this assay, the level of viral activity is measured via a selectable marker whose activity is reflective of the amount of viable virus in the sample, and the IC50 is determined. In other assays, acute infection can be monitored in the PM1 cell line or in primary cells (normal PBMC). In this assay, the level of viral activity can be monitored by determining the p24 concentrations using ELISA. See, for example, Martin et al. (2003) Nature Biotechnology 21:71-76.

A variety of epitopes for HIV-1 neutralizing antibodies are known in the art. Such epitopes are found, for example, in gp160, gp120, or gp41. In specific embodiments, the epitope recognized by the HIV-1 neutralizing antibody is from an HIV-1 envelope polypeptide. Any HIV strain or isolate can be used. See, for example, HIV Molecular Immunology (2006/2007) Korber et al. ed., Los Alamos National Laboratory, Theoretical Biology and Biophysics, Los Alamos, N. Mex. LA-UR 07-4752.

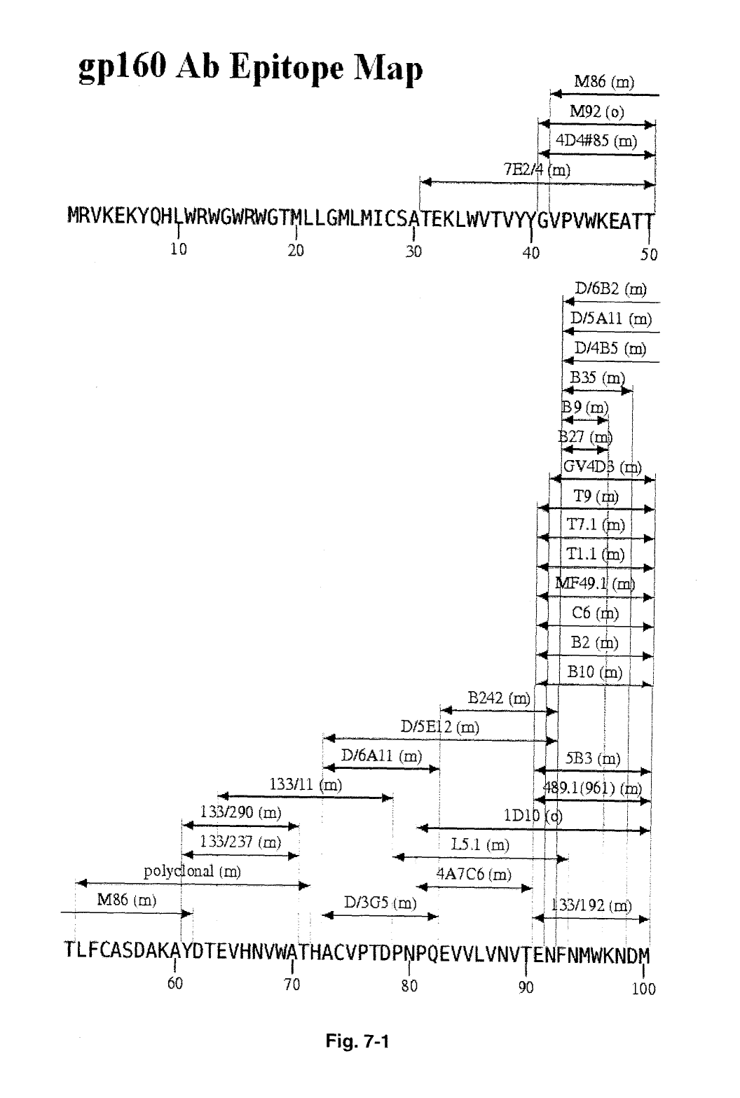

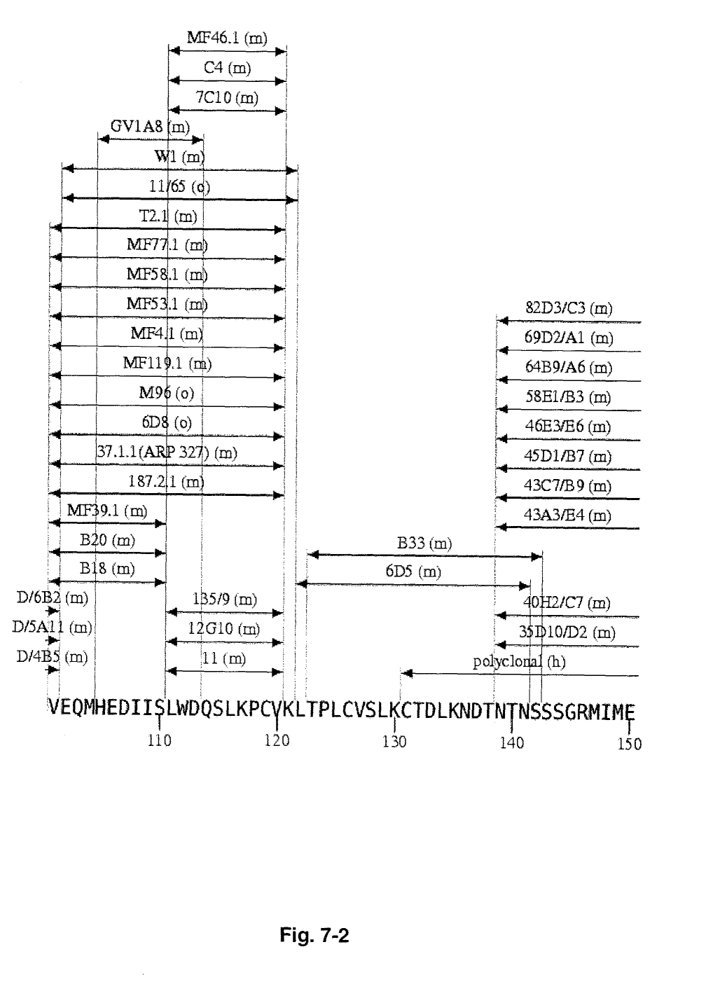

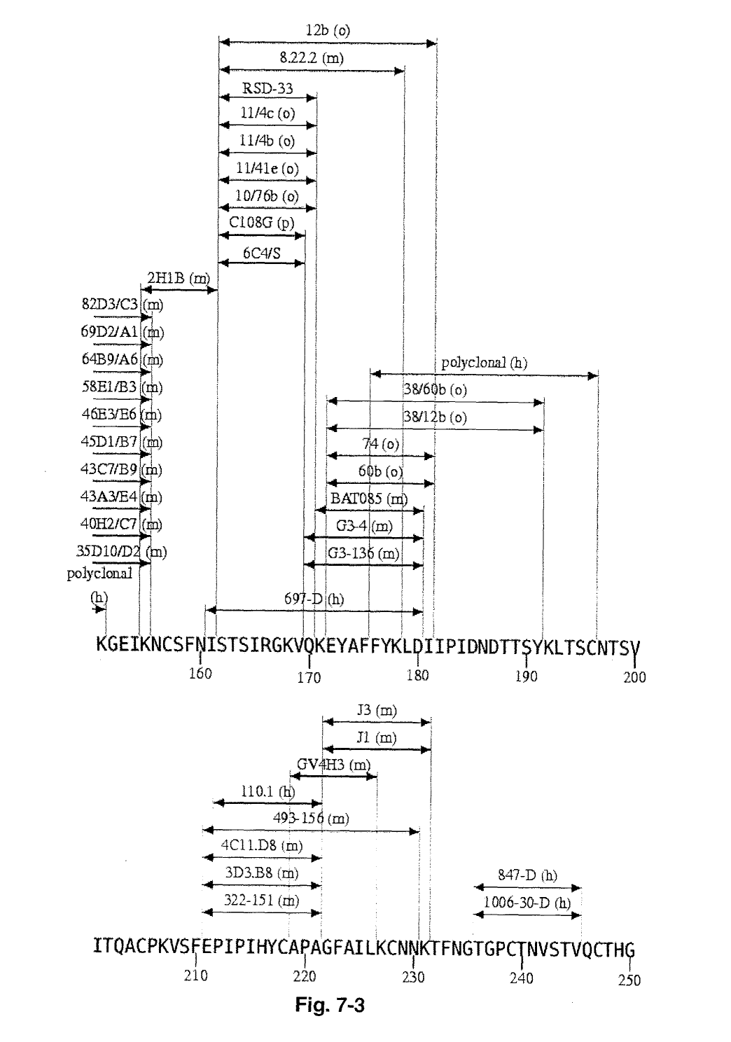

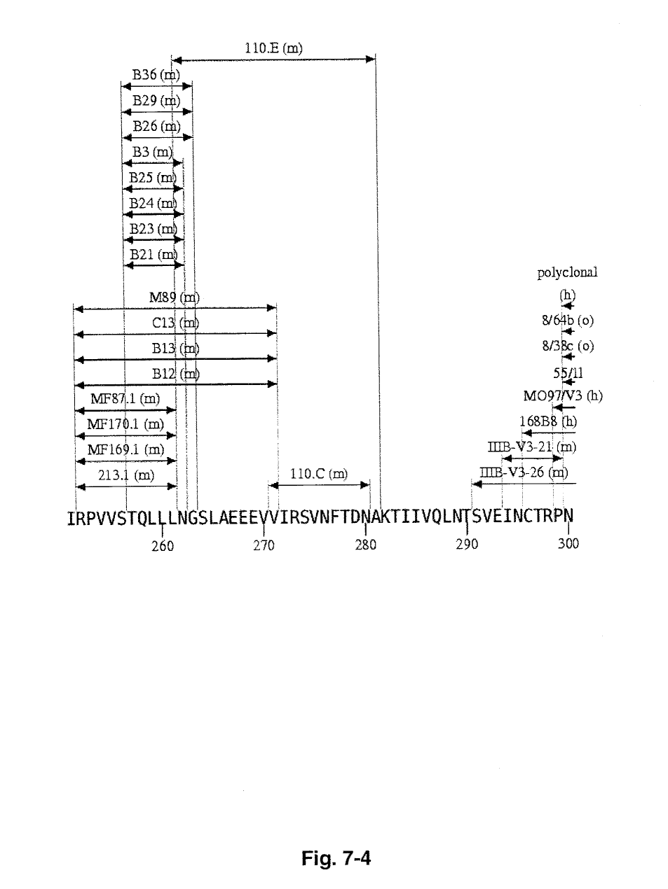

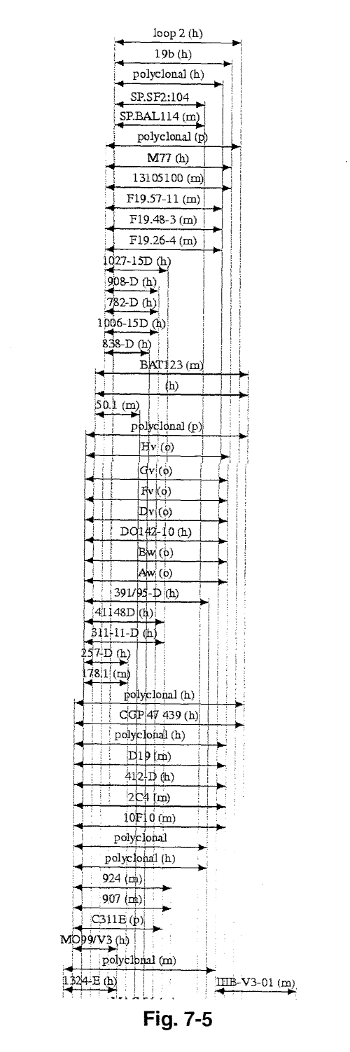

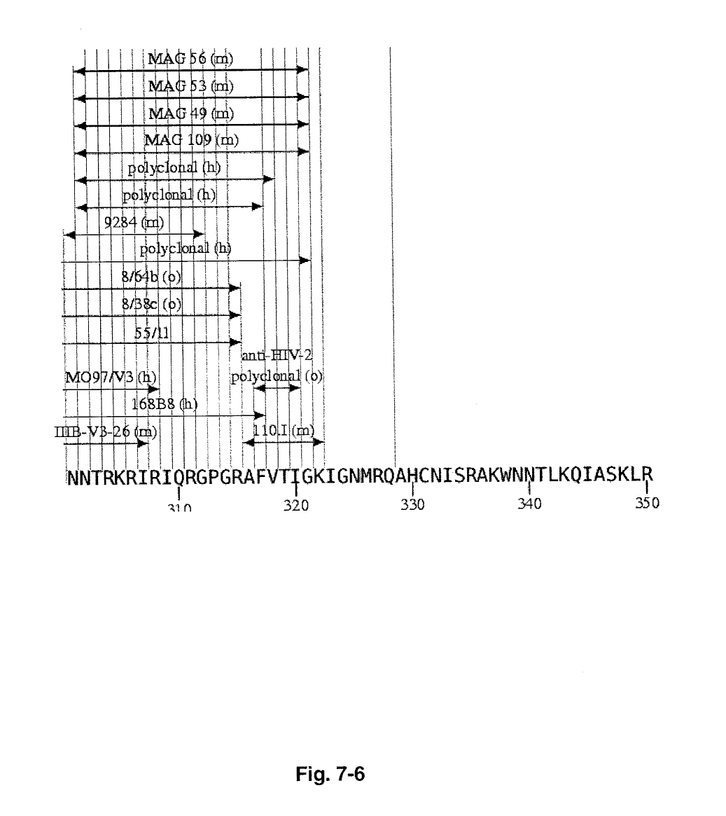

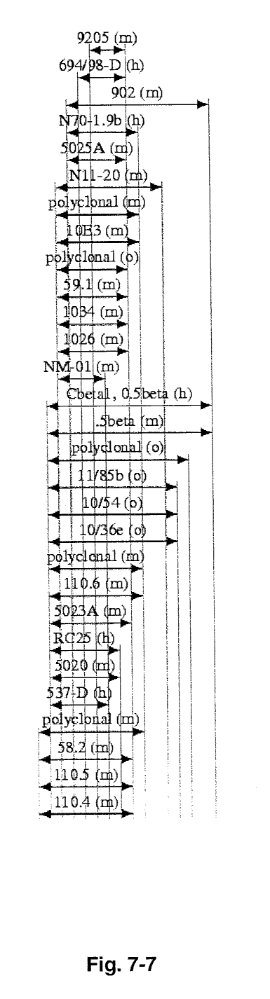

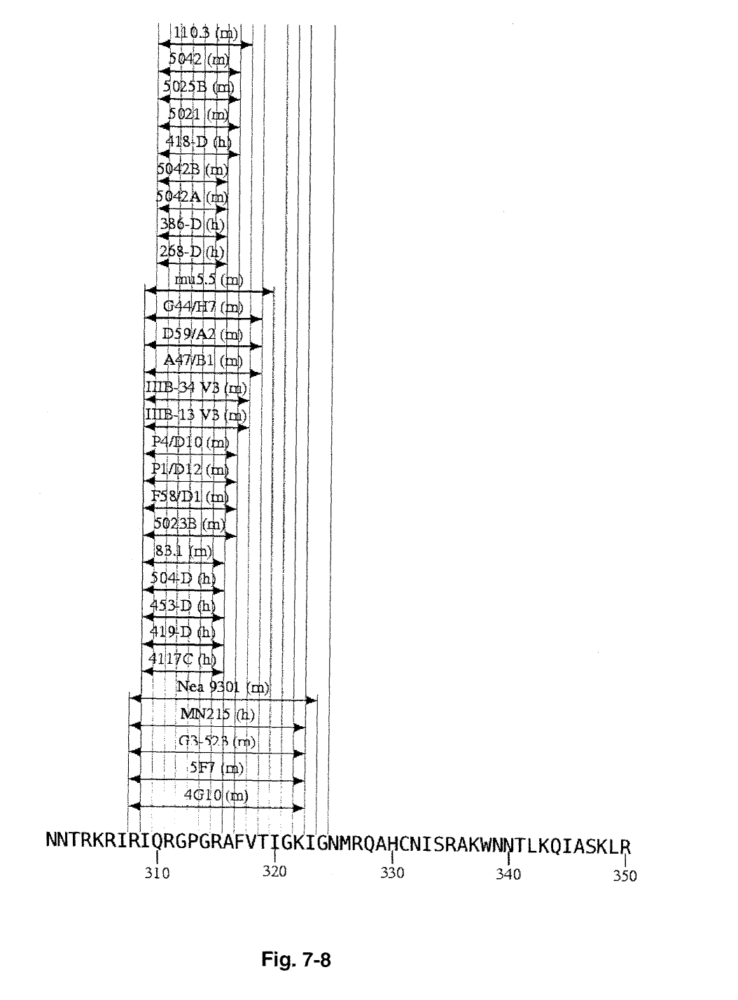

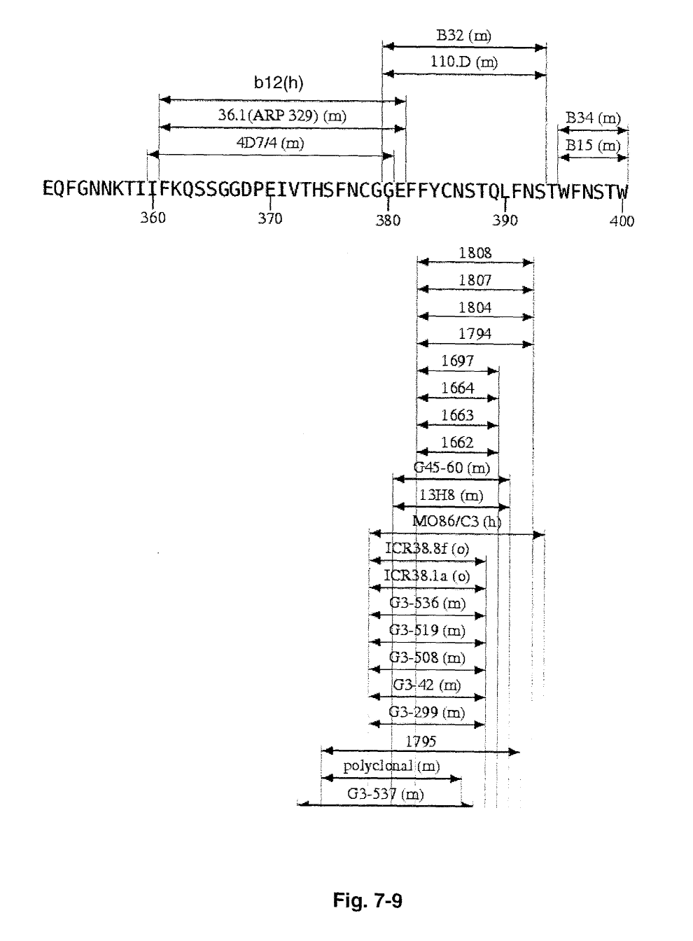

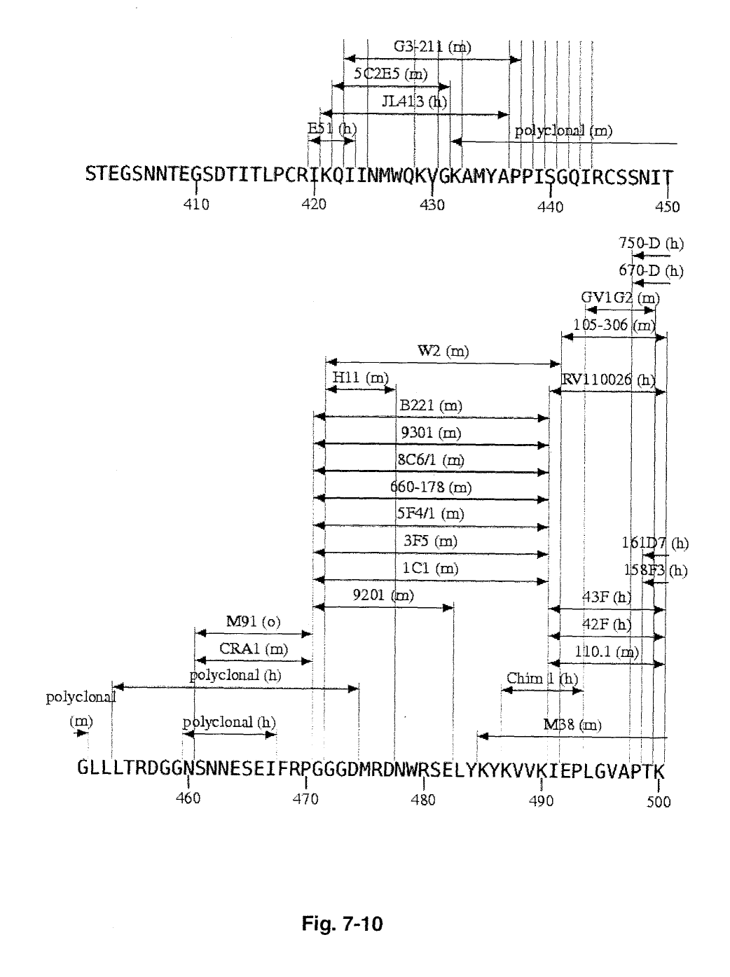

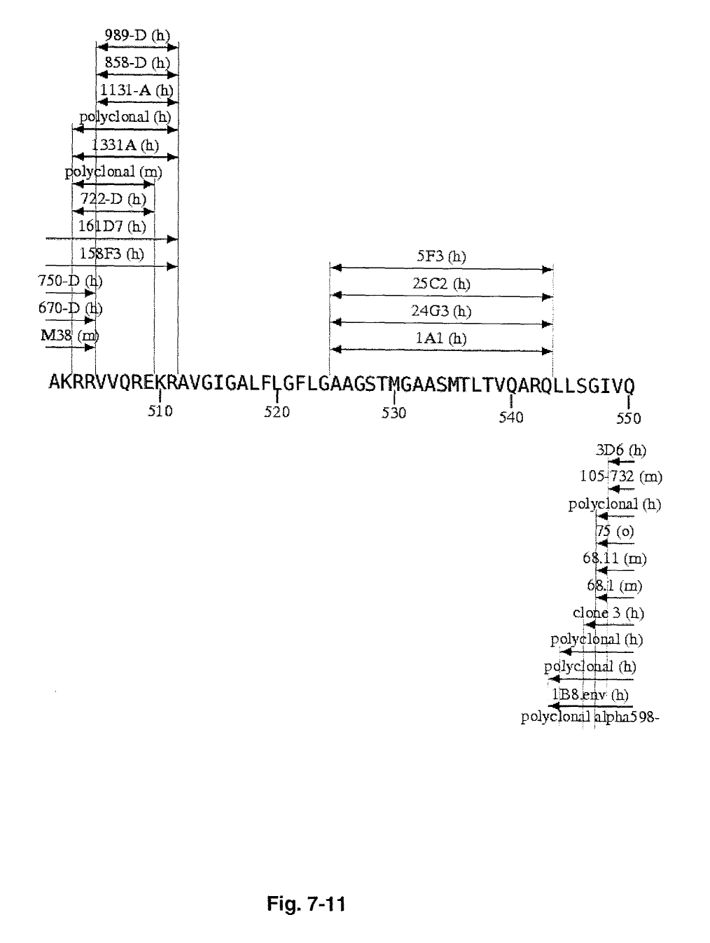

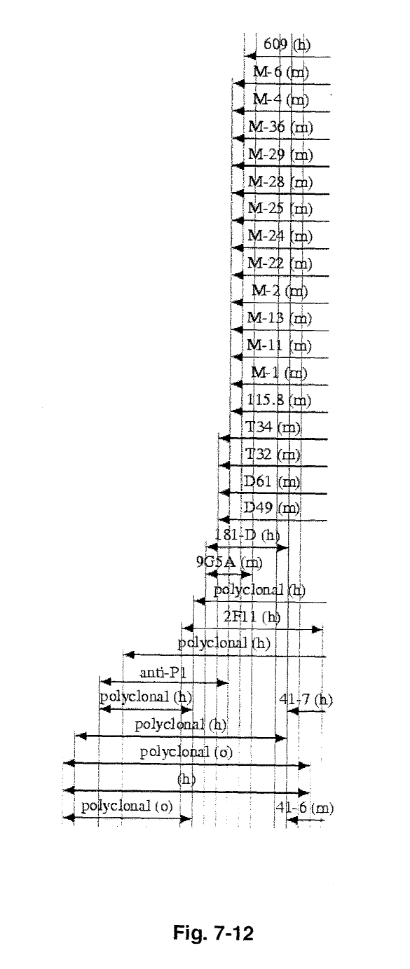

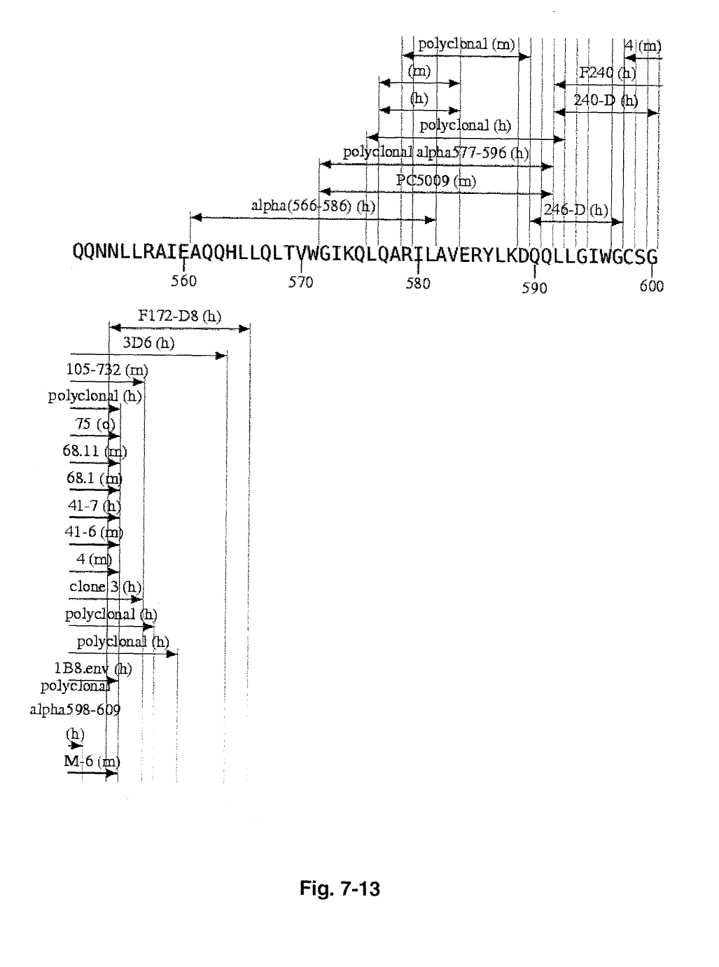

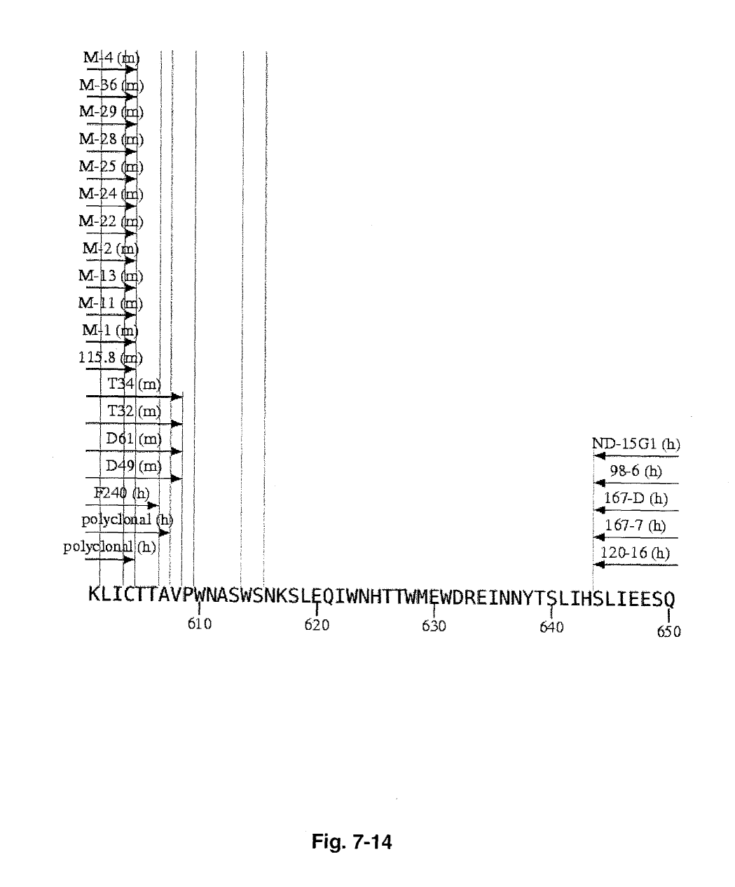

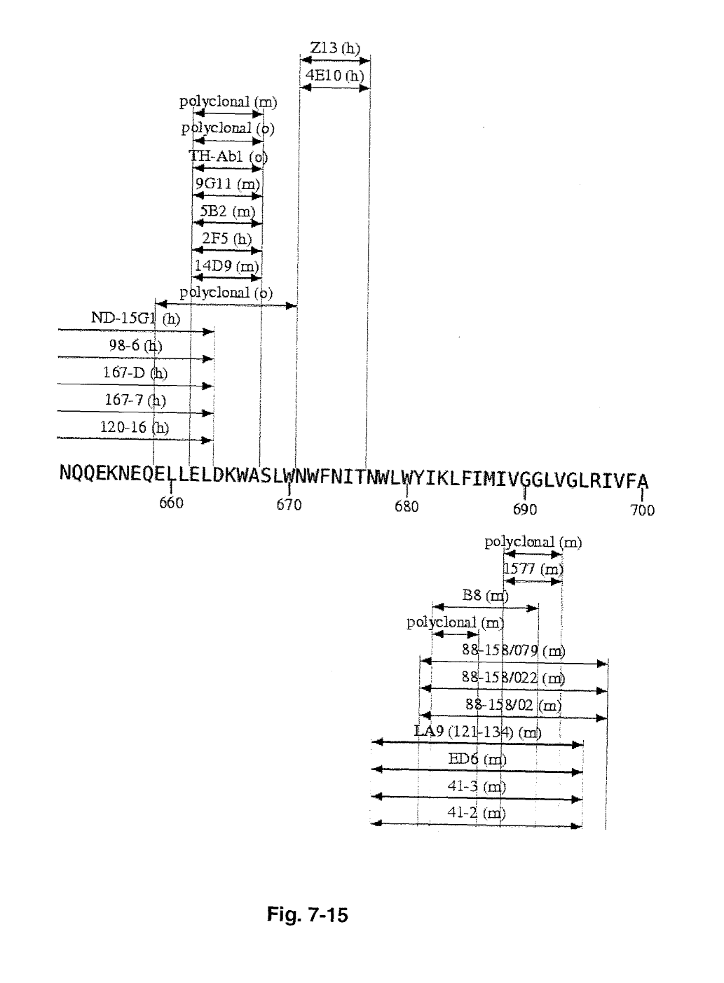

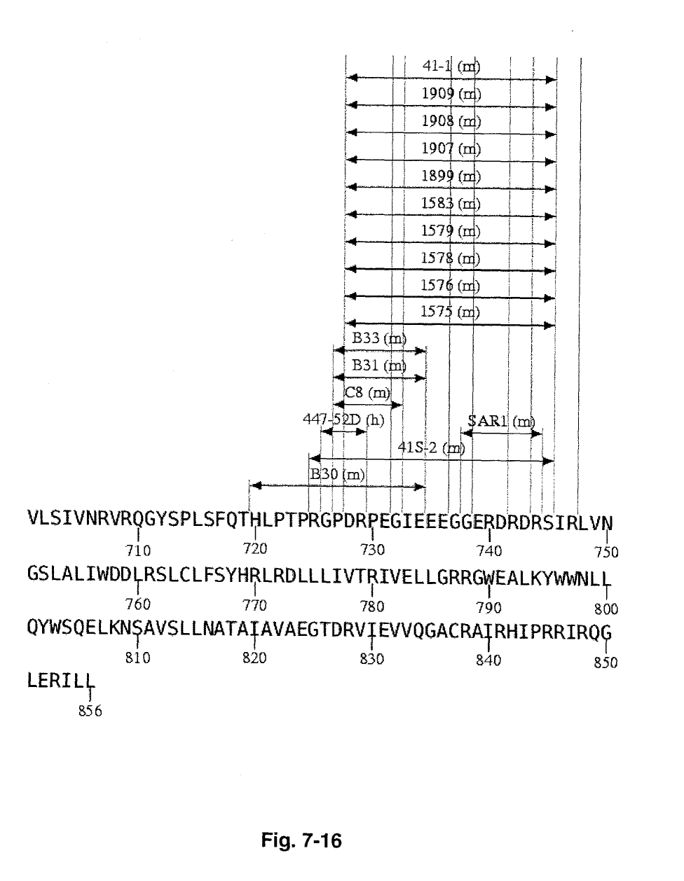

In specific embodiments, the epitope recognized by the HIV-1 neutralizing antibody is found in FIG. 7 titled “gp160 Ab Epitope Map”. The names of MAbs and the location of well-characterized binding sites of 21 amino acids or less are indicated relative to the protein sequences of the HXB2 clone. This map is meant to provide the relative location of epitopes on a given protein, but the HXB2 sequence may not actually bind to the MAb of interest, as it may vary relative to the sequence for which the epitope was defined. Above each binding site, the MAb name is given followed by the species in parenthesis. Human is represented by “h”, non-human primate by “p”, mouse by “m”, and others by “o”.

It is further recognized that immunologically equivalent epitopes for the HIV-1 neutralizing antibodies discussed above are known and can be used in the methods and compositions of the invention. Immunologically equivalent epitopes for 2F5 are known. See, for example, Zwick et al. (2001) J. Virology 75:10892-10900, which disclose immunologically equivalent epitopes of the 2F5 epitope. Such immunologically equivalent epitopes, while differing in their amino acid sequence, continue to be recognized by the 2F5 monoclonal antibody. Immunologically equivalent epitopes for 4E10 are also known. See, for example, Zwick et al. (2001) J. Virology 75:10892-10900. Again, such immunologically equivalent epitopes, while differing in their amino acid sequence continue to be recognized by the 4E10 monoclonal antibody. Accordingly, immunologically equivalent epitopes can differ from the native epitope by at least 1, 2, 3, 4, 5, 6, 7, 8 or more amino acids. The differences can be generated by amino acid substitutions, deletions and insertions. Methods to determine if two epitopes are immunologically equivalent are known in the art. See, for example, Zwick et al. (2001) J. Virology 75:10892-10900.

Exemplary chimeric polynucleotides and polypeptides of the invention include sequences encoding non-HIV polypeptides, or variants thereof, which have been modified to have a heterologous HIV-1 2F5, 2G12, b12, 4E10, or Z13 epitope or a functional variant (immunologically equivalent epitope) thereof as discussed elsewhere herein.

### Immunogenic Compositions

Immunogenic compositions of the invention can include an isolated chimeric polypeptide or active variant thereof or an isolated polynucleotide encoding the chimeric non-HIV polypeptide of the invention or active variant thereof. An isolated chimeric non-HIV polypeptide of the invention is present in an immunogenic composition in an amount sufficient to elicit an immune response against the heterologous epitope upon administration of a suitable dose to a subject. An isolated chimeric polynucleotide encoding a chimeric non-HIV polypeptide of the invention can also be present in the immunogenic composition in an amount sufficient such that administration of a suitable dose to a subject results in the expression of the encoded chimeric non-HIV polypeptide, which stimulates an immune response against the heterologous HIV-1 epitope. As used herein, a “subject” is defined as any animal including any mammal, such as, rodents, rabbits, goats, sheep, non-human primates, humans etc.

### Immunogenic Compositions Comprising the Chimeric Non-HIV Polypeptide or Polynucleotide Encoding Therefor

The invention provides immunogenic compositions comprising a chimeric non-HIV polypeptide of the invention or an active variant or fragment thereof. In one embodiment, an immunogenic composition of the invention includes cells expressing a chimeric non-HIV polypeptide of the invention, a cell lysate, or a fraction thereof, containing the chimeric polypeptide, such as, e.g., a membrane fraction. In other embodiments, the immunogenic composition comprises an isolated chimeric non-HIV polypeptide or variant thereof. These are known as “subunit” vaccines because they constitute only a component part of the HIV. These “subunit vaccines” can prompt the body to produce an anti-HIV immune response.

In other embodiments, the immunogenic chimeric non-HIV polypeptide or active variant thereof can be provided as a virus-derived vaccine. As used herein, the term “virus-derived vaccine” refers to a vaccine containing a recombinantly engineered non-HIV virus that either does not cause disease in human or has been deliberately weakened so that it cannot cause disease. There weakened (attenuated) viruses are used as vectors, or vehicles, to deliver copies of HIV genes into the cells of the body. Once inside cells, the body uses the instructions carried in the copies of HIV genes to produce HIV proteins. These HIV proteins can stimulate an anti-HIV immune response.

Virus-derived vaccines have been prepared using a canarypox virus, a vaccinia virus, the alphavirus VEE, and a replication-defective adenovirus or adenovirus. Other viruses that can be engineered to produce recombinant viruses useful in vaccines include retroviruses that are packaged in cells with amphotropic host range, and attenuated or defective DNA viruses, such as, but not limited to, herpes simplex virus (HSV), papillomavirus, Epstein Barr virus (EBV), adeno-associated virus (AAV), poxvirus, and the like.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Preferred embodiments include sustained-release compositions. An exemplary sustained-release composition has a semi permeable matrix of a solid hydrophobic polymer to which the polypeptide is attached or in which the polypeptide is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

Exemplary sustained release compositions include polypeptides attached, typically via ε-amino groups, to a polyalkylene glycol (e.g., polyethylene glycol [PEG]). Attachment of PEG to proteins is a well-known means of extending in vivo half-life. Any conventional “pegylation” method can be employed, provided the “pegylated” variant retains the desired function(s).

In another embodiment, a sustained-release composition includes a liposomally entrapped polypeptide. Liposomes are small vesicles composed of various types of lipids, phospholipids, and/or surfactants. These components are typically arranged in a bilayer formation, similar to the lipid arrangement of biological membranes. Liposomes containing polypeptides are prepared by known methods.

Immunogenic compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

Immunogenic Compositions Comprising Polynucleotides Encoding the Chimeric Non-HIV Polypeptides or Variants Thereof

An alternative to traditional immunization with a polypeptide antigen involves the direct in vivo introduction of a polynucleotide encoding the antigen into tissues of a subject for expression of the antigen by the cells of the subject's tissue. Polynucleotide-based compositions used to vaccinate a subject are termed “polynucleotide vaccines” or “naked DNA”. As used herein, the term “polynucleotide-vaccine” or a “naked DNA” is a vaccine containing one or more polynucleotides encoding an antigen, wherein administration of the polynucleotide to an organism results in expression of the encoded antigen, followed by an immune response to that antigen. Accordingly, an immunogenic composition comprising a chimeric polynucleotide encoding a chimeric non-HIV polypeptide or variant thereof is provided. Such compositions can include other components including, for example, a storage solution, such as a suitable buffer, e.g., a physiological buffer. In another embodiment, the other component is a pharmaceutically acceptable carrier as described above. The use of polynucleotide vaccines is well known to those skilled in the art.

In other embodiments, the composition comprising the polynucleotide encoding the chimeric non-HIV polypeptide further includes a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with the polynucleotides of the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidyicholine (DOPC). Polynucleotides can also be entrapped in liposomes, as described above for polypeptides.

In another embodiment, polynucleotides are complexed to dendrimers, which can be used to transfect cells. Dendrimer polycations are three dimensional, highly ordered oligomeric and/or polymeric compounds typically foamed on a core molecule or designated initiator by reiterative reaction sequences adding the oligomers and/or polymers and providing an outer surface that is positively charged. Suitable dendrimers include, but are not limited to, “starburst” dendrimers and various dendrimer polycations. Methods for the preparation and use of dendrimers to introduce polynucleotides into cells in vivo are well known to those of skill in the art.

Accordingly, the chimeric polynucleotide of the invention can be provided in an expression cassette for expression in a cell. This section is also applicable to virus-derived vaccines. The cassette can include 5′ and 3′ regulatory sequences operably linked to the chimeric polynucleotide of the invention. “Operably linked” is intended to mean a functional linkage between two or more elements. For example, an operable linkage between a chimeric polynucleotide and a regulatory sequence (i.e., a promoter) is a functional link that allows for expression of the chimeric polynucleotide. Operably linked elements may be contiguous or non-contiguous. When used to refer to the joining of two protein coding regions, by operably linked is intended that the coding regions are in the same reading frame. The cassette may additionally contain at least one additional gene to be cotransformed into the cell of interest. Such an expression cassette is provided with a plurality of restriction sites and/or recombination sites for insertion of the chimeric polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a chimeric polynucleotide of the invention, and a transcriptional and translational termination region (i.e., termination region) functional in the cell type of interest. The regulatory regions (i.e., promoters, transcriptional regulatory regions, and translational termination regions) and/or the chimeric polynucleotide of the invention may be native/analogous to the host cell or to each other. Alternatively, the regulatory regions and/or the chimeric polynucleotide of the invention may be heterologous to the host cell or to each other.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As is well known in the art, a large number of factors can influence the efficiency of expression of antigen genes and/or the immunogenicity of gene-based vaccines. Examples of such factors include the vector, the promoter used to drive antigen gene expression, and the stability of the inserted gene in the plasmid. Depending on their origin, promoters differ in tissue specificity and efficiency in initiating mRNA synthesis. Many DNA vaccines in mammalian systems have relied upon viral promoters derived from cytomegalovirus (CMV).

Additional Components of Immunogenic Compositions

Compositions comprising the polynucleotides or polypeptides can be stored in any standard form, including, e.g., an aqueous solution or a lyophilized cake. Such compositions are typically sterile when administered to cells or subjects. Sterilization of an aqueous solution is readily accomplished by filtration through a sterile filtration membrane. If the composition is stored in lyophilized form, the composition can be filtered before or after lyophilization and reconstitution.

The various immunogenic compositions of the invention can include one or more adjuvant. The term “adjuvant” refers to a compound or mixture that enhances the immune response to an antigen. Exemplary adjuvants include, but are not limited to, Adju-Phos, Adjumer™, albumin-heparin microparticles, Algal Glucan, Algammulin, Alum, Antigen Formulation, AS-2 adjuvant, autologous dendritic cells, autologous PBMC, Avridine®, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-ProteinA D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-γ, Interleukin-1β, Interleukin-12, Interleukin-2, Interleukin-7, ISCOM(s)™, Iscoprep 7.0.3.™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be vaccinated. Preferably, a pharmaceutically acceptable adjuvant is used.

### Methods

The immunogenic compositions of the invention can be employed to generate antibodies that recognize the chimeric non-HIV polypeptide of the invention. The method comprises administering to a subject an immunogenic composition comprising a chimeric non-HIV polypeptide of the invention or administering to the subject a polynucleotide encoding a chimeric non-HIV polypeptide of the invention. As outlined in detail below, immunogenic compositions of the invention can be administered to the subject by any suitable route of administration. Accordingly, in one embodiment, an immunogenic composition is administered to a subject to generate antibodies that recognize the heterologous HIV-1 neutralizing epitope. Such antibodies find use in HIV research. Generally, the subject employed in this embodiment is one typically employed for antibody production. Mammals, such as, rodents, rabbits, goats, sheep, etc., are preferred.

The antibodies generated can be either polyclonal or monoclonal antibodies. Polyclonal antibodies are raised by injecting (e.g. subcutaneous or intramuscular injection) antigenic polypeptides into a suitable animal (e.g., a mouse or a rabbit). The antibodies are then obtained from blood samples taken from the animal. The techniques used to produce polyclonal antibodies are extensively described in the literature. Polyclonal antibodies produced by the subjects can be further purified, for example, by binding to and elution from a matrix that is bound with the polypeptide against which the antibodies were raised. Those of skill in the art will know of various standard techniques for purification and/or concentration of polyclonal, as well as monoclonal, antibodies. Monoclonal antibodies can also be generated using techniques known in the art.

In other methods, the immunogenic compositions of the invention can be used to elicit an immune response in a subject. The method comprises introducing into the subject an effective concentration of an immunogenic composition comprising a chimeric non-HIV polypeptide of the invention or active variant thereof. In further embodiments, the method comprises administering an immunogenic composition comprising a polynucleotide that encodes a chimeric non-HIV polypeptide of the invention or a variant thereof and expressing the chimeric polynucleotide in the subject.

In other methods, the immunogenic compositions of the invention can be used as vaccines. In one method, the immunogenic composition is administered to individuals who are not infected with HIV-1 to reduce the risk of, or prevent, infection (prophylaxis of HIV-1 infection). The immunogenic composition can also be administered to individuals who are already infected with HIV-1, but are still able to mount an immune response. A so-called “therapeutic vaccine” can ameliorate the existing infection (for example, by improving the subject's condition or slowing or preventing disease progression) and/or can provide prophylaxis against infection with additional HIV-1 strains. Accordingly, methods for inhibiting or preventing infection by HIV-1 in a subject are provided. This method comprises administering to the subject an effective concentration of an immunogenic composition comprising the chimeric non-HIV polypeptide of the invention or active variant thereof. In further embodiments, the method comprises administering an immunogenic composition comprising a polynucleotide that encodes a chimeric non-HIV polypeptide of the invention, and expressing the chimeric polynucleotide in the subject.

Polypeptide-based immunogenic compositions are conveniently administered by injection (e.g., subcutaneous, intradermal, intramuscular, intraperitoneal, intravenous, etc.). Alternative routes include oral administration (tablets and the like) and inhalation (e.g., using commercially available nebulizers for liquid formulations or lyophilized or aerosolized formulations). Polypeptide compositions may also be administered via microspheres, liposomes, immune-stimulating complexes (ISCOMs), or other microparticulate delivery systems or sustained release formulations introduced into suitable tissues (such as blood).

As discussed above, polynucleotide-based immunogenic compositions of the invention can be employed to express an encoded polypeptide in vivo, in a subject, thereby eliciting an immune response against the encoded polypeptide. Various methods are available for administering polynucleotides into animals. The selection of a suitable method for introducing a particular polynucleotide into an animal is within the level of skill in the art. Polynucleotides of the invention can also be introduced into a subject by other methods known in the art, e.g., transfection, electroporation, microinjection, transduction, cell fusion, DEAE dextran, calcium phosphate precipitation, lipofection (lysosome fusion), or a DNA vector transporter (see, e.g., Wu et al. (1992) J. Biol. Chem. 267:963-967).

An “effective concentration” is defined herein as an amount of a biologically active agent that produces an intended biological activity. The effective concentration of either the chimeric non-HIV polypeptide or the chimeric non-HIV polynucleotide administered in the immunogenic composition depends on the properties of the particular composition, e.g., the immunogenicity of a particular formulation, administration route, immunization regimen, condition of the subject and the like, and the determination of a suitable dose for a particular set of circumstances is within the level of skill in the art. Different dosages can be used in a series of sequential inoculations. Thus, the practitioner may administer a relatively large dose in a primary inoculation and then boost with relatively smaller doses of the chimeric non-HIV polypeptide.

The immune response against the heterologous epitope of the chimeric polypeptide can be generated by one or more inoculations of a subject with an immunogenic composition of the invention. A first inoculation is termed a “primary inoculation” and subsequent immunizations are termed “booster inoculations”. Booster inoculations generally enhance the immune response, and immunization regimens including at least one booster inoculation are preferred. Any type of immunogenic composition described above may be used for a primary or booster immunization. Thus, for example, an immunogenic composition comprising polynucleotides (e.g., or a virus-derived vaccine) of the invention can be used for a primary immunization, followed by boosting with an immunogenic composition containing polypeptides of the invention, or vice versa. In addition, a primary immunization and one or more booster immunization can provide the same chimeric polypeptide and/or different chimeric polypeptides.

In one embodiment, a suitable immunization regimen includes at least three separate inoculations with one or more immunogenic compositions of the invention, with a second inoculation being administered more than about two, about three to eight, or about four, weeks following the first inoculation. Generally, the third inoculation is administered several months after the second inoculation, and in specific embodiments, more than about five months after the first inoculation, more than about six months to about two years after the first inoculation, or about eight months to about one year after the first inoculation. Periodic inoculations beyond the third are also desirable to enhance the subject's “immune memory.”

The adequacy of the vaccination parameters chosen, e.g., formulation, dose, regimen and the like, can be determined by taking aliquots of serum from the subject and assaying antibody titers during the course of the immunization program. Alternatively, the T cell populations can by monitored by conventional methods. In addition, the clinical condition of the subject can be monitored for the desired effect, e.g., prevention of HIV-1 infection or progression to AIDS, improvement in disease state (e.g., reduction in viral load), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, the dose of the chimeric non-HIV polypeptide or polynucleotide and/or adjuvant can be increased or the route of administration can be changed.

Methods are further provided for a diagnostic assay to monitor HIV-induced disease in a subject and/or to monitor the response of the subject to immunization by an HIV vaccine. By “HIV-induced disease” is intended any disease caused, directly or indirectly, by HIV. An example of an HIV-induced disease is acquired autoimmunodeficiency syndrome (AIDS). The method comprises providing a chimeric non-HIV polypeptide or a functional variant thereof where the chimeric non-HIV polypeptide further comprises at least one heterologous epitope recognized by an HIV-1 binding antibody (i.e., binding, neutralizing, CD4-induced). The chimeric non-HIV polypeptide is contacted with an amount of bodily fluid from the subject; and, the HIV-1 binding antibodies in the bodily fluid of the subject are detected. The detection of the HIV-1 binding antibodies allows the HIV disease in the subject to be monitored. In addition, the detection of the HIV-1 binding antibody also allows the response of the subject to immunization by a HIV vaccine to be monitored. In still other methods, the titer of the HIV-1 binding antibodies is determined.

Additional methods include an assay to isolate additional HIV-1 binding antibody (i.e., having the epitope that the HIV-1 binding antibody interacts with). The method comprises providing a chimeric non-HIV polypeptide or a variant thereof, which comprises a heterologous epitope recognized by an HIV-1 binding antibody and contacting the chimeric non-HIV polypeptide with a composition comprising a candidate HIV-1 binding antibody. Assays are performed to determine if the candidate HIV-1 binding antibody recognizes the HIV-1 epitope present in the chimeric non-HIV polypeptide. In this manner, one can identify the candidate HIV-1 binding antibody. Methods are also known to isolate candidate HIV-binding antibodies from a variety of sources including naive libraries, modified libraries, and libraries produced directly from human donors exhibiting an HIV-specific immune response.

### Generating Broadly Neutralizing Antibodies

In the absence of natural immunity to the AIDS virus, scientific understanding of the disease drives an approach to vaccine development known as “rational vaccine design.” Critical to the development of a successful AIDS vaccine will be our ability to elicit broadly neutralizing antibodies that inactivate diverse viral strains and to generate strong CD4⁺ and CD8⁺ T cell immune responses. Success in eliciting broadly neutralizing antibodies has been limited to date.

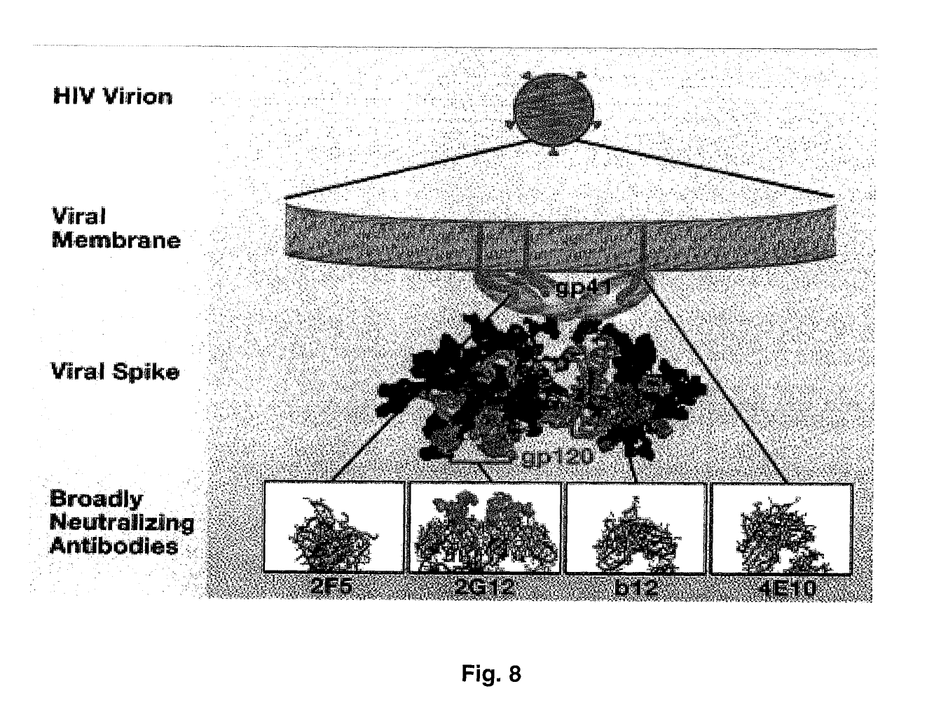

Structural and antigenic characterization of the HIV-1 envelope reveals unprecedented mechanisms for evading the host antibody response. The viral spike is composed of three gp120-gp41 glycoproteins. It binds to CD4 and a coreceptor on the host T cell surface and promotes fusion of HIV-1 and host-cell membranes, enabling virus entry (FIG. 8). Much of its exposed surface is cloaked by N-linked glycan, which is produced by the host cellular machinery and is largely unrecognized by the immune system. This glycan surface provides an evolutionarily efficient means of escape from neutralizing antibodies; a small number of mutations can give rise to significant changes in glycan structures that confer resistance to neutralization. The virus uses other evasive mechanisms: immunodominant regions that are occluded in the native oligomeric spike protein of the virus are exposed in viral debris or in inactive forms of the spike protein. These immunodominant regions generate HIV-specific antibodies, which do not bind to the functional spike. Conformational masking also contributes to the resistance of the virus to neutralizing antibodies. The coreceptor binding site on gp120 of HIV is highly conserved, and neutralizing antibodies develop readily against it. However, on functional viral spikes, the potentially susceptible site of coreceptor binding is formed only after attachment of gp120 to CD4 on the host-cell surface, preventing access of neutralizing antibodies to an otherwise highly conserved binding site. When these mechanisms of humoral evasion are coupled to the extraordinary natural diversity of the virus, the task of generating high titers of broadly reactive, neutralizing antibody in vaccine subjects is daunting.

Referring to FIG. 8, shown is the trimeric HIV-1 spike protein composed of three gp41 subunits, three gp120 core units, N-linked carbohydrate, and sites vulnerable to potential antibody-mediated neutralization. Broadly neutralizing antibodies against HIV interfere with the CD4 binding site of gp120 (antibody b12), the carbohydrate determinants of the spike (antibody 2G12), or conserved domains in the membrane-proximal region of gp120, which mediate fusion of the viral envelope with the target-cell membrane (antibodies 2F5 and 4E10). In the orientation shown, the left two gp120 molecules overlap, and a protomeric core is clearly seen in the rightmost gp120. Note that HIV-1 gp120 is shown here in its CD4 bound conformation; the structure of unliganded SIV gp120 demonstrates that considerable conformational reorganization occurs upon binding of gp120 to the CD4 receptor of host T cells. Inset figures show structures of broadly neutralizing antibodies and, where known, their HIV-1 epitopes.

Fortuitously, the technologies that reveal the challenges of eliciting such antibodies provide insights into potential vulnerabilities. Monoclonal antibody and phage display analyses have identified a few broadly neutralizing antibodies. For example, antibodies such as 2F5, 4E10, 2G12, and b12 neutralize a significant percentage of circulating HIV-1 primary isolates, and their molecular structures and targets are now well characterized (FIG. 8). Why are these antibodies effective?

One answer may be that they recognize functionally constrained, conserved, and exposed structures—that is, the viral spike must find a receptor and then fuse viral and target-cell membranes. These twin functions of “finding” and “fusing” provide constraints on the viral spike, which may be recognized by such antibodies as b12 (CD4 binding) or 2F5 and 4E10 (membrane fusion). The functional rationale for conservation of the 2G12 carbohydrate epitope, which is largely limited to clade B viruses, is less clear and may relate to preserving advantageous interactions with the innate immune system (for example, interaction with the carbohydrate binding receptor DC-SIGN) or constraints on carbohydrate density related to glycan shielding.

The information derived from structural analysis of broadly neutralizing monoclonal antibodies informs vaccine design. Precise characterization of the structures recognized by these antibodies is the first step in creating polypeptides or small molecules that mimic such epitopes. To this end, significant effort has been made to gain an atomic-level understanding of susceptible epitopes and their interaction with neutralizing antibodies. The guiding hypothesis is that the proper presentation of a functionally conserved, susceptible epitope will lead to the elicitation of antibodies that recognize the target epitope and neutralize the virus. To overcome the conformational flexibility of the HIV envelope protein, modern tools of protein design can be used to create mutations that fix gp120 into the form recognized by the CD4 receptor or by broadly neutralizing antibodies. To help focus the immune response, one can remove immunodominant regions, thus paring the envelope to critically conserved regions of the core or outer domain, or one can mask immunodominant regions with carbohydrate to make them immunologically silent. Another strategy regarding epitope presentation involves the creation of epitope-transplant scaffolds. In this scaffolding strategy, the target epitope is transplanted into a foreign scaffold that replicates both the conformation and the surface accessibility of the epitope as recognized by a broadly neutralizing antibody. These approaches apply structural information to vaccine design. Until now, this process remained a working model.

Whether immunogens created by epitope mimicry will allow antibodies to be elicited with properties similar to the original broadly neutralizing antibody will depend on a number of variables: the uniqueness of the template antibody, the degree of structural mimicry between epitope mimetic and antibody bound epitope, and the ability of the humoral immune system to recreate specific immune responses. The tools of conformational stabilization, epitope focusing, and scaffold transplantation have much to contribute to rational vaccine design.

### Epitope-Transplant Scaffolds and Their Design

Several antibodies with broad and potent neutralizing activity against HIV-1 have been characterized. By using a combination of in silico design coupled to feedback from X-ray crystallography, antigenic analysis, and immunizations, we show how to transplant the HIV-1 epitope recognized by the broadly neutralizing antibodies into an appropriate scaffold, while preserving its structure and antigenicity. Immunization with these epitope-transplant scaffold or scaffolds should help to facilitate the re-elicitation of the antibodies with broadly neutralizing characteristics, similar to the template antibody. Such epitope-transplant scaffolds may serve as the basis of an effective HIV-1 vaccine. They should also serve as valuable diagnostics, to identify specifically serum reactivities against the target HIV-1 epitopes. Such scaffolding technology could be applied not only to HIV-1, but to any virus for which a broadly neutralizing antibody and its respective epitope has been characterized at the atomic-level.

Possible uses of the epitope-transplant scaffolds include:
As immunogens that elicit antibodies similar to the template antibody,
As an effective vaccine against HIV-1 or other viruses,
As a diagnostic to decipher the humoral immune response, and
As a screening tool to isolate additional antibodies with activities similar to the template antibody.

### General Design of Epitope-Transplant Scaffolds

We have developed novel computational protocols for structure-based design of immunogens that present specific epitopes within different protein scaffolds. We refer to these protein immunogens as epitope-scaffolds because they contain one or more epitopes embedded in a scaffold. To design epitope-scaffolds, we start with a crystal structure of the epitope or of the epitope in complex with the antibody. The basic strategy is to design proteins that stabilize the crystallized conformation of the epitope and present it to the antibody without steric clash or any other interaction.

We have conceived of three different approaches to the design of epitope-scaffolds. The three approaches are “superposition”, “grafting”, and “de novo”. We now give a brief description of these three methods, then below we provide a highly detailed description of protocols for the first method, superposition.

“Superposition” epitope-scaffolds are based on scaffold proteins having an exposed segment with similar conformation as the target epitope—the backbone atoms in this “superposition-region” can be structurally superposed onto the target epitope with minimal root mean square (rms) deviation of their coordinates. Suitable scaffolds are identified by computationally searching through a library of protein crystal structures; epitope-scaffolds are designed by putting the epitope residues in the superposition region and making additional mutations on the surrounding surface of the scaffold to prevent clash or other interactions with the antibody. The main advantage of superposition is that it is most conservative in terms of design; these epitope-scaffolds require only a limited number of mutations on the surface of known, stable proteins, so the designs can be produced rapidly and a high fraction of the first round designs are likely to fold properly. The main disadvantage is that the superposition method is limited to simple, continuous epitopes, because finding superposition matches to complex epitopes is unlikely.

“Grafting” epitope-scaffolds utilize scaffold proteins that can accommodate replacement of an exposed segment with the crystallized conformation of the target epitope. For each suitable scaffold identified by computationally searching through all protein crystal structures, an exposed segment is replaced by the target epitope and the surrounding sidechains are redesigned (mutated) to accommodate and stabilize the inserted epitope. Finally, as with superposition epitope-scaffolds, mutations are made on the surface of the scaffold and outside the epitope, to prevent clash or other interactions with the antibody. Grafting scaffolds require that the replaced segment and inserted epitope have similar translation and rotation transformations between their N- and C-termini, and that the surrounding peptide backbone does not clash with the inserted epitope. One difference between grafting and superposition is that grafting attempts to mimic the epitope conformation exactly, whereas superposition allows for small structural deviations. Grafting epitope-scaffolds should in principle perfectly mimic the epitope-antibody interaction. Another asset of grafting is that it can be used to treat complex epitopes. The disadvantage of grafting relative to superposition is that grafting requires making a larger number of mutations to the scaffold protein, including mutations in the core, so the designs take more time and a lower fraction of first round designs are expected to fold properly.

“De novo” epitope-scaffolds are computationally designed from scratch to optimally present the crystallized conformation of the epitope. This method follows directly from our design of a novel fold (Kuhlman, B. et al. 2003 Science 302:1364-1368). The de novo method is highly promising for immunogen design because it will allow us to design immunogens that are both minimal in size, so they do not present too many unwanted epitopes, and also highly stable against thermal or chemical denaturation, so they could be employed in a real-world vaccine.

### Superposition Protocols Used to Generate Epitope-Scaffolds

Three protocols, of increasing sophistication, have been used to design superposition scaffolds. Here we describe the main superposition protocol and we note where the protocols differ. Where not specified, all protocols used the same methodology.

(1) Obtain starting information in a format that can be automatically searched by computer:

a) crystal structure of an antibody-epitope complex, and

b) database of protein crystal structures (candidate scaffolds).

Protocols 2 and 3 used the entire PDB (protein data bank) for the database but protocol 1 used a non-redundant selection of the PDB.

(2) Identify all possible sub-ranges of the epitope that might be immunogenically effective on the surface of a scaffold. The useful sub-ranges can be assessed by examining the important epitope-antibody contacts in the crystal structure, and by consulting the literature for relevant data such as alanine-scanning or neutralization of pseudo-viruses. For example, for the 2F5 epitope, with atomic coordinates defined for 14 residues, we focused on 14 different sub-ranges: full length: 1-14; two 13-mers: 1-13, 2-14; three 12-mers: 1-12, 2-13, 3-14; four 11-mers: 1-11, 2-12, 3-13, 4-14; four 10-mers: 2-11, 3-12, 4-13, 5-14; three 9-mers: 3-11, 4-12, 5-13.

(3) For each sub-range of the epitope identified in (2), and for all candidate scaffolds in the database of crystal structures in (1b), carry out the following procedure:

a. Identify structural matches on scaffold surface. For all possible contiguous sub-ranges of the candidate-scaffold that are on the surface of the candidate-scaffold, superpose epitope backbone atoms onto candidate-scaffold backbone atoms and record the rms (root mean square deviation) of the superposition. Whether a residue is on the surface or not is assessed based on the number of neighbors with centroid-centroid distance less than a cutoff. If the superposition_rms/nsuperposed_residues is less than a predetermined cutoff, then this sub-range of the candidate-scaffold is a possible structural match and we proceed to 3b.

b. Filter out scaffolds that clash with antibody. Construct a model of the scaffold-antibody complex in which the scaffold-antibody rigid-body orientations are set by the crystal structure in (1a) and the superposition of scaffold onto epitope. Mutate all residues in the scaffold to glycine, but retain all sidechains of the antibody in their native conformations. Measure the clash (van der waal's repulsive) across the antibody-scaffold_all_gly interface. If this clash is below a pre-determined threshold then proceed to 3c.

c. Optimize epitope-antibody interactions. Transplant epitope sidechains to the structurally aligned positions on the scaffold, but leave the rest of the scaffold as glycine. Optimize the interaction between antibody and epitope sidechains by either: (protocol 1) “repacking” the sidechains of epitope and antibody at the interface, where “repacking” means allowing sidechain conformations to vary among discrete rotamers using a backbone-dependent rotamer library from Dunbrack and Cohen (Dunbrack, R. L. Jr. and Cohen, F. E. 1997 Protein Sci 6:1661-1681) as previously used in ROSETTA (Kuhlman, B. and Baker, D. 2000 Proc Natl Acad Sci USA 97:10383-10388); or (protocol 2) “minimizing” and then “repacking” the sidechains of epitope and antibody at the interface, where “minimizing” means allowing chi angles to vary continuously around their starting values until a local minimum in the total energy is found. (Gray, J. J. et al. 2003 J Mol Biol 331:281-299); or (protocol 3) “minimizing” and then carrying out a “docking” procedure that includes simultaneous optimization of the rigid-body and sidechain conformations using Monte Carlo minimization. The “docking” procedure used for scaffold design is an updated version of the high-resolution refinement protocol described by Gray, J. J. et al 2003 J Mol Biol 331:281-299, and shown in FIG. 1b from same. The optimization in 3c is carried out multiple times for each sub-range of each protein chain that has passed previous filters on structural matching (3a) and minimal clash with antibody (3b).

d. Filter out scaffolds with poor binding energy. Carry out stages 3a-3c for all possible sub-ranges of all candidate-scaffolds. Rank all of the sub-ranges of all of the candidate-scaffolds by binding energy as assessed at stage 3c. (In protocol 1, several rankings were considered simultaneously, for rms, clash, and length of superposition; in protocols 2 and 3 the total binding energy was the sole rank). For all candidate-scaffolds with binding energy greater than a cutoff, proceed to the next stage, design.

e. Design non-epitope scaffold positions contacting the antibody or epitope. For each candidate-scaffold input to this stage, the candidate-scaffold will have all-glycine residues except in the epitope region. At this point we need to decide what residues to use for the rest of the scaffold. First, we simply put on all the native scaffold residues, in their native conformations, at non-epitope positions. Then we automatically identify two types of positions (“inter” and “intra” positions) that must be considered for design. “inter” positions are non-epitope positions in the scaffold that are contacting the antibody. To avoid contacts between the antibody and non-epitope positions on the scaffold, “inter” positions are flagged for redesign and the amino acids allowed are restricted to small amino acids, typically AGST. “intra” positions are non-epitope positions on the scaffold that are contacting the epitope but not contacting the antibody. “intra” positions are flagged for redesign and are allowed to be any amino acid, in an effort to stabilize the conformation of the epitope side-chains that has been optimized in 3c. Following automatic identification of the inter and intra positions, computational design is carried out using RosettaDesign (Kuhlman, B. et al. 2003 Science 302:1364-1368; Kuhlman, B. and Baker, D. 2000 Proc Natl Acad Sci USA 97:10383-10388). Multiple designs (typically 100) are computed for each candidate-scaffold and are ranked by binding energy to the antibody. In protocol 3 the designs were also ranked by scaffold stability in the absence of antibody.

f. Accumulate information about each specific scaffold protein that scores well post-design. Additional information about each scaffold can play an important role in selecting which scaffolds to pursue with experimental testing. We automatically accumulate the following information on each protein: name; species origin; size; oligomerization state according to the RCSB Biological Unit Database; oligomerization state according to the Protein Quaternary Server (PQS) at the European Bioinformatics Institutes (EBI); number of disulfide bonds; average B-factor for backbone atoms over the entire scaffolds and over the epitope region alone; hetero atoms present in the crystal structure of the native scaffold. These pieces of information are used to prioritize scaffolds for further consideration, and also to target some scaffold for further processing. For example, if a scaffold is actually oligomeric in solution (dimeric, trimeric, tetrameric, etc.), then we must perform additional testing to determine if the oligomer will clash with the antibody, or if we can make additional mutations to render the scaffold monomeric. Or, for another example, it is important to know whether a particular scaffold requires a ligand (small molecule) to maintain the desired scaffold structure.

g. Manual analysis and redesign of scaffolds. The last step in the design of scaffolds presenting epitopes is a manual step. The main goals of this final procedure are: prioritize scaffolds based on information automatically assessed in 3f; visual inspection and validation of scaffold structural stability and epitope-antibody interaction; revert mutations back to wild-type if possible; check the literature on each prioritized scaffold for additional considerations.

### Computational Protein Sequence Design Method

The computational protein sequence design method (Kuhlman, B. and Baker, D. 2000 Proc Natl Acad Sci USA 97:10383-10388) utilizes a 3D backbone template (fixed or flexible). In addition, the database consists of a library of approximately 150 rotamers for all amino acids at a given site (Dunbrack, R. L. Jr. and Cohen, F. E. 1997 Protein Sci 6:1661-1681). The method utilizes the Motropolis Monte Carlo Search Procedure that starts from a random sequence and random single amino acid/rotamer substitutions are made. If the energy is lower, the substitution is accepted. If the energy is higher, the substitution is accepted at a small probability, dependent on a Boltzman function. The process is repeated about a million times, with each Monte Carlo run lasting about 5 minutes. Independent runs converge to similar sequences.

The energy function takes into account 1) the Lennard-Jones Potential (favors atoms that are close, but not too close), 2) an implicit solvation model that penalizes buried polar atoms, 3) hydrogen bonding (allows buried polar atoms), 4) electrostatics derived from the probability of two charged amino acids being near each other in the PDB, 5) amino acid preferences for particular regions of Ramachandran space, 6) side chain dihedral angle preferences, and 7) unfolded state energy that assigns each amino acid type an average unfolded state energy.

### Design of Epitope-Scaffolds to Elicit Anti-HIV, Broadly Neutralizing Antibodies

Epitope-scaffolds are computationally designed to elicit anti-HIV, broadly neutralizing antibodies according to the following steps: 1) Display HIV epitope on non-HIV scaffold, 2) Stabilize desired epitope conformation, 3) Bury non-neutralizing face, 4) Eliminate scaffold-antibody interactions, 5) Utilize multiple scaffolds to vary immunogenic background, 6) Utilize dimeric (oligomeric) scaffolds, 7) Design N-linked glycoslylation sites to focus immune response, 8) Optimize thermal stability and solubility, and 9) Optimize other properties desired in a vaccine component.

### Elicitation of 2F5-Like, Broadly Neutralizing Antibodies

A goal is to elicit 2F5-like, broadly neutralizing antibodies. An outline of a scaffold design and testing scheme is as follows. A structural homology search of the protein database (PDB) is conducted (e.g., Cα trace search (MAMMOTH)). Lead hits are modified according to the Rosetta method to build the 2F5 epitope into a region of highest structural homology. The epitope-transplant scaffolds are tested for expression and refolding and binding kinetics to 2F5 are analyzed using Biacore. Binding may be compared to 2F5 binding with non-stabilized immunogen. Selection of scaffolds is based on 2F5 binding analysis. Positive epitope-transplant scaffolds are crystallized and structure solution is determined. The immunogenicity of the epitope-transplant scaffolds and neutralization capacity of antibodies generated against the epitope-transplant scaffolds are determined. To further optimize scaffolds, structural correlates with 2F5 binding, immunogenicity and neutralization are identified.

### The Superposition Protocol for 2F5-Epitope

a. Superpositions are found using ‘pepslide’ in Rosetta. Slide peptide over all chains in protein database (pdb), find superpositions with backbone. Exposed superpositions only. 141,189 superpositions for 2F5-epitope using PDB of 10 Sep. 2005.

b. Assess Ab/scaffold backbone clash when docked according to superposition (scaffold-all-GLY+Ab-all-atoms). Next check clash with biological unit (now automated). 1700 scaffolds thr 2F5-epitope with farep <30.

c. Assess binding energy of epitope-scaffold/Ab (all 1700). Epitope sidechains (native rotamers) onto all-GLY scaffold, Minimize sidechains.

d. Docking to find better rigid-body orientation for epitope-scaffold/Ab dock using pre-minimized structures—dock the best 100 by Eint post-docking: minimize again to use standard score 12.

e. Design scaffold (now automated—can design many scaffolds and rank all)
(1) minimize scaffold/Ab contact outside the epitope
(2) optimize intra-scaffold contacts between epitope sidechains and non-epitope. Playing with distance cutoff, want minimal mutations, rank 100+ complete epitope-scaffolds by Eint.

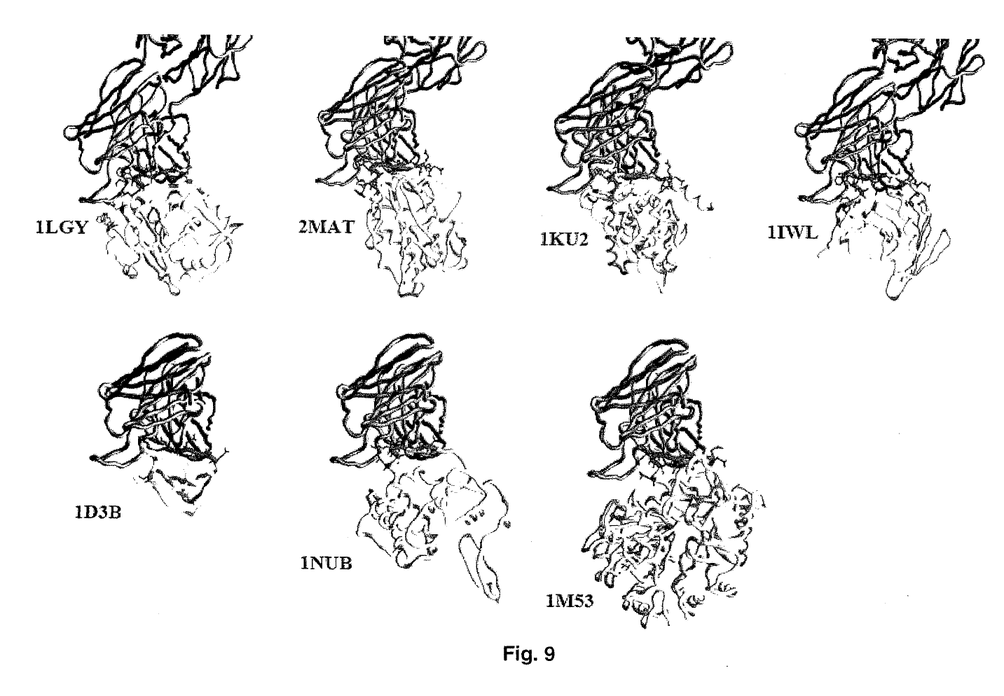

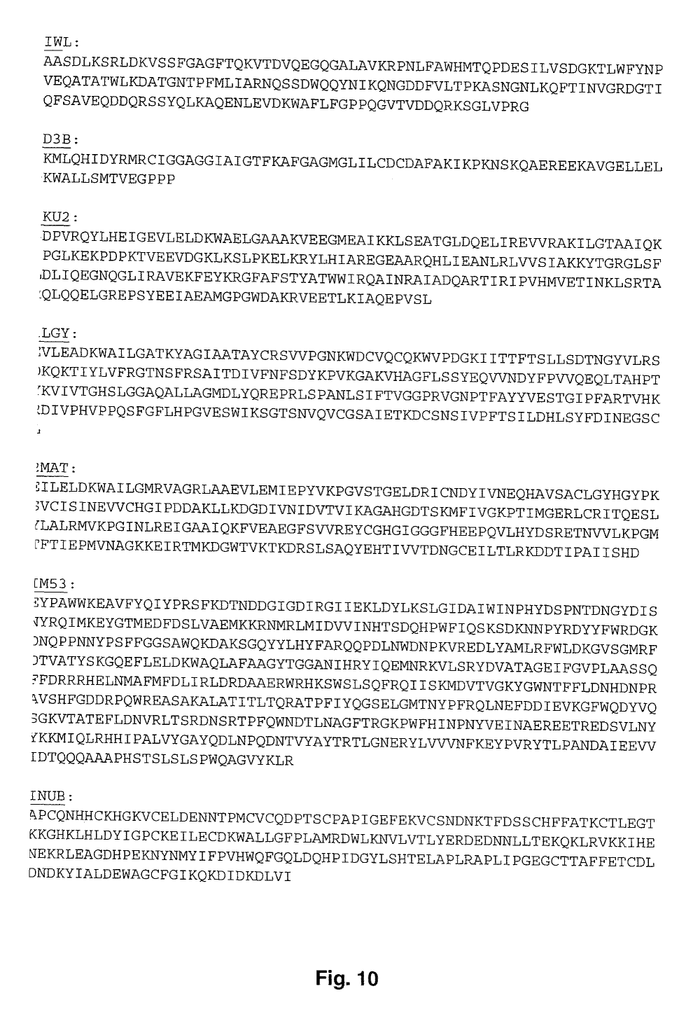

Using the Rosetta superposition protocol, seven initial scaffolds were identified (Table 1). Modeled structures of the epitope scaffolds are shown in FIG. 9. The gp41 scaffold amino acid sequences are given in FIG. 10.

### Structure-Base Stabilization of the 2F5 Antibody Epitope of the HIV-1 gp41 Envelope Glycoprotein for Immunogen Design

An essential component of an effective HIV-1 vaccine is the elicitation of neutralizing antibodies. One of the most broadly neutralizing antibodies is the 2F5 antibody that binds a contiguous epitope on the membrane proximal region (MPR), also called the membrane proximal external region (MPER), of the gp41 envelope glycoprotein. Various attempts to elicit 2F5-like antibodies by different immunogen design strategies have resulted in little to no neutralizing activity (Coeffier, E. et al. 2000 Vaccine 19:684-693). The elucidation of the crystal structure of the 2F5 antibody in complex with its cognate epitope provides new information to guide immunogen design (Ofek, G. et al. 2004 J Virol 78:10724-10723. Similarly, the elucidation of the crystal structure of the b12 antibody in complex with its cognate epitope provides new information to guide immunogen design (Zhou, T. et al. 2007 Nature 445:732-737). We believe that proper presentation of the epitope both by fixing the epitope in the conformation described by the crystal structure and also by occluding its non-binding hydrophobic face will allow us to attain elicitation of 2F5-like neutralizing antibodies. In the present study we have identified several non-HIV proteins of diverse origin that share structure homology with the MPR that can accommodate the 2F5 epitope in the extended conformation described by the crystal structure. We present here preliminary antigenicity and immunogenicity of selected 2F5 scaffolds.

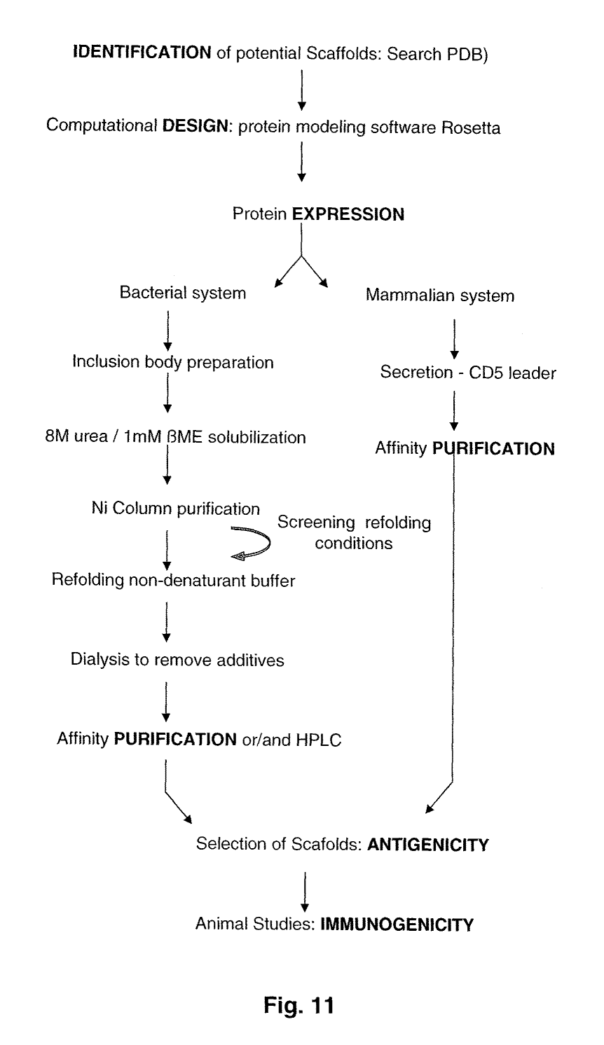

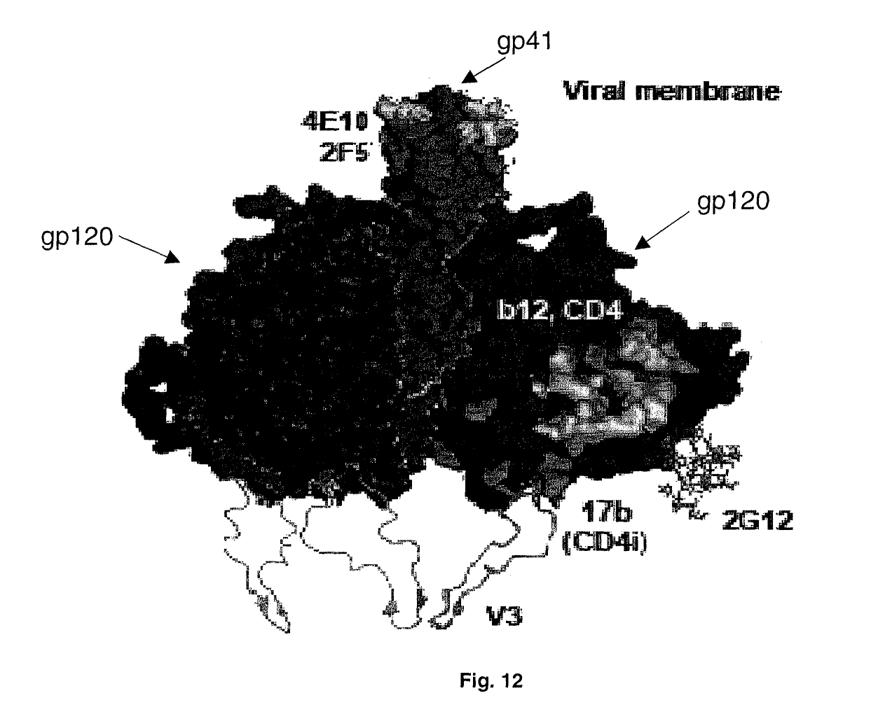

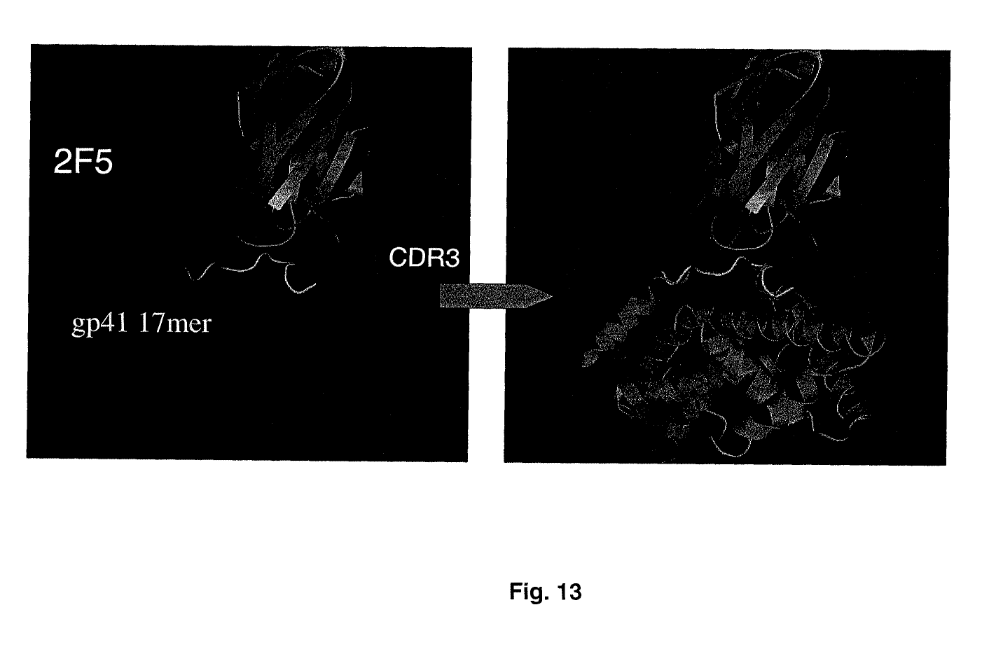

The aim is to elicit HIV-1 MPR-directed neutralizing antibodies. FIG. 11 is a flow-chart describing the basic scheme of the methodology from the identification and design of the scaffolds to antigenicity and immunogenicity. FIG. 12 depicts envelope glycoproteins gp41 and gp120 with the imprint of broadly neutralizing antibody binding sites, including the broadly neutralizing 2F5 antibody. FIG. 13 shows a molecular model of the 2F5 antibody bound to the carbon alpha trace of the gp41 17mer peptide. On the right 2F5e_scaffold_{—}1 and superimposed is the gp41 2F5 epitope in the extended conformation described by the crystal structure.

The results indicated that seven initial epitope scaffolds have been designed to accommodate the gp41 2F5 epitope in the bound conformation (Table 2). The original protein scaffolds have very diverse origins and functions and have undergone several mutations to both accommodate the epitope in the desired conformation and avoid potential clashes with a 2F5-like antibody.

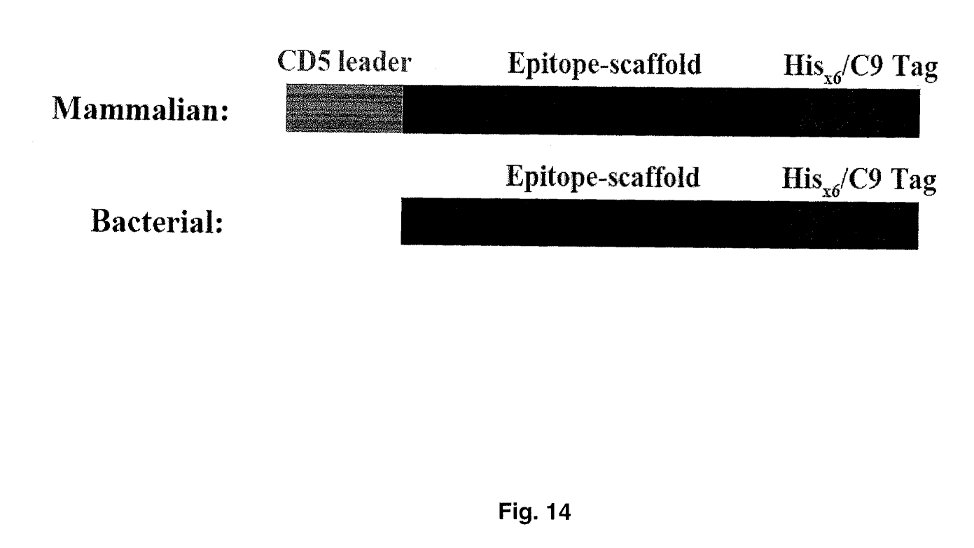

Expression vectors contain the nucleic acid sequences encoding the epitope-transplant scaffolds (FIG. 14). To enable purification, a tag (e.g., His_{X6}/C9 Tag) may be linked to the end of the epitope-transplant scaffold proteins. Mammalian expression vectors contain a leader peptide sequence to permit secretion, such as CD5 leader. The expression vectors are transiently transfected into mammalian cells such as 293 HEK cells. Surface plasmon resonance (SPR) may be used to check supernatants for binding to 2F5 antibodies. For those constructs that are not expressed/secreted/refolded properly in mammalian cells, a bacterial expression vector may be used. Following expression in a bacterial system, the epitope-transplant scaffold proteins are isolated and refolded from inclusion bodies.

Table 2 summarizes the expression systems used to express the seven initial 2F5 epitope-scaffold proteins and their refolding and binding properties. Three of these scaffolds expressed and refolded properly in the mammalian system, while the other four scaffolds were expressed in the bacterial system and underwent a screening process to determine refolding conditions. Five scaffolds bind 2F5 antibody as determined by ELISA and/or Biacore.

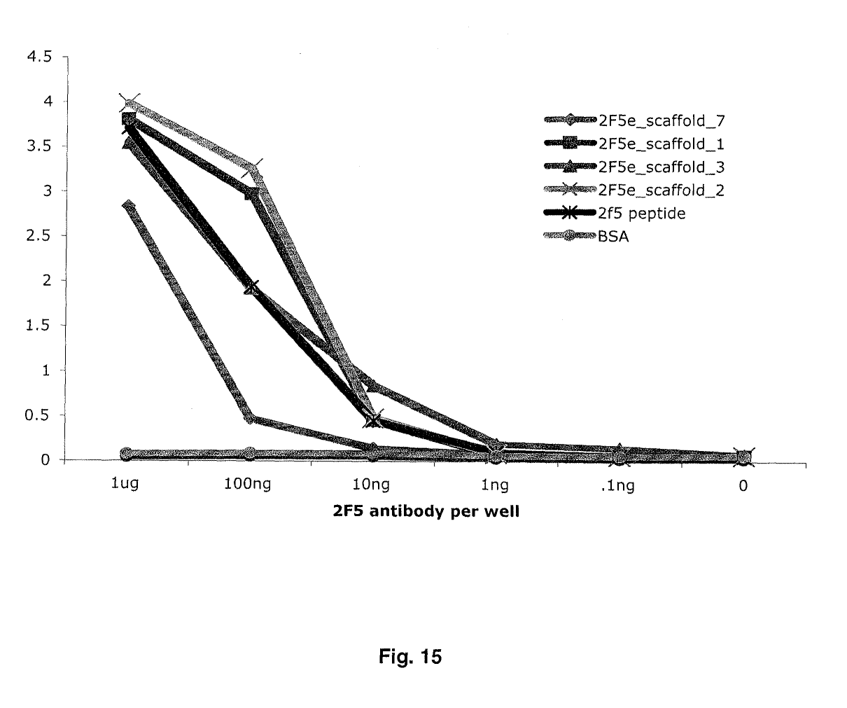

The binding of 2F5 antibody to scaffolds was investigated by ELISA. FIG. 15 shows ELISA data showing the relative binding of the antibody 2F5 to a series of 2F5 scaffolds. BSA and 2F5 peptide are the negative and positive controls respectively.

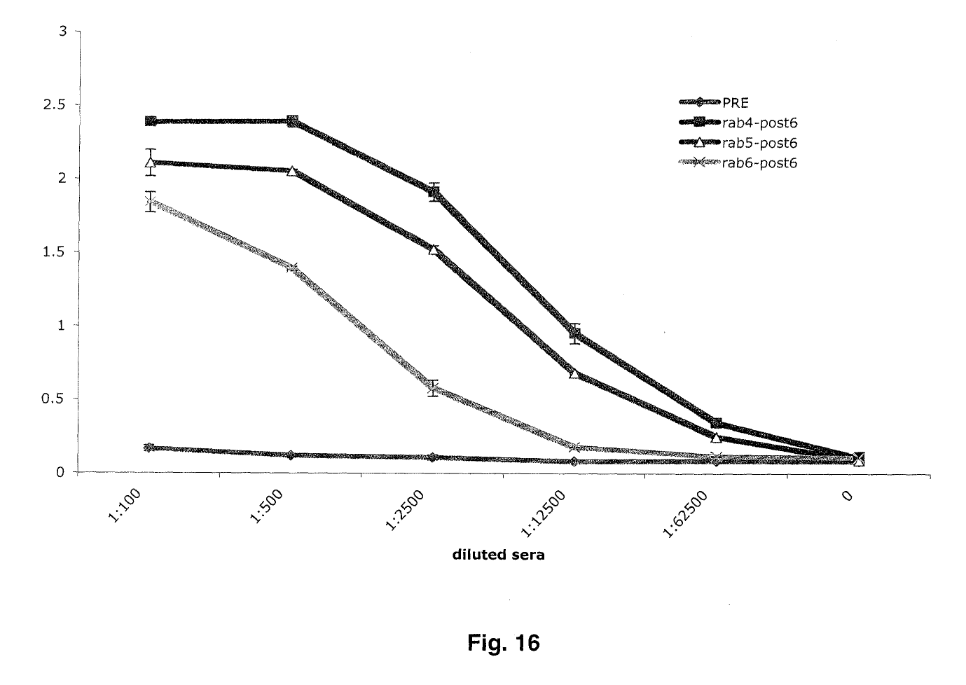

In a preliminary animal study, three rabbits were immunized sequentially with two scaffolds: 2F5e_scaffold_{—}1 and 2F5e_scaffold_{—}2, 4 times with each scaffold every two weeks. Bleeds were collected one week after each immunization. Scaffolds were immunogenic. Our approach of sequential immunizations with different scaffolds aims at focusing the response on the 2F5 epitope scaffolded while minimizing the responses against the irrelevant original scaffold. One way to detect these responses is to test the antisera for binding against a heterologous scaffold (one that the rabbits have not been immunized with) since only the 2F5 epitope and tags are shared among all the scaffolds. FIG. 16 summarizes ELISA data showing specific responses against a heterologous scaffold (2F5e_scaffold_{—}4 on the plate) the rabbits have not been immunized with.

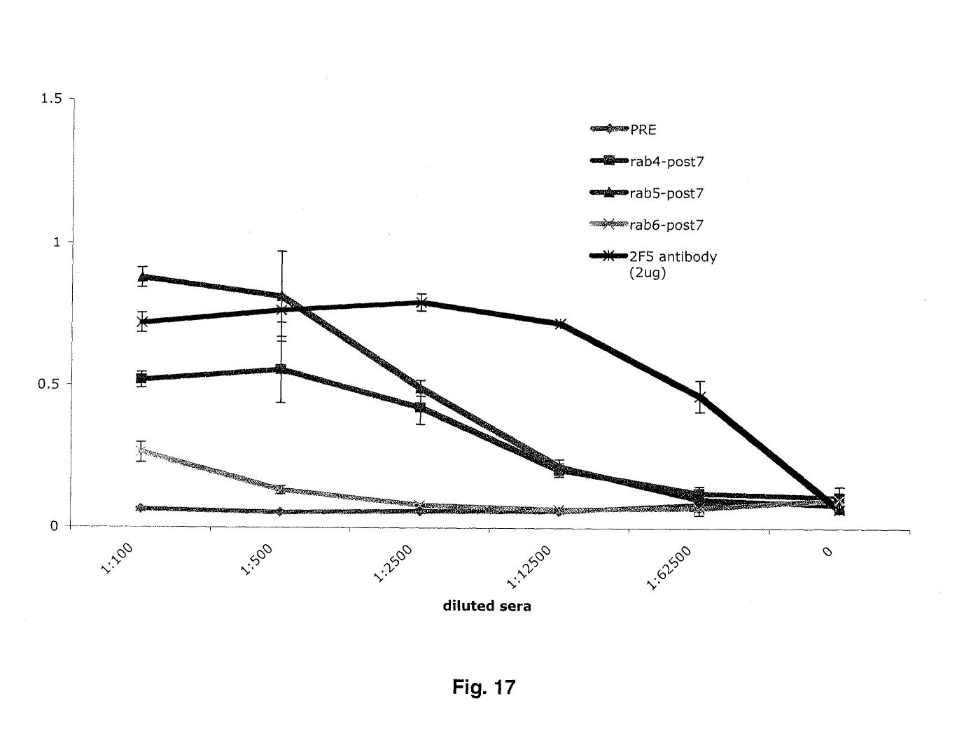

Since the scaffolds also have tags (his and C9) that are shared among all scaffolds we further looked at specific responses against the 2F5 peptide to rule out that the crossreactivity observed with heterologous scaffolds was not directed to tags. FIG. 17 summarizes ELISA data showing specific responses against a 2F5 peptide (2F5 peptide on the plate).

In summary, the generation of selected 2F5 structure-based scaffolds is a novel approach to potentially elicit MPR-directed HIV neutralizing antibodies. Seven initial scaffolds have been designed and expressed successfully. Six scaffolds bind 2F5 by ELISA and/or Biacore. 2F5e_scaffold_{—}1 and 2F5e_scaffold_{—}2 elicited 2F5 epitope specific antibody responses in a three rabbit preliminary experiment when used in sequential immunizations to focus the responses on the scaffolded 2F5 epitope.

### Antigenic Specificity of the Epitope Scaffolds

To determine the degree to which the two structural elements that have been incorporated into the epitope-transplant scaffolds—conformational stabilization of the MPER in its 2F5-bound state and proper surface accessibility of the MPER—are providing antigenic specificity in terms of recognition of the epitope, we have analyzed the binding of non-neutralizing sera to the scaffolds. These experiments were undertaken because one cannot rely solely on the binding affinities of 2F5 to the scaffolds in order to determine whether or not the structural stabilization and surface accessibility of the epitope graft have been properly performed—after all, 2F5 binds to the free, non-structurally-stabilized flexible peptide with binding affinities that are also in the nM range (Table 3).

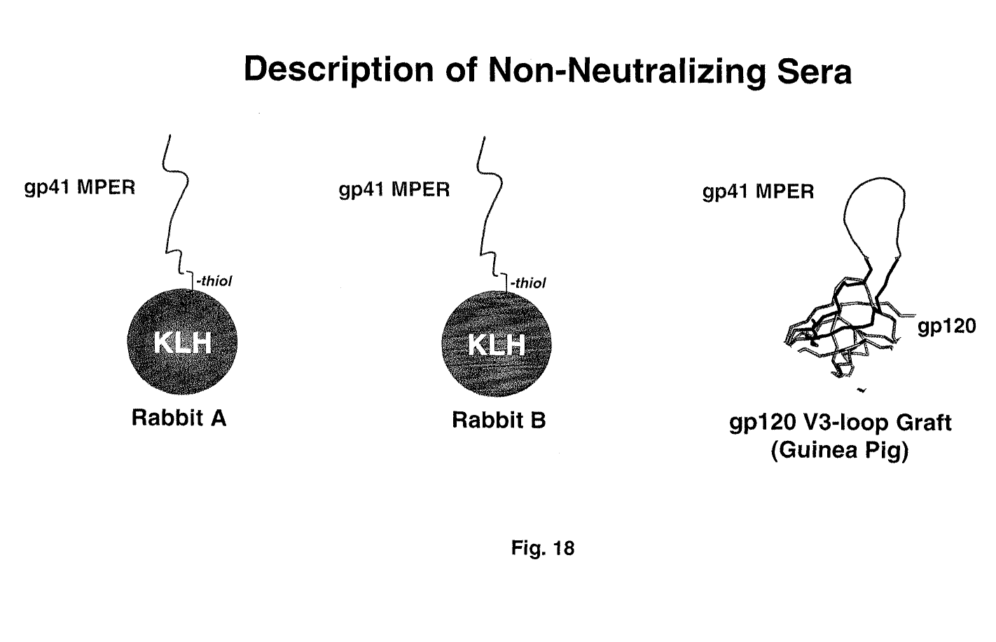

Therefore, non-neutralizing sera from animals that were immunized with either a flexible MPER construct or with a surface loop graft of the MPER were used to determine if the scaffolds truly provide structure-based antigenic specificity. A description of the sera is given in FIG. 18, and includes sera from two rabbits “A” and “B” that were immunized a flexible MPER peptide conjugated to KLH through a thiol linkage, as well as sera from guinea pigs which were immunized with a construct that had the MPER grafted into the V3 loop of the gp120 (Chakrabarti B. K. et al. 2005 Vaccine 23:3434-3445).

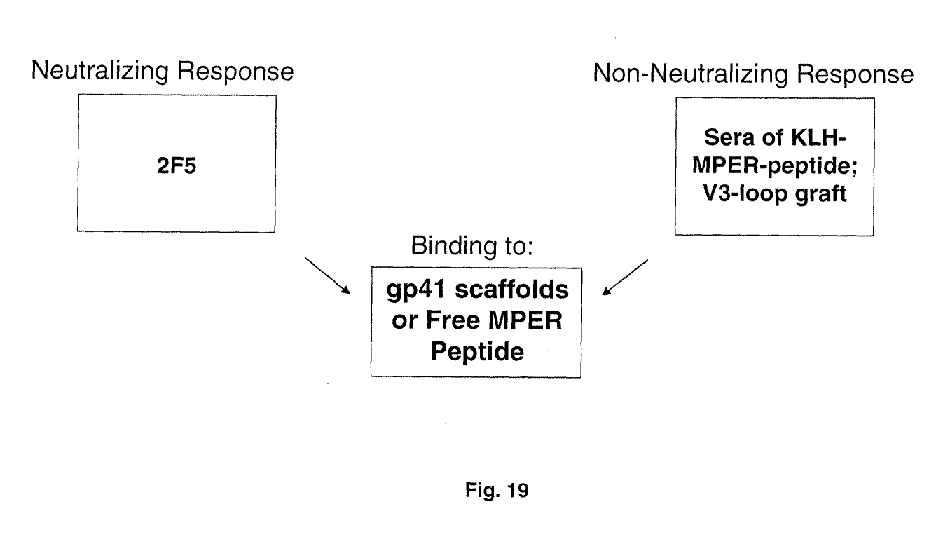

As shown in FIG. 19, the rationale behind the binding studies was to examine the reactivity of a neutralizing response, here represented by the 2F5 antibody, and a non-neutralizing response, represented by the three sera described in FIG. 18, to the scaffolds and to the free MPER peptide in order to compare the degree of antigenic specificity the scaffolds provide. If the non-neutralizing sera do not recognize the scaffolds, but do recognize the free MPER peptide, while 2F5 recognizes them all, this would suggest that the scaffolds are indeed providing true structural antigenic specificity on recognition of the epitope graft, likely in terms of conformation and surface accessibility.

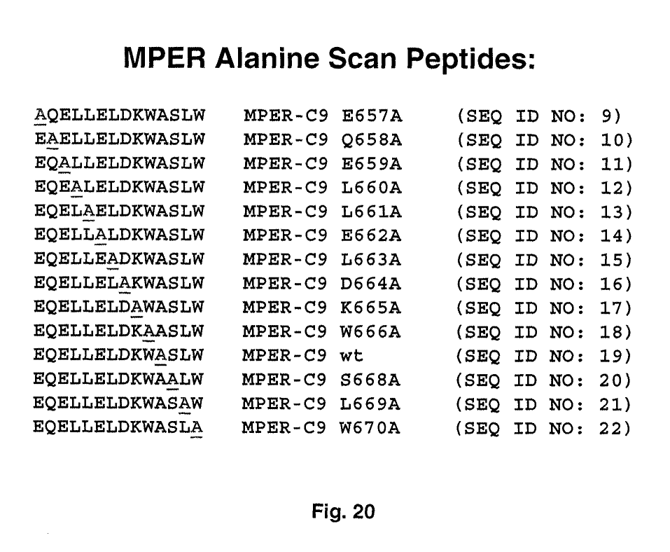

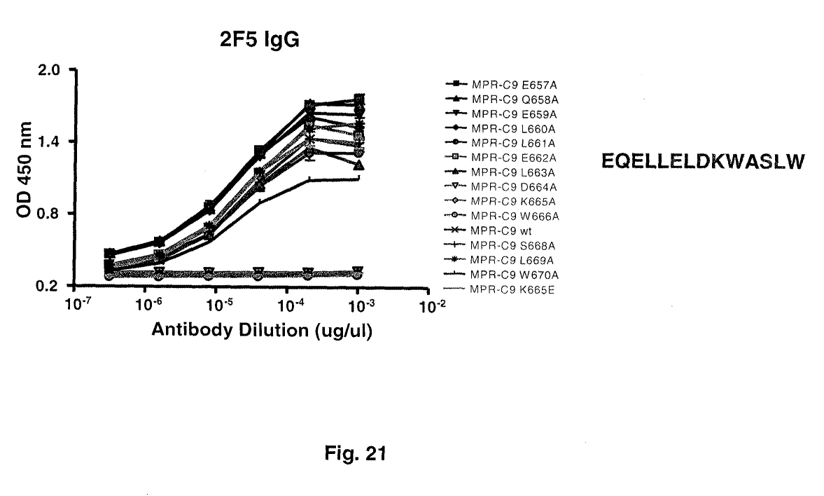

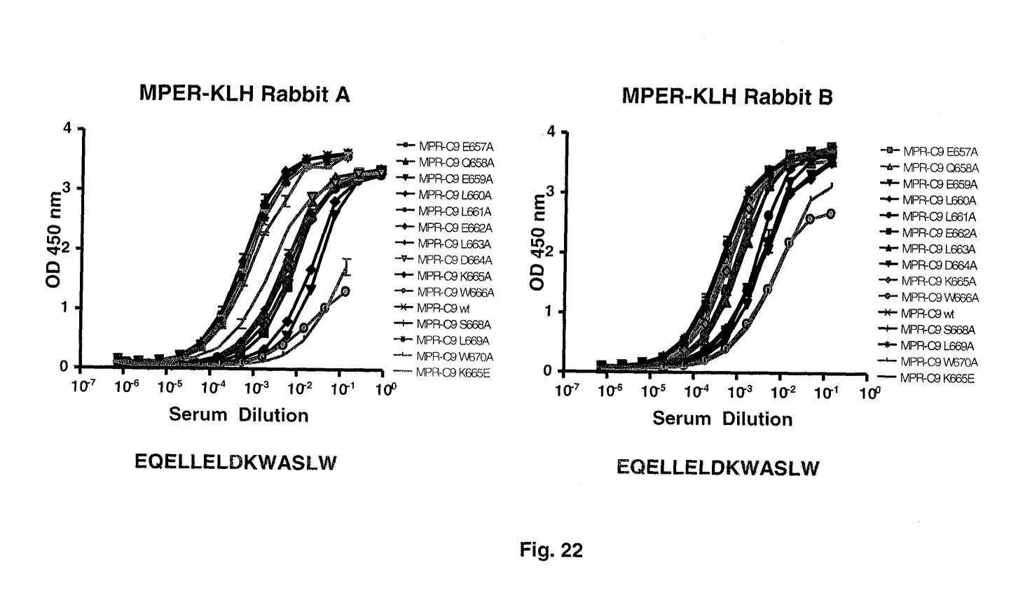

Before embarking on the binding analyses to the scaffolds, however, we wanted to first verify the sensitivity on the non-neutralizing sera to small changes in the MPER sequence. Towards that end, peptides with alanine scan mutations of the entire MPER region were synthesized (FIG. 20) and then examined in an ELISA assay for reactivity to 2F5 (FIG. 21) and reactivity to the non-neutralizing sera from rabbits A and B (FIG. 22). As expected, 2F5 bound to all alanine scan peptides except for those in the DKW core (FIG. 21). As far as the sensitivity of the non-neutralizing sera from rabbits A and B to the alanine mutations in the MPER, though differences were observed in terms of responses to different peptides, overall the sera were not very sensitive to the alanine mutations in the peptides and did show reactivity to the alanine scan peptides (FIG. 22), suggesting that the non-neutralizing sera could in general tolerate small changes in the sequence of the epitope.

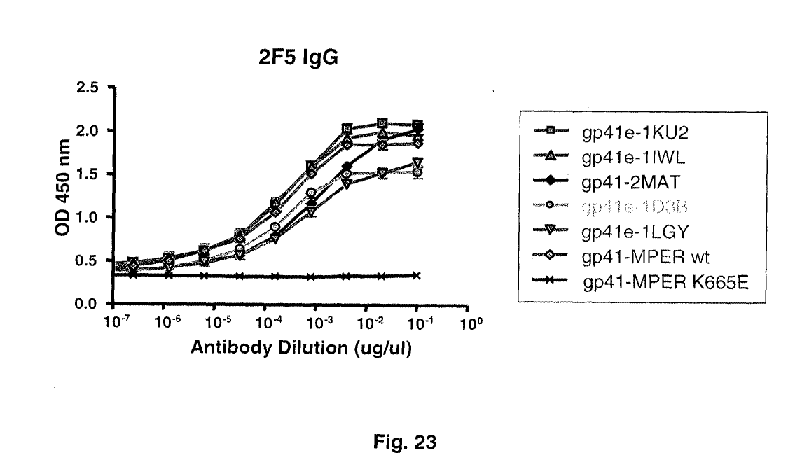

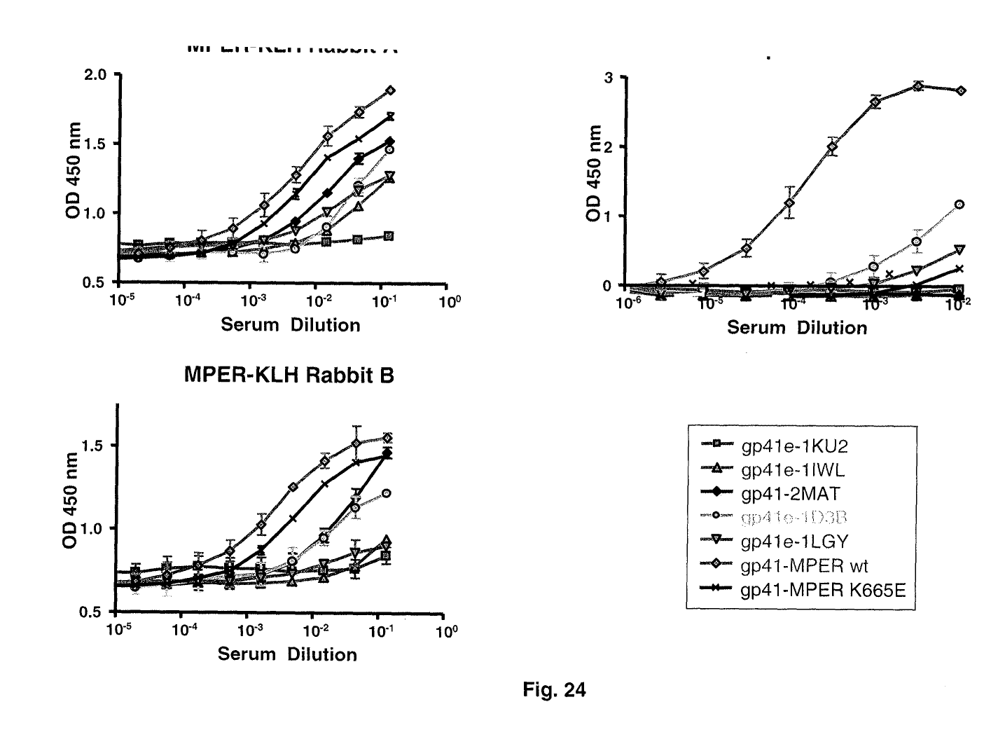

Next we examined the reactivity of the 2F5 antibody in the ELISA context to the scaffolds and to the flexible MPER wild type and mutant K665E peptides (FIG. 23). 2F5 bound to all of the scaffolds and to the flexible wild type peptide almost equally well, while it did not bind to the K665E mutant peptide, as expected. The three non-neutralizing sera, however, as shown in FIG. 24, did not recognize the epitope graft in the scaffolds as well as they recognized the flexible MPER peptides. In some cases, such as the 1KU2 scaffold, the non-neutralizing sera were completely refractory to recognition of the epitope graft, even though the 2F5 antibody bound to this scaffold with nM affinity—suggesting that the 1KU2 scaffold is providing the highest degree of structural antigenic specificity on recognition of the epitope graft. Next in line in terms of restriction on antigenic recognition of the epitope were the 1LGY and 1IWL scaffolds, while the 1D3B and 2MAT scaffolds provided the least degree of restriction on antigenic recognition by the non-neutralizing sera. Though it should be noted that even 1D3B and 2MAT still exhibited in some cases, in terms of serum dilution, more than an order of magnitude higher restriction on antigenic recognition when compared to binding of the non-neutralizing sera to the flexible wild type peptide.

### Comparison to Antigenic Specificity Provided by Synthetic Cyclized MPER Peptides

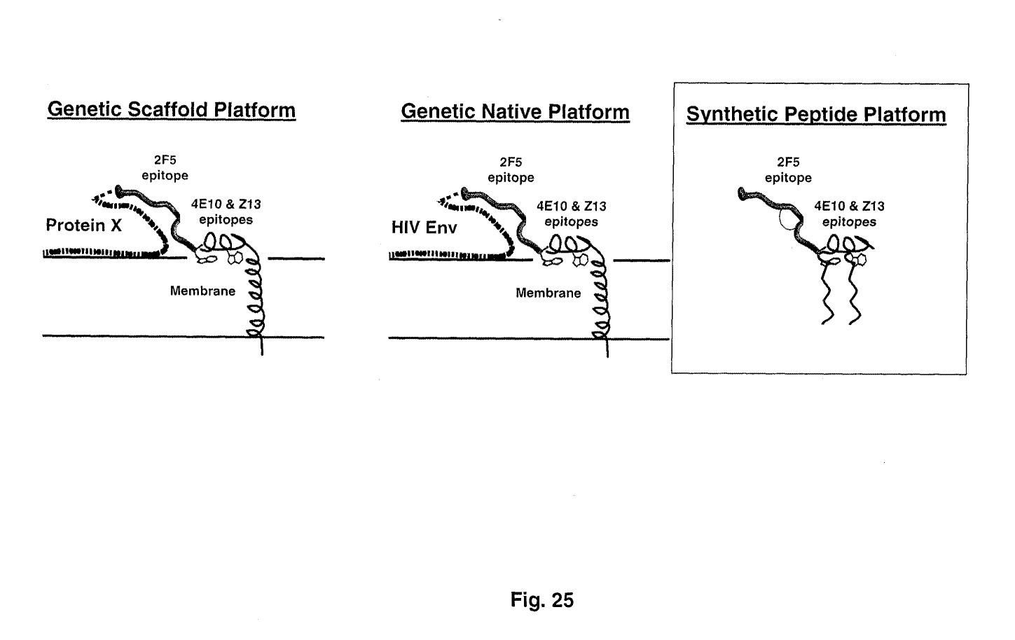

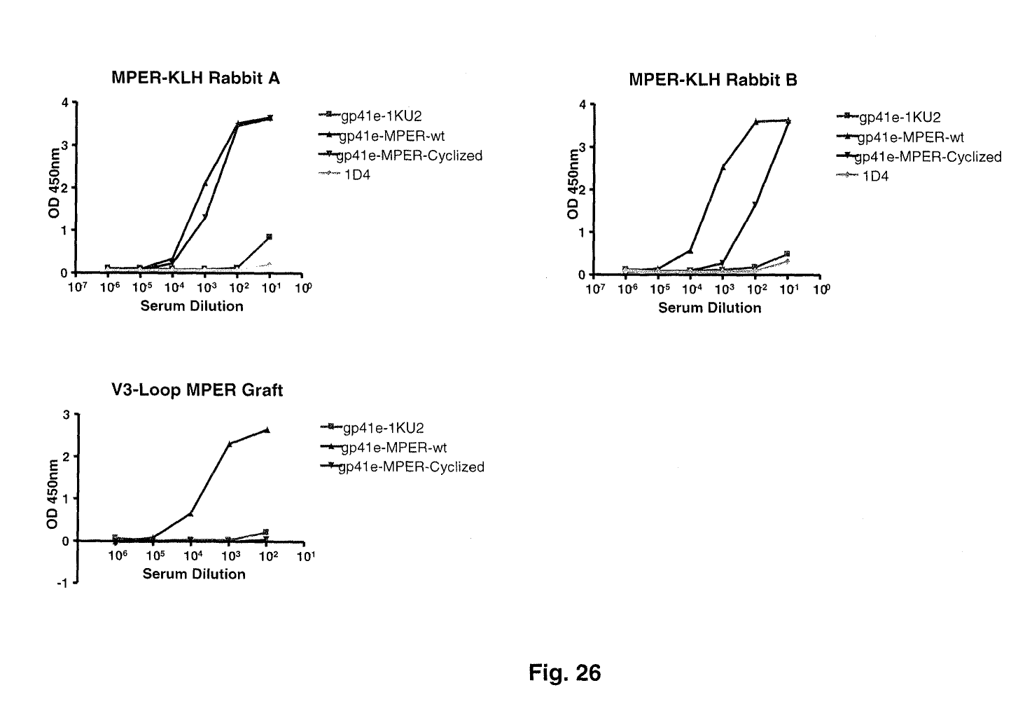

Synthetic cyclized MPER peptides have previously been tested by other investigators. Such synthetic cyclized MPER peptides were mainly designed to properly stabilize the gp41 MPR in its 2F5-bound conformation. FIG. 25 shows contrasting diagrams of the genetic scaffold, genetic native, and synthetic peptide platforms. FIG. 26 shows how recognition of the cyclized MPER compares with the recognition of the 1KU2 scaffold and the free MPER peptide by non-neutralizing sera. The experiments show that the cyclized peptide exhibits antigenic specificity that is in between that provided by the 1KU2 scaffold and the flexible MPER wild type peptide. The sequence of cyclized MPER peptide is EQELLE-c(Dap-DKWD)-SLWGGTETSQVAPA (SEQ ID NO: 105) and is based on McGaughey, G. B. et al. 2003 Biochemistry 42:3214-3223. A summary comparing 2F5 binding by the epitope-transplant scaffolds, MPER wild type peptide and MPER cyclized peptide is provided in Table 3.

### Structural Analysis of gp41e-1KU2

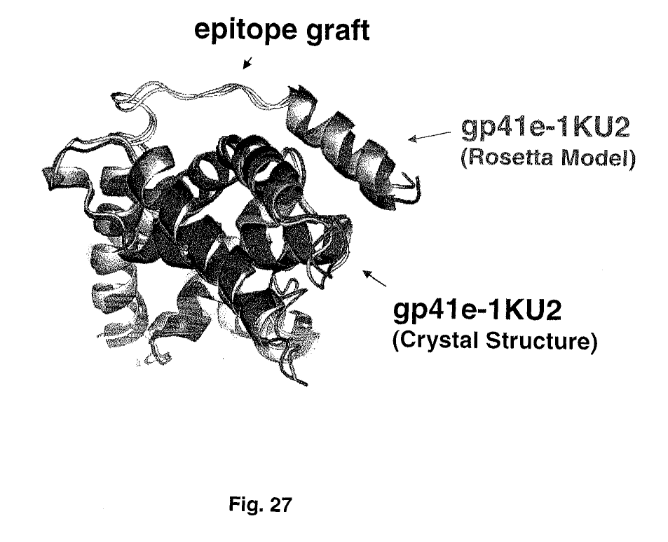

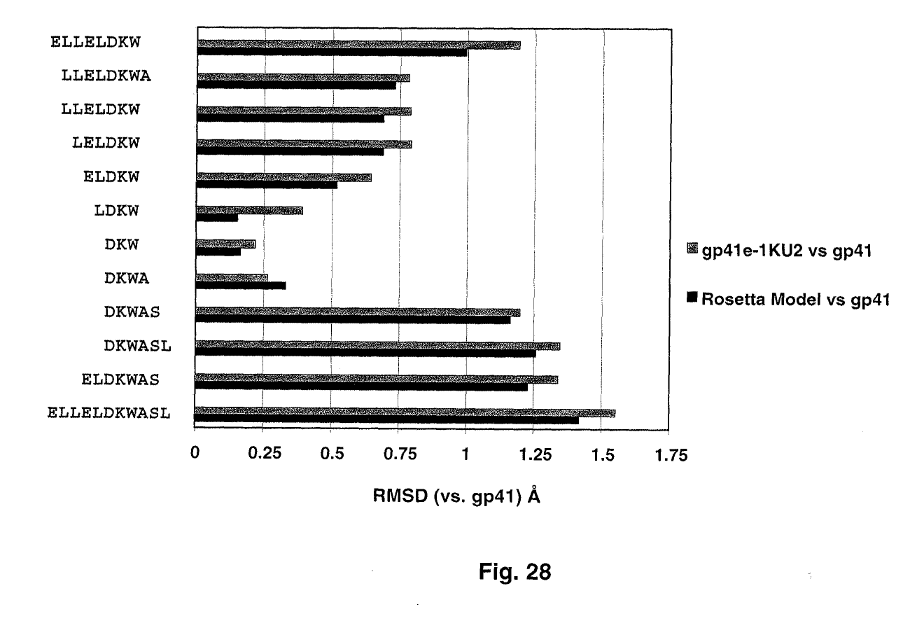

The gp41e-1KU2 was crystallized in its free form to verify the accuracy of the modeling and to verify what would be presented to the immune system. The crystals diffracted to 3 Å, and the structure was solved with molecular replacement using the wild type 1KU2 structure as a search model (with the region of the epitope graft omitted) (FIG. 27). A superposition of the gp41e-1KU2 crystal structure with the gp41e-1KU2 rosetta model shows a high degree of similarity in the epitope graft, with an alpha carbon RMSD of 0.526 Å. A comparison of the gp41e-1KU2 crystal structure and Rosetta model epitope grafts against the 2F5 bound conformation of gp41 was also undertaken using various subsections of the epitope graft (FIG. 28). Root mean square deviation (RMSD) alpha carbon data as given in FIG. 28 provide an indication of how the gp41e-1KU2 epitope graft compares structurally with gp41, within the different subsections of the graft. The crystal structure shows that the highest degree of homology with gp41 is obtained in the LDKWA core of the epitope graft (FIG. 28).

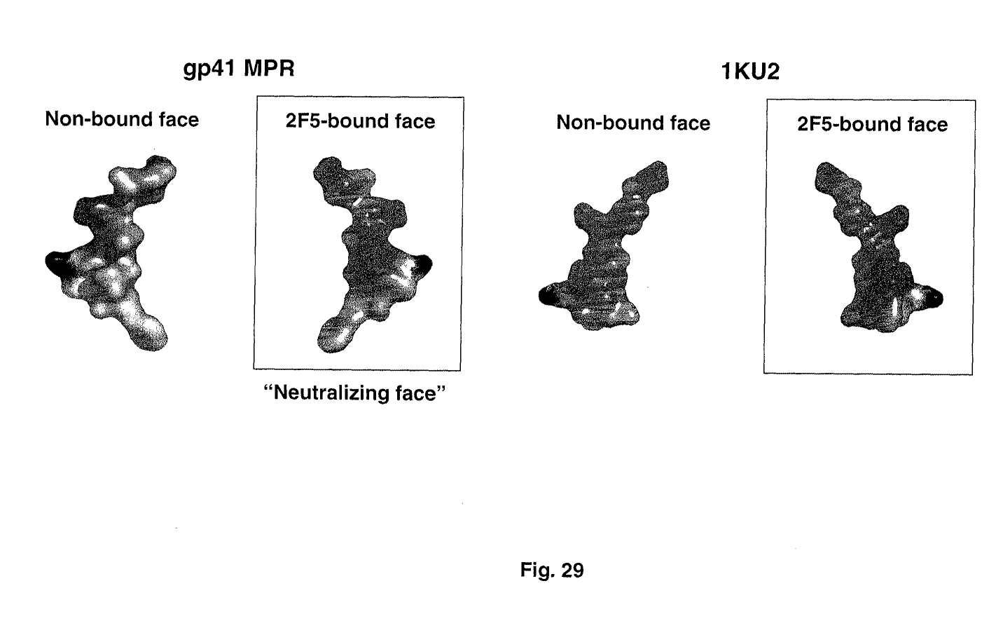

A comparison of the electrostatic characteristics of the 2F5 bound surface of the gp41 MPER with the electrostatic characteristics of the exposed surface of the 1KU2 epitope graft also shows a high degree of similarity (FIG. 29).

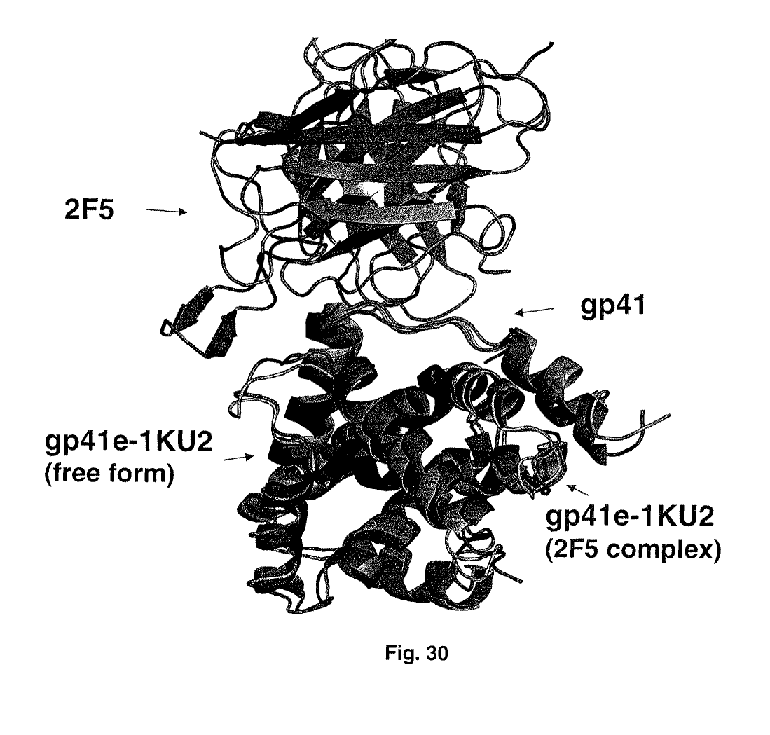

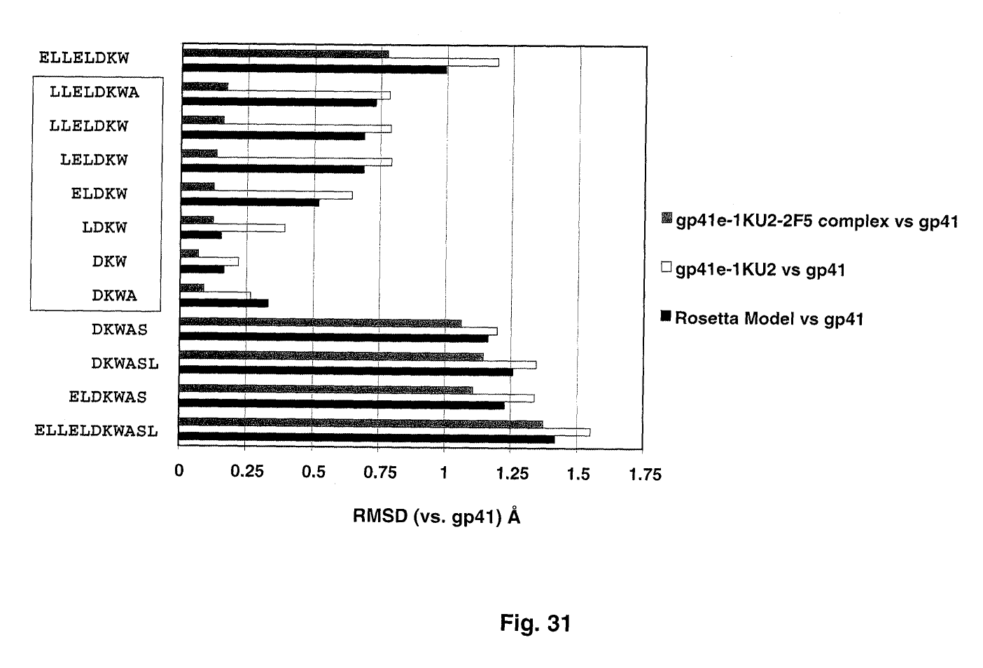

gp41e-1KU2 was also crystallized in complex with the 2F5 Fab to determine to what extent 2F5 induces a fit in the epitope and to ascertain the structural fidelity required by 2F5. The crystals of the gp41e-1KU2-2F5 Fab complex diffracted to 2.8 Å. A superposition of the crystal structures of gp41e-1KU2 in the free form and in the complex with 2F5 is shown in FIG. 30, along with the structure of the gp41 MPER in its 2F5-bound conformation. An RMSD comparison of the C-alpha trace of various subsections of the epitope graft against the 2F5-bound form of the gp41 MPER is shown FIG. 31, and reveals that 2F5 induces a fit in a subset of the gp41 epitope graft encompassing residues LLELDKWA (SEQ ID NO: 25).

The crystal structures of both the free form of gp41e-1KU2 and of the complex with 2F5 provide structural information for optimization and development of future generation scaffolds. For instance, they inform what subsections of the epitope might be most crucial for inclusion.

Determination of the structure of 2F5 in complex with the complete gp140 envelope spike will further inform scaffold design. In addition, it will be possible to better define the functional role of the MPER, and at what fusion stage 2F5 acts. If 2F5 acts on a fusion intermediate then this may be accounted for in an immunization scheme.

Furthermore, immunogens may be developed to account for membrane context and steric accessibility through prime-boost strategies. Our analysis of the 2F5 antibody provides an example of how the atomic-level techniques of modern structural biology can be applied to the development of an effective HIV-1 vaccine.

### Example of a Reductionist Scaffold: 1KU2

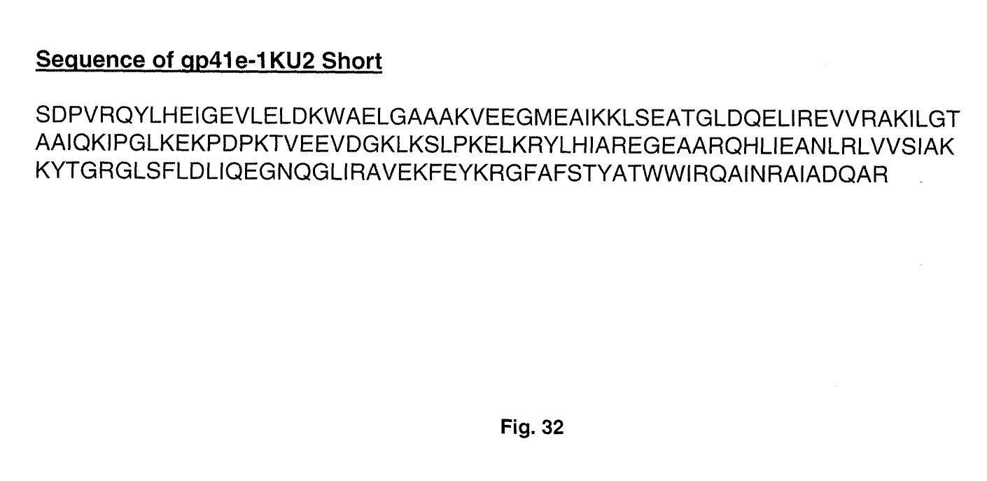

The 1KU2 scaffold was originally synthesized to encompass 240 amino acids (excluding the N-terminal leader sequence and C-terminal tags; or 267 amino when the C-terminal tags are included), with the epitope graft lying at the N-terminus of the protein. Though in most preparations the gp41e-1KU2 scaffold protein remained intact, in some cases proteolytic cleavage was observed to take place at residue Arg179 (as determined by mass spec analysis; FIG. 32). The smaller fragment, obtained both from proteolytic cleavage of the larger form and from insertion of a stop codon after residue Arg179, and subsequent expression and purification, was used for the crystallization of the 41e-1KU2 scaffold, both in its free form and in complex with 2F5 (FIG. 27 and FIG. 30). The reduction of the 1KU2 scaffold from a 267 residue protein (including the tags) to a 179 residue protein that still contains the epitope graft, represents an example of how a scaffold can be reduced to encompass just the relevant domain that contains the epitope.

### Immunogenicity of 2F5 Epitope Scaffolds

The first immunogenicity study was designed to determine if the 2F5 epitope scaffolds are immunogenic. In this study we examined adjuvant effects (Alum and CpG vs. GSK AS01B), the necessity of heterologous T cell helper epitopes and dosage.

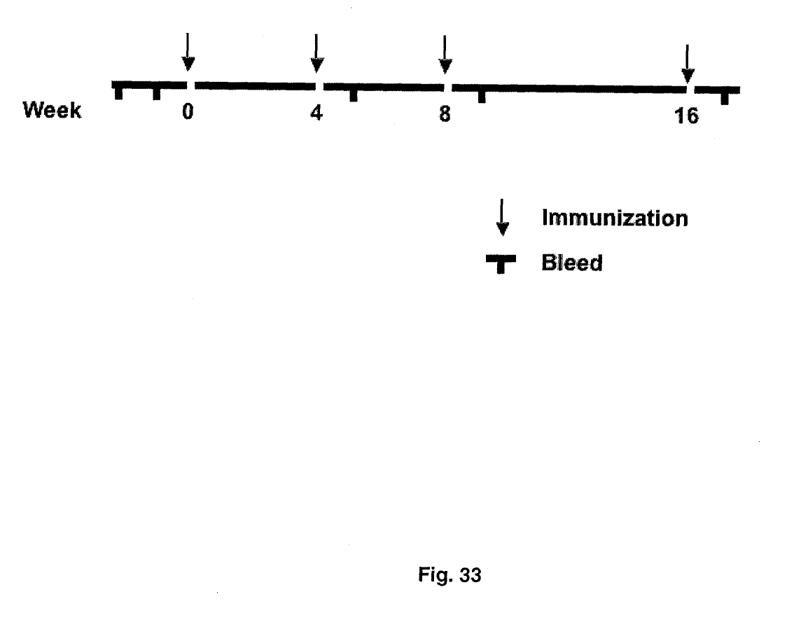

We included 4 guinea pigs per group (some groups include more animals to test dose and adjuvant effects). We immunized 4 times with each scaffold 4 weeks apart, except for the last immunization, which is 8 weeks after the 3rd immunization. Two pre-bleeds were collected before the first immunization and one bleed a week after each immunization starting after the second immunization (FIG. 33). Table 4 summarizes details of the constructs used, dosages and adjuvants. Note that data are shown for the 2F5e-1d3b and 2F5e-1ku2 scaffolds with and without heterologous T cell helper epitopes (TH).

### Analysis of Data

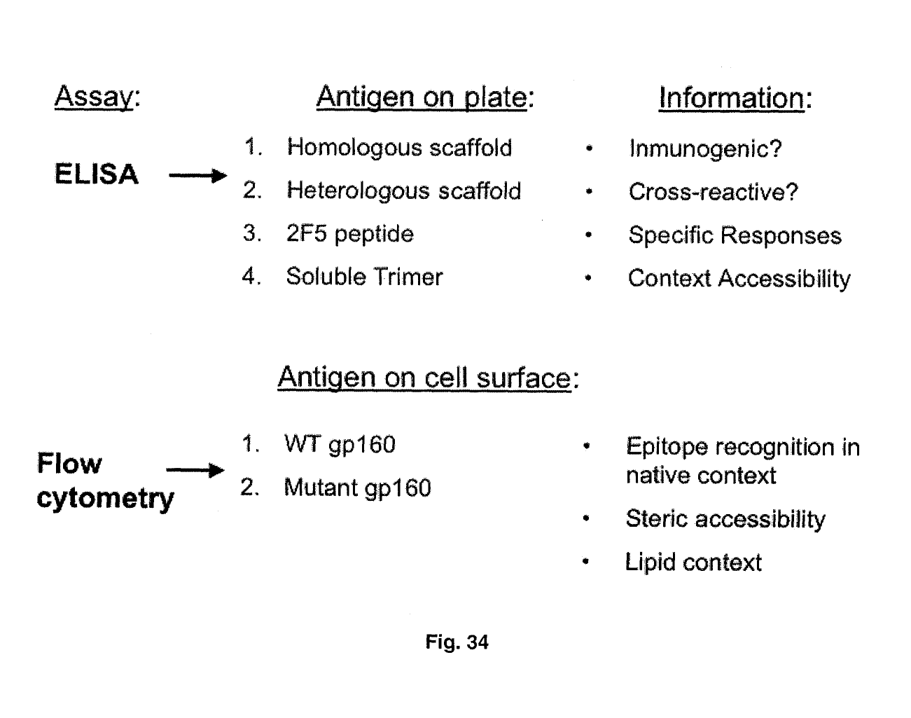

In order to characterize the anti-scaffold serum responses from guinea pigs immunized with the scaffolds we carry out two types of binding assays: ELISA and Flow cytometry assays. These assays allow us to obtain information regarding the immunogenicity, cross-reactivity, specificity and epitope accessibility of the anti-scaffold serum responses. FIG. 34 includes details of the assays used to characterize the anti-scaffold serum responses of guinea pigs immunized with scaffolds and the information that can be obtained from these assays.

### Results

### 1. Immunogenicity

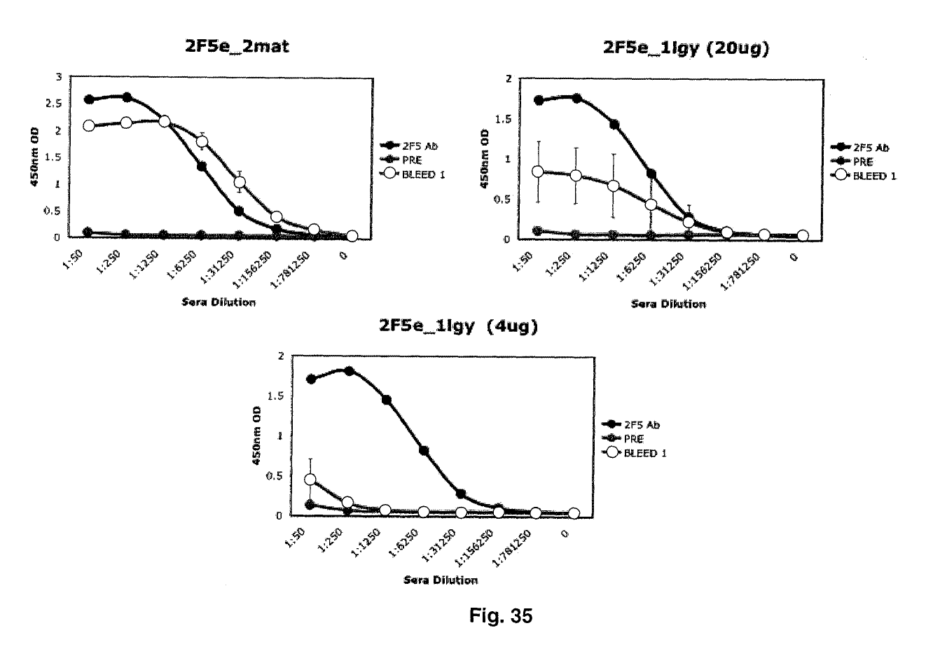

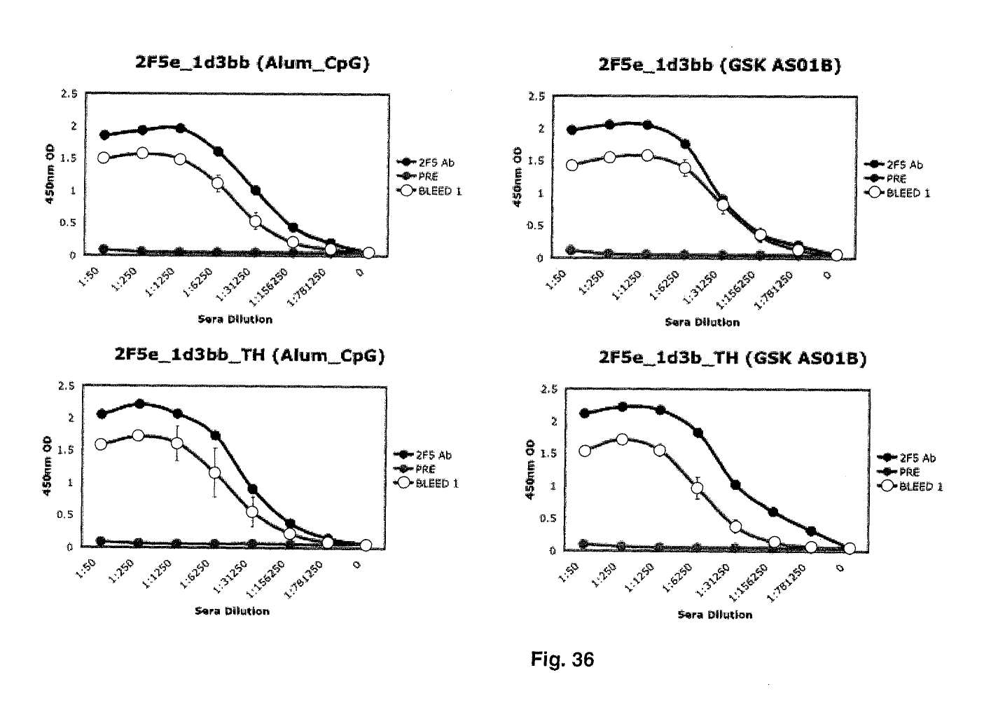

After two immunizations all scaffolds are immunogenic. The binding curves displayed in FIG. 35 and FIG. 36 represent the following: In black, our positive control 2F5 antibody at a starting concentration of 10 μg/ml and serially diluted 1:5. The gray line is our negative control pre-immune serum pooled from all animals belonging to a group and the white binding curves represent the mean values with standard deviation of anti-scaffold serum of all animals of a group after 2 immunizations. All the serum samples are serially diluted 1:5 starting from an initial 1:50 dilution (See FIG. 35 and FIG. 36). The low dose group immunized with 4 μg of scaffold 2F5e_{—}1lgy did not show good binding responses against homologous scaffold suggesting that immunizing with 20 μg of protein is better than with 4 μg (FIG. 35). Binding to homologous scaffold was determined by ELISA. Anti-scaffold serum binding to antigen on the plate is shown after two immunizations.

### 2. Cross-Reactivity

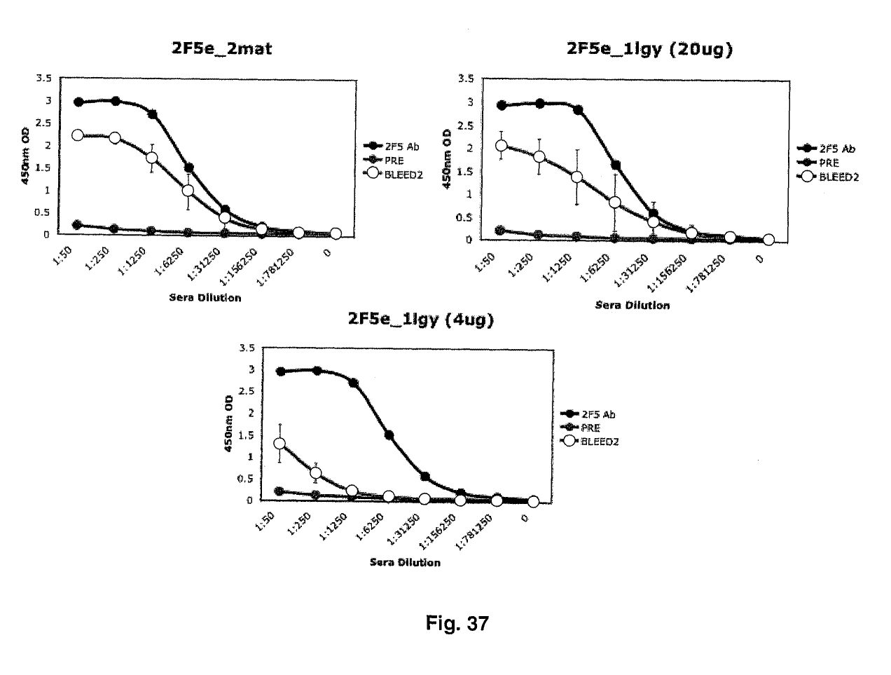

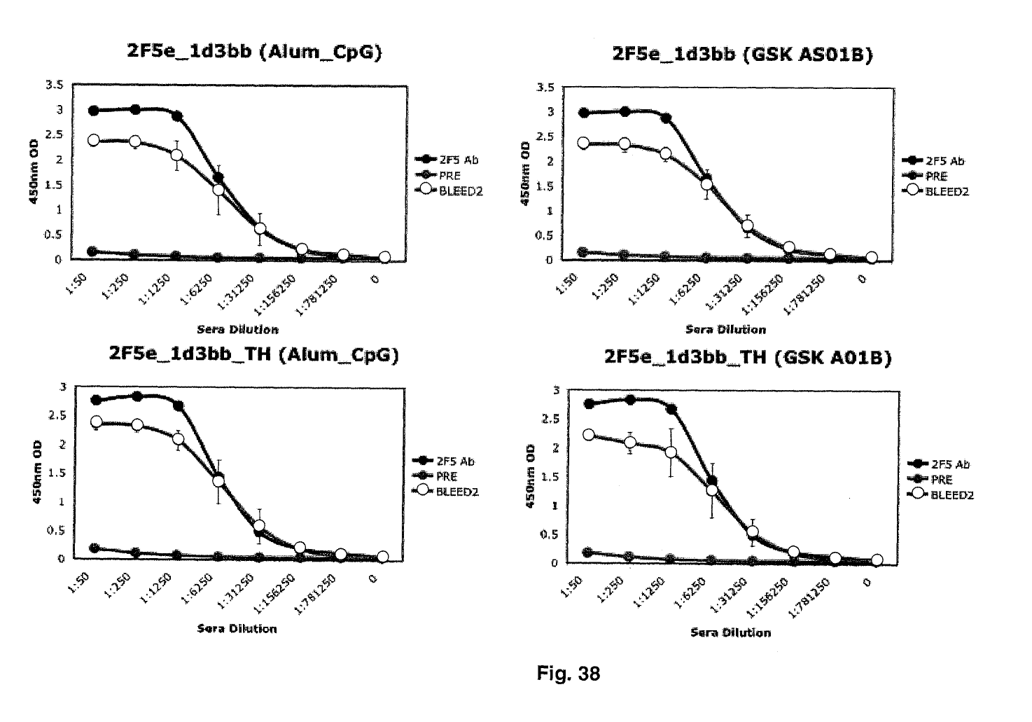

We measured cross-reactivity using a heterologous scaffold on the ELISA plate. A heterologous scaffold is a 2F5 epitope scaffold that was never injected in the group of animals whose serum one is assaying. Since the scaffolds all contain different antigenic backgrounds, and the only shared component is the graft of the 2F5 epitope, measuring binding of anti-scaffold serum to heterologous scaffolds represents a marker of epitope specific responses of immunoglobulins that see the epitope in a conformationally stabilized manner. All scaffolds tested so far: 2F5e_{—}2mat, 2F5e_{—}1lgy, 2F5e_{—}1d3bb and 2F5e_{—}1d3bb_TH show serum responses that cross-react with heterologous scaffold 2F5e_{—}1ku2 (FIG. 37 and FIG. 38). Binding to heterologous scaffold was determined by ELISA. Anti-scaffold serum binding to antigen on the plate is shown after three immunizations.

### 3. Specificity

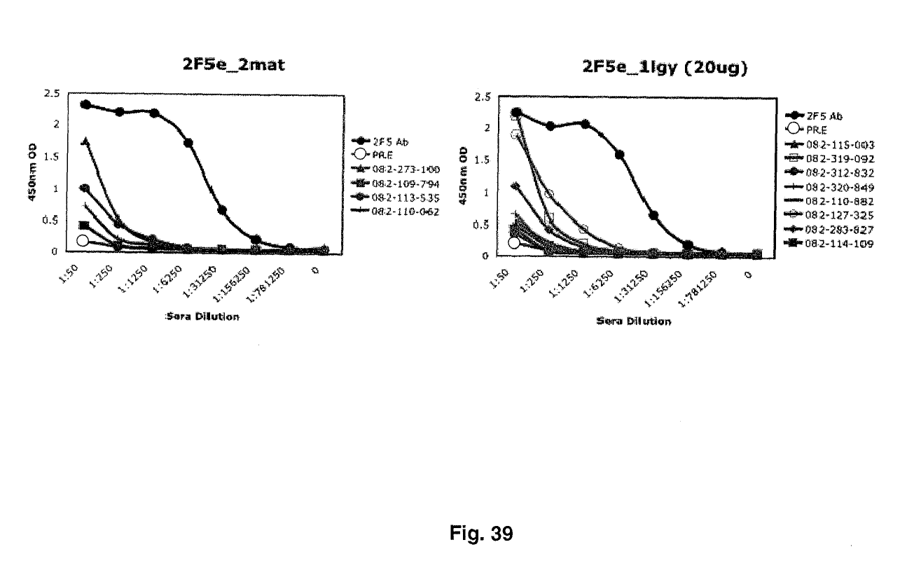

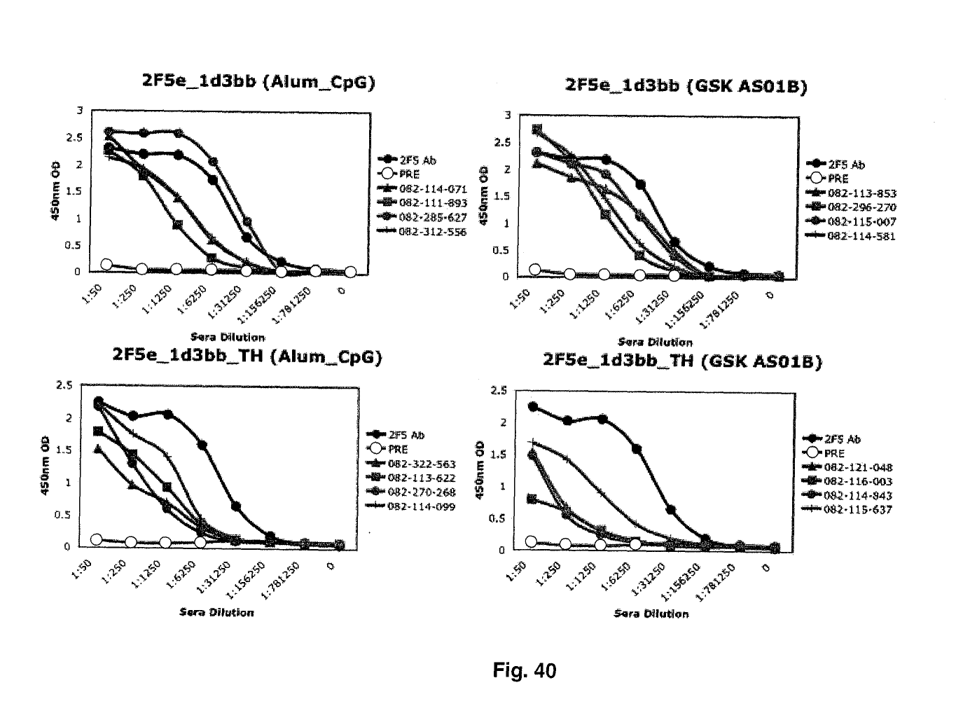

Measuring binding to 2F5 peptide captured on a plate allows us to determine the specificity of the anti-scaffold serum responses. The 2F5 peptide is a surrogate marker for the HIV envelope glycoprotein gp41 epitope. Anti-scaffold serum from animals immunized with scaffolds 2F5e_{—}2mat and 2F5e_{—}1lgy show low binding responses to peptide while animals immunized with 2F5e_{—}1d3bb and 2F5e_{—}1d3bb_TH show high binding responses to peptide after 3 immunizations. In FIG. 39 and FIG. 40, instead of showing the mean value of a group of animals, we display the binding curve of each animal individually. The data show ELISA binding to captured 2F5 peptide.

### 4. Accessibility

The native HIV spike is a hetero-trimeric protein composed of 3 monomers of envelope glycoprotein gp120 and 3 monomers of gp41 that interact non-covalently to form the native HIV spike on the virus. In order for an antibody to bind the 2F5 epitope on gp41 it may have to circumvent steric constraints established by the trimer spike and the proximity of the viral membrane. Both the ELISA assay measuring binding to soluble trimer on the plate, and FACS analysis of the anti-scaffold serum binding to the native spike (WTgp160 and its cleavage defective mutant) helps us to answer the question of accessibility to the epitope in the presence of steric constraints and lipid membrane.

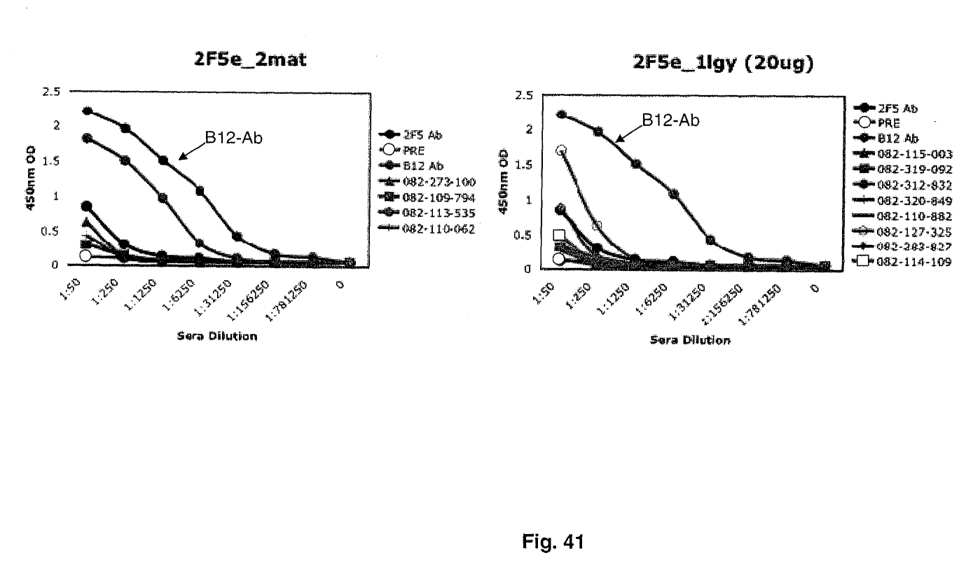

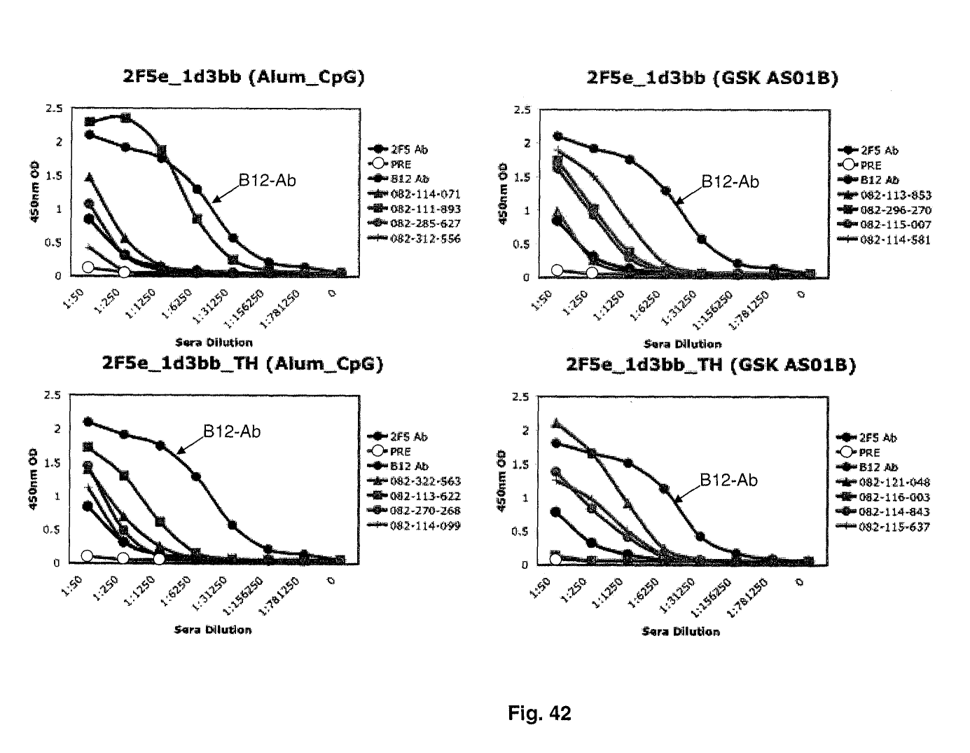

As a second positive control the monoclonal antibody b12 was added to the ELISA assay since its binding to the soluble trimer is higher than that of the 2F5 antibody. The animals immunized with scaffolds 2F5e_{—}2mat and 2F5e_{—}1lgy generated anti-scaffold serum responses that barely bind the soluble trimer by ELISA. Only a few animals show any binding that follow the pattern of 2F5 antibody binding. In contrast, all of the animals immunized with scaffolds 2F5e_{—}1d3bb and 2F5e_{—}1d3bb_TH show binding to soluble trimer in the pattern of 2F5 antibody (See FIG. 41 and FIG. 42). ELISA binding to captured 2F5 peptide are shown.

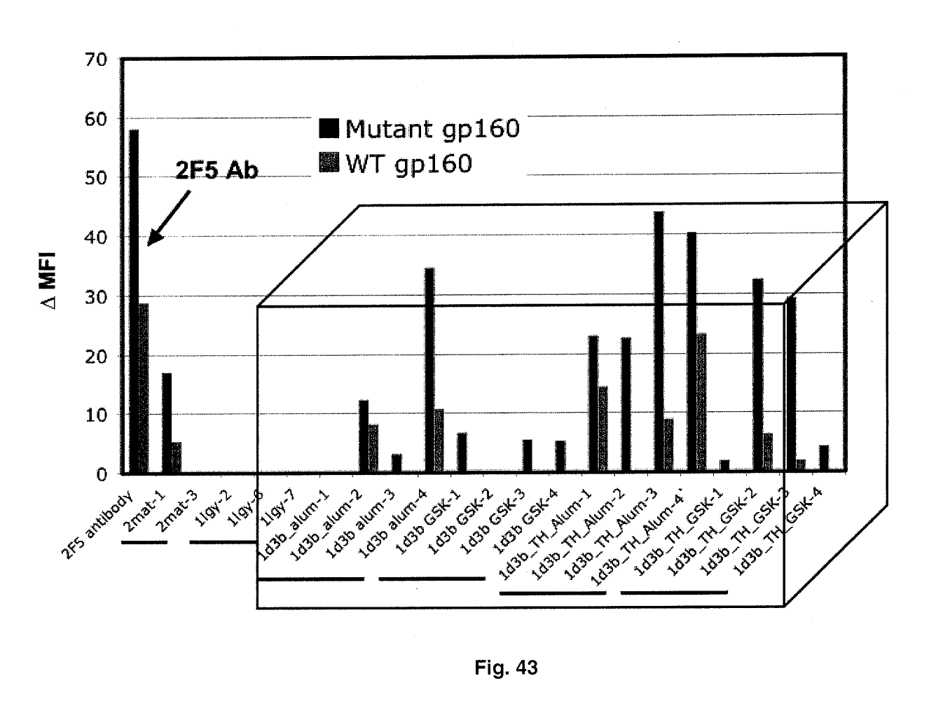

We then selected animals that show binding to both the 2F5 peptide and soluble trimer by ELISA to measure binding to WTgp160 expressed on cell surface. All bleed samples (after 3 immunizations) were diluted 1:100 as well as their respective pre-immune controls. The FACS data is shown as ΔMFI values in the Y axis representing mean fluorescence intensity over background for each animal. The data show that only the serum of animals immunized with scaffolds 2F5e_{—}1d3bb and 2F5e_{—}1d3bb_TH showed binding to the native spike. The binding was always better to the mutant gp160 (cleavage defective) than the WTgp160 following the pattern of binding of 2F5 antibody (FIG. 43).

### Preliminary Data

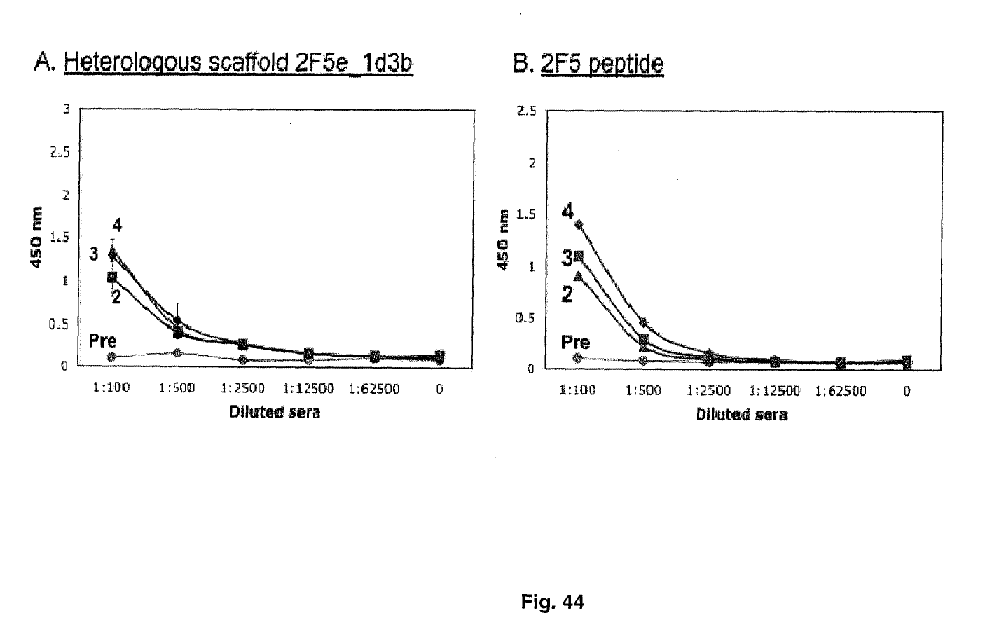

We conducted a pilot experiment initially to determine the immunogenicity of the first available scaffold, 2F5e_{—}1ku2. Three rabbits were immunized four times with scaffold 2F5e_{—}1ku2 (50 μg of protein) every two weeks. CpG (250 μg per animal) and Alum (20 μg per 50 μg of protein) were used as adjuvants. The injections were via the route subcutaneous. Bleeds were collected before the first immunization and subsequently, one week after the second, third and fourth immunizations. As a means to measure specific responses generated to the HIV gp41 2F5 epitope, we carried out ELISA binding assays of the anti-scaffold sera to the heterologous scaffold (2F5e_{—}1d3b). Additionally, we measured anti-scaffold sera responses to a 2F5 epitope peptide on the ELISA plate. Epitope responses were generated, however remained low titer (1:10e4). 2F5e_{—}1ku2 anti-scaffold responses were higher (1:10e5). Pre-immunization sera were used as negative controls (FIG. 44).

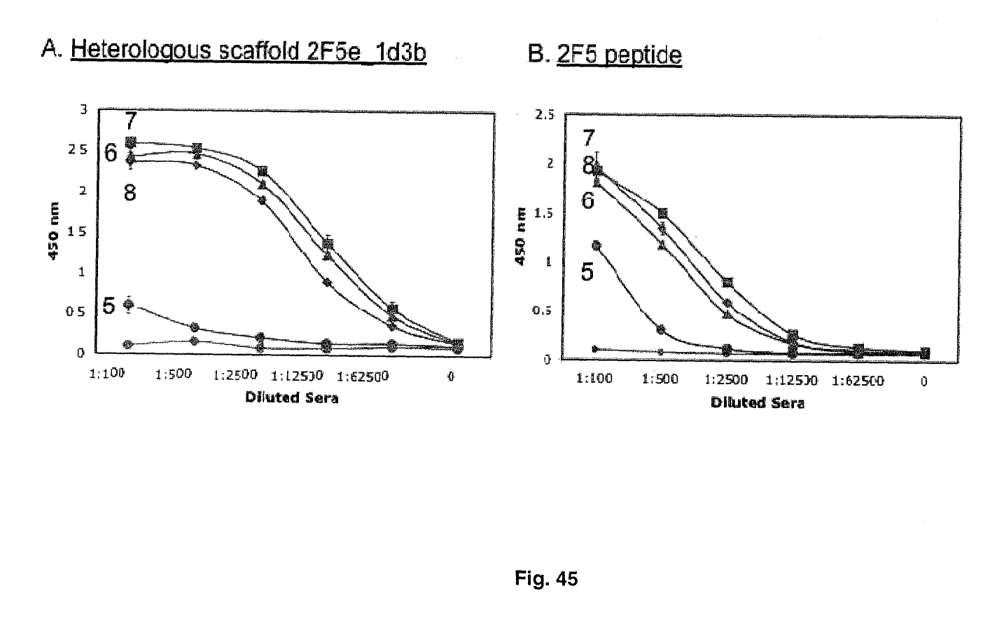

As a second scaffold became available, we immunized the rabbits with a second epitope scaffold (2F5e_{—}1lgy) following the same regimen (4 immunizations every 2 weeks, 50 μg of protein). Anti-scaffold sera were analyzed for binding both to heterologous scaffold and 2F5 epitope peptide. After the second immunization with 2F5e_{—}1lgy the epitope specific responses were greatly magnified reaching end point titers of 1:10e5. This suggests that the approach of sequential immunizations with different 2F5 epitope scaffolds could potentially be used to immuno focus immunological responses on the epitope. Pre-immunization sera were used as negative control (FIG. 45).

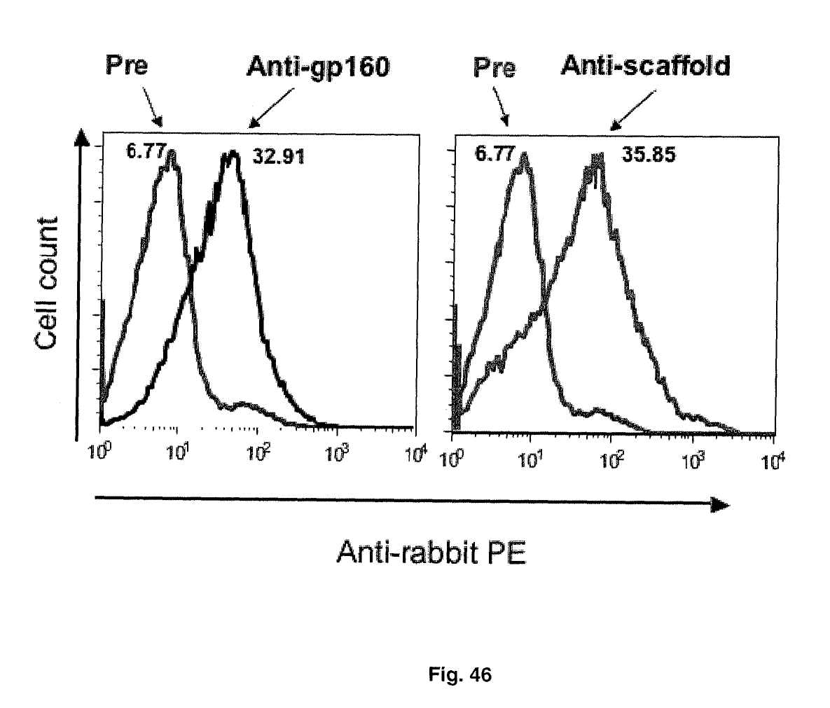

Encouraged by these results, we immunized with a third and last scaffold 2F5_{—}2mat four times two weeks apart. The epitope specific responses observed after the first two sets of immunizations with 2F5e_{—}1ku2 and 2F5e_{—}1lgy did not increase, but seemed to have reached a plateau. This result could mean a number of things: 1) There is no crossreactivity with the third scaffold. 2) The third scaffold 2F5e_{—}2mat was not immunogenic, and we observed weaning titers to scaffold 2F5e_{—}1lgy and/or 3) The titers have reached a maximum level. In order to further confirm these results we further analyzed anti-scaffold sera for binding to MPR peptide expressed on the surface of 293 cells by FACS. The analysis shows that the sera bind MPR expressed on the surface of 293 cells. The pre-immune sera (negative control) do not bind. As a positive control we used sera elicited with gp160 proteoliposomes that have shown crossreactivity with the MPR in previous studies (FIG. 46).

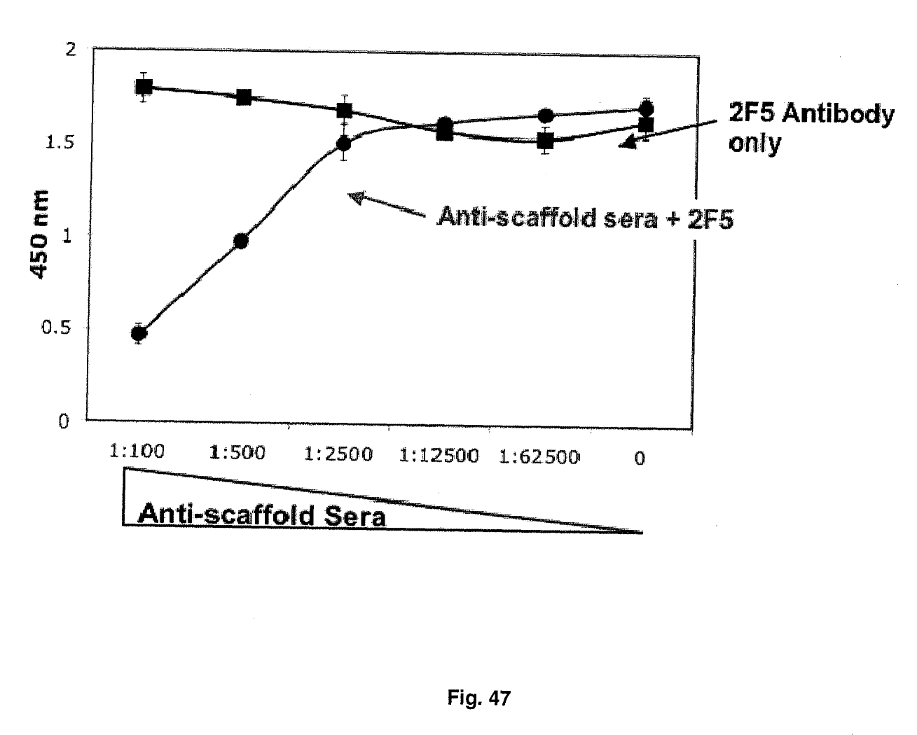

To confirm the specificity of the binding analysis, we conducted a cross-competition ELISA binding assay in which we measured binding of 2F5 antibody to heterologous scaffold 2F5e_{—}1d3b (read-out signal) at a fixed concentration (100 ng/mL). Plotting these data results in a horizontal line since every well contains the same amount of antibody. In a different set of wells we added the same amount of antibody and increasing amounts of anti-scaffold sera (post 7, highest titer) to compete for binding to heterologous scaffold. As the amount of anti-scaffold sera is increased, the read-out signal decreases (FIG. 47), indicating that less 2F5 antibody is able to bind the target site, which is occupied by the anti-scaffold sera.

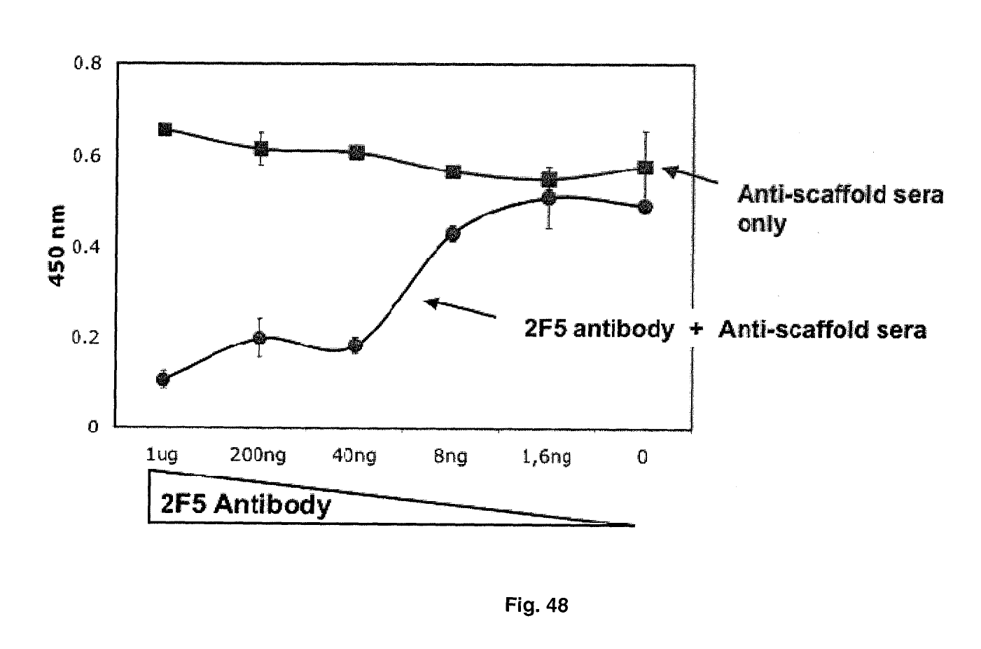

We then reversed the assay so that now one line designates the anti-scaffold sera at a fixed concentration (1:5000 dilution) binding to 2F5 peptide on the ELISA plate. The other line designates a fixed dilution (1:5000) of post 7 sera with increasing amounts of 2F5 antibody competing for binding to the peptide. As the concentration of 2F5 antibody increases the read-out signal decreases (FIG. 48).

These two assays both confirm the specificity of anti-scaffold sera for the 2F5 epitope and validate our heterologous scaffold binding assay as a means to measure 2F5 epitope specific responses generated with 2F5 epitope scaffold immunizations.

### T_{H} Cell Epitopes

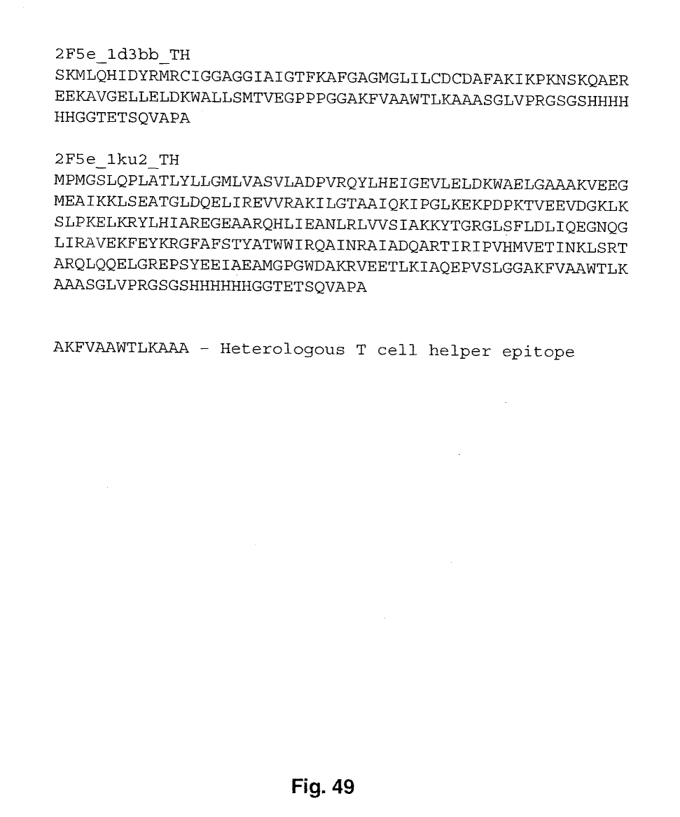

T_{H} cell epitopes can be used to improve scaffold immunogenicity (Alexander J. et al. 1994 Immunity 1: 751-761; Alexander J. et al. 2000 J Immunol 164:1625-1633). For example the universal PADRE T_{H} cell epitope AKFVAAWTLKAAA (SEQ ID NO: 39) can be added to the epitope-transplant scaffold to enhance immunogenicity (FIG. 49).

### b12 Epitope-Transplant Scaffolds

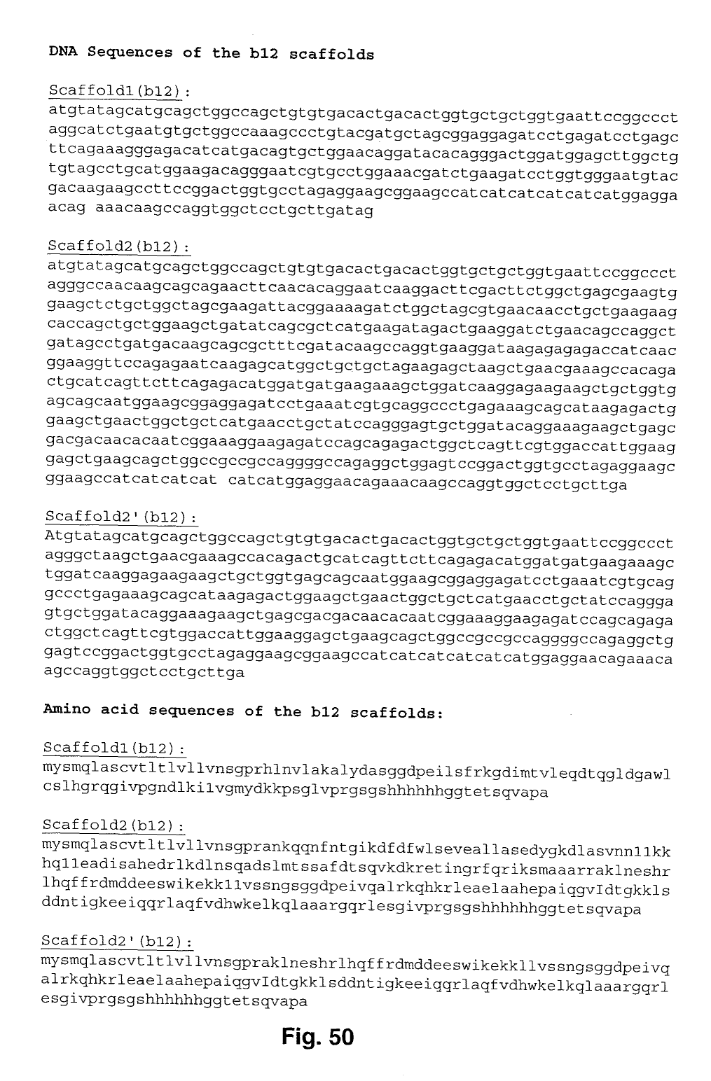

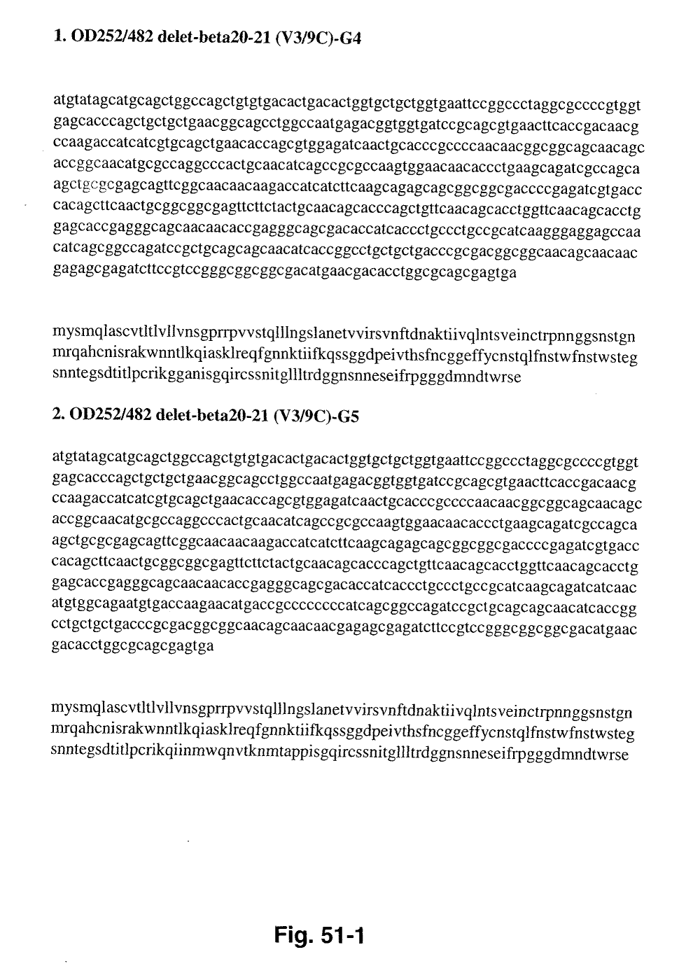

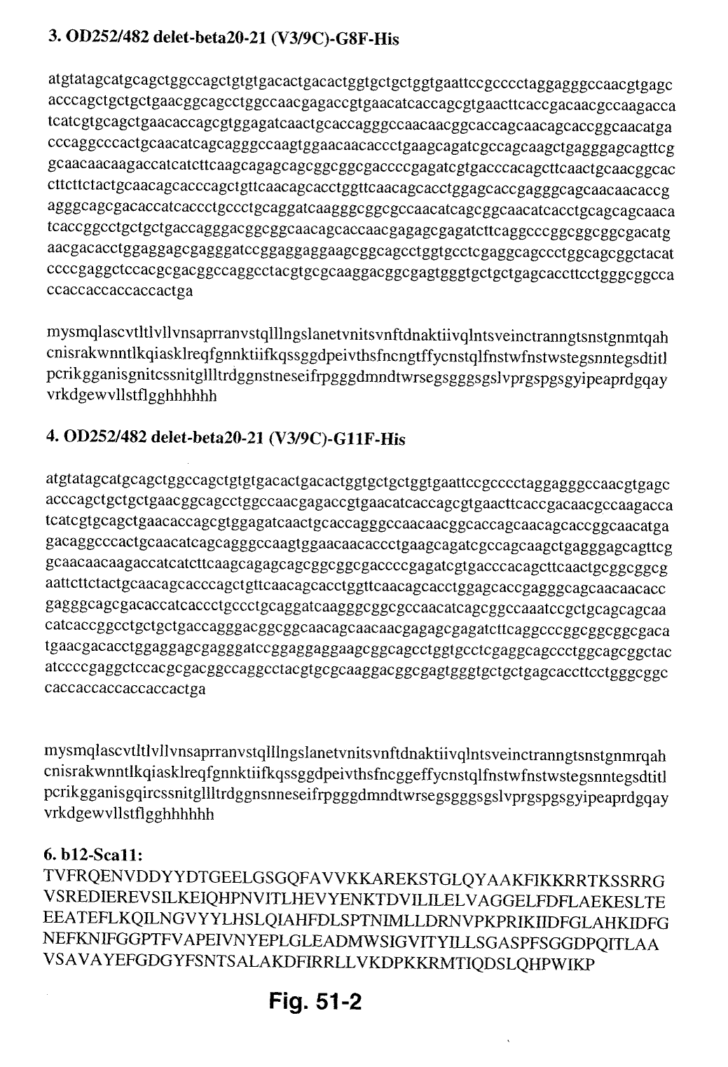

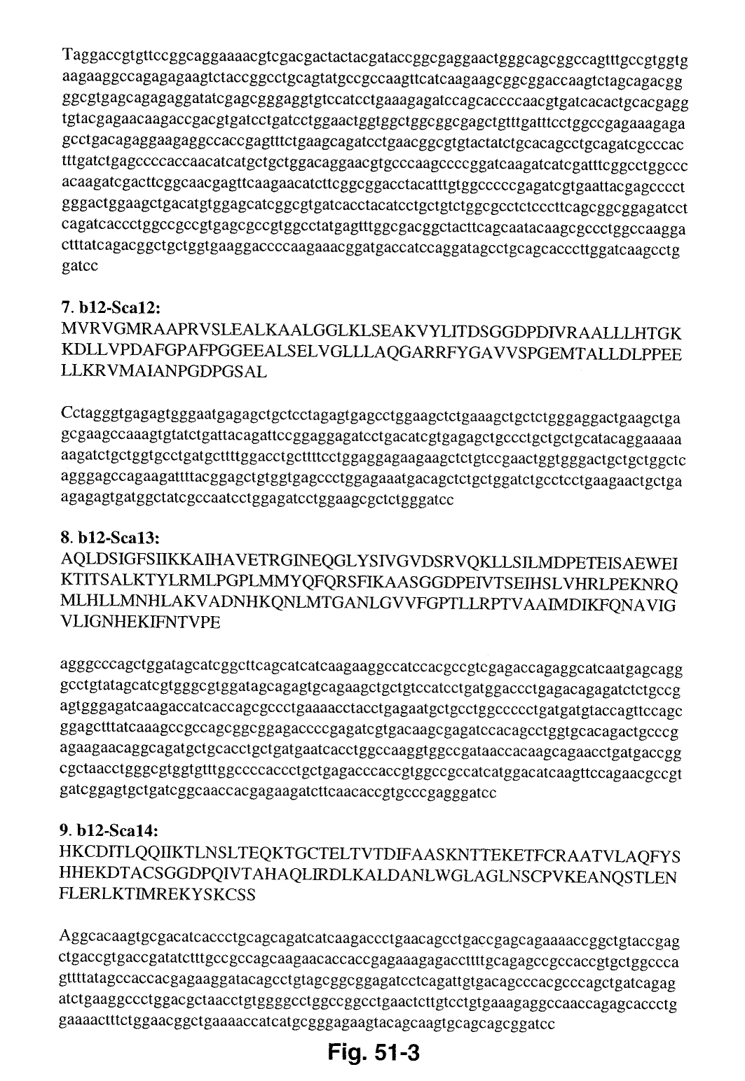

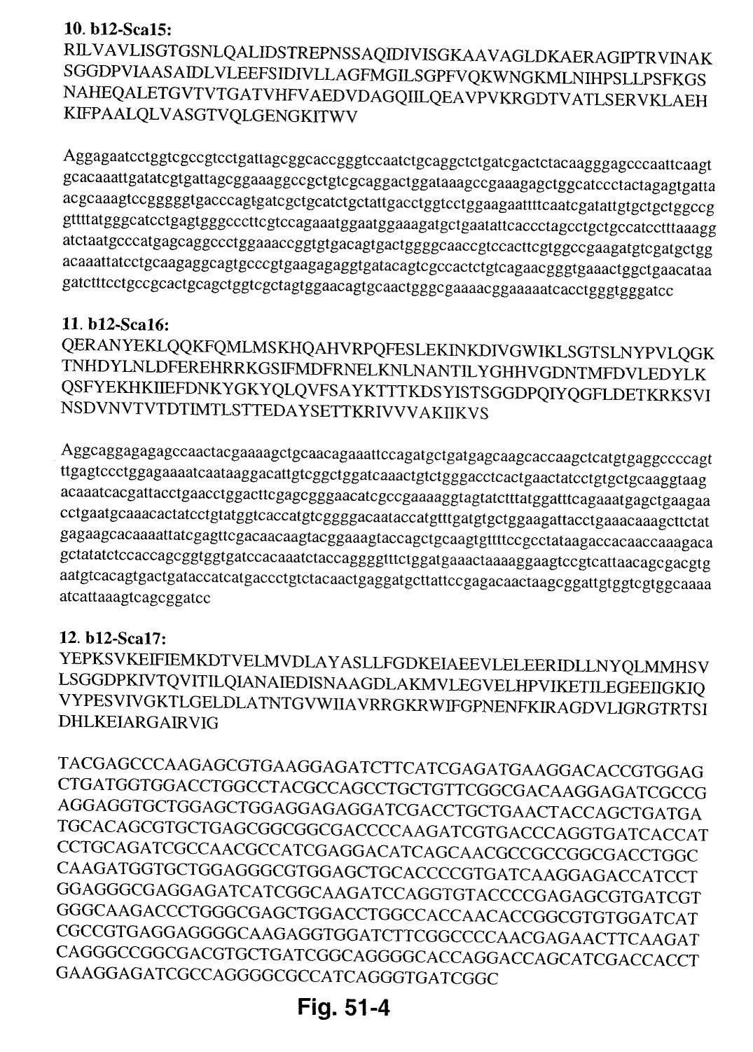

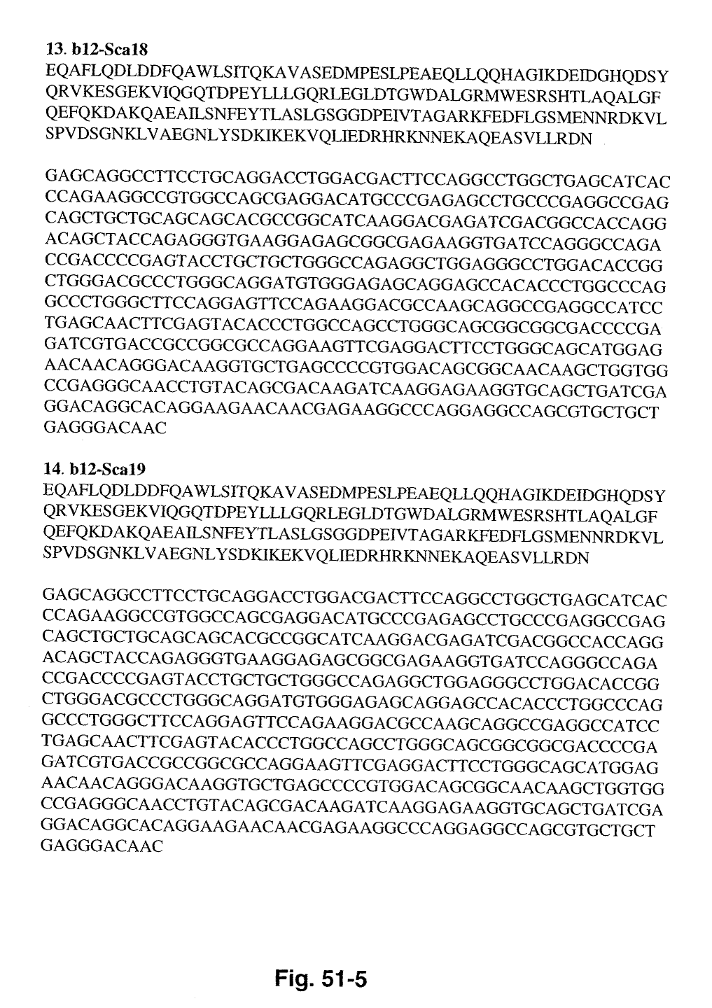

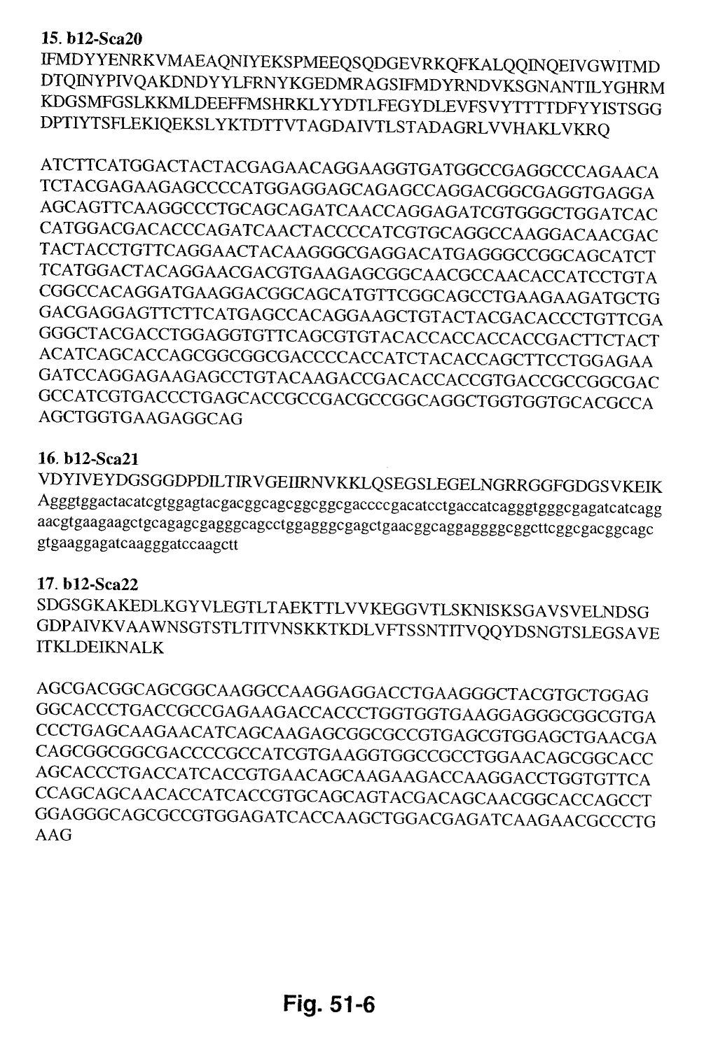

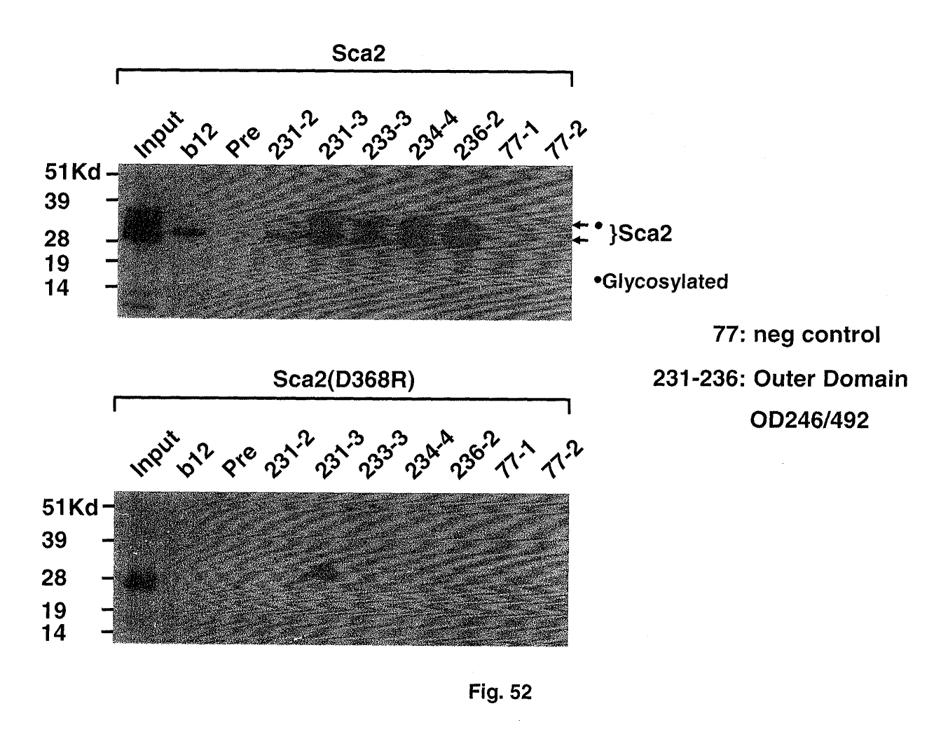

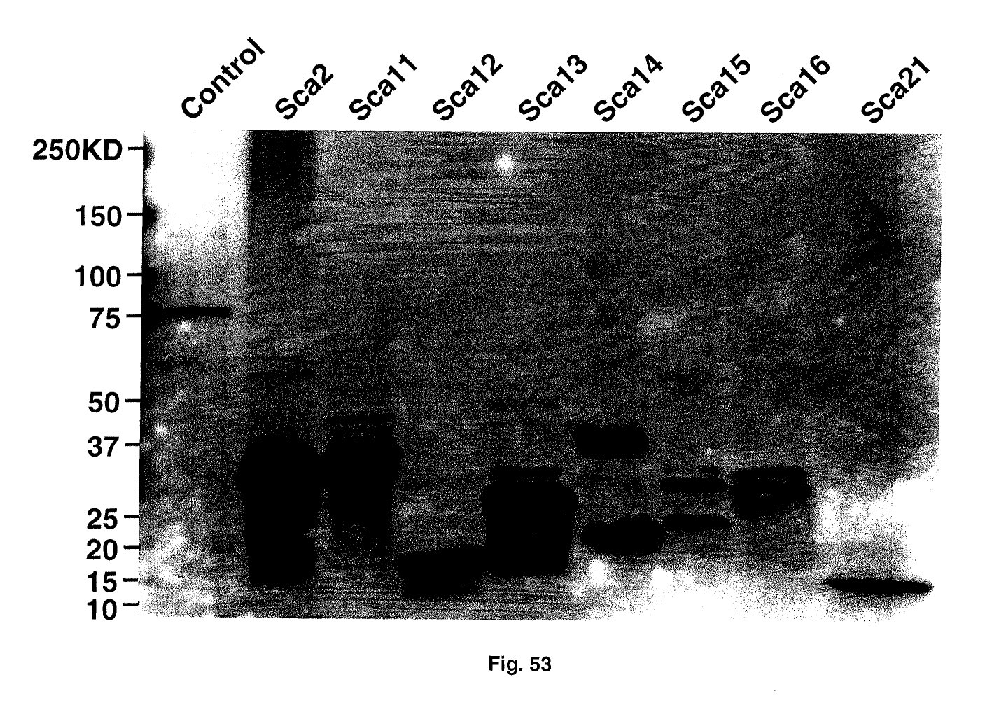

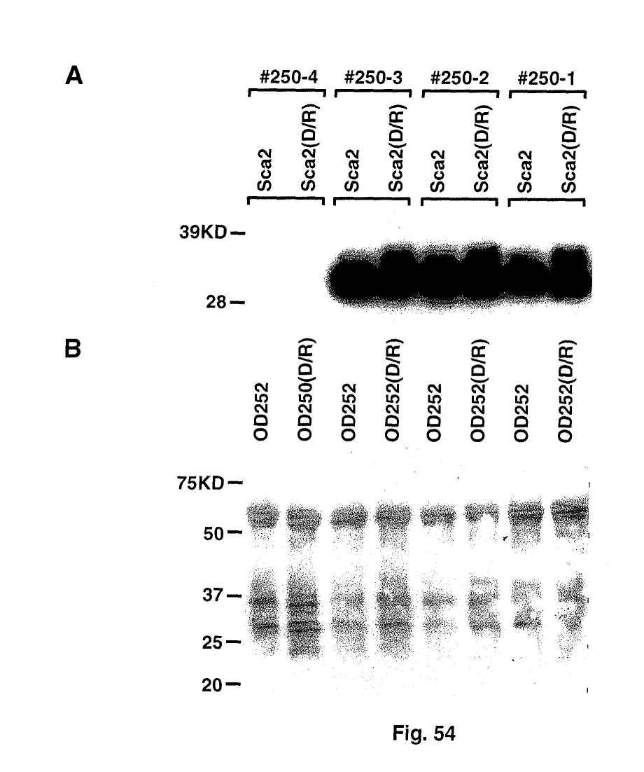

DNA and amino acid sequences for b12 Epitope-Transplant Scaffolds are provided in FIG. 50 and FIG. 51. The specificity of interaction of immune sera with Scaffold 2 (Sca2) is shown in FIG. 52. Expression of b12 scaffolds (Sca2, Sca11-16 and Sca21) in 293 cells is shown in FIG. 53. The effectiveness of antiserum against b12 Sca2 is demonstrated in FIG. 54. Immune sera (#250-1 to 4) from guinea pigs immunized with b12 Sca2 by DNA prime/ADV boosting regimen were able to immuno-precipitate Sca2 and its variant containing mutation D368R, demonstrating that it is immunogenic (FIG. 54A). The same immune sera were not able to interact with OD1(OD252/482) specifically (FIG. 54B).

### Immune Focusing

In one embodiment, the use of epitope-transplant scaffolds in immunization relates to the concept of immune focusing. If an immunogen elicits a number of responses, how might one focus or enhance a particular response? One way is with a prime-boost mechanism. If one has a particular epitope against which one wants an enhanced response, one can boost the original polyclonal response with an epitope-transplant scaffold, which only has the desired epitope in common with the original immunogen. B-cell populations that respond to both immunogens would then be clonally enhanced by the second boost. Such epitope boosting could be further enhanced by additional sequential boosts with immunogens that only retain the desired epitope.

A second way to enhance a particular response is to immunize with a mixture, where each of the molecules in the mixture has a particular epitope, but the immunogens are otherwise antigenically distinct. This second “mixture” approach can be also enhanced by prime-boost, for example, by a boost containing a second mixture of immunogens, where—except for the target epitope—each immunogen is antigenically distinct not only from each other, but also from the first mixture.

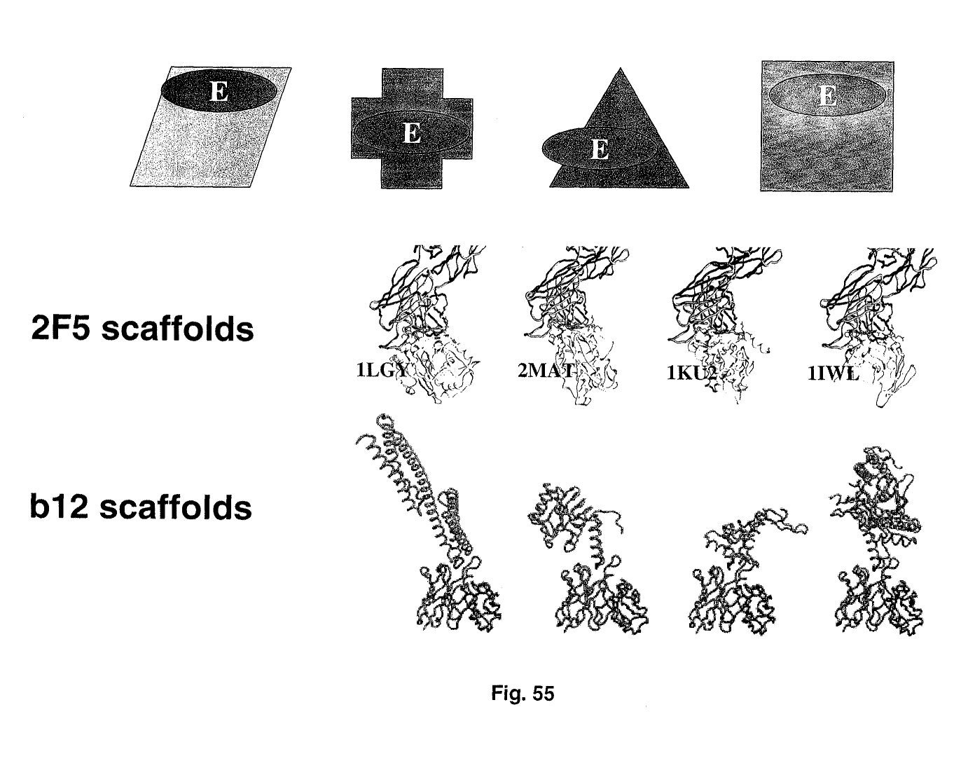

Two solutions are presented to the problem of transplanting epitopes onto scaffolds. One method involves epitope-scaffold transplantation, where the target epitope is transplanted into an entirely different scaffold. As shown in FIG. 55, the target epitope is shown transplanted into four antigenically different scaffolds, each with a different fold.

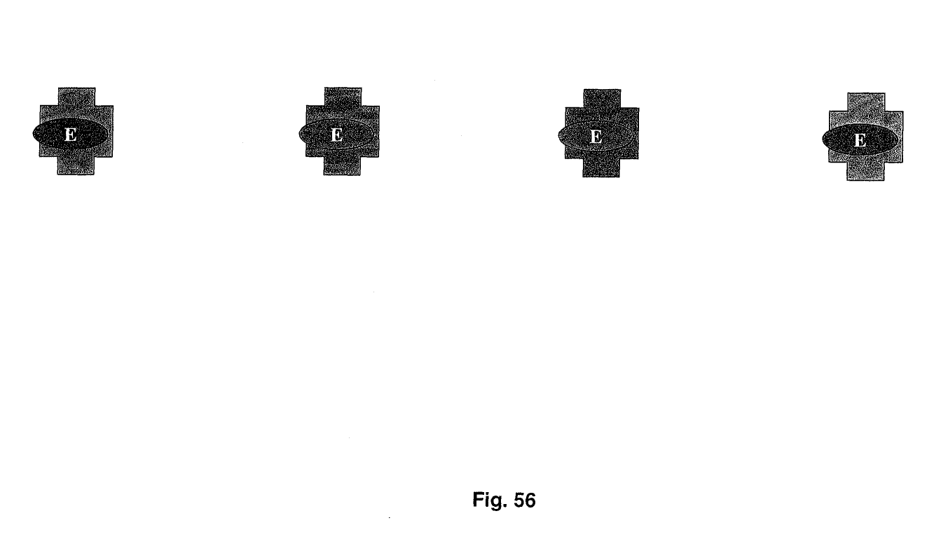

Another method involves antigenic cloaking (or homolog scaffolding), where the immunogen is modified so that every antigenic surface that is not the target epitope is modified. In FIG. 56, the target epitope is shown in four antigenically different backgrounds within the same overall fold.

### Computational Antigenic Cloaking

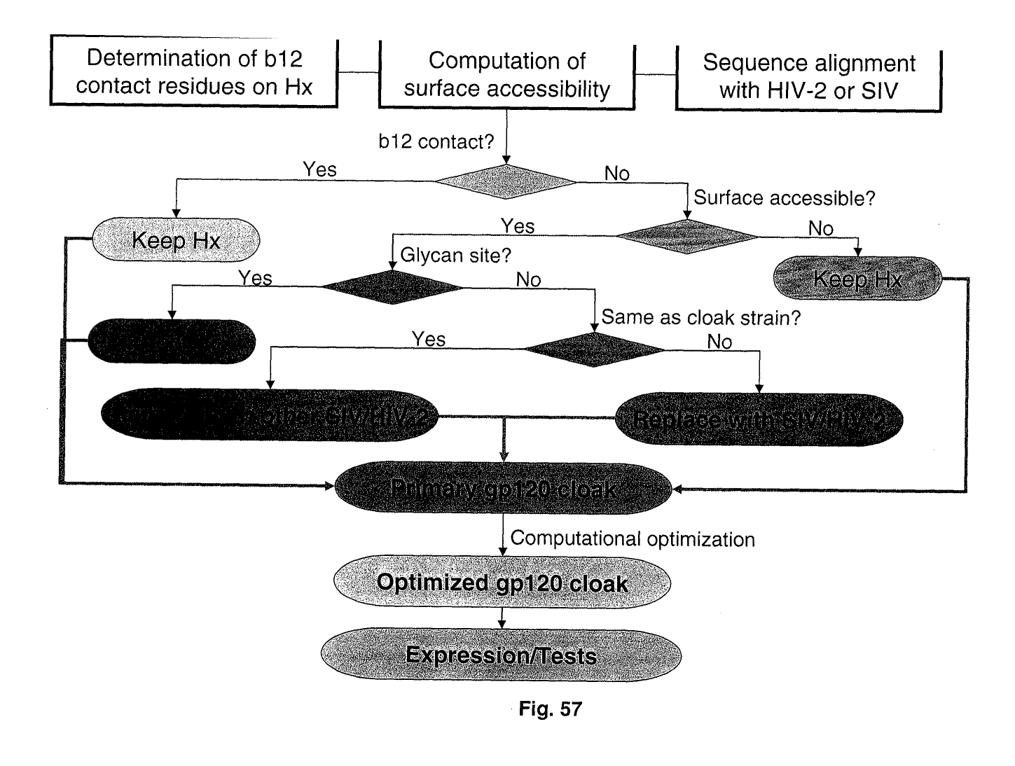

An initial algorithm based on evolutionarily related homolog replacement has been devised. A design flow chart for engineering antigenic cloaking scaffolds is given in FIG. 57. A sequence alignment of the antigen from HIV-1 and the cloaking strain (e.g., HIV-2 or SIV) reveals differences in amino acid sequence. Initially, the residues on the HIV-1 epitope that are contacted by antibody (e.g., by b12) are identified. The surface accessibility of the contacted residues is calculated. If a given residue in the HIV-1 epitope is contacted by the antibody, the residue is maintained. If it is not contacted, the surface accessibility of the residue is taken into account. If the residue is not accessible, it is maintained. If the residue is exposed to the surface, potential glycosylation at the site is investigated. If the residue is part of a glycosylation site, it is maintained. If it is not part of a glycosylation site, the sequence alignment with the cloaking strain is consulted to determine if the residue is the same as in the cloaking strain. If the HIV-1 residue is the same as the cloaking strain, the residue is replaced with an amino acid from an alternative SIV or HIV-2 strain that has a different amino acid at the position. If the HIV-1 residue is not the same as the cloaking strain, it is substituted with the corresponding residue in the cloaking strain. The foregoing steps lead to a primary gp120 cloak construct. The construct may be further modified following computational optimization. Ultimately, the construct is evaluated by expression and functional testing.

A more general computational algorithm for antigenic cloaking is also described here. We note at the outset that antigenic cloaking is not limited to cloaking of gp120 but can be applied to any protein bearing an epitope, including epitope-scaffolds. The necessary input information for computational antigenic cloaking is (1) the structure of an antigen and (2) the positions of residues on the antigen that comprise an epitope. The epitope positions in (2) can be determined from the structure of the complex between the antigen and an antibody using contact analysis as described above. The epitope positions in (2) can also be obtained more indirectly by epitope-mapping experiments such as alanine-scanning or hydrogen-deuterium exchange, so the structure of the antibody/antigen complex is not required even though it is advantageous. With the input information (1) and (2), the non-epitope surface positions are defined as the positions in the native antigen structure at which the side-chains are accessible to solvent and are not included in the epitope positions in (2). The non-epitope surface positions are the positions at which a computational design simulation can select optimal combinations of amino acids to “cloak” the antigen while maintaining folding stability and solubility. Such computational design could be carried out by ROSETTA_DESIGN, for example.

Multiple energetically acceptable “cloaked” antigens can be produced in such simulations, owing to the freedom to accommodate a wide variety of side-chains and side-chain conformations on the solvent-accessible surfaces of proteins while maintaining folding stability and solubility. In one general method, the design simulation can be allowed to choose among all possible amino acids at each non-epitope surface position. A large number of different low-energy sequences will be produced by such an unrestricted simulation, corresponding to a large number of different cloaks. The simulations optionally could be biased by the user, however, to produce cloaked surfaces with specific physico-chemical properties. Cloaked surfaces could be intentionally designed to be generally negatively charged, or generally positively charged, for some simple examples that might be expected to be particularly useful in avoiding cross-reactivity between cloaks. Design simulations could also be programmed to remember previously designed cloaks for a particular antigen and ensure zero or very little similarity between cloaks for the same antigen.

Such design simulations need not be restricted to maintaining the antigen backbone rigidly fixed in the native antigen conformation. Optionally, small variations in the antigen backbone conformation could be generated computationally, which would maintain the structural integrity of the epitope while allowing even greater freedom to design a variety of “cloaked” non-epitope surfaces.

To perform computational antigenic cloaking on glyco-proteins such as gp120, one could simply avoid changing native glycosylation sites as described above, or one could optionally include computational design of glycosylation sites on the non-epitope surface. Design of an N-linked glycosylation site requires at minimum placing a triplet sequence of NXS/T on the protein in a location at which the N is solvent accessible, in which N is asparagine, X is any residues except proline, S/T means serine or threonine. With computational design of glycosylation sites, one can in principle add and/or move glycans around on the non-epitope surface, to enhance cloaking while maintaining folding and stability. We note that computational design of glycosylation sites is not limited to proteins that are already glycosylated; epitope-scaffolds can be designed to contain one or more glycosylation sites on their non-epitope surface as another application of antigenic cloaking.

Finally we note that one has the option to incorporate available information from homologs during computational antigenic cloaking, to assist in maintaining proper folding and solubility of cloaked constructs. In this scenario, a multiple sequence alignment could be constructed for the antigen of interest, and at each non-epitope surface position, the computational design simulation could be restricted to choose among the amino acids present in the sequence alignment for that position. There are many possible options for biasing the selection of amino acids in this case. For example, the design simulation could be biased at any position to favor amino acids that occur more frequently in the multiple sequence alignment for that position.

### Reductionist Scaffolding

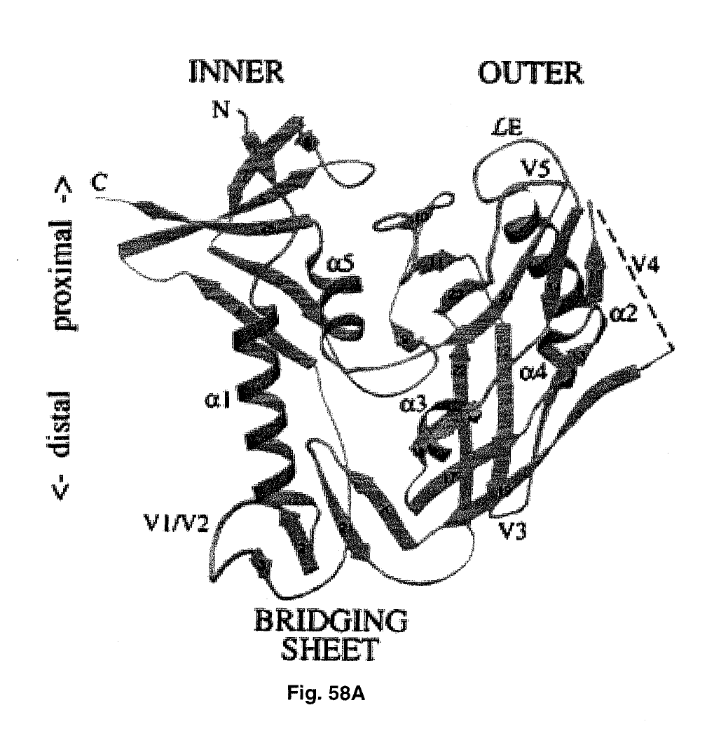

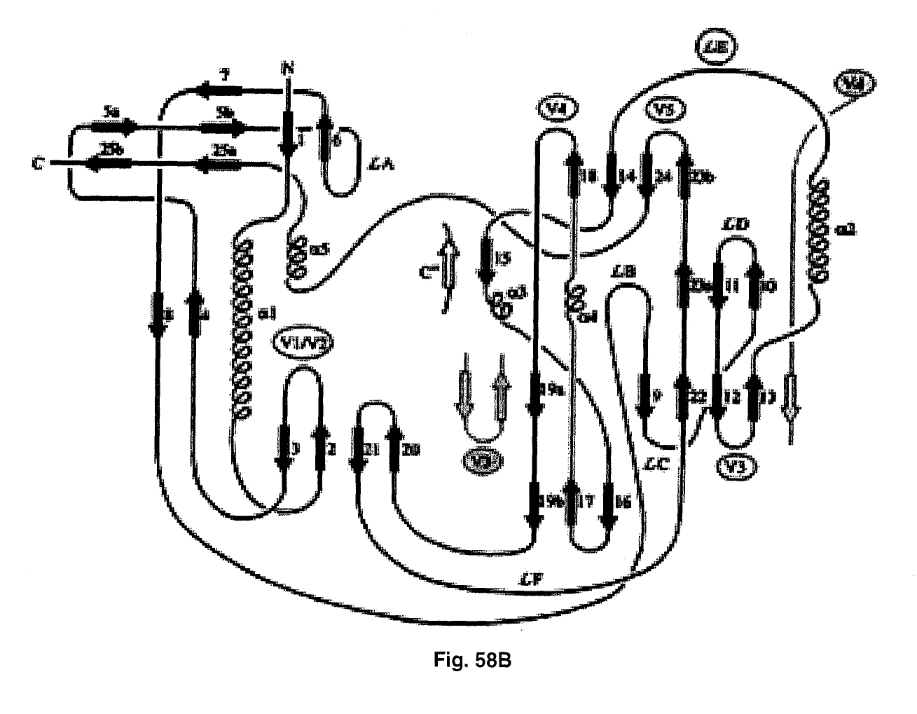

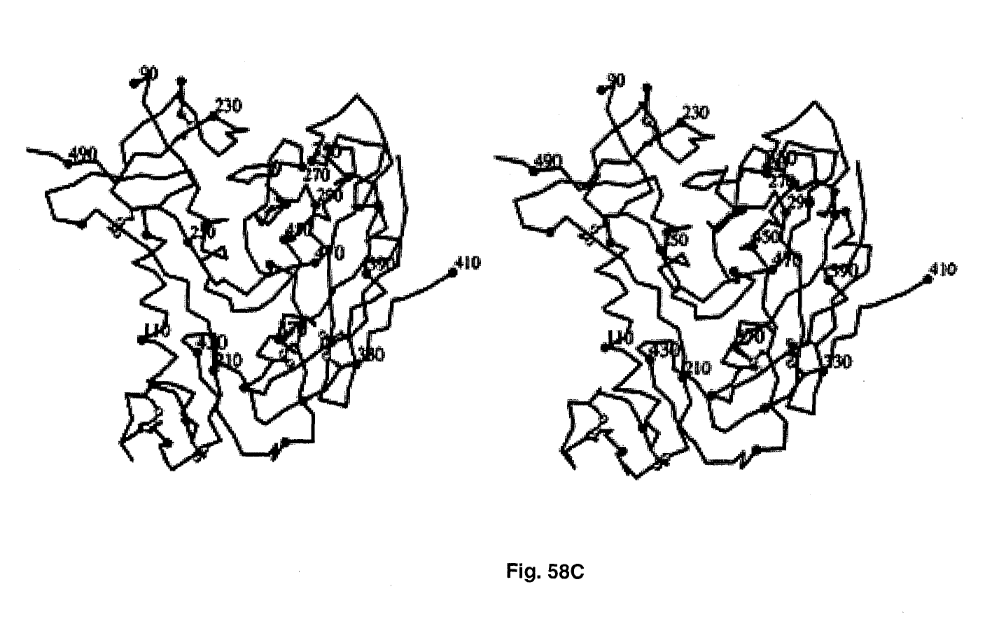

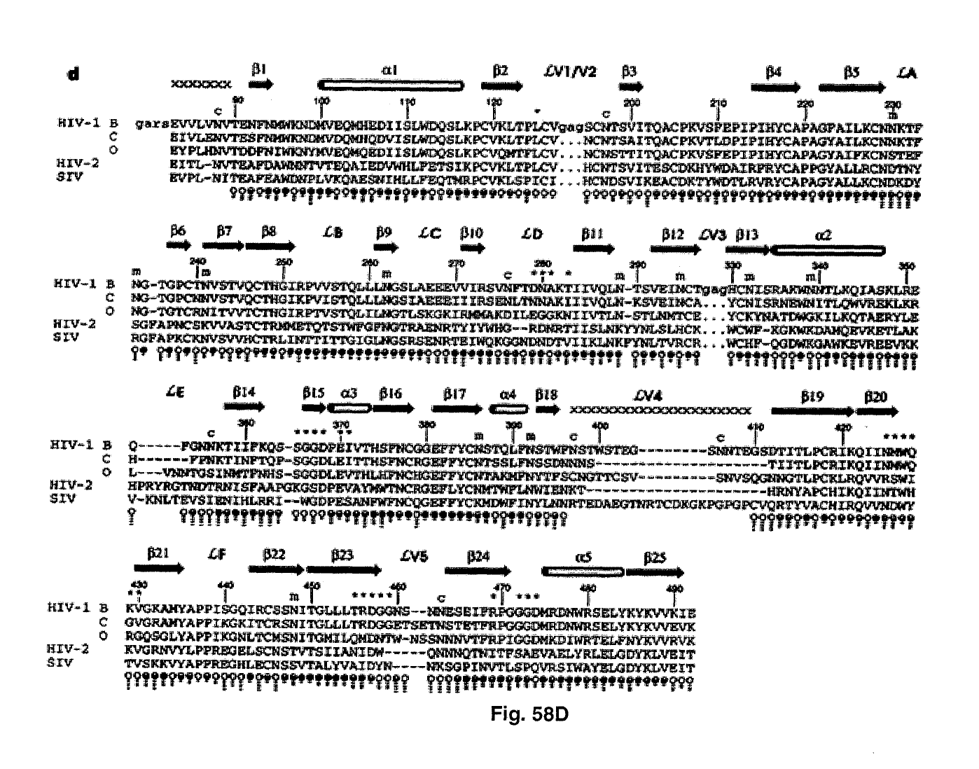

The CD4-bound state of gp120 comprises an inner domain, an outer domain and a four-stranded bridging sheet mini-domain. The deglycosylated core of gp120 as dissected from the ternary complex approximates a prolate ellipsoid with dimensions of 50×50×25 Å, although its overall profile is more heart-shaped than circular. Its backbone structure is shown in FIGS. 58 (a and c). This core gp120 comprises 25 β-strands, 5 α-helices and 10 defined loop segments, all organized with the topology shown in FIG. 58b. Specific spans of structural elements are given in FIG. 58d. The structure confirms the chemically determined disulphide-bridge assignments (FIG. 58c). The polypeptide chain of gp120 is folded into two major domains, plus certain excursions that emanate from this body. The inner domain (inner with respect to the N and C termini) features a two-helix, two-strand bundle with a small five-stranded β-sandwich at its termini-proximal end and a projection at the distal end from which the V1/V2 stem emanates. The outer domain is a stacked double barrel that lies alongside the inner domain so that the outer barrel and inner bundle axes are approximately parallel.

Referring to the structure of core gp120 in FIG. 58 (a-c), the viral membrane would be oriented above, the target membrane below, and the C-terminal tail of CD4 would be coming out of the page. In this view, we describe the left portion of core gp120 as the inner domain, the right portion as the outer domain, and the 4-stranded sheet at the bottom left of gp120 as the bridging sheet. The bridging sheet (β3, β2, β21, β20) can be seen packing primarily over the inner domain, although some surface residues of the outer domain, such as Phe 382, reach in to form part of its hydrophobic core.

Panel a shows a ribbon diagram. α-helices are depicted in black and β-strands in gray, except for strand β15, which makes an antiparallel β-sheet alignment with strand C″ of CD4. Connections are shown as solid lines, except for the disordered V4 loop (dashed line) connecting β18 and β19. Selected parts of the structure are labelled.

Panel b shows a topology diagram. The diagram is arranged to coincide with the orientation of a and c. Helices are shown as corkscrews and labelled α1-α5. β-Strands are shown as arrows: black and labelled represent the 25 β-strands of core gp120; grey and unlabelled represent the continuation of hydrogen bonding across a sheet; white and labelled represents the C″ strand of CD4. Spatial proximity between neighboring strands implies main-chain hydrogen bonding. Loops are labelled ζA-ζF and V1-V5. Labels for loops with high sequence variability are circled. Assignments of secondary structure were made with the Kabsch and Sander algorithm, except for β4 and β8 which are both interrupted mid-strand by side-chain-backbone hydrogen bonds, β9, β15 and β25a, all of which have angles or hydrogen bonds that are slightly non-standard, and α4, which hydrogen bonds as a 3₁₀ helix, with the final residue in β-conformation.

Panel c shows a stereo plot of an α-carbon trace. Every 10th C is marked with a filled circle, and every 20th residue is labelled. Disulphide connections are depicted as ball and stick. The ordered residues 90-396 and 410-492 are shown.

Panel d shows a structure-based sequence alignment. The sequences are shown of HIV-1 B (core gp120 from clade B, strain HXBc2), C (HIV-1 clade C, strain UG268A2), O (HIV-1 clade O, strain ANT70), HIV-2 (strain ROD), and SIV (African green monkey isolate, clone GRI-1). The secondary-structure assignments are shown as arrows and cylinders, with a cross denoting residues that are disordered in the present structure. The ‘gars’ sequence at the N terminus and the ‘gag’ sequence in the V1/V2 and V3 loops are consequences of the gp120 truncation. Solvent accessibility is indicated for each residue by an open circle if the fractional solvent accessibility is greater than 0.4, a half-filled circle if it is 0.1 to 0.4, and a filled circle if it is less than 0.1. Sequence variability among primate immunodeficiency viruses is indicated below the solvent accessibility by the number of horizontal hash marks: 1, residues conserved among all primate immunodeficiency viruses; 2, conserved among all HIV-1 isolates; 3, moderate variation among HIV-1 isolates; and 4, significant variability among HIV-1 isolates. In assessing conservation, all single atom changes were permitted as well as larger substitutions if the character of the side chain was conserved (for example, K to R or F to L). N-linked glycosylation is indicated by ‘m’ for the high-mannose additions and ‘c’ for the complex additions in mammalian cells. Residues of gp120 in direct contact with CD4 are indicated by an asterisk. Direct contact is a more restrictive criterion of interaction than the often-used loss of solvent-accessible surface; residues of gp120 that have lost solvent-accessible surface but are not in direct contact include 123, 124, 126, 257, 278, 282, 364, 471, 475, 476 and 477.

To increase the immunogenicity of gp120, a rational approach is to modify or engineer the gp120 molecule to expose or generate conserved neutralizing epitopes. Some experimental data suggested that this might be achievable. It was reported that removing the V1/V2 variable loops from gp120 rendered the underneath conserved regions more vulnerable to antibody neutralization. It was reported that removal of the V1, V2, and V3 hypervariable loops resulted in a truncated gp120 that was capable of binding to soluble CD4 with an affinity comparable to that of full-length gp120. Moreover, removal of the variable loops increased accessibility of the C1 and C4 regions to monoclonal antibodies. It was also demonstrated that the V1/V2 were dispensable for viral replication but played a role in shielding the receptor binding sites. Recently, investigators reported that removal of the V1-V3 loops resulted in a truncated gp120, designated PR12, which was able to elicit a broadly reactive neutralizing antibody response in rats, although the epitopes of the neutralizing antibodies thus generated await further characterization. On the other hand, it was reported that selective deletion of some glycosylation sites in gp120, thus removing the carbohydrates, resulted in enhanced immunogenicity. For example, investigators have shown that selective removal of N-glycosylation sites of the simian immunodeficiency virus resulted in a mutant virus that was neutralization-sensitive, and that the altered virus was able to raise better antibody responses against the wild-type virus. These results suggest that modifying the antigenic structure of gp120 to produce a core constitutes a promising strategy to improve the immunogenicity of gp120 for an effective HIV vaccine.

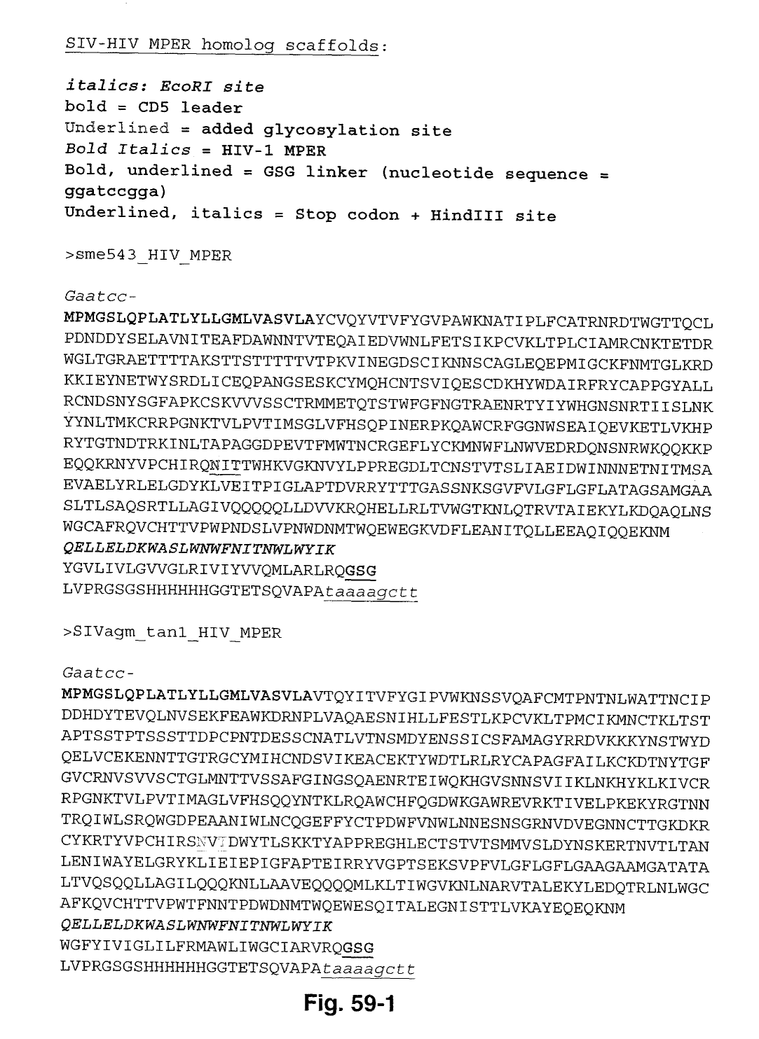

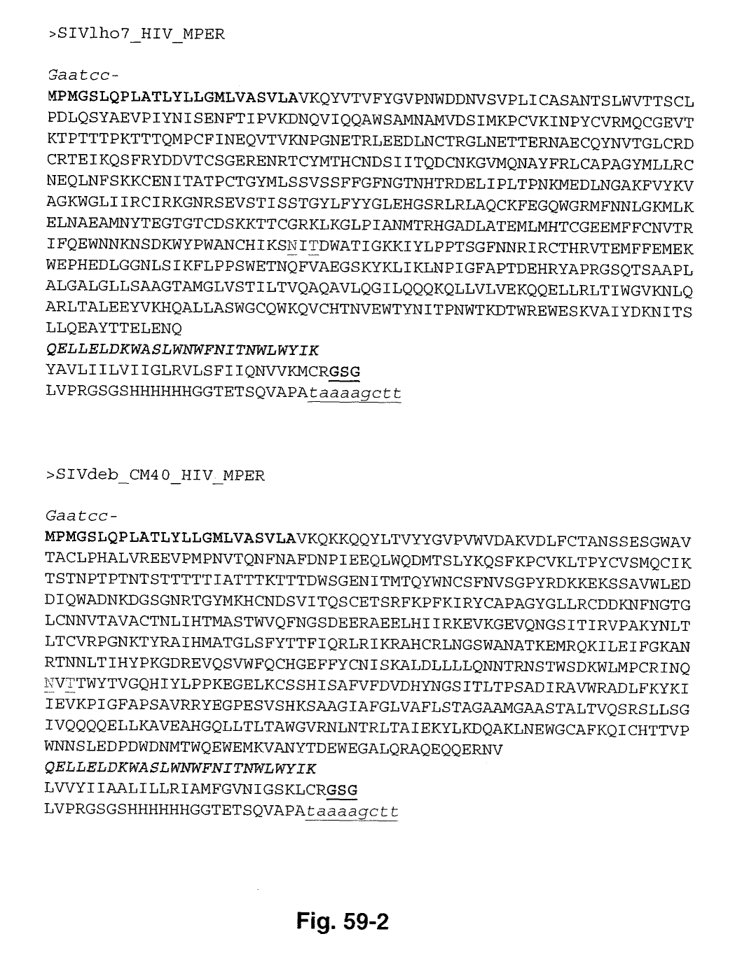

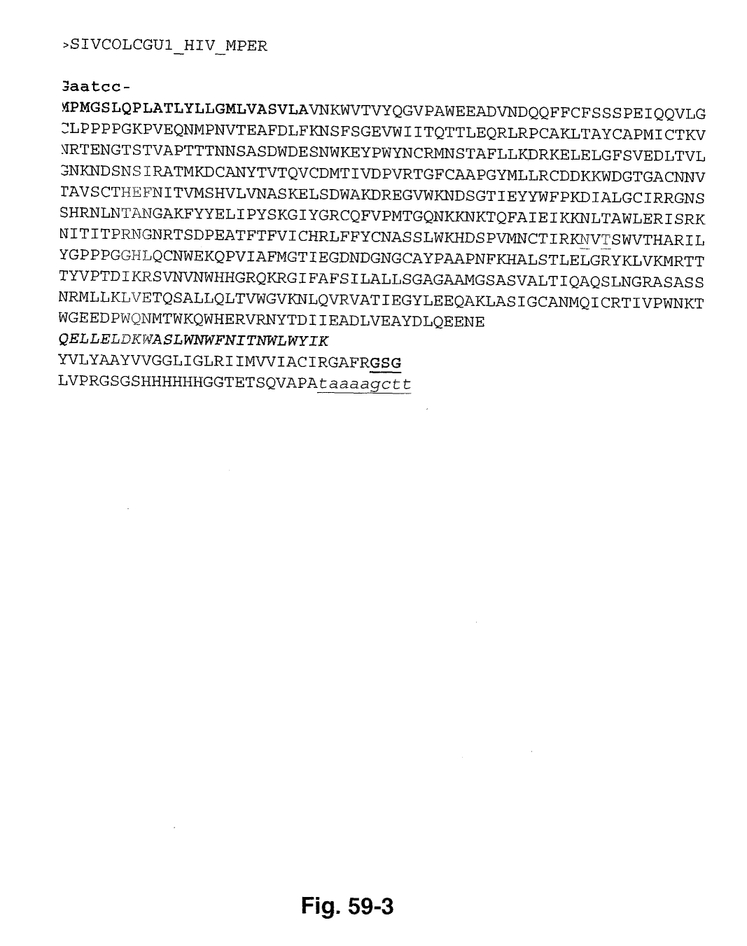

A set of SIV-HIV homolog scaffolds containing gp140 sequence with the membrane proximal portion altered is shown in FIG. 59.

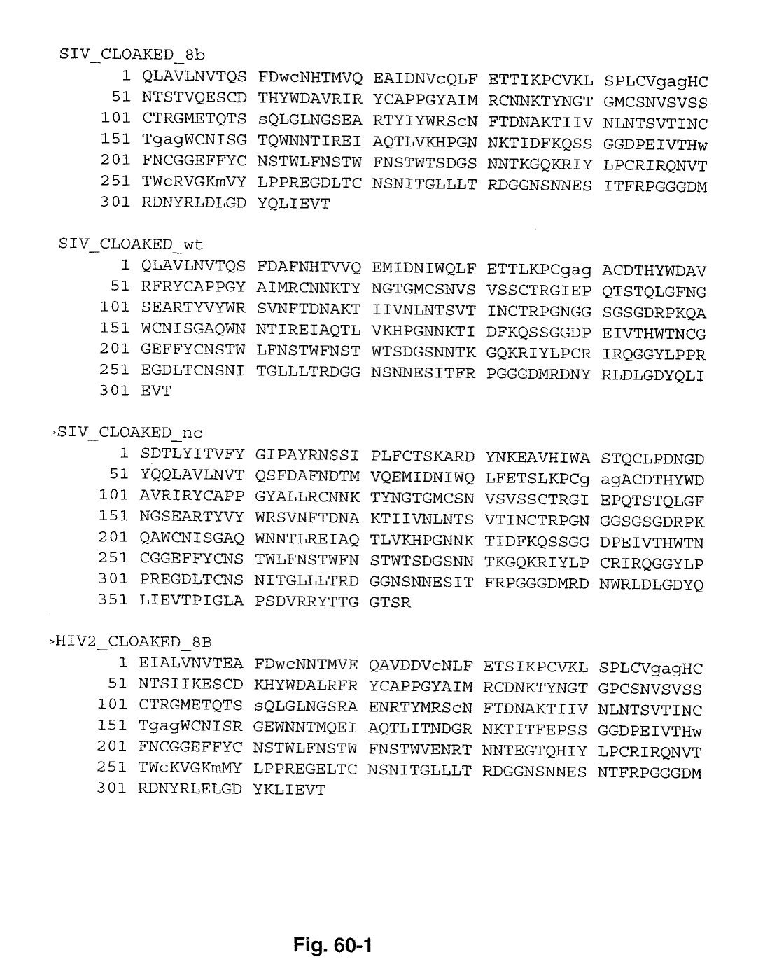

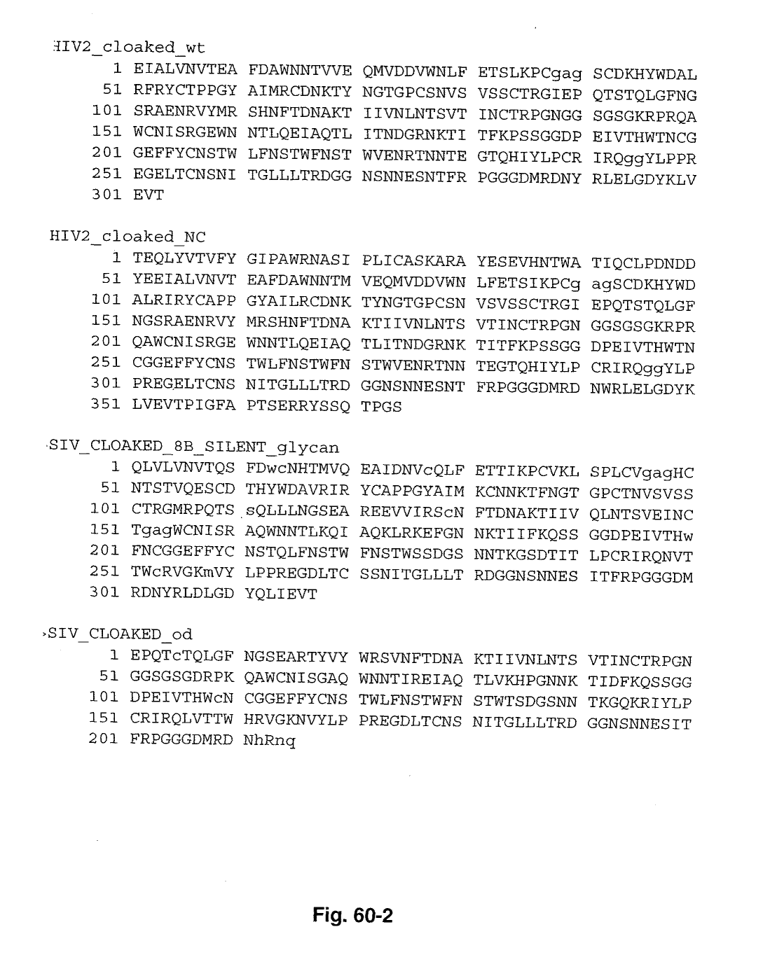

Each of SIVmac239 and HIV-2 7312A cloaks are created in the context of HxBc2 Ds12F123, wild type core with the bridging-sheet removed and new V3 design, and wild type core with the bridging-sheet removed and new V3 design+N/C terminal (See FIG. 60). In addition, a SIV cloak construct in the context of Ds12F123 was created with glycan-covered residues unchanged (used a 7.5 Å radius to calculate the residues). An outer domain SIV cloak was also designed based on the OD1 sequence (252-482) with some of the newly exposed inner-outer domain interface residues changed to reduce hydropobicity, an extra disulfide bond between aa256 and aa276 was also added to this OD cloak to stabilize the N-termini.

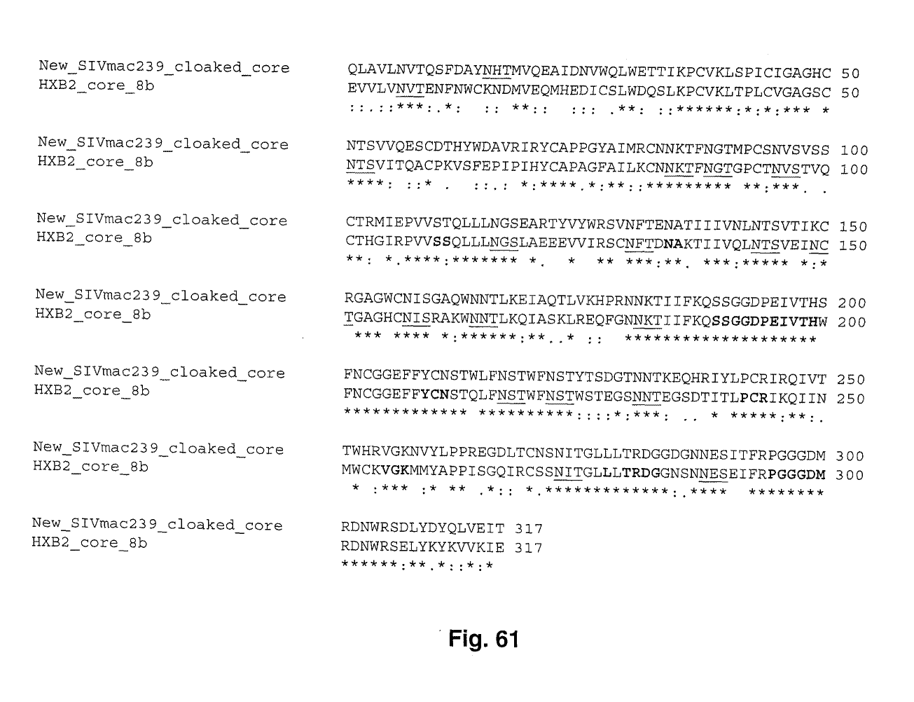

Reductionist scaffolding can be combined with the method of antigenic cloaking. For example, the Hx-8b core can be cloaked in a homologous background such as HIV-2 7312A and SIVmac239. The amino acid sequence of one embodiment termed New_SIVmac239_cloaked_core is aligned with the HXB2_core_{—}8B amino acid sequence in FIG. 61. Amino acid residues that contribute to the b12 epitope are indicated in bold.

### Vaccine Implications

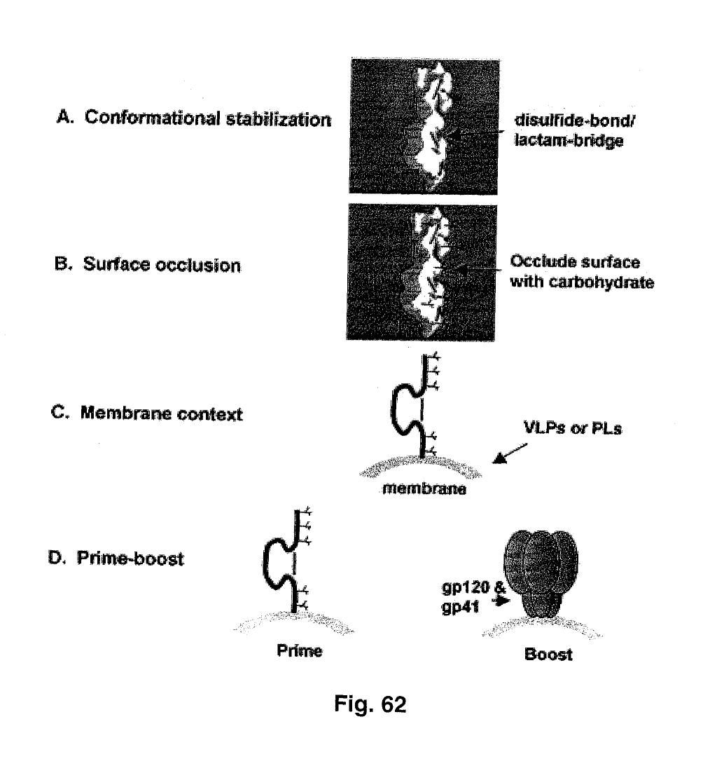

An effective immunization strategy to elicit 2F5-like broadly neutralizing antibodies would likely have to account for viral mechanisms of immune evasion that constrain the membrane-proximal region, namely, conformation, surface occlusion, and membrane proximity, although perhaps not large-scale steric accessibility. The precise conformation that 2F5 recognizes may be difficult to stabilize. Both the upstream six-helix bundle and downstream membrane-bound helix enforce different conformations on the 2F5 epitope. The stabilization of extended structures is also not trivial. Tight turns can be stabilized with designed disulfide or lactam bridges (FIG. 62A), and such approaches are already under way; even so, such turns account for less than half of the 2F5 epitope. One critical question will be the degree of flexibility of the 2F5 epitope in the context of the full envelope ectodomain. Does the entire 2F5-bound conformation observed in this structure have to be stabilized, or is only a critical substructure essential for broad immune recognition? Such questions should be answerable by immunizations with structurally constrained antigens. Alternatively, structures of the 2F5 epitope in a more complete ectodomain context, or even in complex with another neutralizing antibody that recognizes the 2F5 epitope, may also provide answers.

A vaccine immunization strategy is depicted in FIG. 62. Shown is a four-part strategy to elicit 2F5-like antibodies. Panel A demonstrates conformational stabilization of the 2F5-bound extended conformation of gp41. The molecular surface of a potential immunogen is shown, with the surface bound by 2F5 shaded and the surface hidden from 2F5 in white. Disulfide bonds or lactam bridges that stabilize conformation are shown as lines. Panel B shows surface occlusion of the hidden face of gp41. Carbohydrate (black) is shown occluding the hidden hydrophobic surface of gp41 from humoral immune recognition. Due to the size of the epitope, N-linked glycans may be too large to use here, but smaller O-linked glycans may allow more precise masking. Panel C depicts membrane context. To elicit antibodies that are able to accommodate an epitope that is proximal to membrane, one could immunize with a conformationally stabilized, surface-occluded immunogen in the context of membrane, either on virus-like particles (VLPs) or on proteoliposomes (PLs) (see WO 02/056831). Panel D depicts a prime-boost strategy. Various prime-boost strategies could be employed to select only those antibodies that are able to overcome accessibility barriers to the membrane-proximal region. Shown here is one example, with the prime consisting of a conformationally stabilized, surface-occluded immunogen presented in the context of membrane. A boost with the complete Env ectodomain could select antibodies that can bind to the native viral spike.

To account for local surface occlusion, immunogens that induce antibodies that only bind to the 2F5-bound surface would need to be designed. This might be accomplished in a manner similar to that tried for anti-gp120 immunogens, for example, by masking the unbound hidden surface of gp41 with carbohydrate modifications (FIG. 62B). O-linked glycosylation might be preferable in this case due to the smaller size of these glycans, which would interfere less with the 2F5-bound face of the peptide. Alternatively, one could anchor the epitope to a larger molecule or surface in a manner that would leave only the 2F5-bound surface exposed. For example, one could first attach reactive groups on to the hidden face of the gp41 epitope, then bind the 2F5 complex to a nonimmunogenic graphite or plastic surface that reacts with these groups, and then release 2F5. The latter approach not only would eliminate local surface occlusion but also would allow the reactive groups to weld the 2F5-enforced conformation into place.

In terms of membrane proximity, one could present a conformationally stabilized, surface-occluded immunogen in the context of membrane, either on virus-like particles or on PLs (FIG. 62C). Enhanced 2F5 binding observed in the context of a PL membrane suggests that even in a highly artificial context, the presence of membrane recapitulates essential components of 2F5 recognition.

Elicitation of 2F5-like antibodies with any of these immunogens could be enhanced with prime-boost strategies (FIG. 62D). For example, priming with a conformationally stabilized, surface-occluded, membrane-anchored immunogen may elicit high titers of antibodies, only a small portion of which recognize virus. A boost, on the other hand, composed of the complete wild-type envelope ectodomain and presented in a membrane-anchored context, could select antibodies capable of binding wild-type virus. Such prime-boost strategies might be repeated (for example, with diverse strains of HIV or with additional peptides) to enhance antibody specificity and titer.

These immunization strategies (FIG. 62) should account for the constraints on the conserved membrane-proximal epitope suggested by our mechanistic analysis of the 2F5-gp41 crystal structure. The analysis presented here defines a sufficient road map for elicitation of 2F5-like antibodies. Our studies on 2F5 present a paradigm for using structural information from broadly neutralizing antibodies to understand and overcome HIV-1 mechanisms of immune evasion.

### Env/MPER-PLs

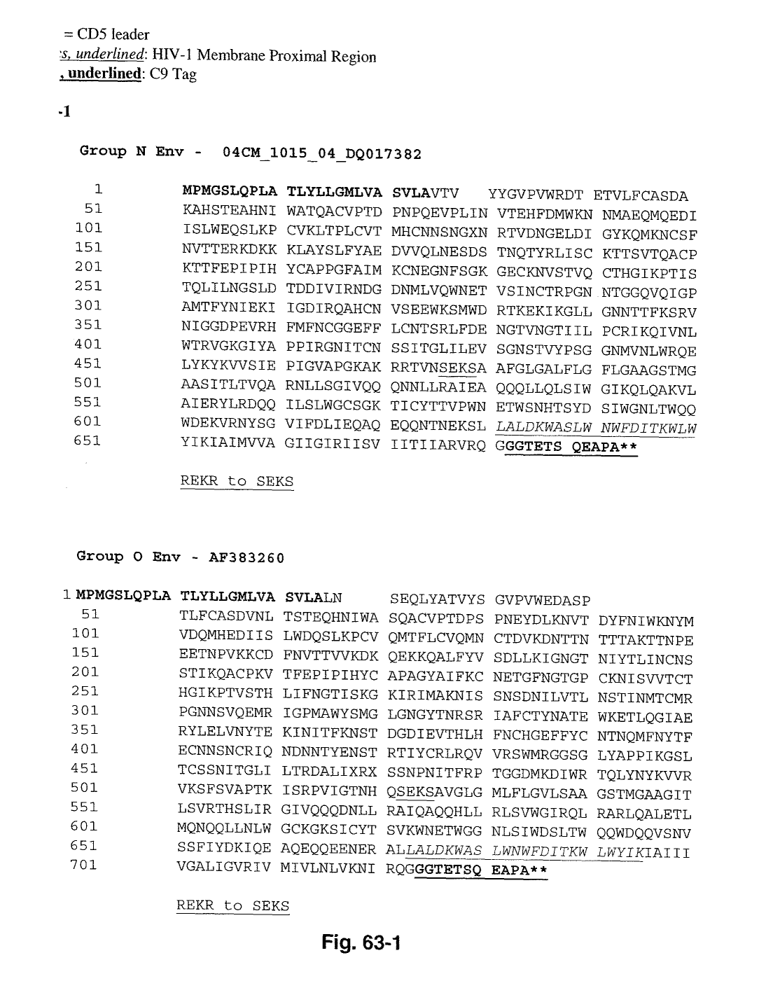

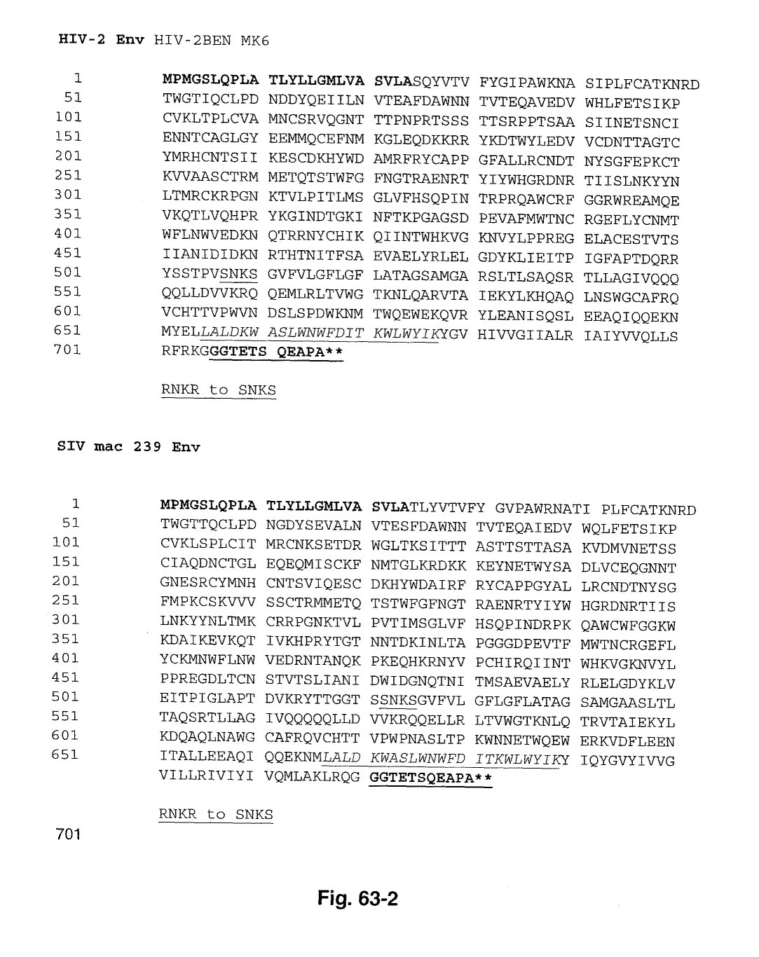

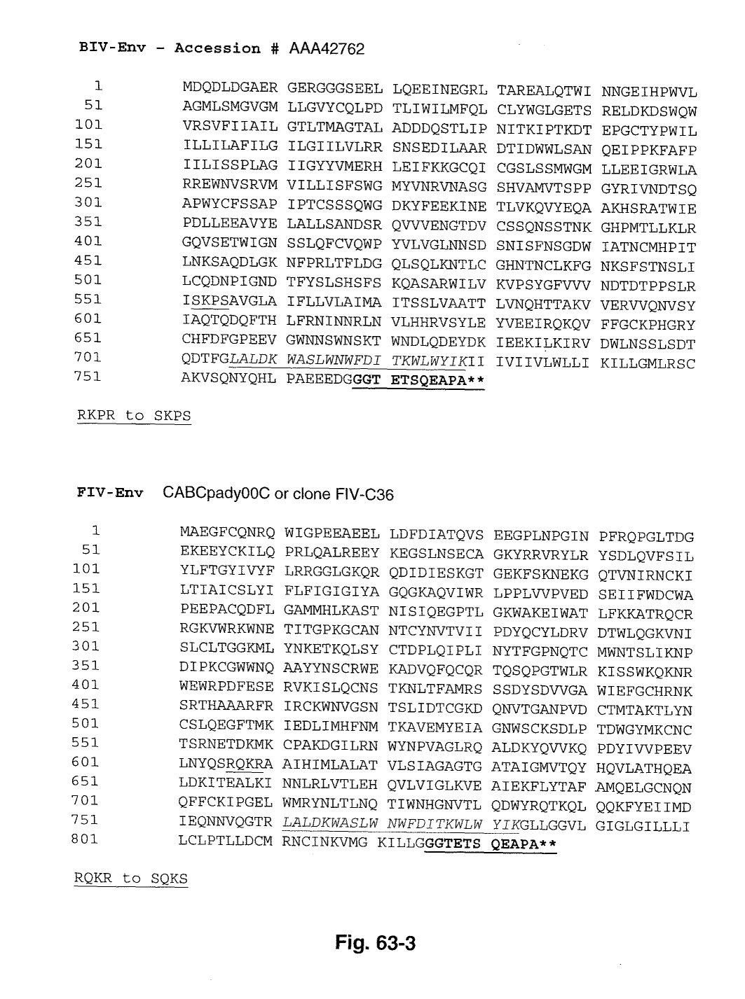

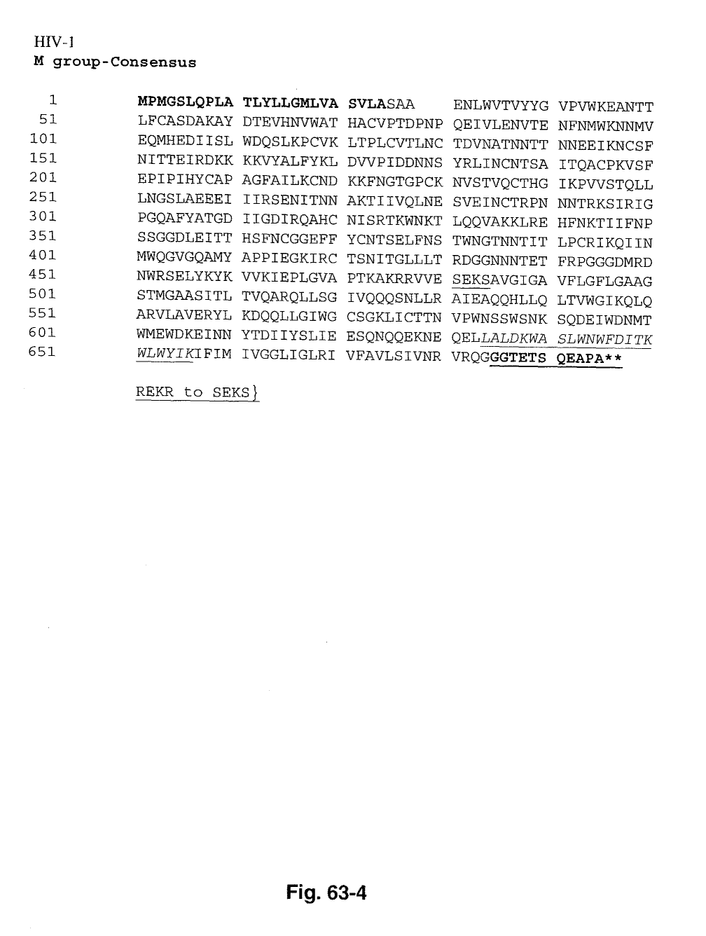

By sequence homology alignment, we have replaced the gp41 membrane proximal regions (MPER) of related but genetically diverse primate lentiviruses with the HIV-1 MPER from the YU2 HIV-1 Group M, clade B strain (FIG. 63). Then we have made these envelope glycoproteins cleavage-defective by modifying known or putative precursor cleavage sites (underlined), truncated their cytoplasmic tails and appended a C9 tag sequence. These will be expressed in mammalian cells to make envelope glycoprotein proteoliposomes (Env PLs) (See WO 02/056831).

In brief, the cells will be lysed in detergent, the Env/MPER glycoproteins captured on solid phase beads using an antibody against the C9 tag linked to the beads, the detergent replaced with lipid to from solid phase Env/MPER proteoliposomes.

The acceptor Envs for the HIV-1 MPER graft come from individual isolates HIV-1 groups O and N, an HIV-2 isolate, an SIV isolate (mac 239), an BIV isolate and a FIV isolate to serve as prototypes. In principle, other isolates or consensus sequence Envs could be used.

The concept would be to present the MPER in a relatively natural envelope glycoprotein context, proximal to a lipid bilayer. The envelope glycoprotein regions outside the MPER would be antigenically diverse enough to not elicit cross-reactive antibodies. The Env/MPER-PLs would be immunized in sequence to enhance antibody responses against the MPER possessed in common by each Env/MPER-PL. Due to limited or no cross-reactivity, this would be the predominant antibody response that would be boosted by such sequential immunization.

### Purification of Putative 2F5-like Antibodies from a Human Patient Using a gp41e-1KU2 (Scaffold) Affinity Column

The general principle and objective was to use epitope scaffolds as a diagnostic to verify presence of antibodies that react with the structurally defined epitope graft in human HIV-1 patient sera, and then use the scaffolds to purify these antibodies for future studies. Previous ELISA experiments found that patient 1679 (and three other bleeds from the same patient) had reactivity against both the 1IWL and 1KU2 scaffolds, with an estimated concentration of only 50 ng/ml.

A 1KU2 Column was prepared for human patient sera antibody purification. Approximately 2OD 1KU2s were conjugated to 2 mls of beads, as follows. 1KU2 was dialyzed against 0.2 M NaHCO3, 0.5 M NaCl, pH 8.3 (o/n). Gel matrix was washed with 10-15 gel volumes of COLD 1 mM HCl. The protein solution was added to the beads (approximately 1.2 mls into 2 mls beads (pH ˜9)). The mixture was incubated for 2-4 hrs at RT on a nutator. Unreacted sites on the gel matrix were blocked with 0.5M ethanolamine, 0.5M NaCl (pH 8.3) for 2 hr at RT. The gel was washed with alternating 3×1 col vol washes of high pH and then low pH buffers (0.1 M Tris-HCl pH 8, 0.5 M NaCl and 0.1 M Acetate pH 3, 0.5 M NaCl). The wash step was repeated 6 times. The prepared 1KU2s beads were divided into 2 columns of 1 ml each (one for the serum samples, and the other for a 2F5 control purification).

Before proceeding with the purification from the valuable serum, a control experiment was performed using the 2F5 antibody. 2F5 IgG was diluted into 8 mls of normal human plasma to achieve a final concentration of 50 ng/ml (or 400 ng total mass of ab). The product was loaded onto column, washed with 50 col vols using 1×PBS/0.5 M NaCl, and eluted with Pierce elution buffer, collecting 100 μl fractions (which were then pooled into 500 μl fractions).

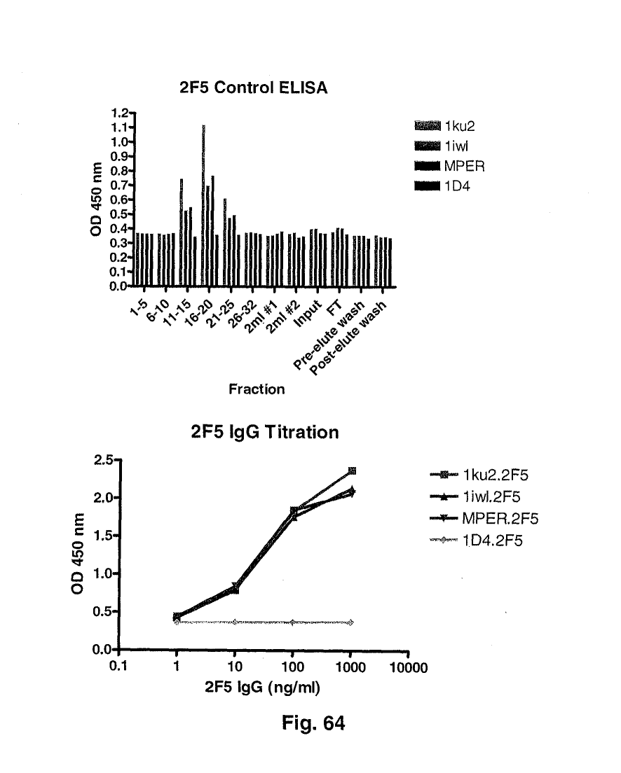

ELISAs were run on the purified fractions, to determine purification yield (See FIG. 64). Based on OD 450 nm reading for 2F5 IgG that was run side by side with the 1KU2-purified 2F5, obtained the estimates for yields given in Table 5. Since the initial amount was ˜400 ng, final yield was ˜44%.

### 1KU2 Affinity Purification of Pooled Bleeds from Patient 1679

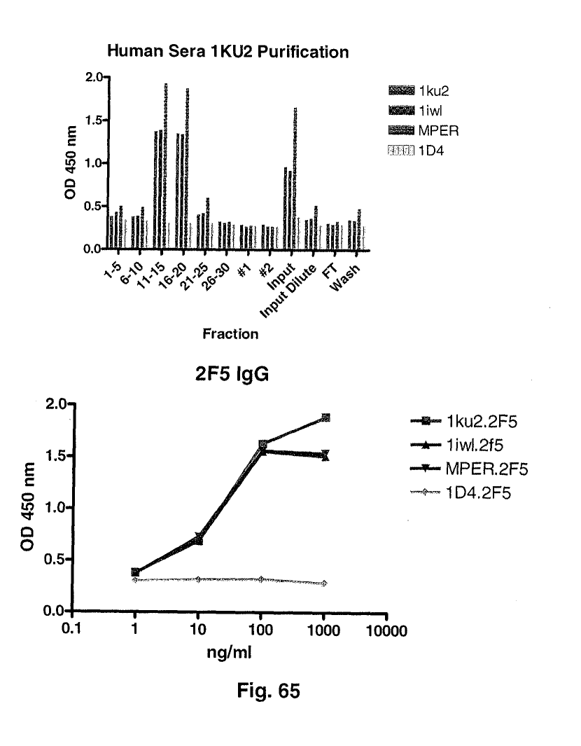

We performed an almost identical protocol as was done for 2F5, except that used 9.2 mls instead of 8 mls (and therefore 460 ng estimated starting mass of 2F5-like ab). See FIG. 65 for results. Based on 2F5 IgG results in this ELISA, we obtained estimates for yields given in Table 6. These purified antibodies can subsequently be tested for neutralization of various HIV-1 strains, and then further purified.

### 2F5 Epitope Scaffolds Immunogenicity Study: Neutralization

We measured the HIV-1 neutralizing activity of anti-scaffold serum after three immunizations using the TZM-b1 cell assay that utilizes a luciferase reporter gene (Luc). TZM-b1 cells are HeLa cells that express CD4, CXCR4 and CCR5 and can sustain HIV infection. These target cells contain a Tat-responsive reporter gene for firefly luciferase under control of an HIV-1 long terminal repeat. Expression of the reporter gene is induced in trans by viral Tat protein soon after infection. Luciferase activity is directly proportional to the amount of input virus. This assay quantifies neutralization as a function of a reduction in Luc reporter gene expression as infection of TZM-b1 cells is blocked by serum.

Anti-scaffolds serum samples that showed detectable binding to 2F5 peptide by ELISA were subjected to the neutralization assay. Values shown in the last column of Table 7 represent serum dilutions required to achieve 50% neutralization of HIV-1 HxB2 pseudovirus.

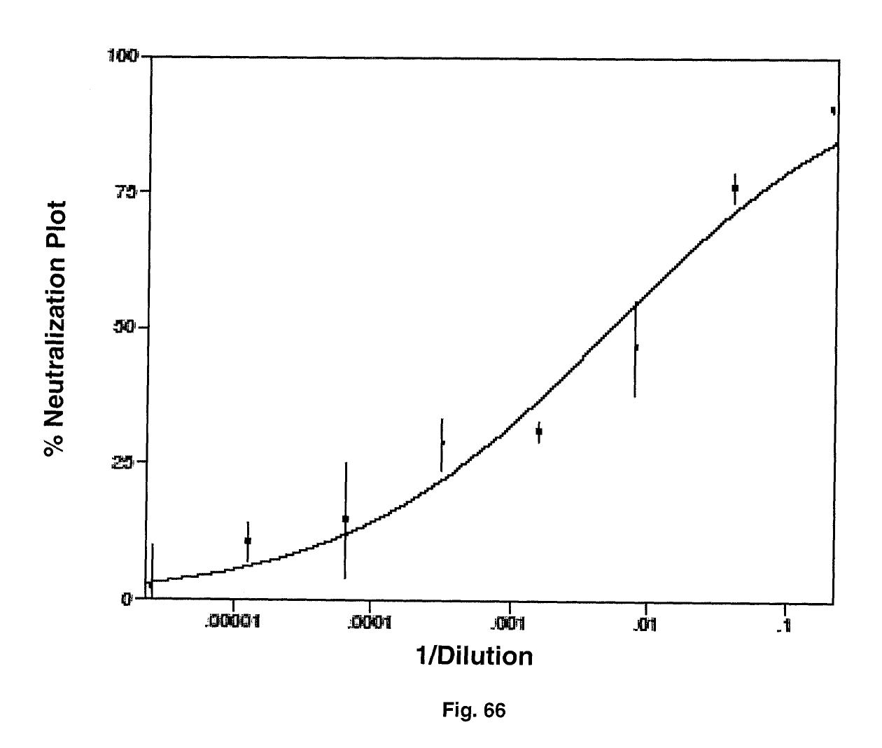

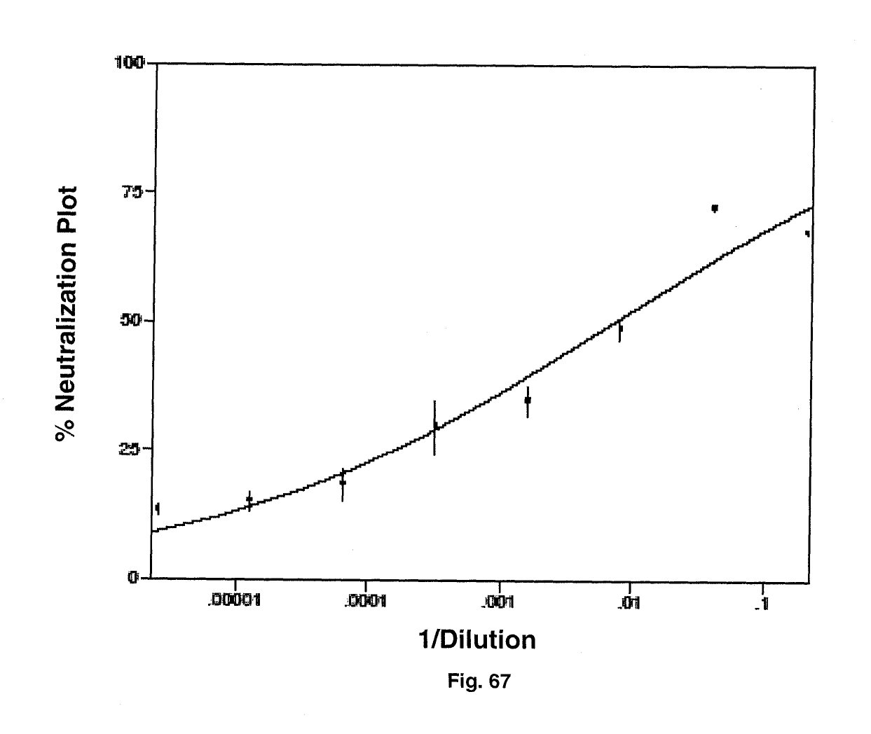

The results show that three of the constructs tested as immunogens (2F5e_{—}1lgy, 2F5e_{—}1d3bb and 2F5e_{—}1d3bb_TH) were capable of eliciting detectable neutralizing responses after three immunizations following the regimen described for the immunogenicity study. Neutralization curves for the highest neutralizing responses are shown in FIG. 66 for Animal study I-003 and in FIG. 67 for animal study E_{—}325. FIG. 66 shows a neutralization curve obtained for a serum sample of animal I-003 after 3 immunizations with scaffold 2F5e_{—}1d3bb_TH. Numerical values for percent neutralization by the serum of animal I-003 at various dilutions are listed in Table 8. FIG. 67 shows a neutralization curve obtained for serum sample of animal E-325 after 3 immunizations with scaffold 2F5e_{—}1lgy. Numerical values for percent neutralization by the serum of animal E-325 at various dilutions are listed in Table 9. These are the neutralization curves for the two animals with the most potent responses.

### Proper Surface Accessibility of the Epitope

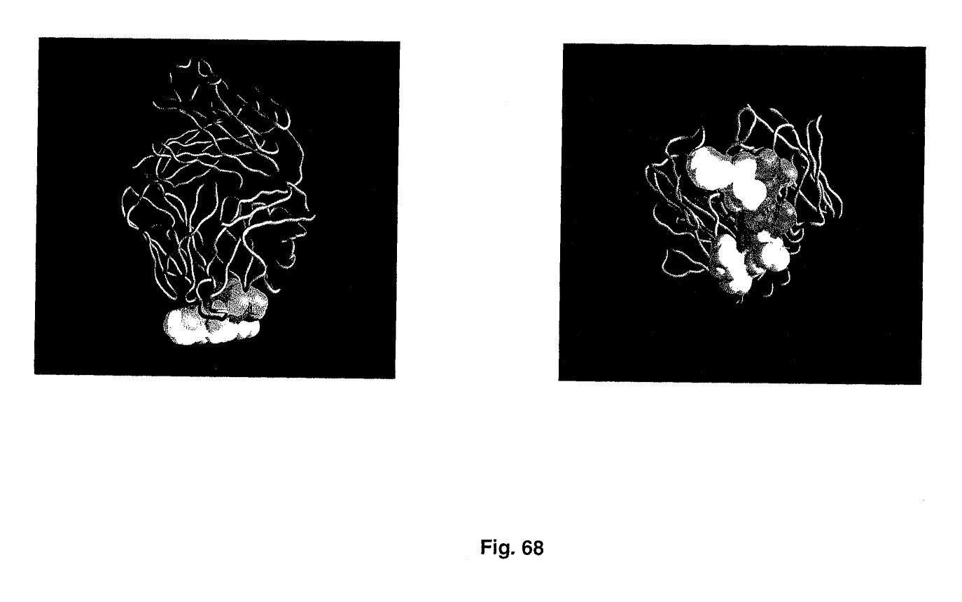

Referring to FIG. 68, the 2F5 fab in ribbon diagram is shown binding to a 14-residue peptide corresponding to its gp41 epitope (solvent accessible surface shown). Solvent accessible surface refers to a surface of a molecule that is freely accessible to a solvating water molecule. The 2F5-bound face is colored light gray, and the unbound face is colored white. Unbound face refers to a surface of a molecule that does not have a decrease in solvent accessible surface when bound by another molecule. The non-bound surface of the five contiguous residues in maximal contact with the antibody are colored dark gray. Contact residues refers to portion of a polypeptide that is occluded when in complex with another molecule; it can also be defined as the portion of a polypeptide within van der Waals radius of the surface of another molecule. Maximal contact refers to the amino acids within a polypeptide with the greatest absolute loss of solvent accessible surface when bound by another molecule. The dark gray surface area is 240.7 square angstroms. The left and right images show a 70 degree rotation around a horizontal axis.

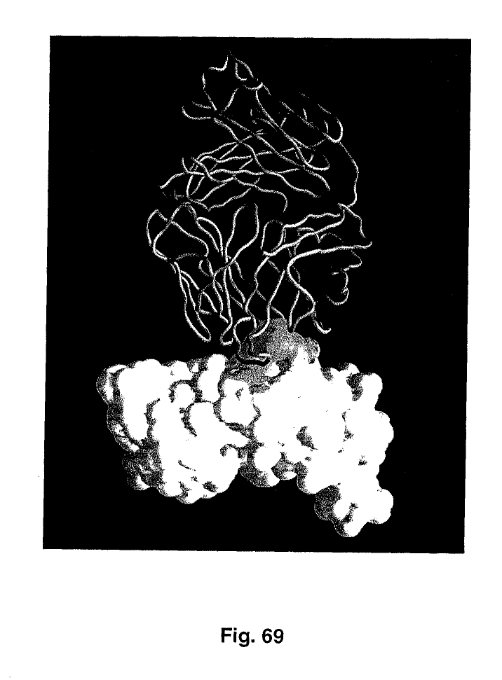

Referring to FIG. 69, a similar picture is shown for 2F5 binding to the gp41e-1KU2 scaffold. As before, the dark gray surface corresponds to the amount of the unbound face contributed by the five contiguous residues in maximal contact with the antibody. Here these residues only have 61.3 square angstroms of solvent accessible surface.

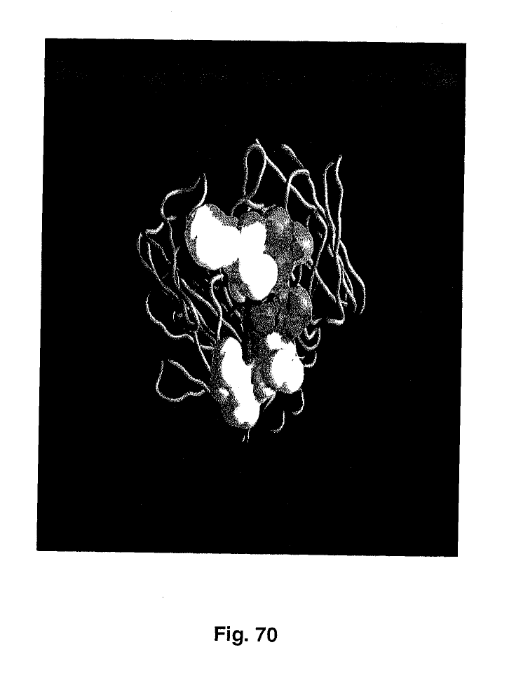

Referring to FIG. 70, a similar picture is shown for 2F5 binding to a cyclized peptide constrained to be in the 2F5-bound conformation through a link between residues 663 and 667 (as modeled from compound number 6 in McGaughey et al. 2003 Biochem. 42, 3214-3223.) The linkage occludes only a small amount of surface. Occluded refers to a portion of a molecule with reduced solvent accessible surface. Occluded by “x” refers to a portion of a molecule, which in the presence of “x”, has reduced solvent accessible surface. As before, the dark gray surface corresponds to the amount of the unbound face contributed by the five contiguous residues in maximal contact with the antibody. Here these residues contribute 206.8 square angstroms of solvent accessible surface.

### Example 1

In the following example, the utility of the computational protocols presently disclosed for structure based design of immunogens that present specific epitopes within different protein scaffolds is demonstrated. The example illustrate the relative ease with which many thousands of potential protein scaffolds may be narrowed to a small number of candidates for subsequent evaluation. The binding affinity of the identified candidates is further probed. This examples is discussed for illustrative purposes and should not be construed to limit the embodiments of the invention.

In the following example, a very early, simple version of the superposition computational protocol is applied to determine candidate protein scaffolds, selected from the entire protein data bank, which have regions of structural homology to 2F5 bound gp41. In an initial operation, the crystal structures of 2F5, 2F5 bound gp41, and protein crystal structures are obtained from either the protein data bank or other crystal structure databases.

Possible locations for superposition of the epitope on the proteins within the data bank were determined using the MAMMOTH structural matching program, and later with the ‘pepslide’ function within Rosetta. Multiple sub-ranges of the 2F5 epitope were used to search the PDB using MAMMOTH. Or, using pepslide, multiple sub-ranges of the 2F5 epitope were slid over substantially all of the polymer backbones contained within the PDB to find superposition locations within the backbone of any of the proteins. A threshold on the superposition RMSD divided by the number of superimposed residues was used to ‘normalize’ matches of different lengths. The threshold used was 0.14. From this approach, many thousands of candidate superposition sites on candidate scaffold proteins for the 2F5 epitope were determined.

Subsequently, the clash between the antibody and the backbones of the candidate scaffold proteins when docked according to superposition was assessed. The non-epitope residues within the protein were mutated to glycine, alanine, or combinations thereof, while the native residues within the antibody were retained. The interface clash was evaluated as the total repulsive in complex of antibody/scaffold minus the total repulsive in antibody minus the total repulsive in scaffold.

The superposition matches were ranked according to their interface clash, and then the best several hundred candidates in this list were examined and filtered. A clash threshold of approximately 200 arbitrary units according to the ROSETTA full atom repulsive score was used to select the initial round of candidate scaffolds. For comparison, the native structure of the 2F5 antibody/2F5 peptide complex has a total interface clash of approximately 5 units with all-atoms present, and only 2 units when the peptide is all-glycine.

Those scaffolds with acceptable interface clash were subsequently filtered for other considerations. The list included candidates whose native oligomerization state is non-monomeric, and such candidates were excluded if other members of the oligomer would clash with the 2F5 antibody. The list also included proteins that bind co-factors or ligands, and these were generally excluded also. Finally, the list contained ‘redundant’ matches to homologs of candidate scaffolds, and multiple matches of different sub-ranges to candidate scaffolds. From such ‘redundant’ candidates, the one with the longest superposition was generally chosen.

Several protein scaffolds determined from the above discussed protocol were subsequently selected for further evaluation. These scaffolds were: 1LGY, 2MAT, 1KU2, 1IWL, 1M53, 1NUB, and 1D3B.

TABLE 1

Seven Initial Scaffolds.

| | | | | Size | Sequence of |
|---|---|---|---|---|---|
| Scaffold | Origin | Description | Localization | (aa) | Graft |
| 1. 1LGY | Rhizopus | Lipase II (lipid | Intracellular | 265 | -EVLEADKWAILG |
| | niveus | metabolisim) | | | |
| 2. 2MAT | E. coli | Methionine | Intracellular | 262 | -EILELDKWAILG |
| | | Aminopeptidase | | | |
| 3. 1KU2 | Thermus | RNA Pol | Intracellular | 240 | -EVLELDKWAELG |
| | aquaticus | sigma23 | | | |
| | | subunit | | | |
| 4. 1IWL | E. coli | Periplasmic | Outer- | 177 | QENLEVDKWAFLF |
| | | lipoprotein- | membrane | | |
| | | binding | | | |
| 5. 1M53 | Klebsiella | Isomaltulose | Intracellular | 563 | QEFLELDKWAQLA |
| | SP.X3 | synthase | | | |
| 6. 1NUB | H. sapiens | Collagen- | Extracellular | 277 | -EILECDKWALLG |
| | | binding protein | | | |
| 7. 1D3B | H. sapiens | RNA-binding | Intracellular | 132 | -ELLELDKWALLS |
| | | protein | | | |
| gp41 MPR | | | | | QELLELDKWASLW |

TABLE 2
Summary of scaffolds and their binding
affinities to the 2F5 antibody.

| Expression | Expression | | Binds to |
|---|---|---|---|
| Vector | system | Refolded? | 2F5? |
| 2F5e_scaffold_1 | Mammalian | Yes | Yes |
| 2F5e_scaffold_2 | Bacterial | Yes | Yes |
| 2F5e_scaffold_3 | Bacterial | Yes | Yes |
| 2F5e_scaffold_4 | Bacterial | Yes | Yes |
| 2F5e_scaffold_5 | Mammalian | Yes | Yes |
| 2F5e_scaffold_6 | Mammalian | Yes | No |
| 2F5e_scaffold_7 | Bacterial | No | — |

TABLE 3
2F5 Binds the Scaffolds with nM Affinity

| | Analyte: 2F5 | | |
|---|---|---|---|
| Scaffold | Ka (1/Ms) 10⁵ | Kd (1/s) 10⁻³ | K_{D} (M) 10⁻⁹ |
| gp41e-1LGY | 3.69 | 3.96 | 10.7 |
| gp41e-2MAT | 0.726 | 0.679 | 9.35 |
| gp41e-1KU2 | 10.9 | 2.87 | 2.63 |
| gp41e-1IWL | 7.43 | 13.9 | 18.8 |
| gp41e-1D3B | 6.85 | 3.74 | 5.45 |
| MPER WT peptide | 5.52 | 3.56 | 6.45 |
| MPER Cyclized peptide | 5.28 | 2.69 | 5.09 |

TABLE 4
Study of the immunogenicity of 2F5 epitope scaffolds

| | # of | | | | |
|---|---|---|---|---|---|
| Group | Guinea Pigs | Immunogen | Adjuvant | | Dose |
| 1 | 4 | 2F5e_2mat | GSK AS01B | | 20 μg |
| 2 | 12 | 2F5e_1lgy | GSK AS01B | | 20 μg/ |
| | | | | | 4 μg |
| 3 | 8 | 2F5e_1d3b | GSK | Alum CpG | 20 μg |
| 4 | 8 | 2F5e_1d3bb_TH | GSK | Alum CpG | 20 μg |
| 5 | 4 | 2F5e_1lwl | Alum + CpG | | 20 μg |
| 6 | 8 | 2F5e_1ku2 | GSK | Alum CpG | 20 μg |
| 7 | 8 | 2F5e_1ku2_TH | GSK | Alum CpG | 20 μg |

TABLE 5
Purified Antibody Yields

| Fractions | OD 450 nm | Estimated ng/ml | ng (x0.5x11) |
|---|---|---|---|
| 11-15 | 0.75 | 7 | 38.5 |
| 16-20 | 1.1 | 20 | 110 |
| 21-25 | 0.6 | 4.8 | 26.4 |
| Total | | | 174.9 |

TABLE 6
Purified Antibody Yields

| Fractions | OD 450 nm | Estimated ng/ml | ng (x0.5x11) |
|---|---|---|---|
| 11-15 | 1.374 | ~55 | 302.5 |
| 16-20 | 1.351 | ~55 | 302.5 |
| Total | | | 605 |

TABLE 7
Neutralization Data

| | | | HxB2.DG.SG3 |
|---|---|---|---|
| Immunogen | Adjuvant | Animal ID | IC50 |
| 2F5e_2mat | GSK AS01B | C-100 | <5 |
| | | C-794 | <5 |
| 2F5e_1lgy | GSK AS01B | E-092 | <5 |
| | | E-325 | 142 |
| | | E-827 | 67 |
| 2F5e_1d3bb | ALUM + CpG | F-071 | 7 |
| | | F-893 | 21 |
| | | F-627 | <5 |
| | | F-556 | <5 |
| | GSK AS01B | G-853 | <5 |
| | | G-270 | 7 |
| | | G-007 | <5 |
| | | G-581 | <5 |
| 2F5e_1d3bb_TH | ALUM + CpG | H-563 | <5 |
| | | H-622 | 7 |
| | | H-268 | 34 |
| | | H-099 | <5 |
| | GSK AS01B | I-048 | <5 |
| | | I-003 | 194 |
| | | I-843 | <5 |
| | | I-637 | <5 |

| | |
|---|---|
| Positive Control 2F5 mAb | 0.01 ug/mL |

TABLE 8
Neutralization by the serum of animal I-003

| Dilution | % Neutralization |
|---|---|
| 5 | 90.6 |
| 25 | 76 |
| 125 | 46.7 |
| 625 | 31.2 |
| 3125 | 28.8 |
| 15625 | 14.5 |
| 78125 | 10.5 |
| 390625 | 2 |

TABLE 9
Neutralization by the serum of animal E-325

| Dilution | % Neutralization |
|---|---|
| 5 | 67.5 |
| 25 | 72.2 |
| 125 | 48.8 |
| 625 | 34.8 |
| 3125 | 29.7 |
| 15625 | 18.4 |
| 78125 | 15.2 |
| 390625 | 13.3 |

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A computer implemented method of designing an epitope-protein scaffold to elicit selected neutralizing antibodies to a pathogen, comprising:
obtaining computer searchable three-dimensional structures of a protein scaffold, a pathogen epitope recognized by an antibody, wherein the epitope is heterologous to the protein scaffold, and a complex of the epitope and the antibody;
superimposing backbone atoms of the epitope on a surface of backbone atoms of the protein scaffold;
calculating a deviation between the superimposed backbone atoms of the protein scaffold and the epitope;
designating the protein scaffold as a candidate scaffold if the deviation between the backbone atoms of the epitope and protein scaffold is less than a first selected threshold;
constructing a three-dimensional representation of an antibody-scaffold complex comprising the antibody and the candidate scaffold, wherein the antibody-candidate scaffold rigid-body orientations are set by the three-dimensional structure of the complex of the epitope and the antibody and the superposition;
removing from consideration candidate scaffolds that exhibit a steric repulsion across the antibody-candidate scaffold interface which is greater than a second selected threshold that interferes with binding of the antibody to the epitope; and
selecting a remaining candidate scaffold and incorporating it into a test epitope-protein scaffold, thereby designing an epitope-protein scaffold.

2. (canceled)

3. The method of claim 1, wherein the steric repulsion is calculated on the antibody-scaffold complex having all side chains of the antibody in their native conformation and all non-epitope candidate scaffold residues mutated to glycine, alanine, or combinations thereof.

4. The method of claim 1, wherein the three-dimensional representation of the antibody-scaffold complex is constructed with the epitope side chains in their native positions on the epitope portion of the candidate scaffold, and the epitope and the antibody are spatially varied to determine a conformation which is a local minimum in the total energy of the of the complex of the epitope and the antibody.

5. The method of claim 4 wherein the spatial variation comprises at least one of:
a repacking operation in which the conformation of the side chains of the epitope and the antibody are varied between discrete rotamers at the interface between the epitope and the antibody.
a minimization operation in which the chi angles of the side chains are allowed to vary continuously about their initial values followed by the repacking operation; and
a rigid body rotation of the candidate scaffold and the antibody with respect to one another.

6. The method of claim 4, further comprising measuring a binding energy of the candidate scaffold and the antibody when the complex of the epitope and the antibody is in the local minimum conformation and wherein candidate scaffolds having a binding energy less than a third selected threshold are discarded.

7. The method of claim 1, further comprising selecting amino acid residues for a first group of non-epitope scaffold positions which contact the antibody and a second group of non-epitope scaffold positions which contact the epitope but do not contact the antibody, wherein the first group residues are selected from small, polar amino acids and wherein the second group residues are selected from A, G, S, and T amino acids.

8. The method of claim 1, further comprising computing the binding energy of the antibody to the candidate scaffold and ranking the candidate scaffolds on the basis of binding energy.

9. The method of claim 1, further comprising:
removing a first portion of the candidate scaffold and attaching a first end of the selected epitope to the candidate scaffold at a first edge of the first removed portion;
adjusting the protein backbone so as to allow bonding between the backbone atoms of a second end of the epitope and a second edge of the removed portion of the candidate scaffold so as to form a grafted epitope protein scaffold;
mutating the candidate scaffold so as to maintain the grafted epitope in its antibody-bound conformation.

10. The method of claim 9, further comprising calculating a distance between the second end of the epitope and the second end of the first removed portion of the candidate scaffold and candidate scaffolds in which the distance is greater than a selected threshold are eliminated from consideration.

11. The method of claim 10, wherein the distance is calculated by the difference between the root mean square (RMS) of the position of a selected number of backbone atoms at the second end of the epitope and the RMS of the position of a selected number of backbone atoms at the second edge of the removed portion of the candidate scaffold.

12. The method of claim 9, wherein adjusting the protein backbone comprises adjusting at least one of bond distance, bond length, backbone dihedral angles, and bond angles of the backbone atoms.

13. The method of claim 9, further comprising eliminating from consideration those grafted protein-epitope scaffolds which are calculated to exhibit a steric repulsion greater than a third selected threshold between at least one of the grafted epitope and the protein scaffold and the protein scaffold and the antibody.

14. The method of claim 1, wherein superimposing comprises
identifying a structural match between the epitope and the protein scaffold;
positioning the epitope within the protein scaffold to generate an epitope-protein scaffold.

15.-16. (canceled)

17. The method of claim 14, wherein positioning the epitope comprises:
removing a first portion of the protein scaffold and attaching a first end of the epitope to the protein scaffold at an edge of the first removed portion;
adjusting the protein backbone so as to allow bonding between the backbone atoms of a second end of the epitope and a second edge of the removed portion of the protein scaffold so as to form a grafted-epitope protein scaffold;
mutating the protein scaffold so as to substantially inhibit changes in the grafted epitope conformation from the native, un-grafted conformation.

18. The method of claim 17 wherein adjusting the protein backbone comprises adjusting the bond distance, bond length, backbone dihedral angles, and bond angles of the backbone atoms.

19.-21. (canceled)

22. An immunogenic composition comprising a chimeric polypeptide comprised of an amino acid sequence comprising a non-HIV polypeptide, which is not from HIV-1, HIV-2 or SIV, or a functional variant thereof, wherein the amino acid sequence further comprises a heterologous epitope recognized by an HIV-1 neutralizing antibody, wherein the heterologous epitope in the absence of the antibody can be structurally superimposed onto the native epitope in the absence of or in complex with the antibody with a root mean square (rms) deviation of their coordinates of less than 0.25 Å/residue, wherein the rms deviation is measured over the polypeptide backbone atoms N, CA, C, O, for at least three consecutive amino acids.

23. (canceled)

24. The immunogenic composition of claim 22, wherein the heterologous epitope in complex with the HIV-1 neutralizing antibody shows an unbound face for the five consecutive residues in maximal contact with the antibody, wherein at least 50% of the unbound face is occluded by non-epitope residues of the chimeric polypeptide.

25. The immunogenic composition of claim 22, wherein the heterologous epitope is from gp41 or gp120.

26. The immunogenic composition of claim 22, wherein the heterologous epitope comprises a 2F5 epitope, a 2G12 epitope, a b12 epitope, a 4E10 epitope, or a Z13 epitope.

27. The immunogenic composition of claim 22, wherein the heterologous epitope is from any HIV strain or isolate.

28. The immunogenic composition of claim 22, wherein the composition further comprises a pharmaceutically acceptable carrier, diluent, or adjuvant.

29. An immunogenic composition comprising a polynucleotide encoding the chimeric polypeptide of claim 22.

30. The immunogenic composition of claim 29 comprised as a canarypox virus, a vaccinia virus, the alphavirus VEE, a replication-defective adenovirus or adenovirus, or a naked DNA.

31. A method of eliciting an immune response in a subject comprising administering to the subject an effective concentration of the immunogenic composition of claim 22, thereby eliciting the immune response in the subject.

32. A method of boosting an immune response in a subject comprising administering to the subject an effective concentration of the immunogenic composition of claim 22 following prior administration of a priming composition comprising the heterologous epitope or polynucleotide encoding the heterologous epitope, and thereby boosting the immune response in the subject.

33. A method for detecting an HIV-1 binding antibody in a subject infected with HIV-1 comprising:
a. providing the immunogenic composition of claim 22;
b. contacting the immunogenic composition with an amount of bodily fluid from the subject; and
c. detecting the HIV-1 binding antibody in the bodily fluid from the subject.

34. The method of claim 33 wherein detecting the HIV-1 binding antibody comprises a competition binding assay.

35. A method to identify an HIV-1 binding antibody comprising:
a. providing the immunogenic composition of claim 22;
b. contacting the immunogenic composition with a composition comprising a candidate HIV-1 binding antibody; and
c. determining if said candidate antibody is an HIV-1 binding antibody.

36. The immunogenic composition of claim 22 comprising a member selected from the group consisting of 1LGY (SEQ ID NO: 5), 2MAT (SEQ ID NO: 6), 1KU2 (SEQ ID NO: 4), 1IWL (SEQ ID NO: 2), 1M53 (SEQ ID NO: 7), 1NUB (SEQ ID NO: 8), 1D3B (SEQ ID NO: 3), (b12)-Sca1 (SEQ ID NO: 41), (b12)-Sca2 (SEQ ID NO: 43), (b12)-Sca2′ (SEQ ID NO: 45), b12-Sca11 (SEQ ID NO: 54), b12-Sca12 (SEQ ID NO: 56), b12-Sca13 SE ID NO: 58), b12-Sca14 (SEQ ID NO: 60), b12-Sca15 (SEQ ID NO: 62), b12-Sca16 (SEQ ID NO: 64), b12-Sca11 (SEQ ID NO: 66), b12-Sca18 (SEQ ID NO: 68), b12-Sca19 (SEQ ID NO: 70), b12-Sca21 (SEQ ID NO: 74), b12-Sca22 (SEQ ID NO: 76), OD252/482 delet-beta20-21 (V3/9C)-G4 (SEQ ID NO: 47), OD252/482 delet-beta20-21 (V3/9C)-G5 (SEQ ID NO: 49), OD252/482 delet-beta20-21 (V3/9C)-G8F-His (SEQ ID NO: 51), and OD252/482 delet-beta20-21 (V3/9C)-G11F-His (SEQ ID NO: 53).

37. The immunogenic composition of claim 22 comprising a member selected from the group consisting of sme543_HIV_MPER (SEQ ID NO: 83), SIVagm_tan1_HIV_MPER (SEQ ID NO: 84), SIVlho7_HIV_MPER (SEQ ID NO: 85), SIVdeb_CM40_HIV_MPER (SEQ ID NO: 86), and SIVCOLCGU1_HIV_MPER (SEQ ID NO: 87).

38. The immunogenic composition of claim 22 comprising a member selected from the group consisting of SIV_CLOAKED_{—}8b (SEQ ID NO: 88), SIV_CLOAKED_wt (SEQ ID NO: 89), SIV_CLOAKED_nc (SEQ ID NO: 90), SIV_CLOAKED_{—}8B (SEQ ID NO: 91), HIV2_cloaked_wt (SEQ ID NO: 92), HIV2_cloaked_NC (SEQ ID NO: 93), SIV_CLOAKED_{—}8B_SILENT_glycan (SEQ ID NO: 94), SIV_CLOAKED_{—}8B_od (SEQ ID NO: 95), and New_SIVmac239_cloaked_core (SEQ ID NO: 96).

39. The immunogenic composition of claim 22 comprising a member selected from the group consisting of Group N Env-04CM_{—}1015_{—}04_DQ017382 (SEQ ID NO: 98), Group O Env-AF383260 (SEQ ID NO: 99), HIV-2 Env HIV-2BEN MK6 (SEQ ID NO: 100), SIV mac 239 Env (SEQ ID NO: 101), BIV-Env—Accession # AAA42762 (SEQ ID NO: 102), FIV-Env CABCpady00C or clone FIV-C36 (SEQ ID NO: 103), and M group-Consensus (SEQ ID NO: 104).

40. (canceled)

41. The method of claim 1, wherein obtaining the three-dimensional structure comprises obtaining atomic coordinates.

42. The method of claim 1, wherein constructing the antibody-scaffold complex comprises constructing a three dimensional model of the atomic coordinates of the complex of the antibody and candidate scaffold.

43. The method of claim 1, further comprising expressing the test protein and determining whether it elicits neutralizing antibodies.

44. The method of claim 1, wherein obtaining the three-dimensional structures comprises obtaining the three-dimensional structures from a computer searchable database.

45. The method of claim 1, wherein the superimposing the backbone atoms comprises superimposing the backbone atoms using a sliding window.

46. The method of claim 1, wherein the epitope is the 2F5 epitope.

47. The method of claim 1, wherein the epitope is the b12 epitope.

48. The method of claim 1, wherein the calculated deviation is a root mean square (RMS) deviation.

49. The method of claim 1, further comprising testing the test epitope-protein scaffold to determine if it elicits neutralizing antibodies to the pathogen.
